SEQUENCE LISTING (26 sequences)

SEQ ID NO: 1 - DNA, 23 bp, Artificial Sequence
  source: /note=“Description of Artificial Sequence Synthetic primer”
tagtccttcctaccccaatttcc

SEQ ID NO: 2 - DNA, 21 bp, Artificial Sequence
  source: /note=“Description of Artificial Sequence Synthetic primer”
ttggtccttagccactccttc

SEQ ID NO: 3 - DNA, 22 bp, Artificial Sequence
  source: /note=“Description of Artificial Sequence Synthetic primer”
gcaactgttcctgaactcaact

SEQ ID NO: 4 - DNA, 21 bp, Artificial Sequence
  source: /note=“Description of Artificial Sequence Synthetic primer”
atcttttggggtccgtcaact

SEQ ID NO: 5 - DNA, 22 bp, Artificial Sequence
  source: /note=“Description of Artificial Sequence Synthetic primer”
ccgaagtcatagccacactcaa

SEQ ID NO: 6 - DNA, 25 bp, Artificial Sequence
  source: /note=“Description of Artificial Sequence Synthetic primer”
gcagtctgtcttctttctccgttac

SEQ ID NO: 7 - DNA, 22 bp, Artificial Sequence
  source: /note=“Description of Artificial Sequence Synthetic primer”
tccgagcagagatcttcaggaa

SEQ ID NO: 8 - DNA, 22 bp, Artificial Sequence
  source: /note=“Description of Artificial Sequence Synthetic primer”
tgcaaccaccactcattctgag

SEQ ID NO: 9 - DNA, 20 bp, Artificial Sequence
  source: /note=“Description of Artificial Sequence Synthetic primer”
gctgctttgcctacctctcc

SEQ ID NO: 10 - DNA, 21 bp, Artificial Sequence
  source: /note=“Description of Artificial Sequence Synthetic primer”
tcgagtgacaaacacgactgc

SEQ ID NO: 11 - DNA, 21 bp, Artificial Sequence
  source: /note=“Description of Artificial Sequence Synthetic primer”
ccaagtgctgccgtcattttc

SEQ ID NO: 12 - DNA, 21 bp, Artificial Sequence
  source: /note=“Description of Artificial Sequence Synthetic primer”
ggctcgcagggatgatttcaa

SEQ ID NO: 13 - DNA, 24 bp, Artificial Sequence
  source: /note=“Description of Artificial Sequence Synthetic primer”
cggttccgatgccctgaggctctt

SEQ ID NO: 14 - DNA, 24 bp, Artificial Sequence
  source: /note=“Description of Artificial Sequence Synthetic primer”
cgtcacacttcatgatggaattga

SEQ ID NO: 15 - DNA, 23 bp, Artificial Sequence
  source: /note=“Description of Artificial Sequence Synthetic primer”
actcacctcttcagaacgaattg

SEQ ID NO: 16 - DNA, 23 bp, Artificial Sequence
  source: /note=“Description of Artificial Sequence Synthetic primer”
ccatctttggaaggttcaggttg

SEQ ID NO: 17 - DNA, 23 bp, Artificial Sequence
  source: /note=“Description of Artificial Sequence Synthetic primer”
atgatggcttattacagtggcaa

SEQ ID NO: 18 - DNA, 20 bp, Artificial Sequence
  source: /note=“Description of Artificial Sequence Synthetic primer”
gtcggagattcgtagctgga

SEQ ID NO: 19 - DNA, 19 bp, Artificial Sequence
  source: /note=“Description of Artificial Sequence Synthetic primer”
aagtgtgaacgtgaagtcc

SEQ ID NO: 20 - DNA, 20 bp, Artificial Sequence
  source: /note=“Description of Artificial Sequence Synthetic primer”
ggatttgtcactgttcagca

SEQ ID NO: 21 - DNA, 20 bp, Artificial Sequence
  source: /note=“Description of Artificial Sequence Synthetic primer”
ctctgctcctcctgttcgac

SEQ ID NO: 22 - DNA, 20 bp, Artificial Sequence
  source: /note=“Description of Artificial Sequence Synthetic primer”
ttaaaagcagccctggtgac

SEQ ID NO: 23 - DNA, 20 bp, Artificial Sequence
  source: /note=“Description of Artificial Sequence Synthetic primer”
ccagcagtcgtctttgtcac

SEQ ID NO: 24 - DNA, 20 bp, Artificial Sequence
  source: /note=“Description of Artificial Sequence Synthetic primer”
ctctgggttggcacacactt

SEQ ID NO: 25 - DNA, 21 bp, Artificial Sequence
  source: /note=“Description of Artificial Sequence Synthetic primer”
gtggcattcaaggagtacctc

SEQ ID NO: 26 - DNA, 22 bp, Artificial Sequence
  source: /note=“Description of Artificial Sequence Synthetic primer”
tgatggccttcgattctggatt

(19) United States
(12) Patent Application Publication
(10) Pub. No.: US 2025/0009743 A1
(43) Pub. Date: Jan. 9, 2025

(54) METHODS OF TREATING CANCERS USING STING AGONISTS

(71) Applicant: Dana-Farber Cancer Institute, Inc., Boston, MA (US)

(72) Inventors: Jean Zhao, Brookline, MA (US); Qiwei Wang, Boston, MA (US); Liya Ding, Chestnut Hill, MA (US)

(21) Appl. No.: 18/274,823

(22) PCT Filed: Feb. 10, 2022
(86) PCT No.: PCT/US22/15951

§ 371 Date: Jul. 28, 2023

Related U.S. Application Data
(60) Provisional application No. 63/148,426, filed on Feb. 11, 2021.

(51) Int. Cl. A61K 31/502; A61K 31/352; A61K 31/381; A61K 31/5025; A61K 31/506; A61K 31/517; A61K 31/7068; A61K 45/06; A61P 35/00

(52) U.S. Cl. CPC A61K 31/502; A61K 31/352; A61K 31/381; A61K 31/5025; A61K 31/506; A61K 31/517; A61K 31/7068; A61K 45/06; A61P 35/00

(57) ABSTRACT

The present invention relates, in part, to methods for using STING agonists to polarize pro-tumor macrophages in a subject with cancer into anti-tumor macrophages, for example to improve effectiveness of PARP inhibition.

### CROSS-REFERENCE TO RELATED APPLICATIONS

This application claims the benefit of U.S. Provisional Application No. 63/148,426, filed Feb. 11, 2021, which is hereby incorporated by reference in its entirety.

### STATEMENT OF RIGHTS

This invention was made with government support under Grant Nos. P50 CA168504, P50 CA165962, and R35 CA210057 awarded by the National Institutes of Health and Grant No. W81XWH-20-1-0118 awarded by the Department of Defense. The government has certain rights in the invention.

### BACKGROUND OF THE INVENTION

Homologous recombination (HR) deficiency confers exquisite sensitivity to poly (ADP-ribose) polymerase (PARP) inhibitors (PARPi), which have been therapeutically exploited in both ovarian and breast tumors carrying loss-of-function mutations in HR pathway genes, most commonly BRCA1 and BRCA2 (BRCA1/2). Based on a substantial progression-free survival (PFS) benefit, three PARPi have gained FDA approval for BRCA-mutated ovarian cancer in both adjuvant and metastatic settings. Most recently, maintenance treatment with olaparib was shown to confer unprecedented overall survival benefit for patients with BRCA-mutated relapsed ovarian cancer. Compared to ovarian cancer, however, PARPi therapy appears to be less effective in BRCA-mutated breast cancer. Nevertheless, the FDA has approved two PARPi—olaparib and talazoparib—as monotherapy for patients with germline BRCA1/2-mutated and HER2-negative advanced breast cancer.

Although these PARPi significantly improved PFS, recent results from the OlympiAD and EMBRACA clinical trials suggest no overall survival benefit for both olaparib and talazoparib in patients with advanced breast cancer carrying germline BRCA1/2 mutations, highlighting the need to understand why BRCA-mutated breast cancers are more refractory to PARPi in the effort to develop strategies to improve responses to PARPi.

Osimertinib (AZD9291) is a third-generation EGFR tyrosine kinase inhibitor (TKI) for patients with non-small cell lung cancer (NSCLC) with EGFR-activating mutations or the acquired T790M mutation resistant to earlier generation EGFR-TKIs. Despite its striking efficacy, the emergence of resistance to osimertinib is inevitable, and overcoming such resistance remains a key challenge in the clinic, novel treatments for overcoming such resistance are therefore needed.

### SUMMARY OF THE INVENTION

The present invention is based, at least in part, on the discovery that STING agonists reprogram M2-like pro-tumor macrophages into M1-like anti-tumor macrophages in a macrophage's STING-dependent manner. This discovery can be exploited in various ways, for example to treat certain cancers that are enriched in M2-like macrophages, or to improve the effectiveness of PARP inhibition, TK inhibitors, and/or inhibitors of DNA synthesis in some cancers. The discovery can be used to overcome or prevent drug resistance in any cancer in which resistance is characterized by an increase in the level or amount of M2-like pro-tumor macrophages in the tumor or tumor microenvironment. In some embodiments, drug resistance may be characterized by recruitment of M2-like pro-tumor macrophages to the cancer or tumor. The discovery can also be used to overcome drug resistance characterized by activation of STAT3 signaling as a result of drug administration. Finally, this discovery can also be used to overcome drug resistance characterized by secretion of hepatocyte growth factor (HGF) in a feed forward manner in the cancer. Additional details regarding HGF and its role in drug resistance can be found in Dong N, et al. M2 macrophages mediate sorafenib resistance by secreting HGF in a feed-forward manner in hepatocellular carcinoma. Br J Cancer. 2019 July; 121 (1): 22-33, hereby incorporated by reference in its entirety.

In some aspects, methods of improving effectiveness of PARP inhibition in a subject with cancer include administering to the subject an effective amount of a STING agonist conjointly with an effective amount of a PARP inhibitor.

A syngeneic genetically-engineered mouse (GEM) model of lung cancer driven by a mutant EGFR shows that while EGFR-mutant tumors are highly sensitive to osimertinib at early stage of tumor growth in a T cell-dependent manner, they become resistant as they progress. Therefore, the present invention, is also based, in part, on the determination that the presence of immunosuppressive tumor-associated macrophages (TAMs) renders tumors resistant to osimertinib. Depletion of TAMs in these tumors rescues the efficacy of osimertinib. Reprograming TAMs with a newly developed STING agonist MSA-2 reinvigorates antitumor immunity, and leads to durable regression of resistant tumors in mice when combined with osimertinib. The results shown herein indicate that a suppressive tumor immune microenvironment can drive resistance of EGFR-mutant tumors to osimertinib. Therefore, provided herein is a new strategy to overcome resistance and improve therapeutic outcomes.

Also provided herein is a method of improving effectiveness of tyrosine kinase inhibitor (TKI) inhibition in a subject with cancer, by administering to the subject an effective amount of a STING agonist conjointly with an effective amount of a tyrosine kinase inhibitor (TKI).

In certain aspects, methods of polarizing pro-tumor macrophages in a subject with cancer into anti-tumor macrophages include administering to the subject an effective amount of a STING agonist. Also provided herein are methods of preventing or reversing drug resistance in a subject with cancer, wherein the drug resistance is a result of polarizing anti-tumor macrophages into pro-tumor macrophages, comprising administering to the subject an effective amount of a STING agonist. The drug resistance may be resistance to PARP inhibition. The drug resistance may be resistance to TK inhibition. The drug resistance may be resistance to DNA synthesis inhibitors.

Also provided herein is a method of improving effectiveness of a DNA synthesis inhibitor in a subject with cancer, by administering to the subject an effective amount of a STING agonist conjointly with an effective amount of a DNA synthesis inhibitor. Exemplary DNA synthesis inhibitors include, but are not limited to, a nucleoside analog, such as gemcitabine, sapacitabine, a cytidine analog, cytarabine, tezacitabine, troxacitabine, DMDC, CNDAC, ECyD, clofarabine, or decitabine. Additional information relating to resistance in gemcitabine can be found in Bulle A, et al. Gemcitabine Recruits M2-Type Tumor-Associated Macrophages into the Stroma of Pancreatic Cancer. Transl Oncol. 2020 March; 13 (3): 100743, hereby incorporated by reference in its entirety.

Numerous embodiments are further provided that can be applied to any aspect of the present invention and/or combined with any other embodiment described herein. For example, in some embodiments, the STING agonist activates STING signaling in macrophages. In some embodiments, the STING agonist (e.g., a tumor cell's cytosolic dsDNAs/cGAMP or intra-tumorally-delivered STING agonists) does not activate STING signaling in intra-tumoral dendritic cells. In some embodiments, the subject has a deficient STING signaling pathway in tumor cells. In some embodiments, the pro-tumor macrophages are M2-like. In some embodiments, the anti-tumor macrophages are M1-like.

Similarly, in some embodiments, administering comprises a systemic delivery of the STING agonist. It is known that early generation STING agonists are not suitable for systemic delivery. In some embodiments, administering is oral, intravenous, or intraperitoneal. In some embodiments, the STING agonist is a modified nucleotide STING agonist. In some embodiments, the STING agonist is selected from DMXAA, MSA-2, SR-717, FAA, CMA, α-Mangostin, BNBC, DSDP, diABZI, bicyclic benzamides, and benzothiophenes. In some embodiments, the PARP inhibitor is selected from olaparib, rucaparib, niraparib, talazoparib, veliparib, pamiparib, CEP 9722, E7016, AG014699, MK4827, BMN-673, iniparib, and 3-aminobenzamide. In some embodiments, the STING agonist and the PARP inhibitor are administered conjointly. In some embodiments, administering conjointly comprises administering the STING agonist before the PARP inhibitor. In some embodiments, administering conjointly comprises administering the STING agonist concurrently with the PARP inhibitor. In some embodiments, the STING agonist and the TK inhibitor are administered conjointly. In some embodiments, administering conjointly comprises administering the STING agonist before the TK inhibitor (e.g., EGFR-TK inhibitor any other TK inhibitor disclosed herein). In some embodiments, administering conjointly comprises administering the STING agonist concurrently with the TK inhibitor (e.g., EGFR-TK inhibitor). The TK inhibitor (e.g., EGFR-TK inhibitor) may be selected from afatinib, dacomitinib, osimertinib (AZD9291), rociletinib (CO-1686), olmutinib (HM61713), nazartinib (EGF816), naquotinib (ASP8273), mavelertinib (PF-0647775), almonertinib, TY-9591, gefitinib, erlotinib and AC0010.

The TK inhibitor, as disclosed herein, may be a vascular endothelial growth factor receptor (VEGF) TK inhibitor; an epidermal growth factor (EGF) receptor TK inhibitor, a platelet derived endothelial growth factor receptor (PDGF) TK inhibitor, or the TK inhibitor may be a fibroblast growth factor (FGF) receptor TK inhibitor. The TK inhibitor may be, for example, axitinib, dasatinib, erlotinib, imatinib, nilotinib, pazopanib, sorafenib, bosutinib, avapritinib, capmatinib, pemigatinib, ripretinib, selpercatinib, selumetinib, tucatinib, entrectinib erdafitinib, fedratinib, pexidartinib, tenosynovial, upadacitinib, zanubrutinib, baricitinib, binimetinib, dacomitinib, fostamatinib, gilteritinib, larotrectinib, lorlatinib, acalabrutinib, brigatinib, midostaurin, neratinib, alectinib, cobimetinib, lenvatinib, osimertinib, ceritinib, nintedanib, afatinib, ibrutinib, trametinib, axitinib, bosutinib, cabozantinib, ponatinib, regorafenib, tofacitinib, crizotinib, ruxolitinib, vandetanib, pazopanib, lapatinib, gefitinibor, or sunitinib.

In some embodiments, the STING agonist and the DNA synthesis inhibitor are administered conjointly. In some embodiments, administering conjointly comprises administering the STING agonist before the DNA synthesis inhibitor. In some embodiments, administering conjointly comprises administering the STING agonist concurrently with the DNA synthesis inhibitor.

Similarly, in some embodiments, the cancer comprises a tumor with an M2 enrichment score higher than 0.27 (e.g., higher than 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, or greater, or any range in between, inclusive, such as 0.27 to 0.60). In some embodiments, the cancer comprises a tumor with an M2 enrichment score of higher than 0.15 (e.g., higher than 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97 0.98, 0.99, or higher). In some embodiments, the cancer comprises a tumor with an M2 enrichment score of between 0.15-0.25, between 0.20-0.30, between 0.25 and 0.3, between 0.3-0.40, between 0.35 and 0.45, between 0.40-0.50, between 0.45 and 0.55, between 0.50-0.60, between 0.55-0.65, between 0.60-0.70, between 0.65-0.75, between 0.70-0.80, between 0.75-0.85, between 0.80-0.90, or any range in between, inclusive, such as 0.15-0.90, 0.30-0.85, etc.

In some embodiments, the cancer comprises a tumor with the ratio of M2/M1 higher than 1.0, such as 1.1, 1.2, 1.3, 1.4, 1.5, 1.6, 1.7, 1.8, 1.9, 1.95, or more, inclusive, such as 1.1-1.3, 1-1.2, 1.2-1.4, 1.3-1.5, 1.4-1.6, 1.5-1.7, 1.6-1.8, 1.7-1.9, 1.8-2.0, 1.0-1.5, 1.5-1.0, 1.2-1.7, 1.4-1.9, 1.5-2.0, or any range in between, inclusive, such as 1.1-2.0, 1.3-1.8, etc. In some embodiments, M1 cells are characterized as CD45+CD11b+F4/80+MHC II highCD206 low, or CD45+CD11b+F4/80+MHC II highCD163 low and/or M2 cells are characterized as CD45+CD11b+F4/80+MHC II lowCD206 high, or CD45+CD11b+F4/80+MHC II lowCD163 high). In some embodiments, the cancer comprises head and neck squamous cell carcinoma (HNSC); a lung cancer, such as non-small cell lung carcinoma (NSCLC); lung squamous cell carcinoma (LUSC); liver cancer, such as hepatocellular carcinoma (HCC); colon cancer; prostate cancer; pancreatic cancer; skin cutaneous melanoma (SKCM); glioblastoma multiforme (GBM); breast invasive carcinoma (BRCA); lung adenocarcinoma (LUAD); kidney renal clear cell carcinoma (KIRC); cervical squamous cell carcinoma and endocervical adenocarcinoma (CESC); diffuse large B-cell lymphoma (DLBC); stomach adenocarcinoma (STAD), ovarian cancer, such as high-grade serous ovarian carcinoma (HGSOC) or homologous recombination proficient (HRP) ovarian cancer; or any homologous recombination proficient (HRP) cancer. The cancer may be any homologous recombination deficient (HRD) cancer, such as HRD ovarian cancer. The cancer may be an HRD cancer or tumor that comprises a mutation in a RAD51, PALB2, ATM, ATR, CHEK2, or FANC gene. The cancer may be an HRD cancer or tumor that is associated with aberrant expression of proteins encoded by a RAD51, PALB2, ATM, ATR, CHEK2, or FANC gene. The cancer may be any cancer that comprises a genetic mutation which upregulates STAT3 signaling and/or polarizes tumor associated macrophages to M2-like macrophages (e.g., a cancer with a mutation in the KRAS gene, such as the KRAS^{G12D} mutation). Further details regarding the KRAS^{G12D} and its association with STAT3 signaling can be found in Dai E et al. Autophagy-dependent ferroptosis drives tumor-associated macrophage polarization via release and uptake of oncogenic KRAS protein. Autophagy. 2020; 16 (11): 2069-2083, hereby incorporated by reference in its entirety. In some embodiments, the cancer comprises breast cancer carrying a BRCA mutation. In some embodiments, the cancer comprises advanced breast cancer carrying germline BRCA1/2 mutations.

A STING agonist also can enhance therapeutic efficacy of PARPi in naive BRCA1-tumors, in which the ratio of M2 and M1 is less than 1.0. In certain embodiments, the subject has BRCA naïve breast or ovarian cancer. In some embodiments, the cancer comprises breast cancer not carrying a BRCA mutation. In some embodiments, the cancer does not comprise an advanced breast cancer carrying germline BRCA1/2 mutations. In some embodiments, the cancer comprises a tumor with the ratio of M2/M1 less than 1.0, such as 0.9, 0.8, 0.7, 0.6, 0.5, 0.4, 0.3, 0.2, or 0.1 or more, inclusive, such as 0.1-0.3, 0.2-0.4, 0.3-0.5, 0.4-0.6, 0.5-0.7, 0.6-0.8, or 0.7-0.9, or any range in between, inclusive, such as 0.1-0.9, 0.3-0.8, etc.

In some embodiments, the cancer comprises a lung cancer carrying an EGFR mutation, such as an EGFR activating mutation or a T790M mutation. In some embodiments, the cancer comprises a lung cancer carrying an exon 19 deletion mutation. In some embodiments, the cancer comprises a lung cancer carrying a single-point substitution mutation L858R in exon 21. In some embodiments, the cancer comprises a non-small cell lung cancer carrying an EGFR mutation, such as an EGFR activating mutation or a T790M mutation.

In some embodiments, the cancer comprises a sub-population of tumors with an M2 enrichment score higher than 0.27 (e.g., 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, or greater, or any range in between, inclusive, such as 0.27 to 0.60). In some embodiments, the cancer comprises a tumor with an M2 enrichment score of higher than 0.15 (e.g., higher than 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97 0.98, 0.99, or higher). In some embodiments, the cancer comprises a tumor with an M2 enrichment score of between 0.15 and 0.25, between 0.20-0.30, between 0.25 and 0.3, between 0.3-0.40, between 0.35 and 0.45, between 0.40-0.50, between 0.45 and 0.55, between 0.50-0.60, between 0.55-0.65, between 0.60-0.70, between 0.65-0.75, between 0.70-0.80, between 0.75-0.85, between 0.80-0.90, or any range in between, inclusive, such as 0.15-0.90, 0.30-0.85, etc.

In some embodiments, the cancer comprises a tumor that has acquired an M2 enrichment score higher than 0.27. In some embodiments, the cancer comprises a tumor that has acquired an M2 enrichment score higher than 0.15 (e.g., higher than 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97 0.98, 0.99, or higher) or a ratio of M2/M1 higher than 1.0 (i.e., higher than 1.1, 1.2, 1.3, 1.4, 1.5, 1.6, 1.7, 1.8, 1.9, or 1.95) over a treatment course. In some embodiments, the subject is a rodent, primate, human, or animal model of cancer, optionally wherein the subject is human. In some embodiments, the subject has a deficiency in activating STING signaling in intra-tumoral dendritic cells.

Similarly, in some embodiments, the M2 enrichment score is determined by GSEA analysis of RNA sequencing data of tumors and tumor microenvironment (e.g., RNA sequencing analysis of bulk tumors, pleural effusion or ascites; single cell RNA sequencing analysis of tumor infiltrating immune cells or immune cells in any types of effusion in cancer patients). In some embodiments, the ratio of M2/M1 is detected by Cyclic Immunofluorescence (CyCIF) or conventional immunohistochemistry analysis of any types of tumor tissues (e.g., paraffin embedded tumors tissues, frozen tumor tissues). In some embodiments, the ratio of M2/M1 is detected by flow cytometry analysis or cytometry by time-of-flight (CyTOF) analysis of tumor-infiltrating immune cells or immune cells in any types of effusion in cancer patients. In some embodiments, M1 and M2 macrophages are determined using any types of phenotypic markers listed in the literature (e.g., macrophage markers: CD11b, F4/80, and CD68; M1 markers: CD80, CD86, MHC-II, TLR2, TLR4, iNOS, SOCS3, IFN-beta, TNF-alpha, CCL2, CCL3, CCL4, CCL5, CCL8, CCL9, CCL10, CCL11; M2 markers: CD163, CD206, CD200R, ARG-1, Ym1/2, Fizz1, IL-6, IL-10, TGF-beta, VEGF, CCL17, CCL22, CCL24, CCR2).

Similarly, in some embodiments, the PARP inhibitor is administered at a dosage of at least 10 mg/kg, at least 15 mg/kg, at least 20 mg/kg, at least 25 mg/kg, at least 30 mg/kg, at least 35 mg/kg, at least 40 mg/kg, at least 45 mg/kg, 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg body weight per dose. In some embodiments, the EGFR-TK inhibitor is administered at a dosage of at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg body weight per dose. Dosages may be administered twice a day, per day, twice a day, twice a week, per week, three times a month, twice a month, or monthly. In some embodiments, the STING agonist is administered at a dosage of at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg body weight per dose. Dosages may be administered twice a day, per day, twice a day, twice a week, per week, three times a month, twice a month, or monthly. In some embodiments, the STING agonist is administered 2-3 times. In some embodiments, the method comprises an additional therapy. In some embodiments, the additional therapy comprises radiation therapy. In some embodiments, the additional therapy comprises chemotherapy (e.g., including paclitaxel, a platinum-based drug (e.g., cisplatin, oxaliplatin), an inhibitor of topoisomerase activity like topoisomerase II (e.g., etoposide), a DNA intercalator (e.g., doxorubicin), and/or a DNA alkylating agent (e.g., temozolomide)). In some embodiments, the additional therapy comprises a DNA damage response (DDR)-targeting agent (e.g., including ATMi, ATRi, CHK1/2i, or Weeli).

In some aspects, methods of selecting a subject with cancer for treatment with a STING agonist include detecting an M2 enrichment score for a tumor from the subject, and selecting the subject if the score is higher than 0.27. In some embodiments, the cancer comprises a tumor that has acquired an M2 enrichment score higher than 0.15 (e.g., higher than 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97 0.98, 0.99, or higher). In some aspects, methods of selecting a subject with cancer for treatment with a STING agonist include detecting an ratio of M2/M1 for a tumor from the subject, and selecting the subject if the score is higher than 1.0 (i.e., higher than 1.1, 1.2, 1.3, 1.4, 1.5, 1.6, 1.7, 1.8, 1.9, or 1.95).

As described above, numerous embodiments are further provided that can be applied to any aspect of the present invention and/or combined with any other embodiment described herein. For example, in some embodiments, the cancer comprises head and neck squamous cell carcinoma (HNSC); a lung cancer, such as lung squamous cell carcinoma (LUSC) or such as a non-small cell lung cancer (NSCLC); liver cancer, such as hepatocellular carcinoma (HCC); colon cancer; prostate cancer; pancreatic cancer; skin cutaneous melanoma (SKCM); glioblastoma multiforme (GBM); breast invasive carcinoma (BRCA); lung adenocarcinoma (LUAD); kidney renal clear cell carcinoma (KIRC); cervical squamous cell carcinoma and endocervical adenocarcinoma (CESC); diffuse large B-cell lymphoma (DLBC); stomach adenocarcinoma (STAD); ovarian cancer, such as high-grade serous ovarian carcinoma (HGSOC) or homologous recombination proficient (HRP) ovarian cancer; or any homologous recombination proficient (HRP) cancer. The cancer may be any homologous recombination deficient (HRD) cancer, such as HRD ovarian cancer. The cancer may be an HRD cancer or tumor that comprises a mutation in a RAD51, PALB2, ATM, ATR, CHEK2, RAD51, or FANC gene. The cancer may be an HRD cancer or tumor that is associated with aberrant expression of proteins encoded by a RAD51, PALB2, ATM, ATR, CHEK2, or FANC genes. The cancer may be any cancer that comprises a genetic mutation which upregulates STAT3 signaling and/or polarizes tumor associated macrophages to M2-like macrophages (e.g., a cancer with a mutation in the KRAS gene, such as the KRAS^{G12D} mutation). In some embodiments, the cancer comprises breast cancer carrying a BRCA mutation (e.g., advanced breast cancer carrying germline BRCA1/2 mutations). In certain embodiments, the subject has BRCA naïve breast or ovarian cancer. In some embodiments, the subject is a rodent, primate, human, or animal model of cancer, optionally wherein the subject is human. In some embodiments, the subject has a deficiency in activating STING signaling in tumor cells. In some embodiments, the subject has a deficient STING signaling pathway in tumor cells, therefore intra-tumoral STING agonists (e.g. dsDNAs/cGAMP released from tumor cells or intra-tumoral delivered STING agonists) cannot activate intra-tumoral dendritic cells and macrophages.

In an aspect, methods of treating a subject with advanced breast cancer carrying germline BRCA1/2 mutations, in which the cancer includes a tumor with an M2 enrichment score higher than 0.27 or a ratio of M2/M1 higher than 1.0, include systemically administering to the subject, optionally wherein the administration is about 10 mg/kg body weight of a STING agonist conjointly with about 50 mg/kg body weight of a PARP inhibitor.

Also provided herein is a method of treating a subject with non-small cell lung cancer carrying germline EGFR mutations, wherein the cancer comprises a tumor with an M2 enrichment score higher than 0.27, comprising administering to the subject a STING agonist conjointly with an EGFR-TK inhibitor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1G show that Brca1-deficient breast tumors have a modest response to olaparib in vivo with immune suppressive TAMs. FIG. 1A shows generation of a syngeneic GEMM of Brca1-deficient breast tumors by intra-ductal injection of adenovirus expressing Cre recombinase (Ad-Cre) directly into the lumen of mammary glands. FIG. 1B shows tumor-free survival of Brca1L/L Trp53L/L mice with or without intra-ductal injection of Ad-Cre (n=6). FIG. 1C shows tumor growth of Brca1−/− Trp53−/− (BP) allografts in FVB mice treated with olaparib or anti-PD-1 as monotherapy or in combination. Control, n=8; anti-PD-1, n=8; olaparib, n=6; olaparib+anti-PD-1, n=10. FIG. 1D shows analysis of BP tumors from FVB mice treated with or without olaparib for 21 days. Tumor-associated macrophages (TAMs, 7AAD-CD45+ CD11b+ F4/80+) were plotted as CD206 versus MHC-II to identify M1-like (MHC-IIhigh CD206−) and M2-like (MHC-IIlow CD206+) polarization phenotype. Each dot represents results from a single tumor. FIG. 1E shows analysis of tumors from FVB mice bearing Brca1-deficient mouse breast or Brca1-deficient mouse ovarian tumors 28 days after tumor cell implantation. Each dot represents results from a single tumor. FIG. 1F shows analysis of TCGA cohorts of patients with BRCA1 mutant (mut) ovarian cancer (n=29) and patients with BRCA1 mut breast cancer (n=30) for M2 immunosuppressive gene signature. FIG. 1G shows analysis of cytokine production of CD8+ T cells co-cultured with TAMs sorted from BP tumors for 2 days (n=4-8). Data are presented as mean±SEM (FIG. 1C and FIG. 1G), mean±SD (FIG. 1F), or median with quartiles (violin plots). Two-way analysis of variance (ANOVA) (FIG. 1C). Two-tailed unpaired t test (FIG. 1D-FIG. 1G). ns, not significant; *P<0.05, **P<0.005, ****P<0.0001.

FIG. 2A-FIG. 2E show the Brca1L/LTrp53L/L mouse model of mammary tumors induced by intra-ductal injection of Ade-Cre. The left panel of FIG. 2A shows RT-qPCR analysis of Brca1 in mouse mammary epithelial cells (MMECs) and Brca1−/− Trp53−/− (BP) mouse mammary tumor cells. The right panel of FIG. 2A shows RNA-seq analysis of Trp53 in BP and MMECs (n=3-5). FIG. 2B shows H&E staining of mammary tumors from Brca1L/LTrp53L/L mice. FIG. 2C shows analysis of BP tumors from FVB mice with or without olaparib treatment, showing assessments of intratumoral CD8+ T cells after 7 days of treatment as well as the tumor-associated macrophages (TAMs) after 21 days of treatment. Each dot represents results from a single tumor. FIG. 2D shows analysis of proportion of effector cells (CD44highCD62Llow) and surface expression of CD25 of CD8+ T cells co-cultured with TAMs (7AAD-CD45+CD11b+F4/80+) sorted from BP tumors for 2 days (n=4-8). FIG. 2E shows gating strategies for flow cytometry analyses described in FIG. 1D, FIG. 1E, and FIG. 2C. Data are presented as mean±SEM, or median with quartiles (violin plots). Two-tailed unpaired t test. ns, not significant; *P<0.05, ***P<0.0005, ****P<0.0001.

FIG. 3A-FIG. 3E show that BRCA-deficient breast tumor cells induce M2-like macrophage polarization in vitro. (FIG. 3A) Diagram of workflow. Top, mouse bone marrow-derived macrophages (BMDMs) co-cultured with BP tumor cells with or without olaparib treatment. Bottom, BMDMs were incubated with 50% conditioned media (CM) harvested from olaprib- or DMSO-treated BP tumor cells. (FIG. 3B) Flow cytometric analysis of BMDMs co-cultured with BP tumor 1 cells. BMDMs (CD11b+) were plotted as CD206 versus MHC-II to identify M1-like (CD206−MHC-II^{high}) and M2-like (CD206+MHC-II^{low}) polarization phenotypes (n=3-6). (FIG. 3C) Heat map of gene expression for antitumor and protumor genes in BMDMs incubated with DMSO vehicle control, olaparib (OL, 5 μM), 50% BP-CM, or 50% OL-treated BP (BP/OL)-CM for 24 hours. (FIG. 3D) RT-qPCR analysis of mouse BMDMs incubated with control medium, 50% BP-CM, or 50% BP/OL-CM for 24 hours (n=3-5). (FIG. 3E) RT-qPCR analysis of THP-1 human macrophages incubated with control medium, 50% tumor cell-CM, or 50% CM of olaparib-treated tumor cells for 24 hours (n=3-6). Data are presented as mean±SEM. One-way analysis of variance (ANOVA) (FIG. 3B, FIG. 3D and FIG. 3E). ns, not significant; *P<0.05, **P<0.005, ***P<0.0005, ****P<0.0001.

FIG. 4A-FIG. 4B show that BRCA1-deficient breast tumor cells induce pro-tumorigenic macrophage polarization in vitro. (FIG. 4A-FIG. 4B) Analysis of mouse BMDMs incubated with control medium or 50% conditioned media (CM) derived from BP or olaparib-treated BP cells (BP/OL) for two days. BMDMs (CD11b+) were plotted as CD206 versus MHC II to identify M1-like (CD206−MHCIIhigh) and M2-like (CD206+MHCIIlow) polarization phenotypes (n=4-7). Data are presented as mean±SEM. One-way analysis of variance (ANOVA). ns, not significant; **P<0.005.

FIG. 5A-FIG. 5J show that TEMs suppress olaparib-induced DNA damage in BRCA1-deficient breast tumor cells and abrogate STING activation in DCs. (FIG. 5A) Diagram of workflow for FIG. 5B-FIG. 5F. Conditioned media from control BMDMs or tumor-educated macrophages (TEMs) were added to tumor cell cultures, followed by olaparib treatment. (FIG. 5B) BP tumor cells were stained using DAPI and an anti-dsDNA antibody after two days of olaparib treatment. The intensity of cytosolic dsDNA was quantified. Scale bar, 50 μm (n=10-26 fields from two independent experiments). (FIG. 5C-FIG. 5D) BP cells (FIG. 5C) or MDA-MB-436 cells (FIG. 5D) were stained with anti-H2AX phospho (Ser139) antibody and analyzed by flow cytometry. MFI, median florescence intensity (n=3). (FIG. 5E-FIG. 5F) BP cells (FIG. 5E) or MDA-MB-436 cells (FIG. 5F) were analyzed for apoptosis (Annexin V+ 7-AAD−) (n=3-6). (FIG. 5G) Apoptotic analyses of BP cells co-cultured with or without tumor-associated macrophages (TAMs) sorted from BP tumors, followed by three days of olaparib treatment (n=3). (FIG. 5H) Diagram of workflow for FIG. 5I and FIG. 5J. Tumor cells were incubated with control medium or conditioned media from BMDMs or TEMs, followed by olaparib treatment. After two days of treatment, olaparib was washed off and mouse bone marrow-derived dendritic cells (DCs) were added onto BP cells to co-culture for 24 hours. (FIG. 5I) Flow cytometric analysis of STING pathway activation (p-TBK1+1 p-IRF3+) of DCs co-cultured with olaparib- or DMSO-treated BP cells (n=4). (FIG. 5J) RT-qPCR analysis for Ifnb, Ccl5 and Cxcl10 expression in DCs isolated from the co-culture (n=4-9). Data are presented as mean±SEM. One-way analysis of variance (ANOVA). ns, not significant; *P<0.05, **P<0.005, ***P<0.0005, ****P<0.0001.

FIG. 6A-FIG. 6E show that tumor cell-educated macrophages (TEMs) suppress synthetic lethal response of BRCA1-deficient breast tumor cells to olaparib. (FIG. 6A) Analysis of MDA-MD-436 cells co-cultured with THP-1 human macrophages with or without 16 hours of treatment with olaparib. MDA-MD-436 tumor cells (CD45−) in the co-culture were analyzed by flow cytometry for the phosphorylation of histone H2AX at Ser139 (n=6). (FIG. 6B) Analysis of BP cells co-cultured with mouse BMDMs with or without olaparib treatment for three days. BP tumor cells (CD11b−) were analyzed by flow cytometry for apoptosis (Annexin V+7-AAD−) (n=5-6). (FIG. 6C) Analysis of MDA-MB-436 cells co-cultured with THP-1 human macrophages with or without olaparib treatment for three days. MDA-MB-436 tumor cells (CD45−) were analyzed by flow cytometry for apoptosis (n=3). (FIG. 6D) Gating strategies for flow cytometric analysis of STING pathway activation (p-TBK1+p-IRF3+) of DCs co-cultured with BP cells described in FIG. 3I. (FIG. 6E) RT-qPCR analysis of BP tumor cells treated with or without olaparib for two days in control medium, 50% naive BMDM-conditioned media (CM), or 50% TEM-CM (n=5-6). Data are presented as mean±SEM. One-way analysis of variance (ANOVA). *P<0.05, **P<0.005, ***P<0.0005, ****P<0.0001.

FIG. 7A-FIG. 7I show that STING agonists reprogram TEMs into an M1-like state and promote activation of DCs by tumor cells upon oalaprib in the presence of TEMs. (FIG. 7A-FIG. 7B) Mouse BMDMs were subjected to transcriptome analysis after treatment for 24 hours with DMSO vehicle control, olaparib (OL, 5 μM), DMXAA (0.05 mg/mL), 50% BP-CM with or without DMXAA, or 50% BP/OL-CM with or without DMXAA. (FIG. 7A) Heat map of antitumor and protumor gene expression of BMDMs. (FIG. 7B) Left, volcano plot showing the significance and magnitude of changes in gene expression of BMDMs treated with BP-CM/DMXAA compared to BP-CM/DMSO. Right, top-ranked up-regulated and down-regulated gene ontology (GO) terms in BMDMs treated with BP-CM/DMXAA. (FIG. 7C) Analysis of control naive BMDMs and BP TEMs treated with or without DMXAA (0.05 mg/mL) for two days. M1-like (CD11b+CD206−MHC-IIhigh) to M2-like (CD11b+CD206+MHC-II^{low}) ratio was analyzed by flow cytometry (n=4). (FIG. 7D) Analysis of STING pathway activation (p-TBK1+p-IRF3+) of control naive BMDMs and BP TEMs treated with or without DMXAA for 24 hours (n=3-4). (FIG. 7E-FIG. 7F) BMDMs, isolated from wildtype (WT) or STING knockout (STING−/−) C57/BL6J mice, were incubated with control medium or 50% BP-CM to generate control naive BMDMs and TEMs, respectively. Cells were then treated with or without DMXAA for 2 days. M1 to M2 ratio (FIG. 7E) and surface level of a co-stimulatory molecule CD86 (FIG. 7F) were analyzed by flow cytometry (n=4). (FIG. 7G) Expression of M2 (CD163) and M1 (CD86) markers in control THP-1 macrophages or MDA-MB-436 tumor cell-educated THP-1 macrophages (TEMs-436) treated with or without ADU-S100 (10 μM) for two days (n=3). (FIG. 7H) Diagram of workflow for (FIG. 7I). Tumor cells were incubated with control medium or conditioned media from naive BMDMs, TEMs or DMXAA-treated TEMs, followed by olaparib treatment. After two days of treatment, drugs were removed and tumor cells 1 were co-cultured with DCs for 24 hours. Cocultrue cells were harvested for flow cytometry. (FIG. 7I) Top, DNA damage response of BP tumor cells to olaparib was analzyed by staining for Ser139-phosphorylated H2AX (n=3-6). Bottom, Flow cytometric analysis of STING pathway activation (p-TBK1+p-IRF3+) in DCs co-cultured with BP tumor cells (n=4). Data are presented as mean±SEM. One-way analysis of variance (ANOVA). ns, not significant; **P<0.005, ***P<0.0005, ****P<0.0001.

FIG. 8A-FIG. 8B show that STING agonists reprogram tumor-cell educated macrophages (TEMs) into an M1-like state in vitro. (FIG. 8A) Mouse BMDMs were subjected to transcriptome analysis after a 24-hour exposure to 50% conditioned media (CM) from olaparib-treated BP tumor cells (BP/OL) with or without DMXAA (0.05 mg/mL). Left, volcano plot showing the significance and magnitude of changes in gene expression comparing macrophages treated with BP/OL-CM/DMXAA versus BP/OL-CM/DMSO. Right, top-ranked up-regulated and down-regulated gene ontology (GO) terms in macrophages treated with BP/OL-CM/DMXAA. (FIG. 8B) Representative images of mouse BMDMs after a 24-hour incubation with medium containing IL4 (20 ng/ml), LPS (100 ng/ml), IFNg (20 ng/mL), olaparib (5 μM), DMXAA (0.05 mg/mL), 50% BP-CM with or without DMXAA, or 50% BP/OL-CM with or without DMXAA.

FIG. 9A-FIG. 9C show that STING agonists improve therapeutic response of orthotopic BP tumors to olaparib in syngeneic immunocompetent mice in vivo. (FIG. 9A) BP tumor growth in FVB mice treated with olaparib (50 mg/kg, i.p., q.d.) or intratumoral injections of DMXAA (10 mg/kg, one dose per week for 3 weeks [total of 3 doses]) as monotherapy or in combination. Control, n=8; olaparib, n=9; DMXAA, n=6; olaparib+DMXAA, n=7. (FIG. 9B and FIG. 9C) Analysis of BP tumors after 21 days of treatments for effector cytokine production by intratumoral CD8+ T cells and CD4+ T cells. Each dot represents results from a single tumor. Data are presented as mean±SEM (tumor volumes), or median with quartiles (violin plots). Two-way analysis of variance (ANOVA) (FIG. 9A). One-way ANOVA (FIG. 9B and FIG. 9C). ns, not significant; *P<0.05, **P<0.005, ***P<0.0005, ****P<0.0001.

FIG. 10A-FIG. 10D show that depletion of TAMs improves therapeutic response of orthotopic BP tumors to olaparib in syngeneic immunocompetent mice in vivo. (FIG. 10A) Tumor growth of Brca1−/−Trp53−/− (BP) allografts in FVB mice treated with olaparib or anti-CSF1R as monotherapy or in combination. Control, n=10; olaparib, n=10; anti-CSF1R, n=10; olaparib+anti-CSF1R, n=10. (FIG. 10B-FIG. 10D) Flow cytometric analyses of intratumoral immune cells of BP tumors after 21 days of treatments. Each dot represents results from a single tumor. Data are presented as mean±SEM (FIG. 10A) or median with quartiles (violin plots, FIG. 10B-FIG. 10D). Two-way analysis of variance (ANOVA) (FIG. 10A). One-way ANOVA (FIG. 10B-FIG. 10D). ns, not significant; *P<0.05, **P<0.005, ***P<0.0005, ****P<0.0001.

FIG. 11A-FIG. 11J show that systemic delivery of STING agonists sensitizes STING-null BP tumors to olaparib in vivo. (FIG. 11A) Western blots for STING and Vinculin in CRISPR/Cas9 control and STING knockout BP tumor cells (BP-sgControl and BP-sgSTING). (FIG. 11B) CellTiter-Glo® analysis showing cell viability of BP-sgControl and BP-sgSTING cells after three days of treatment with serial dilution of olaparib (n=3). (FIG. 11C) Flow cytometric analysis of mouse BMDMs treated with DMSO vehicle control, olaparib (OL, 5 μM), 50% BP-sgSTING-CM, or 50% BP-sgSTING/OL-CM for two days (n=3). (FIG. 11D) ELISA analysis of IFN β in media from BP-sgControl or BP-sgSTING cells with or without two days of olaparib treatment (n=7). (FIG. 11E-FIG. 11F) RT-qPCR analysis of Ccl5 (FIG. 11E) and Cxcl10 (FIG. 11F) in BP-1 sgControl and BP-sgSTING cells treated with or without olaparib for two days (n=3-4). (FIG. 11G) Tumor growth (Left) and survival (Right) of BP-sgControl tumor-bearing FVB mice treated with olaparib (50 mg/kg, i.p., q.d.), DMXAA (10 mg/kg, i.p.) or olaparib+DMXAA. Median survivals are shown in parentheses. Left, Control, n=13; Olaparib, n=7; DMXAA, n=9; Olaparib+DMXAA, n=14. Right, Control, n=8; olaparib, n=5; DMXAA, n=6; olaparib+DMXAA, n=9. (FIG. 11H) Tumor growth (Left) and survival (Right) of BP-sgSTING tumor-bearing FVB mice treated with olaparib (50 mg/kg, i.p., q.d.), DMXAA (10 mg/kg, i.p.) or olaparib+DMXAA. Median survivals are shown in parentheses. Left, Control, n=24; olaparib, n=11; DMXAA, n=9; olaparib+DMXAA, n=19. Right, Control, n=13; olaparib, n=7; DMXAA, n=6; olaparib+DMXAA, n=11. (FIG. 11I-FIG. 11J) BP-sgControl (FIG. 11I) and BP-sgSTING (J) tumor growth in FVB mice treated with olaparib+DMXAA with or without anti-CD8 or anti-IFNAR1 neutralizing antibodies (n=6 per condition). Data are presented as mean±SEM. One-way analysis of variance (ANOVA) (FIG. 11C-FIG. 11F). Two-way ANOVA for tumor growth (FIG. 11G, FIG. 11H, FIG. 11I and FIG. 11J). Log-rank Mantel-Cox test for survival (FIG. 11G and FIG. 11H). ns, not significant; *P<0.05, **P<0.005, ***P<0.0005, ****P<0.0001.

FIG. 12A-FIG. 12B show that systemic delivery of STING agonists sensitizes 1 STING-null BP tumors to olaparib in vivo. (FIG. 12A) Analysis of bone marrow-derived dendritic cells (DCs) co-cultured with DMSO vehicle control-treated or olaparib-treated BP-sgControl or BP-sgSTING tumor cells for 24 hours (n=3). (FIG. 12B) Tumor growth of BP-sgSTING allografts in FVB mice treated with intratumoral injections (IT) of DMXAA (10 mg/kg, one dose per week) as a single agent treatment or in combination with olaparib (50 mg/kg, i.p., q.d.). Control, n=4 tumors; DMXAA, n=4 tumors; DMXAA+Olaparib, n=4 tumors. Data are presented as mean±SEM, or median with quartiles (violin plots). One-way analysis of variance (ANOVA) (FIG. 12A). Two-way ANOVA (FIG. 12B). ns, not significant; *P<0.05, **P<0.005.

FIG. 13 shows that harnessing antitumor immunity with STING agonists overcomes immune suppression and resistance to PARP inhibition in BRCA1-deficient breast cancer. BRCA1-deficient breast tumors elicit pro-tumorigenic macrophage polarization via paracrine activation of macrophage M2-like phenotype. In turn, these tumor-educated macrophages not only exhibit suppressive activity against T cells, but also attenuate PARPi-mediated synthetic lethality and the production of cytosolic double-stranded DNA (dsDNA), thus abrogating the activation of the DNA sensing adaptor STING and rendering BRCA1-deficient breast tumors resistant to PARPi therapy. Exogenous agonists of the STING pathway reprogram the macrophages and trigger innate immune activation of both macrophages and DCs, synergizing with PARPi therapy in inducing tumor cell DNA damage and an adaptative immune response that re-sensitizes tumors to PARPi therapy.

FIG. 14A-FIG. 14D show that systemic delivery of STING agonist MSA-2 sensitizes PARPi-resistant tumors to olaparib in vivo. (FIG. 14A) Generation of PARPi-resistant ovarian cancer tumor. PBM is the control group, PBM-R is the recurrent tumors after long-term olaparib treatment (olaparib-resistant group). (FIG. 14B) M2 like-TAMs enriched in the PBM-R tumors. Flow cytometric analyses of intratumoral and ascites M2 like-TAMs in PBM and PBM-R tumors. (FIG. 14C) STING agonists reprogram PBM-R cultured media-educated macrophages (TEMs) into an M1-like state in vitro. Analysis of control naive BMDMs and PBM-R TEMs treated with or without ADU-S100 (10 μM) or MSA-2 (5 ug/ml) for two days. M1-like (CD11b⁺CD206⁻MHC-II^{high}) to M2-like (CD11b⁺CD206⁺MHC-II^{low}) ratio was analyzed by flow cytometry (n=3). (FIG. 14D) Tumor weight (Left) and excised tumors (Right) from PBM-R tumor-bearing FVB mice treated with vehicle control, Olaparib (50 mg/kg, i.p., q.d.)+anti-PD-1 (200 ug/mouse, i.p., q3d.), MSA-2 (25 mg/kg, i.p., three times per week) or MSA-2+olaparib+anti-PD-1 for two weeks. Two-way ANOVA. *P<0.05, **P<0.01, ***P<0.001.

FIG. 15A-15E show characterization of pro-tumor macrophages in Brca1-deficient ovarian tumors that acquired secondary resistance to PARP inhibition. FIG. 15A shows generation of PARPi-resistant ovarian cancer mouse models by long-term treatment of PBM tumor-bearing mice with olaparib (PBM-R, PBM refractory tumors after olaparib treatment). FIG. 15B shows tumor growth curve of PBM-tumor bearing mice treated with olaparib and vehicle control. FIG. 15C shows a measurement of IC50 value of PARPi-naïve PBM cells and tumor cells isolated from PBM-R tumor-bearing mice, each line derived from a PBM-R tumor-bearing mouse. FIG. 15D shows PBM and PBM-R cells were treated 1 μM olaparib or vehicle control for 24 hours and stained with anti-phospho-H2A.X (Ser139) antibody (γ-H2AX) for flow cytometry analysis (n=3). FIG. 15E shows tumor burden and representative bioluminescence-imaging analysis of PBM or in vitro sensitive PBM-R tumor-bearing mice treated with vehicle control or olaparib. Data are presented as mean±SD. One-way analysis of variance (ANOVA). ****P<0.0001.

FIG. 16 shows copy number variations of DNA repair pathway genes detected in the in vitro PARPi-resistant PBM-R line.

FIG. 17A-FIG. 17F show M2-like macrophages increase in PARPi-resistant Brca1-deficient ovarian cancer. FIG. 17A shows flow cytometry analysis of tumor-infiltrating pro-tumor TAMs (M2-like TAMs) in PBM and PBM-R tumor-bearing mice. FIG. 17B shows flow cytometry analysis of total and M2-like TAMs in the ascites of PBM and PBM-R tumor-bearing mice. FIG. 17C shows a diagram of workflow for D. FIG. 17D shows flow cytometry analysis of bone marrow derived macrophages (BMDMs) cultured in 50% complete medium and 50% PBM-CM or PBM-R-CM three days. FIG. 17E shows a diagram of workflow for F. FIG. 17F shows flow cytometry analysis of bone marrow cells (BMCs) cultured in 50% complete medium and 50% ascites supernatant for 5 days. Data are presented as mean±SD. One-way analysis of variance (ANOVA). ns, not significant; *P<0.05, **P<0.01, ***P<0.001, ****P<0.0001.

FIG. 18 shows flow cytometry analysis of tumor-infiltrating CD45⁺, CD11b⁺, MDSC, CD4⁺, CD8⁺ and Treg cells in PBM and PBM-R tumors.

FIG. 19A-FIG. 19K show STAT3 signaling activation is required for M2-like macrophages polarization in PARPi-resistant Brca1-deficient ovarian cancer. FIG. 19A shows GSEA analysis of RNA sequencing data of PBM and PBM-R tumors. FIG. 19B shows flow cytometry analysis of phosphorylation level of STAT3 (Y705) in PBM and PBM-R tumor cells. FIG. 19C shows representative images of immunohistochemistry (IHC) staining for p-STAT3 (Y705) in PBM and PBM-R tumors. FIG. 19D shows flow cytometry analysis of p-STAT3 in PBM tumor cells treated with indicated concentration of olaparib or vehicle control. FIG. 19E shows flow cytometry analysis of BMDMs cultured in CM from PBM with or without olaparib treatment. FIG. 19F shows an analysis of cytokines in the medium of PBM treated with olaparib or vehicle control. FIG. 19G shows an analysis of mouse BMDMs cultured with or without 50% PBM-R-CM in the presence or absence of indicated neutralizing antibodies for three days (n=3). FIG. 19H shows flow cytometry analysis of BMDMs cultured in CM from PBM-R with or without knockdown of STAT3. FIG. 19I shows tumor burden of mice transplanted with PBM-R tumor cells expressing control or STAT3 shRNAs and treated with olaparib or vehicle control. Flow cytometry analysis of total TAMs and the ratio of M1-like and M2-like microphages (FIG. 19J), CD4⁺ and CD8⁺ effector T cells (FIG. 19K) in olaparib-treated PBM-R tumors expressing control or STAT3 shRNAs in (I). *P<0.05, **P<0.01, ***P<0.001, ****P<0.0001.

FIG. 20A-FIG. 20F show a PARP inhibition upregulates STAT3 signaling and render PARPi-resistance in Brca1-deficient ovarian cancer. FIG. 20A shows GSEA analysis of STAT3 signaling pathway in PBM and PBM-R tumors. FIG. 20B shows western blot analysis of total and phosphorylated STAT3 in PBM cells treated with indicated concentrations of olaparib or vehicle control for 24 hours. FIG. 20C shows western blot analysis of total and phosphorylated Stat3 in PBM and PBM-R cells stably expressing control shRNA or shRNAs targeting Stat3. FIG. 20D shows evaluation of IC50 value in control and Stat3-silenced PBM-R cells after olaparib treatment. Analysis of tumor-infiltrating M1- and M2-like macrophages (FIG. 20E), CD4⁺ and CD8⁺ cells (FIG. 20F) in Stat3-silenced PBM-R tumors treated with olaparib or vehicle control. *P<0.05, **P<0.01, ***P<0.001.

FIG. 21A-FIG. 21N show STING agonism reprograming myeloid cells in vitro and in vivo in a STING-dependent manner. FIG. 21A shows a diagram of workflow for FIG. 21B and FIG. 21H. Bone marrow derived macrophages (BMDMs) were cultured in 50% PBM-R-CM or control medium for 48 hours with or without STING agonists (ADU-S100 or MSA-2) treatment. FIG. 21B shows flow cytometry analysis of macrophage phenotypes in FIG. 21A. FIG. 21C shows diagram of workflow for FIG. 21D. FIG. 21D shows a heat map of differentially expressed genes in myeloid cells (CD45⁺CD11b⁺) collected from the ascites of PBM-R tumor-bearing mice with indicated treatment. FIG. 21E shows GSEA analysis showing upregulated STING signaling pathway in myeloid cells in the ascites of MSA-2 treated PBM-R tumor-bearing mice (FIG. 21F-21H). Flow cytometry analysis of TAMs (FIG. 21F), myeloid DCs (FIG. 21G, left), MHC-I⁺ DCs (G, right), p-TBK-1⁺ DCs (FIG. 21H) and in the ascites of PBM-R tumor-bearing mice treated with control, olaparib, MSA-2 and olaparib+MSA-2 for 24 hours. FIG. 21I shows analysis of the ratio of M1/M2 in WT BMDMs and STING^{-/-} BMDMS cultured in CM from ID8-Brca1^{-/-} treated with olaparib or vehicle control, n=3. FIG. 21J shows a diagram of workflow for (FIG. 21K). FIG. 21K shows an analysis of macrophages (M1/M2) in WT and STING^{-/-} mice injected with ID8-Brca1^{-/-} tumor cells and treated with indicated drugs for 24 h. FIG. 21L shows tumor burden of PBM-R tumor bearing mice treated with control, olaparib, MSA-2 and olaparib in combination with MSA-2 for 14 days (n=7). ns, not significant; *P<0.05, **P<0.01, ***P<0.001, ****P<0.0001. FIG. 21M shows flow cytometry analysis of mouse BMDMs cultured in CM from control or olaparib-treated ID8-Brca1^{+/+} and ID8-Brca1^{-/-} cells in the presence or absence of 5 μg/ml MSA-2 for three days. FIG. 21N shows flow cytometry analysis of human BMDMs cultured in CM from control or olaparib-treated UWB1.289 or UWB1.289+BRCA1 cells in the presence or absence of 5 μg/ml MSA-2 for three days. *P<0.05, **P<0.01, ***P<0.001, ****P<0.0001.

FIG. 22A-FIG. 22D show STING agonism modulates myeloid cells and render PBM-R tumors sensitive to PARP inhibition. FIG. 22A shows flow cytometry analysis of myeloid cells isolated from ascites. FIG. 22B shows volcano plot showing the significance and magnitude of changes in gene expression in mouse dendritic cells treated with STING agonist (DMXAA) and inhibitor of TBK-1 (BX795). FIG. 22C shows top-ranked up-regulated gene ontology (GO) terms in myeloid cells treated MSA-2 or MSA-2 in combination with olaparib. FIG. 22D shows analysis of total and p-TBK-1⁺ myeloid DCs in WT and STING^{-/-} mice injected with ID8-Brca1^{-/-} tumor cells and treated with indicated drugs for 24 h. *P<0.05.

FIG. 23A-FIG. 23G show STING agonism modulates TME and resensitizes PBM-R tumors to PARP inhibition. FIG. 23A shows tumor burden of PBM-R tumor bearing mice treated with control, olaparib, MSA-2 and olaparib in combination with MSA-2 for 14 days (n=7). (FIG. 23B-FIG. 23G) Flow cytometry analysis of tumor infiltrating immune cells in PBM-R tumor-bearing mice as described in (FIG. 23A): (FIG. 23B) TAMs, (FIG. 23C) M1/M2, (FIG. 23D) CD86⁺ (left) and p-TBK-1⁺ (right) in cDCs (CD11c⁺ MHC-II⁺), (FIG. 23E) MHC-I⁺ (left) and p-TBK-1⁺ in myeloid DCs (CD11b⁺ MHC-II⁺), (FIG. 23E) total and TNFα⁺ CD4⁺ T cells and (FIG. 23G) total and TNFα⁺ CD8⁺ T cells. Gray dots: outliers. Two-way analysis of variance (ANOVA). ns, not significant; *P<0.05, **P<0.01, ***P<0.001, ****P<0.0001.

FIG. 24A-FIG. 24F show STING agonism in combination with olaparib increases tumor-infiltrating anti-tumor immune cells in PBM-R tumor-bearing mice. FIG. 24A-FIG. 24F shows flow cytometry analysis of tumor infiltrating immune cells in PBM-R tumor-bearing mice treated with control, olaparib, MSA-2 and olaparib in combination with MSA-2 for 14 days (n=7). (FIG. 24A) CD45⁺, (FIG. 24B) CD11b⁺, (FIG. 24C) cDCs (CD11c⁺ MHC-II⁺), (FIG. 24D) myeloid DCs (CD11b⁺ MHC-II⁺), (FIG. 24E) CD3⁺ and (FIG. 24F) Effector CD4⁺ and CD8⁺ T cells. Gray dots: outliers. Two-way analysis of variance (ANOVA). ns, not significant; *P<0.05, **P<0.01, ***P<0.001, ****P<0.0001.

FIG. 25A-FIG. 25J show overcoming PARPi-resistance in ovarian PDXs with STING agonist treatment. FIG. 25A a diagram of workflow for (B-E). FIG. 25B shows detection of phosphorylation level of STAT3 (Y705) by flow cytometry in human ovarian PDXs with or without olaparib treatment. FIG. 25C shows flow cytometry analysis of human BMDMs cultured in CM from PDXs treated with olaparib or control as described in FIG. 25A for three days. FIG. 25D shows a flow cytometry analysis of human BMDMs cultured in CM from control or STAT3 inhibitor (napabucasin)-treated PDXs. FIG. 25E shows an analysis of human BMDMs cultured in CM from PDXs in the presence or absence of MSA-2. FIG. 25F shows a diagram of workflow for FIG. 25G. FIG. 25G tumor burden of DF86 PDX-bearing mice treated with control, MSA-2, olaparib and MSA-2 in combination with olaparib for 2 weeks. FIG. 25H-FIG. 25J show an analysis of indicated immune cells in the ascites of DF86 PDX-bearing mice as described in FIG. 25G: (FIG. 25H) TAMs (CD11b⁺; CD68⁺); (FIG. 25I) M1/M2, TAMs were analyzed to identify M1-like (CD80⁺; CD163⁻) and M2-like (CD80⁻; CD163⁺) polarization phenotypes; (FIG. 25J) CD14⁺ DCs (CD14⁺HLA-DR⁺). *P<0.05, ** P<0.01, ***P<0.001.

FIG. 26A-FIG. 26F show combined treatment of STING agonist and PARPi in ovarian PDXs. FIG. 26A shows flow cytometry analysis of γ-H2AX in ovarian PDXs treated with olaparib or vehicle control (n=3). FIG. 26B shows tumor burden of DF86 PDX-bearing mice treated with control, MSA-2, olaparib and MSA-2 in combination with olaparib for 2 weeks. FIG. 26C-FIG. 26E show analysis of indicated immune cells in the ascites of DF86 PDX-bearing mice as described in FIG. 26B: (FIG. 26C) TAMs (CD11b⁺; CD68⁺); (FIG. 26D) M1/M2, (FIG. 26D) CD14⁺ DCs (CD14⁺HLA-DR⁺). *P<0.05, **P<0.01, ***P<0.001. FIG. 26F shows a graphical abstract of TAM polarization.

FIG. 27A-FIG. 27E show osimertinib (AZD9291)-elicited adaptive immune activation is necessary for its therapeutic efficacy in vivo. FIG. 27A shows CD8⁺ T cell depletion in FVB mice compromised in vivo therapeutic efficacy of AZD9291 in a GEM model of EGFR-mutant tumors driven by Exon19del/T790M EGFR and loss of Trp53 (referred as PE) (ctrl, n=6; AZD9291, n=6; AZD9291+CD8 antibody, n=8). FIG. 27B shows AZD9291 treatment induced T cell recruitment and activation in tumor microenvironment (TME). Each dot represents results from a single tumor. FIG. 27C shows AZD9291 increased expression of CCL5 and CXCL10 in cultured tumor cells derived from PE GEMM as well as a human EGFR-mutated NSCLC cell line PC9GR4. FIG. 17D shows analysis of clinical data of eight patients with EGFR-mutated advanced NSCLC suggests that the T cell inflamed signature in tumors is significantly enhanced upon EGFR-TKI (osimertinib or erlotinib) treatments in responders (n=4, progression-free survival [PFS]>8 months) but not in non-responders (n=4, PFS <8 months). FIG. 28D shows T cell inflamed score enriched upon EGFR-TKI (osimertinib or erlotinib) treatments is positively correlated with PFS in patients with EGFR-mutated advanced NSCLC received the EGFR-TKI as first-line treatment. Data are presented as mean±SEM (a and c) or median with quartiles (violin plots, b). ns, not significant; *P<0.05, **P<0.01.

FIG. 28A-FIG. 28C show an established immune-suppressive TME inhibits therapeutic efficacy of osimertinib (AZD9291). FIG. 28A shows the therapeutic efficacy of AZD9291 is modest in large tumors. FIG. 28B shows flow cytometry analysis revealed that large tumors (500 mm³) exhibit a more immune-suppressive TME than smaller tumors (100 mm³), as evidenced by the reduction of T cell and DC infiltration, and increased TAM infiltration and M2 polarization of TAMs. Each dot represents results from a single tumor. FIG. 28C shows AZD9291 was not able to elicit T cell activation in large tumors (500 mm³). Data are presented as mean±SEM (a) or median with quartiles (violin plots, b and c). ns, not significant; *P<0.05, **P<0.01, ****P<0.0001.

FIG. 29A-FIG. 29D show tumor-associated macrophages (TAMs) suppress CD8⁺ T cell activation and compromise therapeutic efficacy of osimertinib (AZD9291) in large established tumors. FIG. 29A shows an analysis of clinical data of patients with EGFR-mutated advanced NSCLC showing that the non-responders to TKI (osimertinib or erlotinib) treatments exhibited significantly enriched TAM signature before TKI treatments as compared to the responders. FIG. 29B shows TAM enrichment scores before TKI treatments are negatively correlated with T cell inflamed score upon TKI treatment. FIG. 29C shows TAMs derived from PE GEMM significantly suppressed the activation of CD8⁺ T cell from naïve FVB mice in the coculture. FIG. 29D shows TAM depletion by an anti-CSF1-R antibody enhanced therapeutic efficacy of AZD9291 in large PE tumors (ctrl, n=6; AZD9291, n=7; AZD9291+CSF1 antibody, n=5).Data are presented as median with quartiles (violin plots, c) or mean±SEM (d). ns, not significant; *P<0.05, ***P<0.001, ****P<0.0001.

FIG. 30A-FIG. 30B show systemic delivery of a STING agonist combined with osimertinib induces tumor regression in large established tumors. FIG. 30A shows FVB mice bearing PE tumors were treated with AZD9291, MSA-2 or AZD9291+MSA-2 along with or without an anti-CD8 antibody (ctrl, n=6; AZD9291, n=8, MSA-2, n=8; AZD9291+MSA-2, n=10; AZD9291+MSA-2+CD8 antibody, n=8). FIG. 30B shows combination of AZD9291 and MSA-2, but not single agents, significantly induced immune activation in TME of PE tumors. Each dot represents results from a single tumor. Data are presented as mean±SEM (a) or median with quartiles (violin plots, b and c). ns, not significant; *P<0.05, **P<0.01, ***P<0.0001, ****P<0.0001.

FIG. 31A-FIG. 31D show Osimertinib (AZD9291)-elicited adaptive immune activation is necessary for its therapeutic efficacy in vivo. FIG. 31A shows generation of a GEMM of EGFR-mutant tumors driven by Exon19del/T790M EGFR and loss of Trp53 (referred as PE). FIG. 31B shows western blots showing that phosphorylation of EGFR and ERK in PE tumor cells is inhibited by AZD9291 but not erlotinib. FIG. 31C shows in vivo efficacy of AZD9291 in FVB mice bearing PE tumors (ctrl, n=4; AZD9291 2.5 mg/mg QD, n=4; AZD9291 10 mg/mg QD, n=6). FIG. 31D shows flow cytometry analysis of CD8⁺ T cell abundance in PE tumors and tumor drainage lymph nodes (TDLNs) following the indicated treatments.

Data are presented as mean±SEM or median with quartiles (violin plots). ns, not significant; *P<0.05, ****P<0.0001.

FIG. 32A-FIG. 32B show an established immune-suppressive TME inhibits therapeutic efficacy of osimertinib (AZD9291). FIG. 32 shows representative plots (FIG. 32A) and gating strategies (FIG. 32B) of flow cytometry in FIGS. 28B and C, respectively.

FIG. 33A-FIG. 33C show tumor-associated macrophages (TAMs) suppress CD8⁺ T cell activation and compromise therapeutic efficacy of osimertinib (AZD9291) in large established tumors. FIG. 33A shows two cohorts of patients with NSCLC (GSK cohort and GSE31210 cohort) were categorized into high-TAM (above the median value) and low-TAM (below the median value) groups based on TAM enrichment scores inferred by the tumor transcriptome data. Kaplan Meier survival plots showing the overall survival (OS) in patients with EGFR-mutant and EGFR-wild-type NSCLC, respectively, after the surgical treatments. FIG. 33B shows an anti-CSF1-R antibody monotherapy failed to control tumor growth in FVB mice bearing PE tumors. FIG. 33C shows an anti-CSF1-R antibody monotherapy significantly reduced the abundance of TAMs but not the TAM polarization. Each dot represents results from a single tumor. Data are presented as mean±SEM (a) or median with quartiles (violin plots, 33B). ns, not significant; ***P<0.0001.

FIG. 34A-FIG. 34B show systemic delivery of a STING agonist combined with osimertinib induces tumor regression in large established tumors. FIG. 34A shows analysis of cytokine production of CD8⁺ T cells co-cultured with TAMs. TAMs isolated from treatment-naïve ETP tumors were treated with or without MSA-2 for one day. After washing off MSA-2, TAMs were co-cultured with CD8⁺ T cells isolated from naïve FVB mice for two days. FIG. 34B shows flowing cytometry analysis of CD8⁺ and CD4⁺ T cells in tumor drainage lymph nodes (TDLNs) of PE tumors-bearing FVB mice subjected to the indicated treatments. Each dot represents results from a single tumor. Data are presented as median with quartiles (violin plots, FIGS. 34B and C). ns, not significant; **P<0.01.

FIG. 35 shows that STING agonists like MSA-2 enhance anti-tumor activity of olaparib in PBM-tumor bearing mice. Tumor burden of PBM tumor-bearing mice treated with indicated agents was measured by bioluminescence (ROI, region of intensity) after 2 weeks of treatment (control, n=6; olaparib, n=7, olaparib+MSA-2, n=4). Ordinary one-way ANOVA analysis. **, P<0.01; ***, P<0.001. The murine ovarian tumor model PBM used for this experiment was previously generated (Ding L, Kim H J, Wang Q, Kearns M, Jiang T, Ohlson C E, Li B B, Xie S, Liu J F, Stover E H, Howitt B E, Bronson R T, Lazo S, Roberts T M, Freeman G J, Konstantinopoulos P A, Matulonis U A, Zhao J J. PARP Inhibition Elicits STING-Dependent Antitumor Immunity in Brca1-Deficient Ovarian Cancer. Cell Rep. 2018 Dec. 11;25 (11): 2972-2980.e5. doi: 10.1016/j.celrep.2018.11.054. PMID: 30540933; PMCID: PMC6366450). About 2×10⁵ PBM tumor cells were orthotopically transplanted into syngeneic FVB/NJ mice. Ten days after transplantation, Tumor-bearing mice were equivalently divided into control and treatment groups according to the luminescent intensity. Tumor burden of PBM tumor-bearing mice was measured by bioluminescence (ROI, region of intensity). Olaparib (AZD2281) was administered daily by i.p. (intraperitoneal) injection at dose of 50 mg/kg body weight. MSA-2 was prepared by diluting 50 mg/ml stock in DMSO with PBS (pH 8.0) and administered every other day (three times a week) by i.p. injection at dose of 25 mg/kg body weight.

### DETAILED DESCRIPTION OF THE INVENTION

The present invention is based, at least in part, on discoveries that build on the findings that, in addition to tumor cell-intrinsic synthetic lethality, the immune response triggered by PARP inhibition is also required for effective response in vivo. Through the provided examples, a major mechanism by which BRCA1-deficient breast tumors typically progress through treatment with PARP inhibitors has been uncovered, in contrast to the much higher rates of success observed for BRCA-deficient ovarian cancer. Key findings further demonstrated in the examples, and leading to the underlying discoveries, include the following: (1) BRCA1-mutant breast tumors are refractory to PARP inhibition in vivo in immune-competent mice; (2) BRCA1-mutant breast cancer (both murine and human) cells render tumor-associated macrophages (TAMs) pro-tumorigenic (M2-like) both in vivo and in vitro, which is independent of PARP inhibitor treatment; (3) M2-like TAMs directly suppress T cell activation; however, PD-1 blockade does not add therapeutic benefit to PARP inhibition; (4) M2-like TAMs suppress the tumor cell's DNA damage and cell death in response to PARP inhibition, resulting in reduced production of cytosolic dsDNA and synthetic lethality, thereby dampening STING-dependent activation of dendritic cells (DCs) and macrophages; (5) STING agonists reprogram M2-like TAMs into M1-like anti-tumor macrophages in a macrophage's STING-dependent manner; (6) STING agonists promote DCs activation induced by BP cells upon olaparib in the presence of M2-like TAMs in vitro; (7) STING agonists restore anti-tumor immunity to the tumor immune microenvironment (TIME) in vivo, thus sensitizing BRCA1-deficient breast tumors to olaparib; (8) While STING-deficient/BRCA1-deficient breast tumors fail to respond to the combination of olaparib and a STING agonist administered intratumorally, they respond effectively to systemic delivery of STING agonist in combination with PARP inhibitor and comparably to STING-proficient tumors.

The provided data is thus different from the majority of other studies suggesting that STING agonists act in the tumor cells, but is consistent with a pointing to the importance of STING activation in macrophages and DCs.

Targeting pro-tumorigenic M2-like TAMs has been actively pursued in the field of cancer immunotherapy with various agents, such as small molecule inhibitors or monoclonal antibodies against CSF1/CSF1R or TGFβ/TGFβR, to deplete or suppress TAMs. Many of these agents have been evaluated in early phase clinical trials with little success. The provided examples show, for the first time, that M2-like TAMs suppress olaparib-induced lethal DNA damage and abrogate STING activation, and that a STING agonist is able to efficiently reprogram M2-like TAMs to M1-like anti-tumor macrophages in a macrophage's STING-dependent fashion. Thus, a STING agonist can not only restore the synthetic lethal response to PARP inhibition, but also reshape the TIME to promote an immunogenic anti-tumor response that synergizes with PARP inhibition in BRCA-mutant breast cancers. In some embodiments, immunotherapy (e.g., blockade/inhibitors of immune checkpoints like PD-1, PD-L1, PD-L2, CTLA-4, and the like, such as blocking antibodies well-known in the art) may be used in addition to the therapeutic agents and methods described herein.

In addition, the provided demonstration that STING activation in immune cells is sufficient to elicit anti-tumor immunity suggests a novel therapeutic approach for treating a significant fraction of patients with cancers deficient in tumor cell-intrinsic STING. Hence, the translational implications of these findings are highly significant, as they provide a strong and timely rationale for combining systemic administration of STING agonists with PARP inhibitors in the treatment of BRCA-deficient breast cancers, particularly in light of two recent publications in Science reporting antitumor activities of two non-nucleotide STING agonists that can be administered systemically (PMID 32820094 and 32820126).

This disclosure, therefore, provides an important conceptual shift in the understanding of resistance to PARP inhibitors, which has mostly been described in terms of restoration of homologous recombination.

Accordingly, the present invention provides methods of improving effectiveness of PARP inhibition in a subject with cancer, of polarizing pro-tumor macrophages in a subject with cancer into anti-tumor macrophages, and of selecting a subject with cancer for treatment with a STING agonist.

A syngeneic genetically-engineered mouse (GEM) model of lung cancer driven by a mutant EGFR shows that while EGFR-mutant tumors are highly sensitive to osimertinib at early stage of tumor growth in a T cell-dependent manner, they become resistant as they progress. Therefore, the present invention, is also based, in part, on the determination that the presence of immunosuppressive tumor-associated macrophages (TAMs) render tumors resistance to osimertinib. Depletion of TAMs in these tumors rescues the efficacy of osimertinib. Reprograming TAMs with a newly developed STING agonist MSA-2 reinvigorates antitumor immunity, and leads to durable regression of resistant tumors in mice when combined with osimertinib. The results shown herein suggest that a suppressive tumor immune microenvironment can drive resistance of EGFR-mutant tumors to osimertinib. Therefore provided herein is a new strategy to overcome resistance and improve therapeutic outcomes.

Therefore, also provided herein is a method of improving effectiveness of tyrosine kinase inhibitor (TKI) inhibition in a subject with cancer, by administering to the subject an effective amount of a STING agonist conjointly with an effective amount of a tyrosine kinase inhibitor (TKI).

In certain aspects, methods of polarizing pro-tumor macrophages in a subject with cancer into anti-tumor macrophages include administering to the subject an effective amount of a STING agonist. Also provided herein are methods of preventing or reversing drug resistance in a subject with cancer, wherein the drug resistance is a result of polarizing anti-tumor macrophages into pro-tumor macrophages, comprising administering to the subject an effective amount of a STING agonist. Also provided herein is a method of improving effectiveness of a DNA synthesis inhibitor in a subject with cancer, by administering to the subject an effective amount of a STING agonist conjointly with an effective amount of a DNA synthesis inhibitor.

### I. Definitions

The articles “a” and “an” are used herein to refer to one or to more than one (i.e. to at least one) of the grammatical object of the article. By way of example, “an element” means one element or more than one element.

The term “administering” is intended to include modes and routes of administration which allow an agent to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

Unless otherwise specified herein, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CHI domains; (ii) a F(ab′)₂ fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16:778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P. et al. (1993) Proc. Natl. Acad. Sci. USA 90:6444-6448; Poljak, R. J. et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M. et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S. M. et al. (1994) Mol. Immunol. 31:1047-1058). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g., humanized, chimeric, etc.). Antibodies may also be fully human. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts. In addition, antibodies can be “humanized,” which includes antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies encompassed by the present invention may include amino acid residues not encoded by human germline immunoglobulin sequences (e.g., mutations introduced by random or site-specific mutagenesis in vitro or by somatic mutation in vivo), for example in the CDRs. The term “humanized antibody,” as used herein, also includes antibodies in which CDR sequences derived from the germline of another mammalian species, such as a mouse, have been grafted onto human framework sequences.

A “blocking” antibody is one which inhibits or reduces at least one biological activity of the antigen(s) it binds. In certain embodiments, the blocking antibodies or fragments thereof described herein substantially or completely inhibit a given biological activity of the antigen(s). Blocking antibodies are alternatively referred to herein with the prefix “anti” with respect to a target of them (e.g., anti-PARP for an antibody that binds to PARP).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

As used herein, the term “DNA synthesis inhibitor” includes, but is not limited to, two types of therapeutic agents used to inhibit DNA synthesis. The first category includes purine and pyrimidine nucleoside analogs that directly inhibit DNA polymerase activity. The second category includes DNA damaging agents including cisplatin and chlorambucil that modify the composition and structure of the nucleic acid substrate to indirectly inhibit DNA synthesis. Additional details regarding DNA synthesis inhibitors can be found in Berdis A J. Inhibiting DNA Polymerases as a Therapeutic Intervention against Cancer. Front Mol Biosci. 2017 Nov. 21; 4:78, hereby incorporated by reference in its entirety.

As used herein, an epidermal growth factor receptor tyrosine kinase (EGFR-TKI) inhibitor includes, but is not limited to, any tyrosine kinase inhibitor that inhibits the activity of, or lowers the expression levels of an EGFR peptide, or a tyrosine kinase inhibitor that blocks the activity of EGFR peptide or receptor. EGFR is found on the surface of some normal cells and is involved in cell growth. It may also be found at high levels on some types of cancer cells. An EGFR-TKI may also be called an EGFR inhibitor, epidermal growth factor receptor inhibitor, or epidermal growth factor receptor tyrosine kinase inhibitor. The TK inhibitor (e.g., EGFR-TK inhibitor) used in any method disclosed herein may be selected from afatinib, dacomitinib, osimertinib (AZD9291), rociletinib (CO-1686), olmutinib (HM61713), nazartinib (EGF816), naquotinib (ASP8273), mavelertinib (PF-0647775), almonertinib, TY-9591, gefitinib, erlotinib and AC0010.

As used herein, an EGFR activating mutation includes, but is not limited to, any activating mutation that confers sensitivity to EGFR TKIs. These mutations include any mutation that are present in the tyrosine kinase (TK) domain of the EGFR gene. Such mutations include, for example, point mutations, deletion mutations, insertion mutations, missense mutations, or frameshift mutations. Additional exemplary mutations include exon 19 deletion mutations, single-point substitution mutation L858R in exon 21, and the point mutation T790M.

Homologous recombination repair (HRR) pathway deficiency (HRD) is involved in the tumorigenesis and progression of cancers, including high-grade serous ovarian carcinoma (HGSOC) as well as in the sensitivity to platinum chemotherapy drugs. Homologous recombination (HR) comprises a series of interrelated pathways that function in the repair of DNA double-stranded breaks (DSBs) and interstrand crosslinks (ICLs). In addition, recombination provides critical support for DNA replication in the recovery of stalled or broken replication forks, contributing to tolerance of DNA damage. As used herein an “HRD cancer”, includes any cancer which exhibits impaired ability of tumor cells to repair DNA double-strand breaks (DSBs) via homologous recombination. Mutations in genes such as RAD51, PALB2, ATM, ATR, CHEK2, RAD51, and FANC can cause a HRD cancer. Conversely, the cancer may be a homologous recombination DNA repair proficiency (HRP) cancer. An HRP cancer is any cancer that retains the ability of cancer cells to successfully perform homologous recombination DNA repair (HRR).

As used herein, a PARP inhibitor includes, but is not limited to, any agent that inhibits the activity of or lowers the expression levels of a PARP peptide. PARP inhibitors, as disclosed herein, also include any agent that blocks the PARP enzyme. The PARP family has many essential functions in cellular processes, including the regulation of transcription, apoptosis and the DNA damage response. PARP1 possesses poly (ADP-ribose) activity and when activated by DNA damage, adds branched PAR chains to facilitate the recruitment of other repair proteins to promote the repair of DNA single-strand breaks. Exemplary PARP inhibitors include olaparib, rucaparib, niraparib, talazoparib, veliparib, pamiparib, CEP 9722, E7016, AG014699, MK4827, BMN-673, iniparib, and 3-aminobenzamide.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

As used herein, a tyrosine kinase inhibitor (TKI) includes any agent that inhibits the expression or activity of tyrosine kinases. Kinase inhibitors are either irreversible or reversible. The irreversible kinase inhibitors tend to covalently bind and block the ATP site resulting in irreversible inhibition. The reversible kinase inhibitors can further subdivide into four major subtypes based on the confirmation of the binding pocket as well as the DFG motif. The TK inhibitor may be type I, type II, type III, type IV, or type V. Type I inhibitors competitively bind to the ATP-binding site of active TKs. The arrangement of the DFG motif in type I inhibitors has the aspartate residue facing into the catalytic site of the kinase. Type II inhibitors bind to inactive kinases, usually at the ATP-binding site. The DFG motif in type II inhibitors protrudes outward away from the ATP-binding site. Due to the outward rotation of the DFG motif, many type II inhibitors can also exploit regions adjacent to the ATP-binding site that would otherwise be inaccessible. Type III inhibitors do not interact with the ATP-binding pocket. Type III inhibitors exclusively bind to allosteric pockets adjacent to the ATP-binding region. Type IV inhibitors bind allosteric sites far removed from the ATP-binding pocket. Type V inhibitors: refer to a proposed subset of kinase inhibitors that exhibit multiple binding modes. The TK inhibitor may be a vascular endothelial growth factor receptor (VEGF) TK inhibitor; an epidermal growth factor (EGF) receptor TK inhibitor, a platelet derived endothelial growth factor receptor (PDGF) TK inhibitor, or the TK inhibitor may be a fibroblast growth factor (FGF) receptor TK inhibitor.

As used herein, the term “inhibiting” and grammatical equivalents thereof refer decrease, limiting, and/or blocking a particular action, function, or interaction. A reduced level of a given output or parameter need not, although it may, mean an absolute absence of the output or parameter. The invention does not require, and is not limited to, methods that wholly eliminate the output or parameter. The given output or parameter can be determined using methods well-known in the art, including, without limitation, immunohistochemical, molecular biological, cell biological, clinical, and biochemical assays, as discussed herein and in the examples. The opposite terms “promoting,” “increasing,” and grammatical equivalents thereof refer to the increase in the level of a given output or parameter that is the reverse of that described for inhibition or decrease.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

As used herein, the term “nucleotide-based second messenger” refers to a second messenger having a relatively small number (e.g., one, two, or three) of nucleotides or derivatives thereof that transduces signals originating from changes in the environment or in intracellular conditions into appropriate cellular responses. It can be circular or linear. In one embodiment, the nucleotide-based second messenger is a cyclic dinucleotide which includes but is not limited to a cyclic di-purine (e.g., cyclic di-AMP, cyclic di-GMP, cyclic AMP-GMP), a cyclic pyrimidine (e.g., cyclic di-UMP or cyclic UMP-CMP), or a cyclic pruine-pyrimidine hybrid (e.g., cyclic UMP-AMP or cyclic UMP-GMP). In another embodiment, the nucleotide-based second messenger is a cyclic trinucleotide (e.g., cyclic AMP-AMP-GMP). Several bona fide nucleotide signaling pathways, (p)ppGpp, cAMP, cGMP, c-di-AMP, c-di-GMP and cGAMP, have been characterized with respect to basic pathway modules and phenotypic and physiological output (Martin-Rodriguez et al. (2017) Curr Top Med Chem 17:1928-1944). In prokaryotes cyclic di-GMP has emerged as an important and ubiquitous second messenger regulating bacterial life-style transitions relevant for biofilm formation, virulence, and many other bacterial functions (Pesavento et al. (2009) Curr Opin Microbiol 12:170-176).

The nucleotide-based second messenger may contain modified or unnatural nucleotides. The modified nucleotides can be naturally occurring modified RNA base analogs (Limbach et al. (1994) Nucleic Acids Res 22:2183-2196; Cantara et al. (2011) Nucleic Acids Res 39: D195-D201; Czerwoniec et al. (2009) Nucleic Acids Res 37: D118-D121; Grosjean et al. (1998) Modification and Editing of RNA. ASM Press, Washington DC.), including but not limited to N⁶-Methyladenosine-5′-Triphosphate, 5-Methylcytidine-5′-Triphosphate, 2′-O-Methyladenosine-5′-Triphosphate, 2′-O-Methylcytidine-5′-Triphosphate, 2′-O-Methylguanosine-5′-Triphosphate, 2′-O-Methyluridine-5′-Triphosphate, Pseudouridine-5′-Triphosphate, Inosine-5′-Triphosphate, 2′-O-Methylinosine-5′-Triphosphate, 5-Methyluridine-5′-Triphosphate, 4-Thiouridine-5′-Triphosphate, 2-Thiouridine-5′-Triphosphate, 5,6-Dihydrouridine-5′-Triphosphate, 2-Thiocytidine-5′-Triphosphate, 2′-O-Methylpseudouridine-5′-Triphosphate, N¹-Methyladenosine-5′-Triphosphate, 2′-O-Methyl-5-methyluridine-5′-Triphosphate, N⁴-Methylcytidine-5′-Triphosphate, N¹-Methylpseudouridine-5′-Triphosphate, 5,6-Dihydro-5-Methyluridine-5′-Triphosphate, 5-Formylcytidine-5′-Triphosphate, 5-Hydroxymethylcytidine-5′-Triphosphate, 5-Hydroxycytidine-5′-Triphosphate, 5-Hydroxyuridine-5′-Triphosphate, 5-Methoxyuridine-5′-Triphosphate, and 5-Carboxymethylesteruridine-5′-Triphosphate.

Unnatural nucleotides include but are not limited to 2′ Fluoro and 2′ O-Methyl NTPs, for example, 2′-Amino-2′-deoxyadenosine-5′-Triphosphate, 2′-Amino-2′-deoxycytidine-5′-Triphosphate, 2′-Amino-2′-deoxyuridine-5′-Triphosphate, 2′-Azido-2′-deoxyadenosine-5′-Triphosphate, 2′-Azido-2′-deoxycytidine-5′-Triphosphate, 2′-Azido-2′-deoxyguanosine-5′-Triphosphate, 2′-Azido-2′-deoxyuridine-5′-Triphosphate, 2′-Fluoro-2′-deoxyadenosine-5′-Triphosphate, 2′-Fluoro-2′-deoxycytidine-5′-Triphosphate, 2′-Fluoro-2′-deoxyguanosine-5′-Triphosphate, 2′-Fluoro-2′-deoxyuridine-5′-Triphosphate, 2′-Fluorothymidine-5′-Triphosphate, 2′-Fluoro-2′-deoxyadenosine-5′-Triphosphate, 2′-Fluoro-2′-deoxycytidine-5′-Triphosphate, 2′-Fluoro-2′-deoxyguanosine-5′-Triphosphate, 2′-Fluoro-2′-deoxyuridine-5′-Triphosphate, 2′-Fluorothymidine-5′-Triphosphate, 2′-Fluoro-2′-deoxyadenosine-5′-Triphosphate, 2′-Fluoro-2′-deoxycytidine-5′-Triphosphate, 2′-Fluoro-2′-deoxyguanosine-5′-Triphosphate, 2′-Fluoro-2′-deoxyuridine-5′-Triphosphate, 2′-O-Methyladenosine-5′-Triphosphate, 2′-O-Methylcytidine-5′-Triphosphate, 2′-O-Methylguanosine-5′-Triphosphate, 2′-O-Methyluridine-5′-Triphosphate, Pseudouridine-5′-Triphosphate, 2′-O-Methylinosine-5′-Triphosphate, 2′-Amino-2′-deoxycytidine-5′-Triphosphate, 2′-Amino-2′-deoxyuridine-5′-Triphosphate, 2′-Azido-2′-deoxycytidine-5′-Triphosphate, 2′-Azido-2′-deoxyuridine-5′-Triphosphate, 2′-O-Methylpseudouridine-5′-Triphosphate, 2′-O-Methyl-5-methyluridine-5′-Triphosphate, 2′-Azido-2′-deoxyadenosine-5′-Triphosphate, 2′-Amino-2′-deoxyadenosine-5′-Triphosphate, 2′-Fluoro-thymidine-5′-Triphosphate, 2′-Azido-2′-deoxyguanosine-5′-Triphosphate, N⁴-Methylcytidine-5′-Triphosphate, 2′-O-Methyladenosine-5′-Triphosphate, 2′-O-Methylcytidine-5′-Triphosphate, 2′-O-Methylguanosine-5′-Triphosphate, 2′-O-Methyluridine-5′-Triphosphate, 2′-Amino-2′-deoxyadenosine-5′-Triphosphate, 2′-Amino-2′-deoxycytidine-5′-Triphosphate, 2′-Amino-2′-deoxyuridine-5′-Triphosphate, Araadenosine-5′-Triphosphate, Aracytidine-5′-Triphosphate, Araguanosine-5′-Triphosphate, Arauridine-5′-Triphosphate, 2′-Azido-2′-deoxyadenosine-5′-Triphosphate, 2′-Azido-2′-deoxycytidine-5′-Triphosphate, 2′-Azido-2′-deoxyguanosine-5′-Triphosphate,2′-Azido-2′-deoxyuridine-5′-Triphosphate,2′-Fluoro-2′-deoxyadenosine-5′-Triphosphate, 2′-Fluoro-2′-deoxycytidine-5′-Triphosphate, 2′-Fluoro-2′-deoxyguanosine-5′-Triphosphate, 2′-Fluoro-2′-deoxyuridine-5′-Triphosphate, 2′-Fluorothymidine-5′-Triphosphate,2′-O-Methyladenosine-5′-Triphosphate, 2′-O-Methylcytidine-5′-Triphosphate, 2′-O-Methylguanosine-5′-Triphosphate, 2′-O-Methyluridine-5′-Triphosphate,2′-Fluoro-2′-deoxyadenosine-5′-Triphosphate,2′-Fluoro-2′-deoxycytidine-5′-Triphosphate, 2′-Fluoro-2′-deoxyguanosine-5′-Triphosphate, 2′-Fluoro-2′-deoxyuridine-5′-Triphosphate, 2′-Fluorothymidine-5′-Triphosphate, 2′-O-Methyladenosine-5′-Triphosphate, 2′-O-Methylcytidine-5′-Triphosphate,2′-O-Methylguanosine-5′-Triphosphate, and 2′-O-Methyluridine-5′-Triphosphate.

As used herein, the term “domain” means a functional portion, segment or region of a protein, or polypeptide. “Interaction domain” refers specifically to a portion, segment or region of a protein, polypeptide or protein fragment that is responsible for the physical affinity of that protein, protein fragment or isolated domain for another protein, protein fragment or isolated domain.

If not stated otherwise, the term “compound” as used herein are include but are not limited to peptides, nucleic acids, carbohydrates, natural product extract libraries, organic molecules, preferentially small organic molecules, inorganic molecules, including but not limited to chemicals, metals and organometallic molecules.

The terms “derivatives”, “analogs” or “variants” as used herein include, but are not limited, to molecules comprising regions that are substantially homologous to the modified CD-NTase polypeptide, in various embodiments, by at least 30%, 40%, 50%, 60%, 70%, 80%, 90%, 95% or 99% identity over an amino acid sequence of identical size or when compared to an aligned sequence in which the alignment is done by a computer homology program known in the art, or whose encoding nucleic acid is capable of hybridizing to a sequence encoding the component protein under stringent, moderately stringent, or nonstringent conditions. It means a protein which is the outcome of a modification of the naturally occurring protein, by amino acid substitutions, deletions and additions, respectively, which derivatives still exhibit the biological function of the naturally occurring protein although not necessarily to the same degree. The biological function of such proteins can e.g. be examined by suitable available in vitro assays as provided in the invention.

The term “functionally active” as used herein refers to a polypeptide, namely a fragment or derivative, having structural, regulatory, or biochemical functions of the protein according to the embodiment of which this polypeptide, namely fragment or derivative is related to.

The term “activity” when used in connection with proteins or molecular complexes means any physiological or biochemical activities displayed by or associated with a particular protein or molecular complex including but not limited to activities exhibited in biological processes and cellular functions, ability to interact with or bind another molecule or a moiety thereof, binding affinity or specificity to certain molecules, in vitro or in vivo stability (e.g., protein degradation rate, or in the case of molecular complexes ability to maintain the form of molecular complex), antigenicity and immunogenecity, enzymatic activities, etc. Such activities may be detected or assayed by any of a variety of suitable methods as will be apparent to skilled artisans.

As used herein, the term “interaction antagonist” means a compound that interferes with, blocks, disrupts or destabilizes a protein-protein interaction or a protein-DNA interaction; blocks or interferes with the formation of a molecular complex, or destabilizes, disrupts or dissociates an existing molecular complex.

The term “interaction agonist” as used herein means a compound that triggers, initiates, propagates, nucleates, or otherwise enhances the formation of a protein-protein interaction or a protein-DNA interaction; triggers, initiates, propagates, nucleates, or otherwise enhances the formation of a molecular complex; or stabilizes an existing molecular complex.

The term “STING” or “stimulator of interferon genes”, also known as transmembrane protein 173 (TMEM173), refers to a five transmembrane protein that functions as a major regulator of the innate immune response to viral and bacterial infections. STING is a cytosolic receptor that senses both exogenous and endogenous cytosolic cyclic dinucleotides (CDNs), activating TBK1/IRF3 (interferon regulatory factor 3), NF-κB (nuclear factor κB), and STAT6 (signal transducer and activator of transcription 6) signaling pathways to induce robust type I interferon and proinflammatory cytokine responses. The term “STING” is intended to include fragments, variants (e.g., allelic variants) and derivatives thereof. Representative human STING cDNA and human STING protein sequences are well-known in the art and are publicly available from the National Center for Biotechnology Information (NCBI). Human STING isoforms include the longer isoform 1 (NM_198282.3 and NP_938023.1), and the shorter isoform 2 (NM_001301738.1 and NP_001288667.1; which has a shorter 5′ UTR and lacks an exon in the 3′ coding region which results in a shorter and distinct C-terminus compared to variant 1). Nucleic acid and polypeptide sequences of STING orthologs in organisms other than humans are well-known and include, for example, chimpanzee STING (XM_016953921.1 and XP_016809410.1; XM_009449784.2 and XP_009448059.1; XM_001135484.3 and XP_001135484.1), monkey STING (XM_015141010.1 and XP_014996496.1), dog STING (XM_022408269.1 and XP_022263977.1; XM_005617260.3 and XP_005617317.1; XM_022408249.1 and XP_022263957.1; XM_005617262.3 and XP_005617319.1; XM_005617258.3 and XP_005617315.1; XM_022408253.1 and XP_022263961.1; XM_005617257.3 and XP_005617314.1; XM_022408240.1 and XP_022263948.1; XM_005617259.3 and XP_005617316.1; XM_022408259.1 and XP_022263967.1; XM_022408265.1 and XP_022263973.1), cattle STING (NM_001046357.2 and NP_001039822.1), mouse STING (NM_001289591.1 and NP_001276520.1; NM_001289592.1 and NP_001276521.1; NM_028261.1 and NP_082537.1), and rat STING (NM_001109122.1 and NP_001102592.1).

STING agonists have been shown as useful therapies to treat cancer. Agonists of STING well-known in the art and include, for example, MK-1454, STING agonist-1 (MedChem Express Cat No. HY-19711), cyclic dinucleotides (CDNs) such as cyclic di-AMP (c-di-AMP), cyclic-di-GMP (c-di-GMP), cGMP-AMP (2′3′cGAMP or 3′3′cGAMP), or 10-carboxymethyl-9-acridanone (CMA) (Ohkuri et al. (2015) Oncoimmunology 4 (4): e999523), rationally designed synthetic CDN derivative molecules (Fu et al. (2015) Sci Transl Med. 7 (283): 283ra52. doi: 10.1126/scitranslmed.aaa4306), and 5,6-dimethyl-xanthenone-4-acetic acid (DMXAA) (Corrales et al. (2015) Cell Rep. 11 (7): 1018-1030). These agonists bind to and activate STING, leading to a potent type I IFN response. On the other hand, targeting the cGAS-STING pathway with small molecule inhibitors would benefit for the treatment of severe debilitating diseases such as inflammatory and autoimmune diseases associated with excessive type I IFNs production due to aberrant DNA sensing and signaling. STING inhibitors are also known and include, for example, CCCP (MedChem Express, Cat No. HY-100941) and 2-bromopalmitate (Tao et al. (2016) IUBMB Life. 68 (11): 858-870). It is to be noted that the term can further be used to refer to any combination of features described herein regarding STING molecules. For example, any combination of sequence composition, percentage identify, sequence length, domain structure, functional activity, etc. can be used to describe a STING molecule encompassed by the present invention.

The term “STING pathway” or “cGAS-STING pathway” refers to a STING-regulated innate immune pathway, which mediates cytosolic DNA-induced signalling events. Cytosolic DNA binds to and activates cGAS, which catalyzes the synthesis of 2′3′-cGAMP from ATP and GTP. 2′3′-cGAMP binds to the ER adaptor STING, which traffics to the ER-Golgi intermediate compartment (ERGIC) and the Golgi apparatus. STING then activates IKK and TBK1. TBK 1 phosphorylates STING, which in turn recruits IRF3 for phosphorylation by TBK1. Phosphorylated IRF3 dimerizes and then enters the nucleus, where it functions with NF-kB to turn on the expression of type I interferons and other immunomodulatory molecules. The cGAS-STING pathway not only mediates protective immune defense against infection by a large variety of DNA-containing pathogens but also detects tumor-derived DNA and generates intrinsic antitumor immunity. However, aberrant activation of the cGAS-STING pathway by self DNA can also lead to autoimmune and inflammatory disease.

The term “cGAS” or “Cyclic GMP-AMP Synthase”, also known as Mab-21 Domain-Containing Protein 1, refers to nucleotidyltransferase that catalyzes the formation of cyclic GMP-AMP (cGAMP) from ATP and GTP (Sun et al. (2013) Science 339:786-791; Krazusch et al. (2013) Cell Rep 3:1362-1368; Civril et al. (2013) Nature 498:332-227; Ablasser et al. (2013) Nature 503:530-534; Kranzusch et al. (2014) (ell 158:1011-1021). cGAS involves both the formation of a 2,5 phosphodiester linkage at the GpA step and the formation of a 3,5 phosphodiester linkage at the ApG step, producing c[G(2,5)pA(3,5)p] (Tao et al. (2017) J Immunol 198:3627-3636; Lee et al. (2017) FEBS Lett. 591:954-961). cGAS acts as a key cytosolic DNA sensor, the presence of double-stranded DNA (dsDNA) in the cytoplasm being a danger signal that triggers the immune responses (Tao et al. (2017) J Immunol 198:3627-3636). cGAS binds cytosolic DNA directly, leading to activation and synthesis of cGAMP, a second messenger that binds to and activates TMEM173/STING, thereby triggering type-I interferon production (Tao et al. (2017) J Immunol 198:3627-3636; Wang et al. (2017) Immunity 46:393-404). cGAS has antiviral activity by sensing the presence of dsDNA from DNA viruses in the cytoplasm (Tao et al. (2017) J Immunol 198:3627-3636). cGAS also acts as an innate immune sensor of infection by retroviruses, such as HIV-1, by detecting the presence of reverse-transcribed DNA in the cytosol (Gao et al. (2013) Science 341:903-906). The detection of retroviral reverse-transcribed DNA in the cytosol may be indirect and be mediated via interaction with PQBP1, which directly binds reverse-transcribed retroviral DNA (Yoh et al. (2015) Cell 161:1293-1305). cGAS also detects the presence of DNA from bacteria, such as M.tuberculosis (Wassermann et al. (2015) Cell Host Microbe 17:799-810). cGAMP can be transferred from producing cells to neighboring cells through gap junctions, leading to promote TMEM173/STING activation and convey immune response to connecting cells (Ablasser et al. (2013) Nature 503:530-534). cGAMP can also be transferred between cells by virtue of packaging within viral particles contributing to IFN-induction in newly infected cells in a cGAS-independent but TMEM173/STING-dependent manner (Gentili et al. (2015) Science 349:1232-1236). In addition to antiviral activity, cGAS is also involved in the response to cellular stresses, such as senescence, DNA damage or genome instability (Mackenzie et al. (2017) Nature 548:461-465; Harding et al. (2017) Nature 548:466-470). cGAS acts as a regulator of cellular senescence by binding to cytosolic chromatin fragments that are present in senescent cells, leading to trigger type-I interferon production via TMEM173/STING and promote cellular senescence. cGAS is also involved in the inflammatory response to genome instability and double-stranded DNA breaks. cGAS acts by localizing to micronuclei arising from genome instability (PubMed: 28738408; Harding et al. (2017) Nature 548:466-470). Micronuclei, which is frequently found in cancer cells, is consist of chromatin surrounded by its own nuclear membrane. Following breakdown of the micronuclear envelope, a process associated with chromothripsis, MB21D1/cGAS binds self-DNA exposed to the cytosol, leading to cGAMP synthesis and subsequent activation of TMEM173/STING and type-I interferon production (Mackenzie et al. (2017) Nature 548:461-465; Harding et al. (2017) Nature 548:466-470). In one embodiment, human cGAS has 522 amino acids with a molecular mass of 58814 Da. cGAS is a monomer in the absence of DNA and when bound to dsDNA (Tao et al. (2017).J Immunol 198:3627-3636). cGAS interacts with PQBP1 (via WW domain) (Yoh et al. (2015) Cell 161:1293-1305). cGAS also interacts with TRIM14 and this interaction stabilizes cGAS/MB21D1 and promotes type I interferon production (Chen et al. (2016) Mol Cell 64:105-119). cGAS also interacts with herpes virus 8/HHV-8 protein ORF52, and this interaction inhibits cGAS enzymatic activity.

The term “cGAS” is intended to include fragments, variants (e.g., allelic variants) and derivatives thereof. Representative human cGAS cDNA and human cGAS protein sequences are well-known in the art and are publicly available from the National Center for Biotechnology Information (NCBI). Human cGAS isoforms include the protein (NP_612450.2) encoded by the transcript (NM_138441.2). Nucleic acid and polypeptide sequences of cGAS orthologs in organisms other than humans are well-known and include, for example, chimpanzee cGAS (XM_009451553.3 and XP_009449828.1; and XM_009451552.3 and XP_009449827.1), Monkey cGAS (NM_001318175.1 and NP_001305104.1), cattle cGAS (XM_024996918.1 and XP_024852686.1, XM_005210662.4 and XP_005210719.2, and XM_002690020.6 and XP_002690066.3), mouse cGAS (NM_173386.5 and NP_775562.2), rat cGAS (XM_006243439.3 and XP_006243501.2), and chicken cGAS (XM_419881.6 and XP_419881.4).

Anti-cGAS antibodies suitable for detecting cGAS protein are well-known in the art and include, for example, antibody TA340293 (Origene), antibodies NBP1-86761 and NBP1-70755 (Novus Biologicals, Littleton, CO), antibodies ab224144 and ab 176177 (AbCam, Cambridge, MA), antibody 26-664 (ProSci), etc. In addition, reagents are well-known for detecting cGAS. Multiple clinical tests of cGAS are available in NIH Genetic Testing Registry (GTR®) (e.g., GTR Test ID: GTR000540854.2, offered by Fulgent Clinical Diagnostics Lab (Temple City, CA)). Moreover, mutilple siRNA, shRNA, CRISPR constructs for reducing cGAS expression can be found in the commercial product lists of the above-referenced companies, such as siRNA product #sc-95512 from Santa Cruz Biotechnology, RNAi products SR314484 and TL305813V, and CRISPR product KN212386 (Origene), and multiple CRISPR products from GenScript (Piscataway, NJ). It is to be noted that the term can further be used to refer to any combination of features described herein regarding cGAS molecules. For example, any combination of sequence composition, percentage identify, sequence length, domain structure, functional activity, etc. can be used to describe a cGAS molecule encompassed by the present invention.

An “antagonist” is one which attenuates, decreases, or inhibits at least one biological activity of at least one protein, such as a receptor. In certain embodiments, the antagonist substantially or completely attenuates or inhibits a given biological activity of at least one protein described herein.

The term “mode of administration” includes any approach of contacting a desired target (e.g., cells, a subject) with a desired agent (e.g., a therapeutic agent). The route of administration, as used herein, is a particular form of the mode of administration, and it specifically covers the routes by which agents are administered to a subject or by which biophysical agents are contacted with a biological material.

The term “subject” refers to any healthy animal, mammal or human, or any animal, mammal or human afflicted with a cancer. The term “subject” is interchangeable with “patient.”

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically-effective amount” and “effective amount” as used herein means that amount of a compound, material, or composition comprising a compound encompassed by the present invention which is effective for producing some desired therapeutic effect in at least a sub-population of cells in an animal at a reasonable benefit/risk ratio applicable to any medical treatment. Toxicity and therapeutic efficacy of subject compounds can be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to administration of a suitable control agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to administration of a suitable control agent. Also, Similarly, the IC50 (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to administration of a suitable control agent.

### II. Subjects

In some embodiments, the subject is a mammal (e.g., mouse, rat, primate, non-human mammal, domestic animal, such as a dog, cat, cow, horse, and the like), and is preferably a human. In other embodiments, the subject is an animal model of a cancer. In addition, cells can be used according to the methods described herein, whether in vitro, ex vivo, or in vivo, such as cells from such subjects.

In some embodiments encompassed by the methods of the present invention, the subject has not undergone treatment, such as via PARP inhibitors. In other embodiments, the subject has undergone treatment, such as with PARP inhibitors. In some embodiments encompassed by the methods of the present invention, the subject has not undergone treatment, such as treatment with TK inhibitors. In some embodiments, the subject has undergone treatment, such as with TK inhibitors. In some embodiments encompassed by the methods of the present invention, the subject has not undergone treatment, such as treatment with DNA synthesis inhibitors. In some embodiments, the subject has undergone treatment, such as with DNA synthesis inhibitors. In some embodiments, subjects are those that have a tumor with an M2 enrichment score higher than 0.27, or any other enrichment score disclosed herein.

This mentioned M2 enrichment score, also recited in some of the claims, in some embodiments, is according to the TCGA data analysis of cancer types with enrichment score of M2-like TAMs in line with the M2 signature data from Pan et al., 2017, Immunity 47, 284 297. The yellow dotted line in the first figure of this article is an average enrichment score of M2 gene signature (0.27), and the abbreviations used in the figure are ACC: Adrenocortical carcinoma; BLCA: Bladder Urothelial Carcinoma; BRCA: Breast invasive carcinoma; CESC: Cervical squamous cell carcinoma and endocervical adenocarcinoma; COAD: Colon adenocarcinoma; DLBC: Lymphoid Neoplasm Diffuse Large B-cell Lymphoma; GBM: Glioblastoma multiforme; HNSC: Head and Neck squamous cell carcinoma; KICH: Kidney Chromophobe; KIRC: Kidney renal clear cell carcinoma; KIRP: Kidney renal papillary cell carcinoma; LAML: Acute Myeloid Leukemia; LGG: Brain Lower Grade Glioma; LIHC: Liver hepatocellular carcinoma; LUAD: Lung adenocarcinoma; LUSC: Lung squamous cell carcinoma; OV: Ovarian serous cystadenocarcinoma; PRAD: Prostate adenocarcinoma; READ: Rectum adenocarcinoma; SKCM: Skin Cutaneous Melanoma; STAD: Stomach adenocarcinoma; THCA: Thyroid carcinoma; UCEC: Uterine Corpus Endometrial Carcinoma; and UCS: Uterine Carcinosarcoma.

Accordingly, in some embodiments, the subject has head and neck squamous cell carcinoma (HNSC); lung cancer, such as non-small cell lung cancer (NSCLC) or lung squamous cell carcinoma (LUSC); liver cancer, such as hepatocellular carcinoma (HCC); colon cancer; prostate cancer; pancreatic cancer; skin cutaneous melanoma (SKCM); glioblastoma multiforme (GBM); breast invasive carcinoma (BRCA); lung adenocarcinoma (LUAD); kidney renal clear cell carcinoma (KIRC); cervical squamous cell carcinoma and endocervical adenocarcinoma (CESC); diffuse large B-cell lymphoma (DLBC); stomach adenocarcinoma (STAD), or ovarian cancer, such as high-grade serous ovarian carcinoma (HGSOC) ovarian cancer, such as high-grade serous ovarian carcinoma (HGSOC) or homologous recombination proficient (HRP) ovarian cancer; or any homologous recombination proficient (HRP) cancer. The cancer may be any homologous recombination deficient (HRD) cancer, such as HRD ovarian cancer. The cancer may be an HRD cancer or tumor that comprises a mutation in a RAD51, PALB2, ATM, ATR, CHEK2, RAD51, or FANC gene. The cancer may be any cancer that comprises a genetic mutation which upregulates STAT3 signaling and/or polarizes tumor associated macrophages to M2-like macrophages (e.g., a cancer with a mutation in the KRAS gene, such as the KRAS^{G12D} mutation).

In certain embodiments, the subject has breast cancer carrying a BRCA mutation (e.g., advanced breast cancer carrying germline BRCA1/2 mutations). In certain embodiments, the subject has BRCA naïve breast or ovarian cancer.

In certain embodiments, the subject has a cancer (e.g., a lung cancer) carrying an EGFR mutation. The EGFR mutation may be an activating mutation, or any mutation that confers resistance to TKIs.

In some embodiments, the subject has a cancer that includes a sub-population of tumors with an M2 enrichment score higher than 0.27 (e.g., even if the cancer itself is one of those with an M2 enrichment score below 0.27). In some embodiments, the subject has a cancer that includes a sub-population of tumors with an M2 enrichment score higher than 0.15 (e.g., higher than 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97 0.98, 0.99, or higher) (e.g., even if the cancer itself is one of those with an M2 enrichment score below 0.15). In some embodiments, the subject has a cancer that includes a tumor that has acquired an M2 enrichment score higher than 0.27 over a treatment course (e.g., even if the cancer itself is one of those with an M2 enrichment score below 0.27). In some embodiments, the subject has a cancer that includes a tumor that has acquired an M2 enrichment score higher than 0.15 over a treatment course (e.g., higher than 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97 0.98, 0.99, or higher) (e.g., even if the cancer itself is one of those with an M2 enrichment score below 0.15). In some embodiments, the subject has a deficiency in activating STING signaling in intra-tumoral dendritic cells.

### III. Therapeutic Agents

In some embodiments, the agents used are therapeutic agents that are STING agonists. These agents can activate STING signaling, for example in macrophages (e.g., those that are external to a tumor).

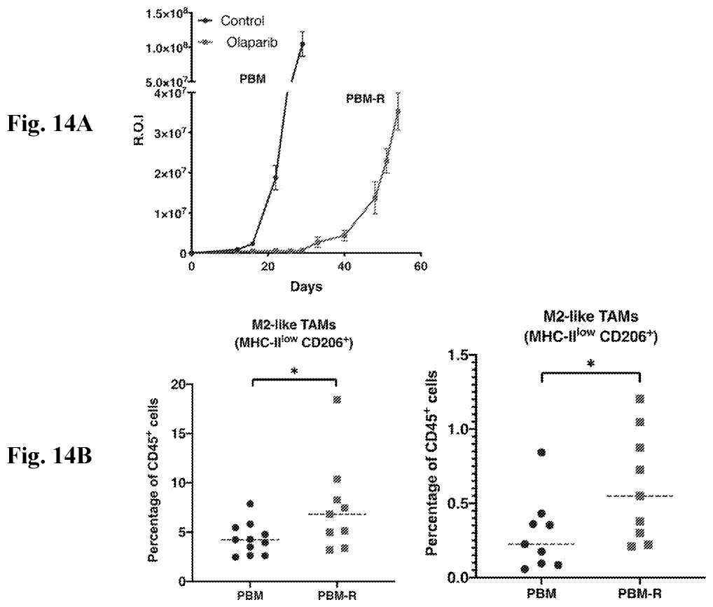

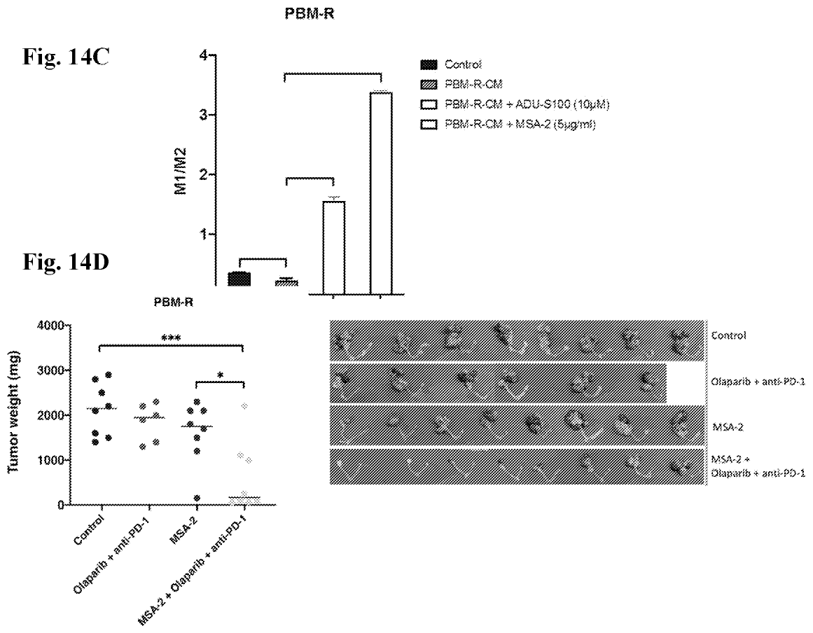

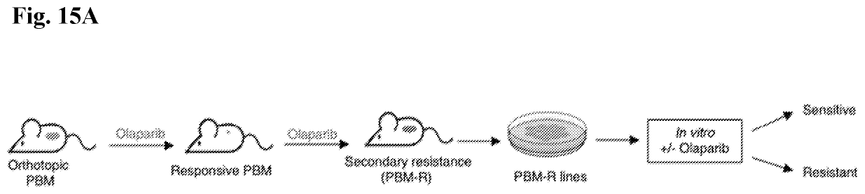

In some embodiments, the STING agonist includes is a modified nucleotide STING agonist. In some embodiments, the STING agonist is selected from DMXAA, MSA-2, SR-717, FAA, CMA, α-Mangostin, BNBC, DSDP, diABZI, bicyclic benzamides, and benzothiophenes. In some embodiments, any suitable STING agonist can be used (e.g., a STING agonist that is sufficiently suitable for systemic administration, such as a STING agonist that is not a natural ligand of STING). Various further details about STING agonists in general can be found in the literature, for example at Chin et al., Antitumor activity of a systemic STING-activating non-nucleotide cGAMP mimetic, Science 369:993 (2020) and Pan et al., An orally available non-nucleotide STING agonist with antitumor activity, Science 369:935 (2020). Data related to DMXAA can be found in Example 1, and data related to MSA-2 can be found in FIG. 14A-FIG. 14D.

In certain embodiments, the STING agonist can be conjointly administered (e.g., separately or together, at different times or at the same time) with another therapeutic agent. N particular, the STING agonist can be conjointly administered with a PARP inhibitor. The PARP inhibitor, in some embodiments, is selected from olaparib, rucaparib, niraparib, talazoparib, veliparib, pamiparib, CEP 9722, E7016, AG014699, MK4827, BMN-673, iniparib, and 3-aminobenzamide. In some embodiments, administering conjointly comprises administering the STING agonist before the PARP inhibitor. In some embodiments, administering conjointly comprises administering the STING agonist concurrently with the PARP inhibitor.

In certain embodiments, the STING agonist can be conjointly administered (e.g., separately or together, at different times or at the same time) with another therapeutic agent. In particular, the STING agonist can be conjointly administered with a TKI (e.g., EGFR-TKI or any other TKI disclosed herein). The TKI (e.g., EGFR-TKI or any other TKI disclosed herein), in some embodiments, is selected from afatinib, dacomitinib, osimertinib (AZD9291), rociletinib (CO-1686), olmutinib (HM61713), nazartinib (EGF816), naquotinib (ASP8273), mavelertinib (PF-0647775), almonertinib, TY-9591, gefitinib, erlotinib and AC0010. In some embodiments, administering conjointly comprises administering the STING agonist before the TKI (e.g., an EGFR-TKI or any other TKI disclosed herein). In some embodiments, administering conjointly comprises administering the STING agonist concurrently with the TKI (e.g., EGFR-TKI or any other TKI disclosed herein).

In certain embodiments, the STING agonist can be conjointly administered (e.g., separately or together, at different times or at the same time) with another therapeutic agent. In particular, the STING agonist can be conjointly administered with a TK inhibitor. In some embodiments, administering conjointly comprises administering the STING agonist before the TK inhibitor. In some embodiments, administering conjointly comprises administering the STING agonist concurrently with the TK inhibitor. The TK inhibitor may be a vascular endothelial growth factor receptor (VEGF) TK inhibitor; an epidermal growth factor (EGF) receptor TK inhibitor, a platelet derived endothelial growth factor receptor (PDGF) TK inhibitor, or the TK inhibitor may be a fibroblast growth factor (FGF) receptor TK inhibitor. The TK inhibitor may be, for example, axitinib, dasatinib, erlotinib, imatinib, nilotinib, pazopanib, sorafenib, bosutinib, avapritinib, capmatinib, pemigatinib, ripretinib, selpercatinib, selumetinib, tucatinib, entrectinib erdafitinib, fedratinib, pexidartinib, tenosynovial, upadacitinib, zanubrutinib, baricitinib, binimetinib, dacomitinib, fostamatinib, gilteritinib, larotrectinib, lorlatinib, acalabrutinib, brigatinib, midostaurin, neratinib, alectinib, cobimetinib, lenvatinib, osimertinib, ceritinib, nintedanib, afatinib, ibrutinib, trametinib, axitinib, bosutinib, cabozantinib, ponatinib, regorafenib, tofacitinib, crizotinib, ruxolitinib, vandetanib, pazopanib, lapatinib, gefitinibor, or sunitinib.

In certain embodiments, the STING agonist can be conjointly administered (e.g., separately or together, at different times or at the same time) with another therapeutic agent. In particular, the STING agonist can be conjointly administered with a DNA synthesis inhibitor. In some embodiments, administering conjointly comprises administering the STING agonist before the DNA synthesis inhibitor. In some embodiments, administering conjointly comprises administering the STING agonist concurrently with the DNA synthesis inhibitor. Exemplary DNA synthesis inhibitors include, but are not limited to, a nucleoside analog, such as gemcitabine, sapacitabine, a cytidine analog, cytarabine, tezacitabine, troxacitabine, DMDC, CNDAC, ECyD, clofarabine, or decitabine.

Additional therapeutic agents for a combination therapy include radiation therapy, chemotherapy (e.g., with paclitaxel, a platinum-based drug (e.g., cisplatin, oxaliplatin), an inhibitor of topoisomerase activity like topoisomerase II (e.g., etoposide), a DNA intercalator (e.g., doxorubicin), and/or a DNA alkylating agent (e.g., temozolomide)), or a DNA damage response (DDR)-targeting agent (e.g., ATMi, ATRi, CHK1/2i, or Weeli). Temozolomide is the only FDA-approved first line therapy for glioblastoma (GMB), which is a high M2 tumor, yet only provides progress-free survival (PFS) without overall survival (OS) (see, e.g., Fernandes et al. (2017) Current Standards of Care in Glioblastoma Therapy, Chapter 11 of Glioblastoma, De Vleeschouwer S., Ed., Codon Publications (Brisbane, Australia) (2017)).

### IV. Therapeutic Methods

One aspect encompassed by the present invention pertains to methods of treating a cancer, for example by improving effectiveness of PARP inhibition in a subject with cancer. Such methods, in some embodiments, include administering to the subject an effective amount of a STING agonist conjointly with an effective amount of a PARP inhibitor.

One aspect encompassed by the present invention pertains to methods of treating a cancer, for example by improving effectiveness of TK (e.g., EGFR-TKI) inhibition in a subject with cancer. Such methods, in some embodiments, include administering to the subject an effective amount of a STING agonist conjointly with an effective amount of a TK (e.g., EGFR-TKI) inhibitor.

Another aspect encompassed by the present invention pertains to methods of treating a cancer, for example by improving effectiveness of DNA synthesis inhibition in a subject with cancer. Such methods, in some embodiments, include administering to the subject an effective amount of a STING agonist conjointly with an effective amount of a DNA synthesis inhibitor.

In some embodiments, administering comprises a systemic delivery of the STING agonist (e.g., oral, intravenous, or intraperitoneal).

An aspect encompassed by the present invention pertains to methods of polarizing pro-tumor macrophages in a subject with cancer into anti-tumor macrophages, comprising administering to the subject an effective amount of a STING agonist. In some such aspects, the STING agonist activates STING signaling in macrophages. In some such aspects, the STING agonist does not activate STING signaling in intra-tumoral dendritic cells. In some embodiments of these aspects, the pro-tumor macrophages are M2-like. In some embodiments of these aspects, the anti-tumor macrophages are M1-like.

In some embodiments, the PARP inhibitor is administered at a suitable dosage (e.g., at least 5, 10, 15, 20, 25, 30, 35, 40, 45, 50, 55, 60, 65, 70, 75, 80, 85, 90, 95,100 mg/kg body weight per day, or any other value or range between these values). The suitable dosage may be administered twice a day, per day, twice a week, once a week, three times a month, twice a month or monthly. In some embodiments, the PARP inhibitor is administered multiple times, e.g., at least 2-3 times, at least four times, at least five times, at least six times, at least seven times, or at least ten times.

In some embodiments, the TK inhibitor is administered at a suitable dosage (e.g., at least 5, 10, 15, 20, 25, 30, 35, 40, 45, 50, 55, 60, 65, 70, 75, 80, 85, 90, 95, 100 mg/kg body weight per day, or any other value or range between these values). The suitable dosage may be administered twice a day, per day, twice a week, once a week, three times a month, twice a month or monthly. In some embodiments, the TK inhibitor is administered multiple times, e.g., at least 2-3 times, at least four times, at least five times, at least six times, at least seven times, or at least ten times.

In some embodiments, the DNA synthesis inhibitor is administered at a suitable dosage (e.g., at least 100 mg/m2/wk, 250 mg/m2/wk, 500 mg/m2/wk, 750 mg/m2/wk, 1,000 mg/m2/wk, 1,500 mg/m2/wk, 1,750 mg/m2/wk, 2,000 mg/m2/wk, 2,200 mg/m2/wk, or any other value or range between these values). The suitable dosage may be administered twice a day, per day, twice a week, once a week, three times a month, twice a month or monthly. In some embodiments, the DNA synthesis inhibitor is administered multiple times, e.g., at least 2-3 times, at least four times, at least five times, at least six times, at least seven times, or at least ten times.

In some embodiments, the STING agonist is administered at a suitable dosage (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 mg/kg body weight per week, or any other value or range between these values). The suitable dosage may be administered twice a day, per day, twice a week, once a week, three times a month, twice a month or monthly. In some embodiments, the STING agonist is administered multiple times, e.g., at least 2-3 times, at least four times, at least five times, at least six times, at least seven times, or at least ten times.

As disclosed herein, the STING agonist may be administered conjointly with a PARP inhibitor. Also disclosed herein, the STING agonist may be administered conjointly with a TK inhibitor.

### V. Clinical Efficacy

Clinical efficacy can be measured by any method known in the art. For example, the benefit from a therapy with STING agonist, alone or in combination with another agent, such as a PARP inhibitor, TK inhibitor, or a DNA synthesis inhibitor, relates to progression free survival. As another example, the benefit from a STING agonist can relate to tumor volume, which can be measured via suitable methods.

The benefit from using agents encompassed by the present invention can be determined by measuring the level of cytotoxicity in a biological material. The benefit from using agents encompassed by the present invention can be assessed by measuring transcription profiles, viability curves, microscopic images, biosynthetic activity levels, redox levels, and the like. The benefit from using agents encompassed by the present invention can also be determined by measuring the amount of side effects from the STING agonist treatment.

In some embodiments, clinical efficacy of the therapeutic treatments described herein can be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular therapeutic regimen is at least 25%, 30%, 35%, 40%, 45%, 50%, 55%, 60%, 65%, 70%, 75%, 80%, 85%, or more.

Additional criteria for evaluating the response to therapies are related to “survival,” which includes all of the following: survival until mortality, also known as overall survival (wherein said mortality can be either irrespective of cause or tumor related); “recurrence-free survival” (wherein the term recurrence shall include both localized and distant recurrence); disease free survival. The length of said survival can be calculated by reference to a defined start point (e.g., time of diagnosis or start of treatment) and end point (e.g., death, recurrence). In addition, criteria for efficacy of treatment can be expanded to include response to therapy, probability of survival, and probability of recurrence.

For example, in order to determine appropriate threshold values, a particular STING agonist therapeutic regimen can be administered to a population of subjects and the outcome can be correlated to biomarker measurements that were determined prior to administration of any therapy. The outcome measurement can be pathologic response to therapy. Alternatively, outcome measures, such as overall survival and disease-free survival can be monitored over a period of time for subjects following therapies for whom biomarker measurement values are known. In certain embodiments, the same doses of therapy agents, if any, are administered to each subject. In related embodiments, the doses administered are standard doses known in the art for those agents used in therapies. The period of time for which subjects are monitored can vary. For example, subjects can be monitored for at least 2, 4, 6, 8, 10, 12, 14, 16, 18, 20, 25, 30, 35, 40, 45, 50, 55, or 60 months.

### VI. Administration of Agents

The agents encompassed by the present invention (e.g., STING agonists) are administered to subjects in a biologically compatible form suitable for pharmaceutical administration in vivo, to enhance their effects. By “biologically compatible form suitable for administration in vivo” is meant a form to be administered in which any toxic effects are outweighed by the therapeutic effects. The term “subject” is intended to include living organisms in which an immune response can be elicited, e.g., mammals. Examples of subjects include humans, dogs, cats, mice, rats, and transgenic species thereof. Administration of an agent as described herein can be in any pharmacological form including a therapeutically active amount of an agent alone or in combination with a pharmaceutically acceptable carrier.

Administration of a therapeutically active amount of the therapeutic composition encompassed by the present invention is defined as an amount effective, at dosages and for periods of time necessary, to achieve the desired result. For example, a therapeutically active amount of an agent can vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

Agents encompassed by the present invention can be administered either alone or in combination with an additional therapy. In the combination therapy, a STING agonist encompassed by the present invention and another agent, such as a PARP inhibitor, a TK inhibitor, or a DNA synthesis inhibitor can be delivered to the same or different cells and can be delivered at the same or different times. The agents encompassed by the present invention can be incorporated into pharmaceutical compositions suitable for administration. Such compositions can comprise one or more agents or one or more molecules that result in the production of such one or more agents and a pharmaceutically acceptable carrier.

The therapeutic agents described herein can be administered using a mode or route of administration that delivers them to the particular locations in the body, or systemically. In some embodiments, the mode of administration is systemic, such as oral, intravenous, or intraperitoneal.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which can inactivate the compound. For example, for administration of agents, by other than parenteral administration, it can be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

As described in detail below, the pharmaceutical compositions encompassed by the present invention can be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intra-vaginally or intra-rectally, for example, as a pessary, cream or foam; or (5) aerosol, for example, as an aqueous aerosol, liposomal preparation or solid particles containing the compound.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity, or expression and/or activity of the complex encompassed by the present invention. These salts can be prepared in situ during the final isolation and purification of the therapeutic agents, or by separately reacting a purified therapeutic agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods encompassed by the present invention can contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the therapeutic agents, or by separately reacting the purified therapeutic agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods encompassed by the present invention include those suitable for oral, nasal, topical (including buccal and sublingual), rectal, vaginal, aerosol and/or parenteral administration. The formulations can conveniently be presented in unit dosage form and can be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a therapeutic agent with liquid carriers, or finely divided solid carriers, or both, and then, if necessary, shaping the product.

Formulations suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a therapeutic agent as an active ingredient. A compound can also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered peptide or peptidomimetic moistened with an inert liquid diluent.

Tablets, and other solid dosage forms, such as dragees, capsules, pills and granules, can optionally be scored or prepared with coatings and shells, such as enteric coatings and other coatings well-known in the pharmaceutical-formulating art. They can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, or by incorporating sterilizing agents in the form of sterile solid compositions, which can be dissolved in sterile water, or some other sterile injectable medium immediately before use. These compositions can also optionally contain opacifying agents and can be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration can be presented as a suppository, which can be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component can be mixed under sterile conditions with a pharmaceutically-acceptable carrier, and with any preservatives, buffers, or propellants which can be required.

The ointments, pastes, creams and gels can contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agents disclosed herein can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions encompassed by the present invention suitable for parenteral administration comprise one or more therapeutic agents in combination with one or more pharmaceutically-acceptable sterile isotonic aqueous or nonaqueous solutions, dispersions, suspensions or emulsions, or sterile powders which can be reconstituted into sterile injectable solutions or dispersions just prior to use, which can contain antioxidants, buffers, bacteriostats, solutes which render the formulation isotonic with the blood of the intended recipient or suspending or thickening agents.

Examples of suitable aqueous and nonaqueous carriers which can be employed in the pharmaceutical compositions encompassed by the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

In some cases, in order to prolong the effect of a drug, it is desirable to slow the absorption of the drug from subcutaneous or intramuscular injection. This can be accomplished by the use of a liquid suspension of crystalline or amorphous material having poor water solubility. The rate of absorption of the drug then depends upon its rate of dissolution, which, in turn, can depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the therapeutic agents encompassed by the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in pharmaceutical compositions encompassed by the present invention can be determined by the methods encompassed by the present invention to obtain an amount of the active ingredient, which is effective to achieve the desired therapeutic response for a particular subject, composition, and mode of administration, without being toxic to the subject.

The nucleic acid molecules encompassed by the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In one embodiment, an agent encompassed by the present invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors can influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment can increase or decrease over the course of a particular treatment. Changes in dosage can result from the results of diagnostic assays.

### EXAMPLES

### Example 1: Overcoming Immunosuppression and Therapeutic Resistance to PARP Inhibition in BRCA1-Deficient Breast Cancer with STING Agonists

PARP inhibitors (PARPi) have drastically changed the treatment landscape of advanced ovarian tumors with BRCA mutations. However, the impact of this class of inhibitors in patients with advanced BRCA-mutant breast cancer is relatively modest. Using a syngeneic genetically engineered mouse model of breast tumor driven by Brca1 deficiency, the provided experiments show that tumor-associated macrophages (TAMs) abrogate PARPi efficacy both in vivo and in vitro. Mechanistically, BRCA1-deficient breast tumor cells induce protumor polarization of TAMs, which in turn suppress PARPi-elicited DNA damage in tumor cells, leading to reduced production of cytosolic ds-DNA and synthetic lethality, hence impairing STING-dependent antitumor immunity. STING agonists reprogram M2-like tumor-educated macrophages (TEMs) into an M1-like anti-tumor state in a macrophage STING-dependent fashion. Systemic administration of STING agonists breaches multiple layers of tumor cell-mediated suppression on macrophages and dendritic cells, and synergizes with PARPi. The therapeutic synergy of this combination is mediated by a type I IFN response and CD8+ T cells, but does not rely on tumor cell-intrinsic STING. The data illustrate the importance of targeting innate immune suppression to facilitate PARPi-mediated engagement of antitumor immunity in breast cancer.

The results uncovered a novel mechanism by which BRCA1-deficient breast tumors exert therapeutic resistance to PARP inhibitors through the induction of an immunosuppressive microenvironment dominated by pro-tumor macrophages that inhibit both anti-tumor immune response and synthetic lethality in tumor cells. Pharmacological activation of the STING pathway in immune cells overcomes resistance and sensitizes tumor cells to PARP inhibition.

### INTRODUCTION

Homologous recombination (HR) deficiency confers exquisite sensitivity to poly (ADP-ribose) polymerase (PARP) inhibitors (PARPi), which have been therapeutically exploited in both ovarian and breast tumors carrying loss-of-function mutations in HR pathway genes, most commonly BRCA1 and BRCA2 (BRCA1/2) (Lord C J, Ashworth A.Science 2017;355 (6330): 1152-8). Based on a substantial progression-free survival (PFS) benefit, three PARPi have gained FDA approval for BRCA-mutated ovarian cancer in both adjuvant and metastatic settings (Matulonis U A, et al. Ann Oncol 2016; 27 (6): 1013; Moore K, et al. N Engl J Med 2018; 379 (26): 2495-505; Swisher E M, et al. Lancet Oncol 2017; 18 (1): 75-87; Del Campo J M, et al. J Clin Oncol 2019; 37 (32): 2968-73 9). Most recently, maintenance treatment with olaparib was shown to confer unprecedented overall survival benefit for patients with BRCA-mutated relapsed ovarian cancer (Poveda A, et al. Journal of Clinical Oncology 2020; 38). Compared to ovarian cancer, however, PARPi therapy appears to be less effective in BRCA-mutated breast cancer. Nevertheless, the FDA has approved two PARPi—olaparib and talazoparib—as monotherapy for patients with germline BRCA1/2-mutated and HER2-negative advanced breast cancer (Robson M, et al. N Engl J Med 2017;377 (6): 523-33; Litton J K, et al. N Engl J Med 2018; 379 (8): 753-63). Although these PARPi significantly improved PFS, recent results from the OlympiAD and EMBRACA clinical trials suggest no overall survival benefit for both olaparib and talazoparib in patients with advanced breast cancer carrying germline BRCA1/2 mutations (Robson M E, et al. Ann Oncol 2019; 30 (4): 558-66; Litton J K, et al. American Association for Cancer Research (AACR) Virtual Annual Meeting I2020), highlighting the need to understand why BRCA-mutated breast cancers are more refractory to PARPi in the effort to develop strategies to improve responses to PARPi.

The understanding of the mechanisms underlying therapeutic efficacy of PARPi is still evolving. Since first described in 2005 (Bryant H E, et al. Nature 2005;434 (7035): 913-7; Farmer H, et al. Nature 2005;434 (7035): 917-21)), PARPi have been shown to exert synthetic lethality in HR-deficient tumor cells via multiple mechanisms, including inhibiting base excision repair (BER), trapping of PARP-DNA complexes, activating error-prone non-homologous end joining (NHEJ), and interfering with PARP1/POLQ-mediated alternative end joining (alt-EJ) (Konstantinopoulos P A, et al. Cancer Discov 2015;5 (11): 1137-54; Scott C L, et al. J Clin Oncol 2015;33 (12): 1397-406). Recently, various experiments demonstrated that, in addition to direct cytotoxicity via synthetic lethality, the immune response triggered by PARPi is also required for tumor elimination in vivo (Ding L, et al. Cell Rep 2018; 25 (11): 2972-80; Pantelidou C, et al. Cancer Discov 2019; 9 (6): 722-37; Chabanon R M, et al. J Clin Invest 2019; 129 (3): 1211-28). Using a genetically-engineered mouse model (GEMM) of Brca1-deficient ovarian cancer, experiments showed that treatment with PARPi leads to release of double-stranded DNA (dsDNA) by tumor cells, which activates stimulator of interferon genes (STING) signaling in intratumoral dendritic cells (DCs), thus triggering a type I interferon (IFN) response and subsequent induction of anti-tumor CD8+ T cells. Activation of the STING pathway occurs through production of cyclic dinucleotides by Cyclic GMP-AMP Synthase (cGAS), which acts as a sensor for cytosolic dsDNA in tumor and immune cells (Li T, J Exp Med 2018; 215 (5): 1287-99). Because of their ability to induce DNA double-strand breaks that result in release of dsDNA fragments from the nucleus, PARPi have also been shown to activate tumor cell-intrinsic immunity (Pantelidou C, et al. Cancer Discov 2019; 9 (6): 722-37; Chabanon R M, et al. J Clin Invest 2019; 129 (3): 1211-28). However, the importance of STING in immune cells and tumor cells in cancer treatment remains unclear.

Notably, clinical outcome in breast cancer is strongly affected by the tumor immune microenvironment (TIME) (Savas P, Loi S. Cancer Cell 2020; 37 (5): 623-4; Salmon H, et al. Nat Rev Cancer 2019; 19 (4): 215-27; Li W, Tanikawa T, et al. Cell Metab 2018; 28 (1): 87-103). Multiple studies have shown that advanced breast tumors often exhibit a pre-existing immune suppressive TIME characterized by significantly reduced tumor-infiltrating lymphocyte (TIL) levels and increased expression of immune suppressive genes, which correlates with reduced response to chemotherapy and immunotherapy (Li W, et al. Cell Metab 2018; 28 (1): 87-103; Hutchinson K E, et al. Clin Cancer Res 2020; 26 (3): 657-68; Savas P, Loi S. Clin Cancer Res 2020; 26 (3): 526-8). While increasing evidence has indicated the immunomodulatory properties of PARPi in vivo, it is currently unclear whether an already established immune suppressive TIME in BRCA-mutant breast cancer may influence the efficacy of PARPi.

Tumor-associated macrophages (TAMs) constitute one of the most abundant and diverse immune populations that can be found in solid tumors. Although TAM phenotypes and functions are highly plastic and diverse, macrophages can be broadly classified as anti-tumorigenic (M1-polarized) or pro-tumorigenic (M2-polarized) based on their ability to either suppress or promote tumor growth (DeNardo D G, Ruffell B. Nat Rev Immunol 2019; 19 (6): 369-82). M2-polarized TAMs can exert immune suppression via multiple mechanisms, including recruitment of immunosuppressive immune cells such as regulatory T cells (Tregs) and direct inhibition of immune effector cells such as natural killer (NK) cells and cytotoxic T cells (Cassetta L, Pollard J W. Nat Rev Drug Discov 2018; 17 (12): 887-904). Of note, multiple clinical studies have shown that high TAM infiltration associates with poor prognosis in the majority of cancer types (Ruffell B, Coussens L M. Cancer Cell 2015;27 (4): 462-72).

In this study, results demonstrate that the response of BRCA1-deficient breast tumors to PARPi is strongly limited by immunosuppressive TAMs, which not only directly inhibit CD8+ T cells but also suppress PARPi-mediated tumor cell DNA damage, resulting in reduced cytosolic dsDNA and synthetic lethality, thereby dampening STING-dependent activation of antigen-presenting cells (APCs). Addition of an exogenous STING agonist shifts TAMs away from a pro-tumorigenic phenotype, restores the synthetic lethal response to PARPi, and effectively activates APCs. Consequently, systemic delivery of STING agonists shows strong therapeutic synergy with PARP inhibition in Brca1-deficient mouse models of breast cancer regardless of tumor cell-intrinsic STING expression. The findings reveal new approaches to reverse 1 the resistance of BRCA1-mutant breast cancers to PARPi therapy.

### Results

### Brca1-Deficient Breast Tumors Show Modest Response to Olaparib In Vivo

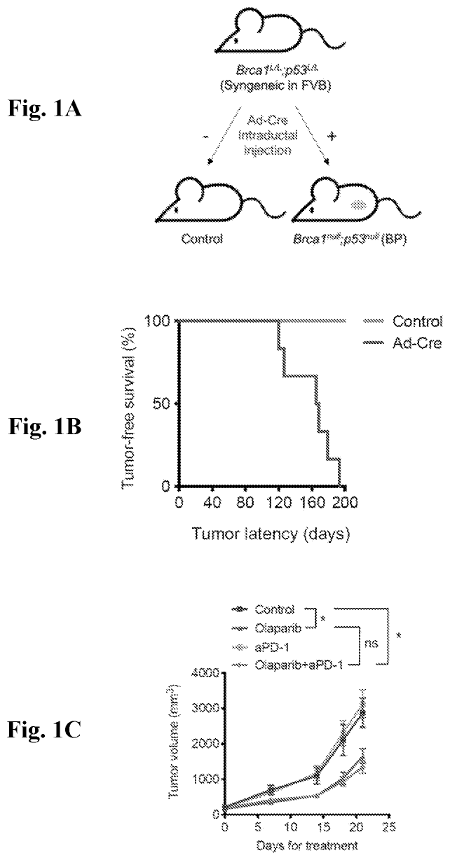

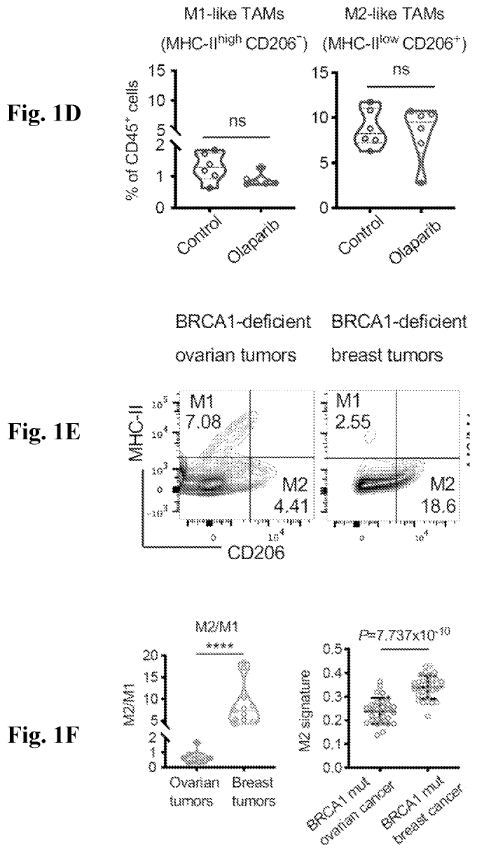

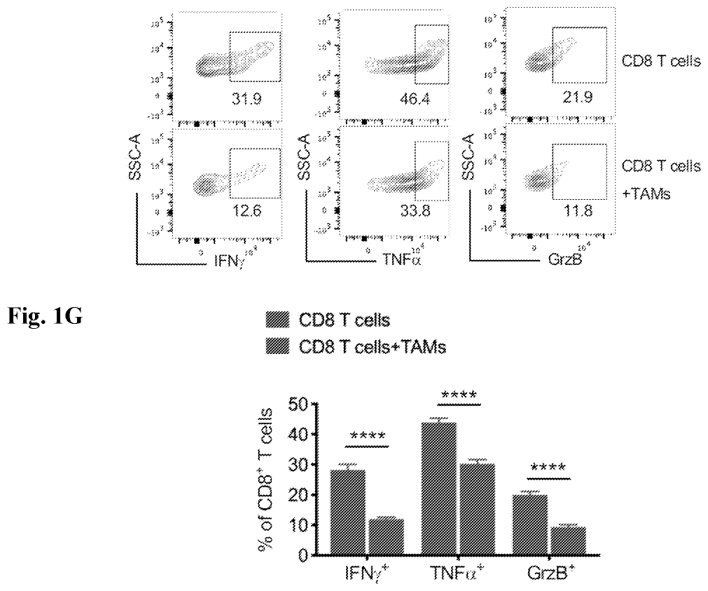

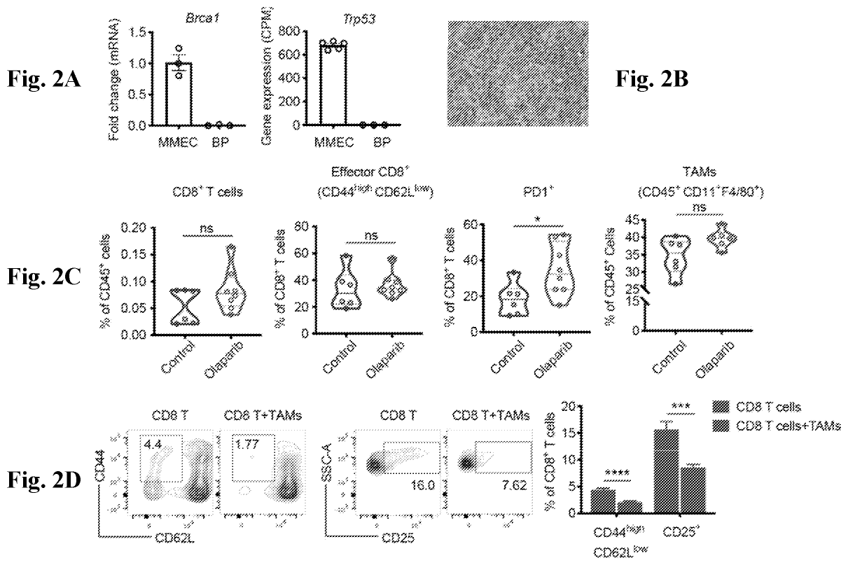

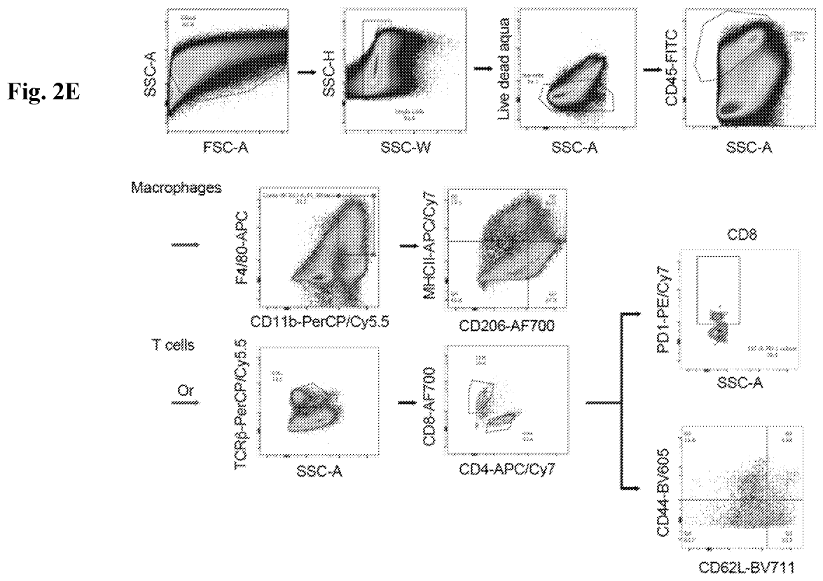

To investigate response to PARPi in Brca1-deficient breast cancer, a syngeneic GEMM of breast tumor driven by concurrent ablation of Brca1 and Trp53 (referred as BP) was developed. These BP breast tumors were generated through intraductal injection of adenovirus expressing Cre recombinase into the mammary ducts of FVB females carrying homozygously floxed alleles of both Brca1 and Trp53 (Brca1L/L; Trp53L/L, FIG. 1A and FIG. 2A). These mice developed mammary tumors around 4-7 months (median latency of 167 days) with 100% penetration (FIG. 1B). Histology analysis revealed that these BP tumors were poorly differentiated adenocarcinomas (FIG. 2B), resembling advanced BRCA-deficient breast cancer in the clinic (Palacios J, et al. Clin Cancer Res 2003; 9 (10 Pt 1): 3606-14). Moreover, TP53 mutations are frequently found in advanced breast cancers with BRCA1 deficiency (Holstege H, et al. Cancer Res 2009; 69 (8): 3625-33). Thus, this BP tumor model recapitulates both the genetics and pathology of advanced BRCA1-deficient breast tumors in patients.

Notably, primary tumor cells derived from BP tumors can be cultured in vitro as well as allografted back into the mammary fat pads of syngeneic immunocompetent hosts, allowing detailed studies of tumor cell-intrinsic activities, as well as their interactions with the host immune system and their responses to therapeutic interventions. To disappointment, when FVB mice bearing orthotopic BP tumors were treated with olaparib, BP tumors exhibited an initial slower growth than control tumors but nevertheless progressed through treatment and presented growth rates comparable to control tumors at later time points (FIG. 1C). While there was a statistically significant reduction in tumor size compared to control tumors, the effect of olaparib on Brca1-deficient breast tumors was modest, in contrast to its remarkable effect on a comparable mouse model of high-grade serous ovarian cancer (HGSOC) driven by concurrent ablation of Brca1 and Trp53 and overexpression of cMyc that was recently developed, where it was found that ovarian tumors had a dramatic response to olaparib with a robust activation of anti-tumor CD8+ T cell response in the TIME, which was essential for the therapeutic efficacy of PARP inhibition.

### BP Tumor-Associated Macrophages 1 Mediate Immune Suppression

To investigate how the TIME affects response to PARPi in vivo, alterations in the BP TIME in response to PARP inhibition was sought to assess. Analysis of TILs revealed that CD8+ T cells and effector CD8+ T cells from the treatment group were not significantly changed after seven days of treatment (FIGS. 2C and 2E), suggesting that olaparib was not able to trigger robust T cell activation in the BP TIME. Interestingly, the proportion of PD-1+CD8+ T cells was increased in BP tumors after seven days of treatment with olaparib (FIGS. 2C and 2E), indicating increased exhaustion of CD8+ T cells in tumors over the course of olaparib treatment. However, blocking PD-1 with a monoclonal antibody against mouse PD-1 was not able to improve BP tumor response to olaparib (FIG. 1C), suggesting that BP tumors are protected from T cell-mediated destruction.

Experiments subsequently investigated other types of immune cells in the BP TIME. Flow cytometric analysis revealed that BP tumors had a large population of tumor-associated macrophages (TAMs, CD45+CD11b+F4/80+), which was not significantly affected by olaparib (FIGS. 2C and 2E). Notably, the proportion of M2-like (MHC-IILow CD206+) macrophages was more than five-fold higher than that of M1-like (MHC-IIHigh CD206−) macrophages in tumors from both control and olaparib-treated groups (FIG. 1D and FIG. 2E), suggesting an immune suppressive population of TAMs was developed in the BP TIME independently of olaparib treatment.

Considering the marked differences in response to PARPi between the BRCA1-null breast and ovarian mouse tumor models, and given the high levels of M2-like macrophages in BP breast tumors, it was hypothesized that extrinsic factors may suppress CD8+ T cell activation and thereby dampen response to treatment in breast tumors. Hence, TAMs isolated from mouse breast and ovarian BRCA1-null tumors were compared. Notably, TAM M2 polarization was much stronger in BRCA1-null breast tumors than in BRCA1-deficient ovarian tumors, as evidenced by the M2/M1 ratios (FIG. 1E). This result is consistent with clinical data showing that BRCA1-mutant breast tumors have significantly higher enrichment scores of M2 macrophage gene signature (Pan W, et al. Immunity 2017;47 (2): 284-97) than BRCA1-mutant ovarian tumors (FIG. 1F). Together, these data suggest that M2-like macrophages in BRCA1-null breast tumors may contribute to the resistance to PARPi.

To confirm the immunosuppressive function of TAMs in BRCA1-mutant 1 breast tumors, TAMs from BP tumors (14 days after transplantation) were sorted and co-cultured with splenic CD8+ T cells isolated from naive mice. Indeed, CD8+ T cells co-cultured with TAMs had significantly reduced IFNg, TNFa and Gramzyme B production, as well as decreased expression of CD25 and lower levels of effector cells (CD44highCD62Llow), compared to control T cells (FIG. 1G and FIG. 2D).

### Both Murine and Human BRCA1-Deficient Breast Tumor Cells Induce M2-Like Macrophage Polarization In Vitro

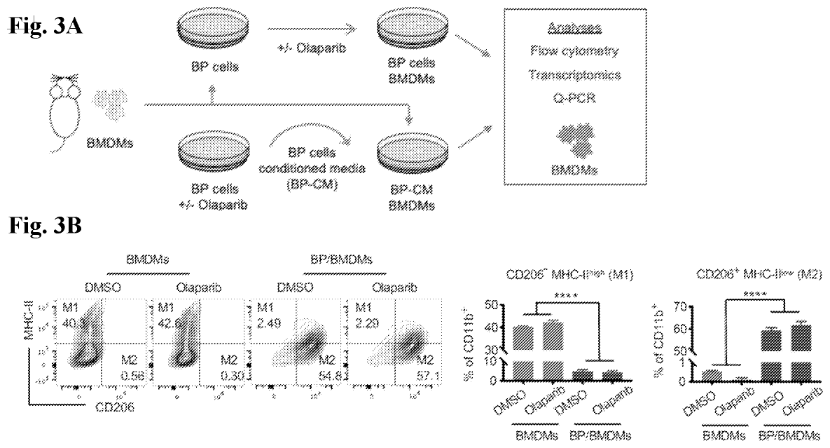

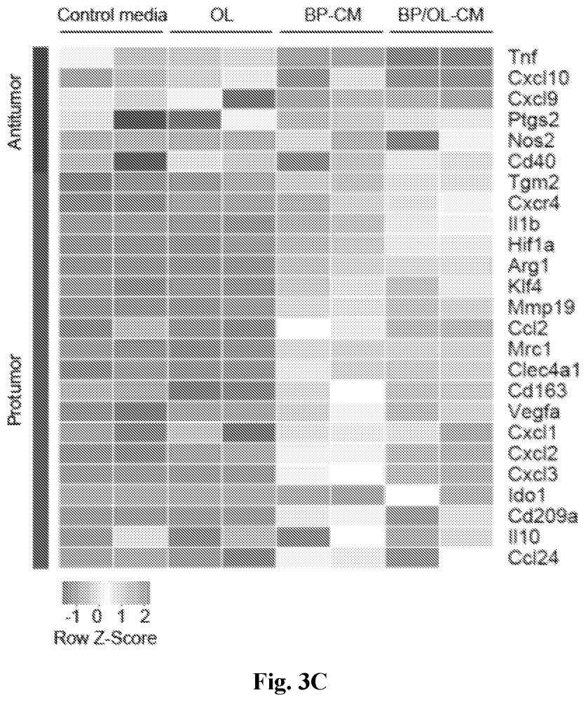

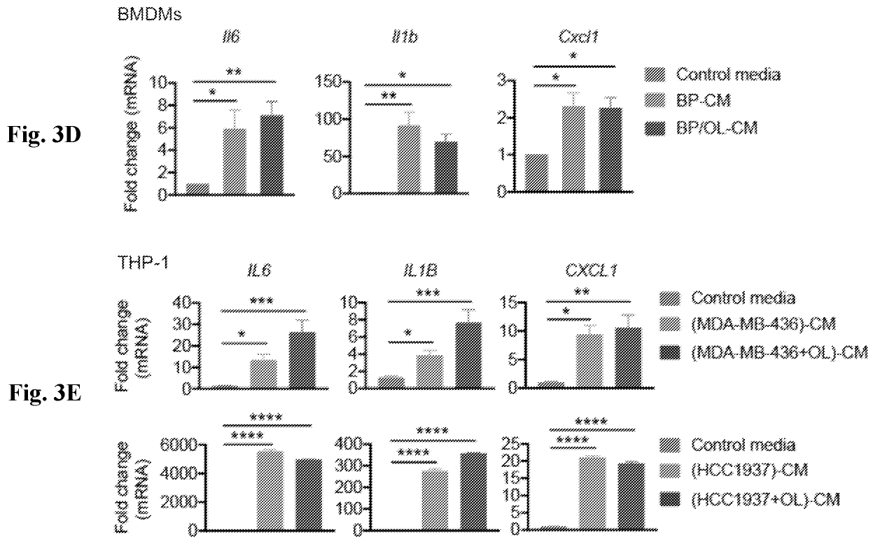

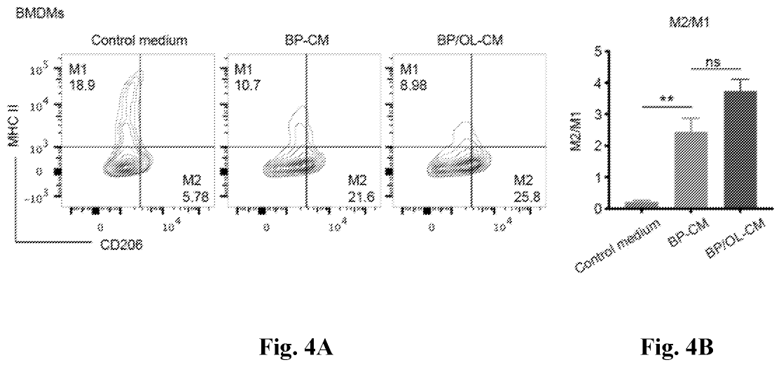

Given the highly M2-like nature of TAMs in BP tumors, experiments next examined the interaction of macrophages and BP tumor cells in an in vitro system in the presence or absence of olaparib. A co-culture system with mouse bone marrow-derived macrophages (BMDMs) and primary BP tumor cells was established for two days with or without olaparib (FIG. 3A, the top panel). Strikingly, it was found that there was a dramatic reduction of the M1-like population (from ˜40% to ˜2%) with a concurrent substantial increase in the M2-like macrophage population (from ˜0.5% to ˜60%) (FIG. 3B). Notably, olaparib alone had little effect on macrophage polarization (FIG. 3B). To explore this further, BMDMs were incubated with conditioned media (CM) from BP tumor cells (BP-CM), or olaparib-treated BP cells (BP/OL-CM) (FIG. 3A, the lower panel). Consistent with the co-culture system above, both BP-CM and BP/OL-CM strongly promoted an M2-like polarization that resulted in a substantial induction of the M2/M1 ratio (FIGS. 4A and 4B). These data suggest that BP tumor cells can modulate the macrophage phenotype through tumor cell-derived soluble factors.

Next RNA-seq analysis of BMDMs treated with control medium, olaparib, BP-CM or BP/OL-CM was conducted. As shown in FIG. 3C, olaparib had no significant direct effects on BMDMs. By contrast, BP-CM strongly up-regulated expression of genes associated with an M2-like/pro-tumorigenic phenotype (e.g., Ccl2, Vegfa, Arg1 and Mrc1), while it down-regulated expression of key M1-like/anti-tumorigenic genes (e.g., Tnf and Cxcl10). Notably, BMDMs had a similar transcriptional profile when they were treated with BP/OL-CM to that seen with BP-CM (FIG. 3C), It was further confirmed that the expression levels of M2-like associated genes, including Il6, Il1b and Cxcl1, were significantly increased in BMDMs treated with BP-CM or BP/OL-CM via RT-qPCR (FIG. 3D), demonstrating that BP tumor cells largely contribured 1 to the induction of M2-like macrophages. To assess whether this M2 macrophage polarization induced by murine BP breast tumor cells could be recapitulated by human BRCA1-mutant breast cancer cells, human THP-1 macrophages were treated with CM collected from BRCA1-mutant breast cancer cell lines MDA-MB-436 or HCC1937 and assessed expression of key genes associated with an M2-like phenotype. Notably, THP-1 macrophages treated with tumor cell CM significantly up-regulated IL6, IL1B and CXCL1 gene expression, which was not further affected by treating the tumor cells with olaparib (FIG. 3E). Together, the data suggest that both murine and human BRCA1-deficient breast tumor cells can “educate” macrophages to become M2-like pro-tumorigenic macrophages independent of PARPi. Hereafter, these macrophages co-cultured with tumor cells or incubated with their CM are referered to as tumor cell-educated macrophages (TEMs).

### TEMs Suppress Olaparib-Induced DNA Damage in BRCA1-Deficient Breast Tumor Cells and Abrogate STING Activation in DCs and Tumor Cells In Vitro

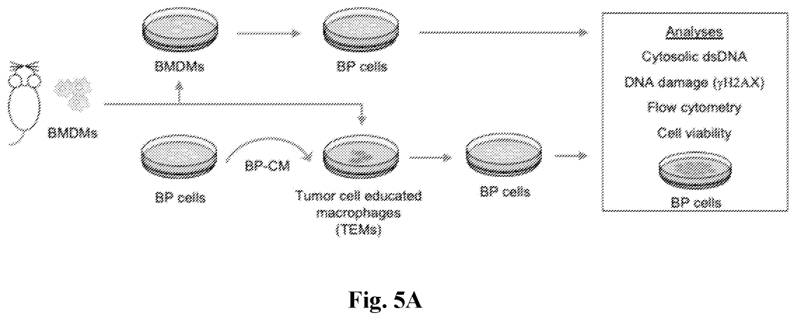

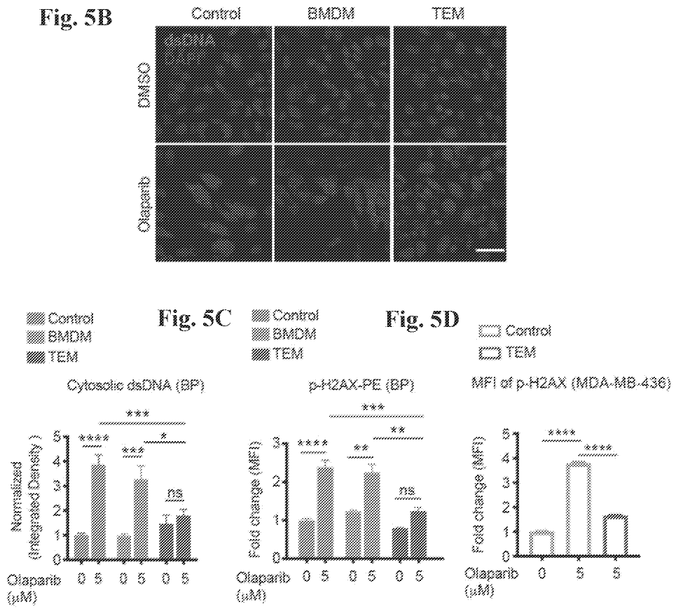

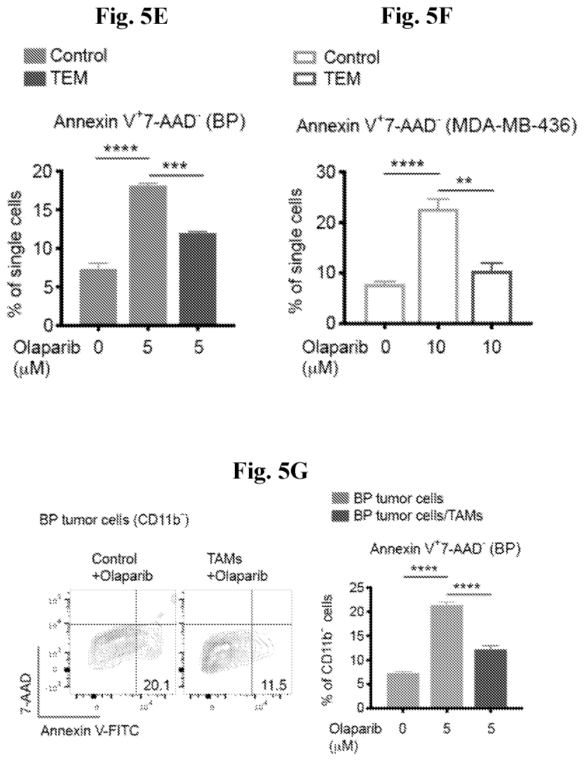

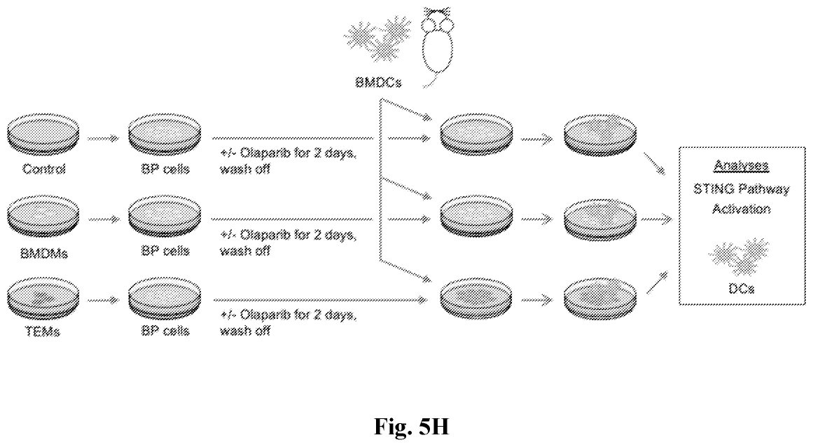

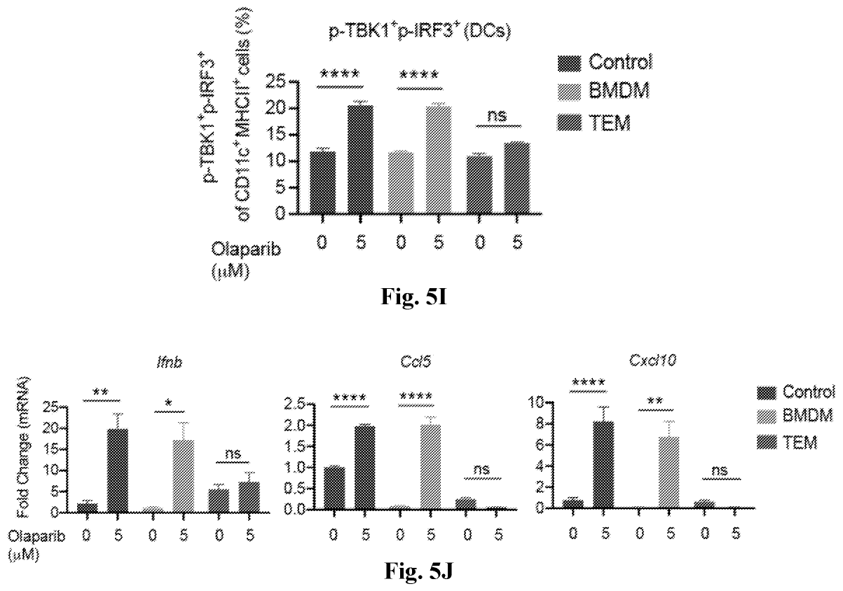

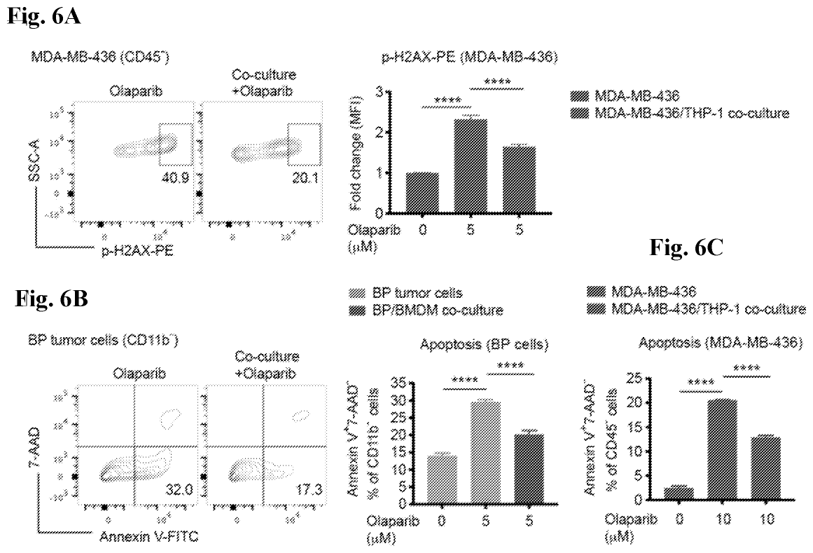

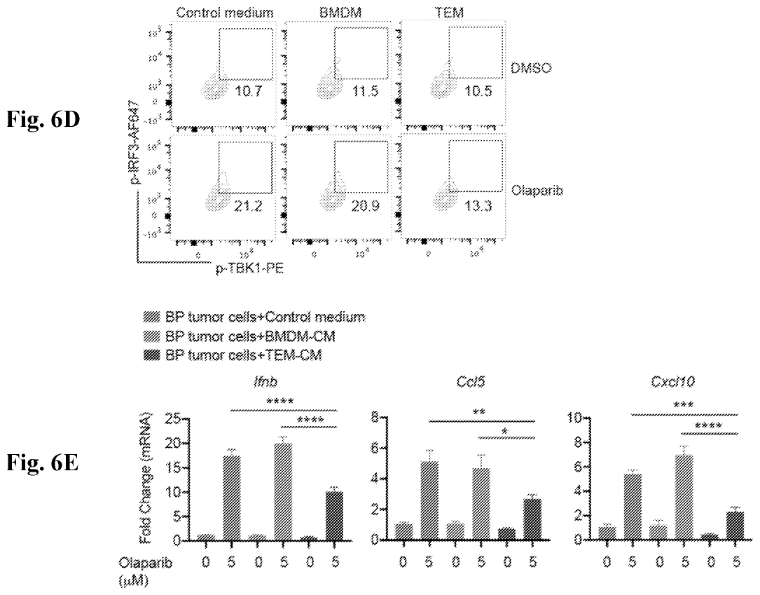

Next, how these M2-like TEMs affect tumor cell activity (FIG. 5A) was investigated. Since the synthetic lethal response of BRCA1-deficient tumor cells to PARPi is driven by highly deleterious DNA double-strand breaks (DSBs), experiments first tested how TEMs affect PARPi-induced DSBs in tumor cells. BP tumor cells were incubated with TEM-conditioned medium or naive BMDM-conditioned medium, followed by olaparib treatment. Immunofluorescence (IF) analysis with an antibody against double-stranded DNA (dsDNA) revealed that olaparib induced accumulation of cytosolic dsDNAs in BP cells (FIG. 5B). TEMs, but not naive BMDMs, abrogated the production of cytosolic dsDNAs in BP cells upon olaparib treatment (FIG. 5B). The DNA damage was further assessed by measuring histone H2AX phosphorylation at Serine 139 (γ-H2AX), a surrogate marker and an early cellular response to DNA DSBs (30). Notably, BP tumor cells responded to olaparib with a two-fold increase of γ-H2AX (FIG. 5C). Consistenly, TEMs, but not naive BMDMs, reduced γ-H2AX up-regulation in BP cells following olaparib treatment (FIG. 5C). Similar results were also observed in the MDA-MB-436 tumor cells incubated with MDA-MB-436-educated THP-1 macrophages. It was found that γ-H2AX up-regulation induced by olaparib was significantly inhibited in MDA-MB-436 cells by tumor cell-educated THP-1 macrophages (FIG. 5D and FIG. 6A). Moreover, it was found that TEMs decreased olaparib-induced tumor cell apoptosis (Annexin V+ 7-AAD−) by approximately 40%-50% in BP tumor cells or human BRCA1-mutant breast cancer cells (FIG. 5E-5F and FIG. 6B-6C). Since these TEMs are derived from in vitro culture systems, TAMs were also directly harvested from BP tumors to assess their effect on tumor cells ex vivo. Indeed, TAMs significantly reduced BP cell death in response to olaparib (FIG. 5G). These data suggest that BRCA1-deficient breast tumor cells polarize macrophages into M2-like TAMs or TEMs, which in turn suppress the synthetic lethal response of tumor cells to olaparib.

It was recently reported that dsDNAs derived from BRCA1-deficient ovarian tumor cells treated with PARPi triggered STING-dependent activation of DCs, which was a critical step for PARPi to activate anti-tumor immunity. To test whether this also occurs in BRCA1-deficient breast tumor cells, and to assess the effects of TEMs on this process, bone marrow-derived DCs (BMDCs) were co-cultured rwith BP tumor cells pre-incubated with control medium or conditioned media from naive BMDMs or TEMs, followed by olapalib treatment and washing off (FIG. 5H). Consistent with BRCA1-deficient ovarian tumor cells, olaparib-treated BP cells activated the STING pathway in DCs, as indicated by concurrently increased levels of both TANK Binding Kinase 1 (TBK1) and IFN regulatory factor 3 (IRF3) phosphorylation (p-TBK1+ p-IRF3+) (FIG. 5I and FIG. 6D). In contrast, TEMs, but not naive BMDMs, inhibited the capacity of olaparib-treated BP cells to activate the STING pathway in DCs (FIG. 5I and FIG. 6D). Consistently, the up-regulation of pro-inflammatory cytokines (Ifnb, Ccl5 and Cxcl10) found in DCs with an activated STING pathway was blunted when they were co-cultured with BP cells pre-incubated with TEMs (FIG. 5J). In parallel, tumor cell-intrinsic immunity induced by PARPi as reported in was also evaluated. It was found that BP tumor cells showed a marked up-regulation of pro-inflammatory cytokines (Ifnb, Ccl5 and Cxcl10) upon olaparib treatment in control medium or with naive BMDMs, but this up-regulation of inflammatory cytokines was significantly reduced by TEMs (FIG. 6E). Together, the results demonstrate that BRCA1-deficient breast tumor cells induce pro-tumorigenic reprogramming of macrophages, which in turn suppress DNA damage and synthetic lethality in tumor cells upon PARP inhibition. This suppressed DNA damage and, consequently, diminished cytosolic dsDNAs in tumor cells, leads to reduced activation of the STING pathway in both DCs and tumor cells, thus weakening anti-tumor immunity.

### STING Agonists can Reprogram TEMs to an M1-Like 1 State in a Macrophage STING-Dependent Fashion

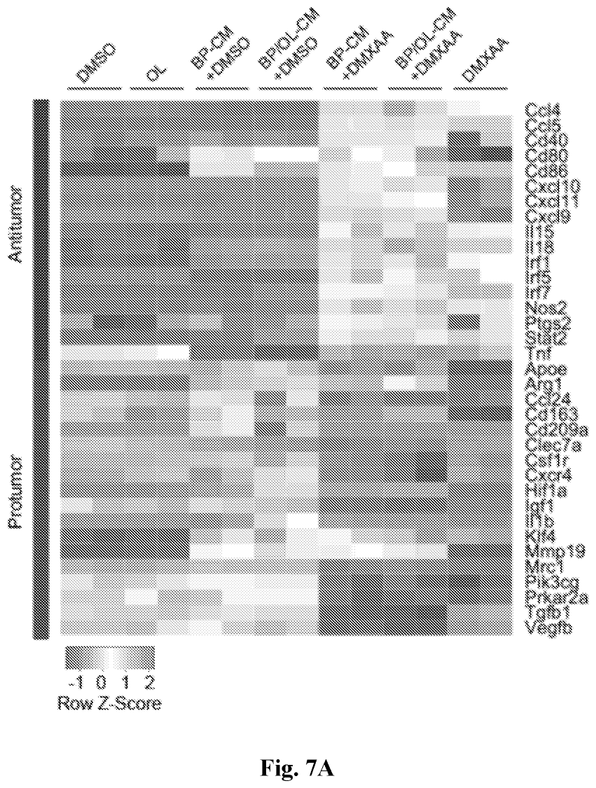

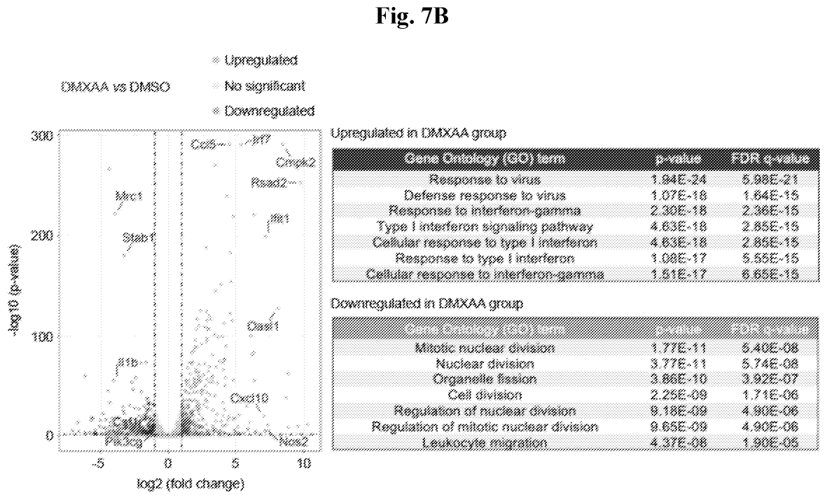

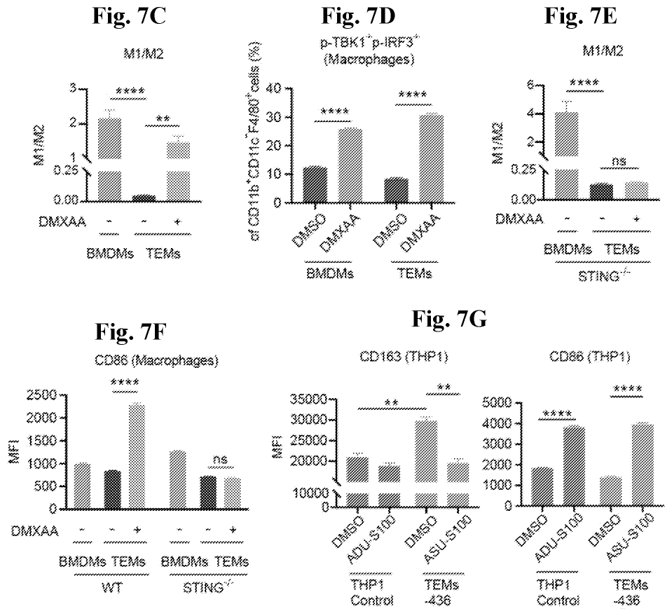

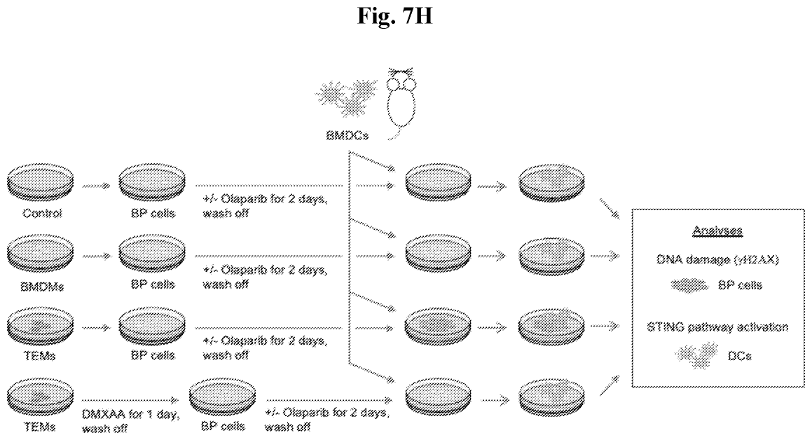

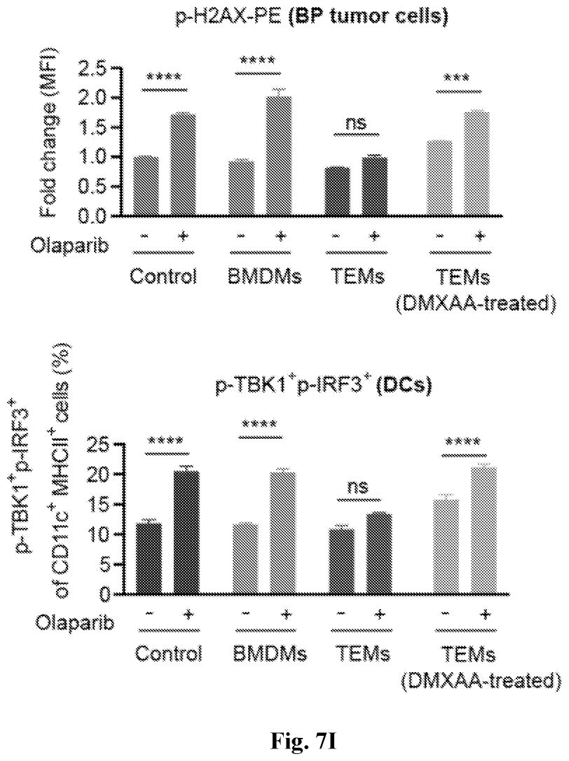

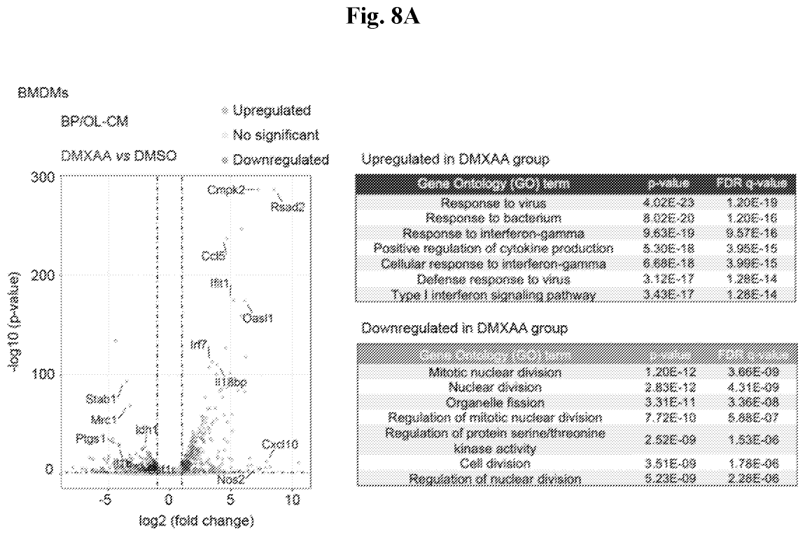

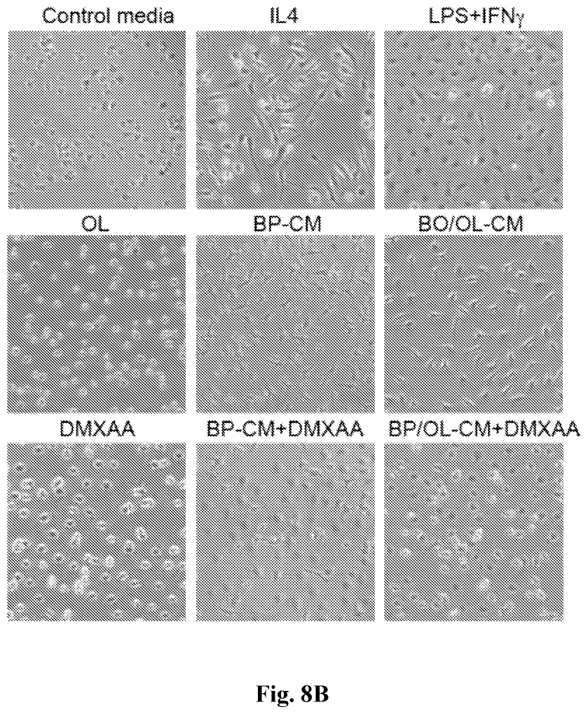

Given the finding that STING pathway activation via tumor cell-derived dsDNA is reduced by the interaction of tumor cells and macrophages, it was hypothesized that an exogenous STING agonist might be used to overcome this immunosuppression and potentiate PARPi efficacy. To investigate whether DMXAA, a potent murine STING agonist (Gao P, et al. Cell 2013; 154 (4): 748-62; Corrales L, et al. Cell Rep 2015;11 (7): 1018-30)), could inhibit pro-tumorigenic polarization of macrophages by BP tumor cells, the RNA-seq analysis of BMDMs treated with control, olaparib, BP-CM or BP/OL-CM (shown in FIG. 3C) was repeated but in the presence of DMXAA (FIG. 7A). Transcriptomic analysis showed robust up-regulated expression of genes associated with pro-tumorigenic M2 polarization, including Arg1, Csf1r, Il1b, Mrc1, Pik3cg, Ptgs1, Stab1 and Tgfb1, in BMDMs cultured with BP-CM or BP/OL-CM as compared to control (FIG. 7A). Strikingly, DMXAA not only suppressed the expression of those pro-tumorigenic genes, but also strongly stimulated expression of genes associated with an anti-tumorigenic M1 signature (e.g., Ccl5, Cxcl10, Cd40, Nos2 and Tnf) (FIG. 7A). Gene ontology (GO) analysis revealed that DMXAA significantly increased “response to virus”, “response to interferon-gamma” and “type I interferon signaling pathway” signals and concurrently decreased “mitotic nuclear division” and “organelle fission” biological processes in these macrophages (FIG. 7B and FIG. 8A). In keeping with changes in gene expression, macrophage activation by STING agonism was also reflected in morphological changes. As shown on FIG. 8B, BMDMs incubated with BP-CM showed an elongated or stellate morphology, which was similar to the M2-like macrophages induced by IL4. In contrast, addition of DMXAA changed these cells to a “fried egg” shape (round-shaped cells with large nuclei centered in the cytoplasm), which was similar to the morphology of M1-like macrophages induced by combined LPS and IFNg (Young D A, et al,. J Immunol 1990; 145 (2): 607-15). These data suggest that a STING agonist can prevent polarization of BMDMs into M2-like macrophages.

Experiments next investigated whether STING agonists can reverse M2-like TEMs to M1-like macrophages, as well as the role of the STING pathway in macrophages during STING agonist-mediated reprograming of macrophages. Remarkably, DMXAA reversed TEMs to M1-like macrophages and significantly increased TEM STING pathway activation (FIG. 7C-7D). To determine whether macrophage STING is required for this reprograming, BMDMs from STING-knockout (KO) mice (Sting^{gt/gt}) were isolated and co-cultured with BP cells ex vivo. STING−/− 1 BMDMs were readily polarized to M2-like TEMs when treated with BP cells (FIG. 7E), suggesting that STING is not required for M2-like macrophage polarization. Notably, these STING−/− TEMs were not reversed to M1-like macrophages upon DMXAA treatment (FIG. 7E), suggesting that STING is required for M1-like macrophage reprograming. Furthermore, STING−/− TEMs also failed to increase the expression of the co-stimulatory molecule CD86 (FIG. 7F). Next, whether STING agonists could also reprogram human TEMs was tested by first co-culturing THP-1 macrophages with MDA-MB-436 tumor cells, and then treating these TEMs with a STING agonist. ADU-S100, a human STING agonist, not only suppressed CD163, a scavenger receptor with important roles in activation of protumor macrophages (Shiraishi D, et al. Cancer Res 2018; 78 (12): 3255-66; Giurisato E, et al. Proc Natl Acad Sci USA 2018; 115 (12): E2801-E10), but also significantly increased the expression of CD86 on THP-1 macrophages (FIG. 7G). Together, the data suggest that STING agonists can reprogram macrophages into M1-like antitumor macrophages, which is dependent on macrophage STING.

### STING Agonists can Promote Activation of DCs by BP Cells Upon Olaparib in the Presence of TEMs In Vitro

Next, whether STING agonist can relieve TEM-mediated suppression of DNA damage in BP cells in response to PARPi was assessed, which would result in increased dsDNA release by BP cells, which in turn could promote activation of DCs. This experiment was performed with multiple controls and conditions in parallel as illustrated in FIG. 7H. As expected, BP cells incubated in control medium or treated with naive BMDMs had increased gH2AX upon olaparib treatment, and their co-cultured DCs had activated STING pathway (FIG. 7H-I). While BP cells treated with TEMs did not have increased gH2AX and their co-cultured DCs failed to have activated STING pathway, pre-treatment of TEMs with DMXAA restored BP cells' DNA damage in response to olaparib, as well as the activation of STING pathway in co-cultured DCs (FIG. 7H-71). Together, these data provide a strong rationale for the combination of PARPi with a STING agonist for the treatment of BRCA1-deficient breast tumors.

### STING Agonists Improve Therapeutic Response of Orthotopic BP Tumors to Olaparib in Syngeneic Immunocompetent Mice In Vivo

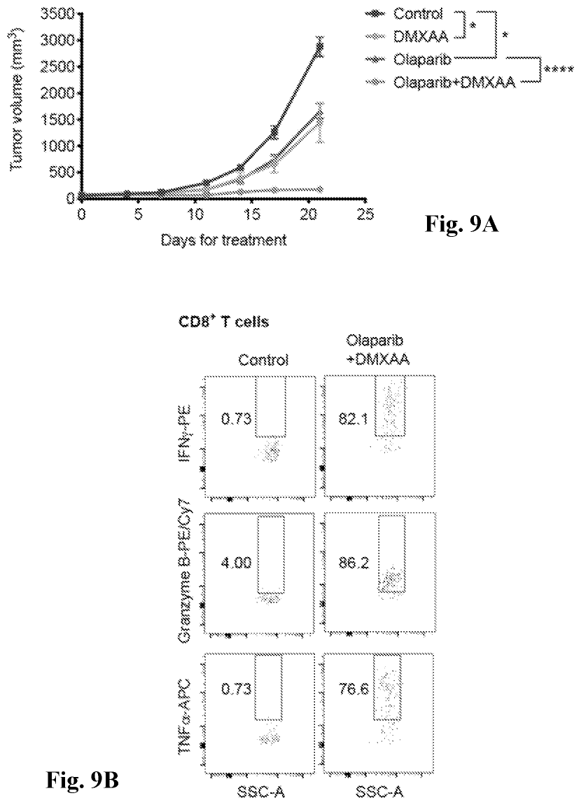

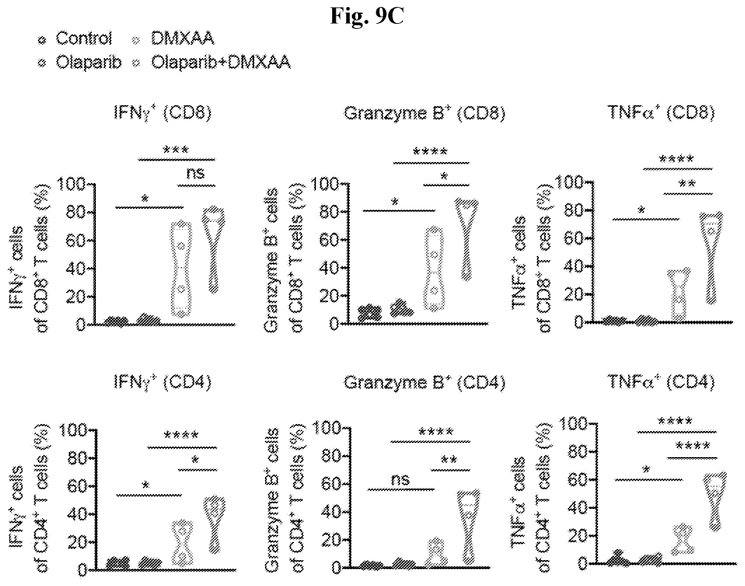

To determine whether stimulation of STING could potentiate anti-tumor activity 1 of PARPi in vivo, a cohort of FVB female mice bearing orthotopic BP tumors was established. When tumor volumes reached around 100 mm3, tumor-bearing mice were randomized into four groups and subjected to control, olaparib, DMXAA or combination of olaparib and DMXAA. DMXAA was administered into BP tumor-bearing mice via intratumoral (i.t.) injection, as this delivery method has shown promising potential. A relatively low dose of DMXAA (10 mg/kg) was administered once per week for three weeks (total of 3 doses). While DMXAA and olaparib monotherapy induced modest tumor growth inhibition, combined treatment strongly suppressed tumor growth (FIG. 9A). Analysis of the tumor immune infiltrate showed that DMXAA monotherapy strongly up-regulated production of anti-tumor cytokines (e.g., IFNg, Granzyme B and TNFa) in both CD8+ and CD4+ T cells, which was significantly enhanced by combining with olaparib therapy (FIG. 9B and FIG. 9C). Collectively, the results suggest that STING agonists overcome immune suppression and markedly improve the response of Brca1-deficient breast tumors to olaparib in vivo.

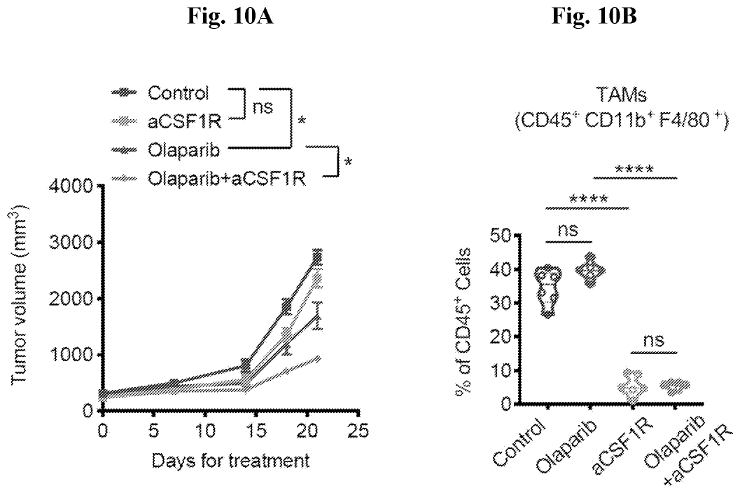

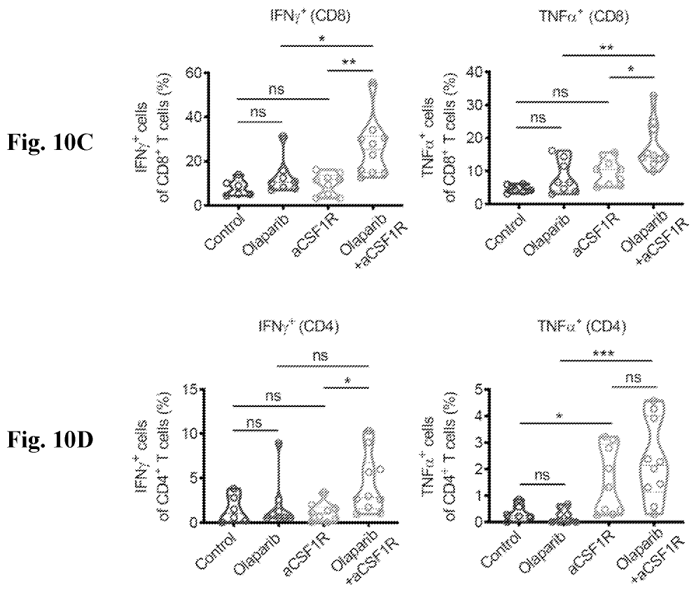

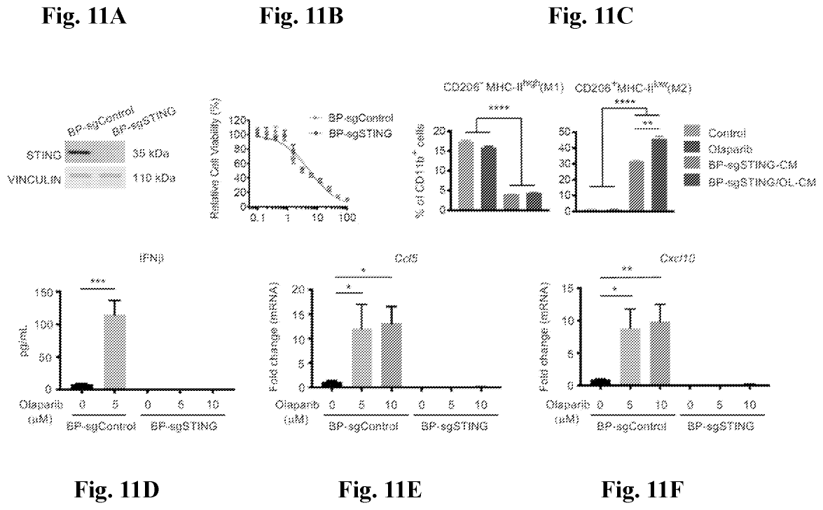

In parallel, the therapeutic efficacy of PARPi in combination with colony-stimulating factor 1 receptor (CSF1R) monoclonal antibody against mouse CSF1R was also evaluated in orthotopic allografts of BP tumors. Many CSF1R antagonists are in pre-clinical and clinical development to combat immunosuppression because of their activity in depleting TAMs by blocking macrophage survival signaling (Cannarile M A, et al. J Immunother Cancer 2017;5 (1): 53). While anti-CSF1R monotherapy had no significant effect in inhibiting BP tumor growth, combined treatment with olaparib and anti-CSF1R significantly attenuated tumor growth and improved the efficacy of olaparib (FIG. 10A). However, the therapeutic efficacy of combined olaparib and CSF1R antibody was significantly lower than that of combined olaparib and DMXAA (T/C %=39.8% [olaparib+aCSFIR] vs. 6% [olaparib+DMXAA] after 21 days of treatment; T/C %=100×[median tumor volume of treated group]/[median tumor volume of control group]) (FIG. 9A and FIG. 10A). Analysis of tumors treated with anti-CSF1R antibody indeed revealed markedly reduced abundance of TAMs (FIG. 10B), but the levels of intratumoral CD8+ and CD4+ T cell activation were lower in tumors treated with the combination of olaparib and anti-CSF1R compared to tumors treated with the combination of olaparib and DMXAA (FIG. 9B-9C and FIG. 10C-10D). Together, the data indicate that STING agonist-mediated TAM reprograming provides a novel and potentially superior approach to harnessing TAMs for the treatment of immunosuppressive cancers.

### Systemic Delivery of STING Agonists Sensitizes STING-1 Null BP Tumors to Olaparib In Vivo

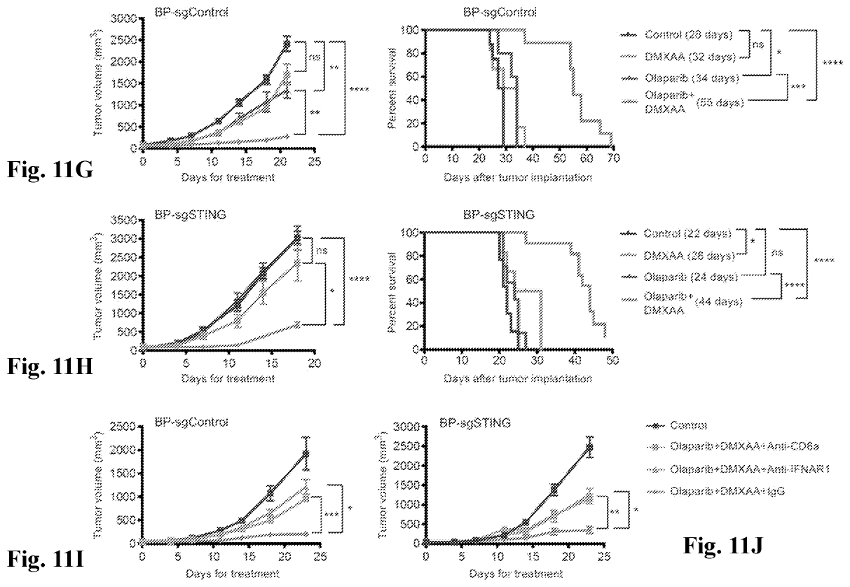

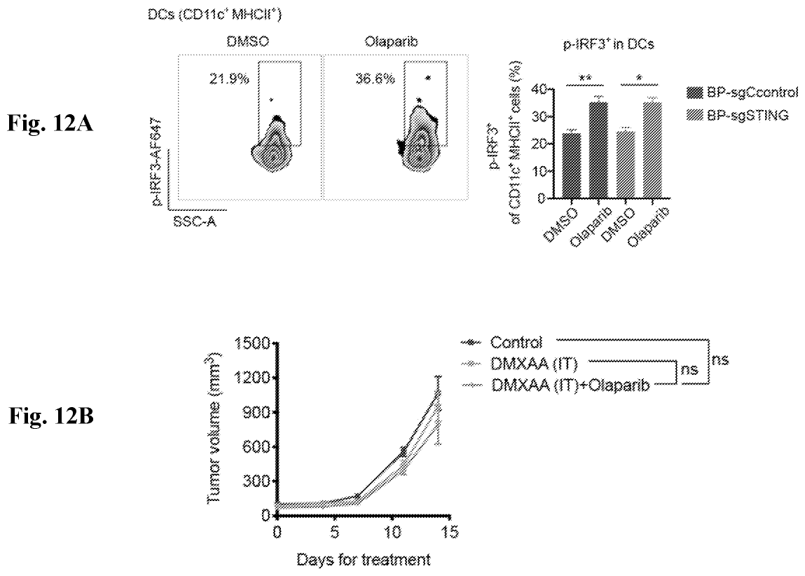

STING expression is frequently suppressed or lost in the majority of cancers (Konno H, et al. Oncogene 2018; 37 (15): 2037-51). Experiments sought to investigate whether STING in tumor cells is required for the therapeutic response to combined olaparib and STING agonist. STING-null BP cells were generated via CRISPR/Cas9-mediated gene editing, and the resulting cells were termed BP-sgSTING or BP-sgControl (FIG. 11A). Loss of tumor cell-intrinsic STING did not affect sensitivity to olaparib in vitro (FIG. 11B). Likewise, STING ablation in tumor cells did not affect STING pathway activation in DCs co-cultured with Olaparib-treated BP cells, as this likely depends on dsDNA released from dying and apoptotic tumor cells (FIG. 12A), and BP-sgSTING tumor cells (either with or without olaparib treatment) were still able to strongly promote M2-like polarization of macrophages (FIG. 11C). As expected, BP-sgSTING cells failed to up-regulate IFNB, Ccl5 and Cxcl10 in response to olaparib treatment (FIG. 11D-F), confirming loss of the STING pathway activation in tumor cells.

Next in vivo studies of STING-null BP tumors were performed in response to PARPi in combination with a STING agonist. Notably, unlike STING-WT BP tumor response to DMXAA and its combination with olaparib (FIG. 9A-FIG. 9C), BP-sgSTING tumors were completely refractory to DMXAA via i.t. injection as a single agent or in combination with olaparib (FIG. 12B), consistent with recently reported findings (Sen T, et al. Cancer Discov 2019; 9 (5): 646-61). These data indicate that reduced tumor-intrinsic immunity together with an immunosuppressive TIME likely abrogated local activation of anti-tumor immune response, thus rendering resistance to PARPi and i.t. administration of DMXAA.

Hence, experiments asked whether systemic administration of DMXAA could overcome such resistance. DMXAA (10 mg/kg) was administered weekly via intraperitoneal (i.p.) injection. Whereas i.p. injection of DMXAA as a single agent had no significant impact on the tumor growth of BP-sgControl or BP-sgSTING tumors, the combination of systemic DMXAA administration with olaparib resulted in strong inhibition of tumor growth, and significantly prolonged survival of both BP-sgControl and BP-sgSTING tumor-bearing mice (FIGS. 11G and 11H). Then an additional experiment was carried out by treating tumor-bearing mice with the combination of olaparib and systemic DMXAA in the presence or absence of IFNAR1 or CD8 blocking antibodies. As shown in FIGS. 11I and 11J, the efficacy of the combination therapy was significantly mitigated, but not completely prevented, by IFNAR1 or CD8 neutralization. These results suggest that both innate and adaptive immune function contribute to the antitumor activity driven by 1 olaparib in combination with STING agonist. Together, the data provide compelling evidence that combination of olaparib with systemic delivery of STING agonists overcomes the resistance of STING-null BRCA-deficient breast tumors to PARPi.

### Discussion

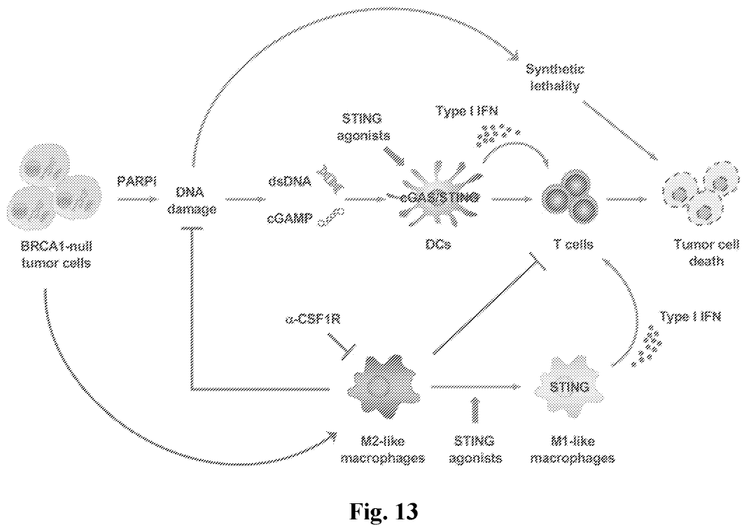

PARPi have markedly improved overall survival of patients with BRCA-mutated ovarian cancer, but not patients with BRCA-mutated breast cancer. Here is reported a previously undefined role of TAMs in orchestrating the resistance to PARPi in syngeneic GEMMs of Brca1-deficient breast tumor. Through a series of studies, results here demonstrate that BRCA-deficient breast tumor cells strongly induce up-regulation of pro-tumorigenic genes in TAMs through paracrine activation of macrophage M2-like phenotypes. These TAMs not only suppress CD8+ T cell activation, but also significantly reduce DNA damage in PARPi-treated tumor cells, thus attenuating synthetic lethal responses as well as reducing dsDNA-mediated, STING-dependent activation of DCs. Treatment with exogenous STING agonists reprograms TAMs, activates DCs and synergizes with PARPi in inducing intratumoral T cell responses and inhibiting tumor growth (FIG. 13). These are remarkable findings, since current understanding of PARPi resistance is mainly focused on tumor cell-intrinsic mechanisms, including the cellular availability of PARPi, restoration of HR or PARylation, and DNA replication fork protection (Noordermeer S M, van Attikum H. Trends Cell Biol 2019; 29 (10): 820-34). The studies provide insights into the role of macrophages in BRCA1-deficient breast cancer therapy and demonstrate a conceptually different mechanism of tumor cell-extrinsic, immune-mediated resistance to PARPi.

In agreement with these results, there is growing evidence showing TAM-mediated resistance to cytotoxic chemotherapies (Pathria P, et al. Trends Immunol 2019; 40 (4): 310-27). For example, recent studies report that TAMs promote chemoresistance by suppressing taxol-induced mitotic arrest (Olson O C, Kim H, Quail D F, Foley E A, Joyce J A. Tumor-Associated Macrophages Suppress the Cytotoxic Activity of Antimitotic Agents. Cell Rep 2017;19 (1): 101-13 doi 10.1016/j.celrep.2017.03.038) or by inhibiting gemcitabine via release of pyrimidines that outcompete drug uptake and metabolism (Halbrook C J, Pontious C, Kovalenko I, Lapienyte L, Dreyer S, Lee H J, et al. Macrophage-Released Pyrimidines Inhibit Gemcitabine Therapy in Pancreatic Cancer. Cell Metab 2019; 29 (6): 1390-9 e6). Despite these findings, therapeutic targeting of TAMs is challenging. CSF1R blockade is an effective approach to deplete TAMs, which depend on CSF1/CSF1R signaling for survival (Ries C H, Cannarile M A, Hoves S, Benz J, Wartha K, Runza V, et al. Targeting tumor-associated macrophages with anti-CSF-1R antibody reveals a strategy for cancer therapy. Cancer Cell 2014; 25 (6): 846-59). However, efforts to reprogram an immunosuppressive TIME with anti-CSF1R therapy have shown limited clinical benefit in advanced solid tumors (Lopez-Yrigoyen M, et al., Macrophage targeting in cancer. Ann N Y Acad Sci 2020; Papadopoulos K P, et al. Clin Cancer Res 2017;23 (19): 5703-10). Several mechanisms may be at play to limit the efficacy of anti-CSF1R therapy. For example, CSF1R blockade has been reported to indiscriminately deplete

TAMs, including a fraction of pro-inflammatory macrophages, 1 but spare a subset with pro-angiogenic features (Zhang L, et al. Cell 2020; 181 (2): 442-59 e29). In addition, CSF1R inhibition has been shown to attract Tregs (Gyori D, et al. JCI Insight 2018; 3 (11)) or up-regulate granulocyte-specific chemokine expression in cancer-associated fibroblasts, leading to the recruitment of granulocytic myeloid-derived suppressor cells (MDSCs) (Kumar V, et al. Cancer Cell 2017;32 (5): 654-68).

In contrast to the TAM depletion strategy, reprograming TAMs into an anti-tumor state may be a superior approach to harness the immune system against cancer. Studies have shown that dying tumor cells containing STING agonists, such as dsDNA, may trigger anti-tumor immunity by activating STING signaling in macrophages (Ahn J, et al. Cancer Cell 2018; 33 (5): 862-73; Zhou Y, et al. Immunity 2020; 52 (2): 357-73). However, this pathway may be silenced by the rapid degradation of tumor-derived DNA during TAM-mediated phagocytotic clearance of apoptotic tumor cells (Xu M M, et al. Immunity 2017;47 (2): 363-73). Here, results showed that a small molecule STING agonist efficiently reprograms the TAM phenotype from a pro-tumorigenic to an anti-tumorigenic state characterized by induction of type I IFN responses and expression of the co-stimulatory molecule CD86, which may stimulate T cell cross-priming and trigger a robust adaptive antitumor immunity. Moreover, while TAMs significantly suppress PARPi-induced synthetic lethal response in BRCA1-deficient breast tumor cells, STING agonists reprogram these TAMs and inhibit TAM-mediated suppression of synthetic lethality, thus rendering tumors more susceptible to PARPi therapy. Indeed, the results demonstrated that PARPi in combination with STING agonists has superior anti-tumor efficacy than PARPi combined with anti-CSF1R in the syngeneic GEMMs of Brca1-deficient breast tumors.

While it has been shown the central role of the STING pathway in PARPi-triggered anti-tumor immunity, the relative contribution of STING signaling in tumor cells and immune cells is not completely understood. It has been demonstrated that tumor DNA damage can be sensed by host immune cells, predominately DCs, leading to STING-dependent IFNβ production and anti-tumor T cell response (Corrales L, et al. J Clin Invest 2016; 126 (7): 2404-11; Deng L, et al. Immunity 2014; 41 (5): 843-52; Mender I, et al. Cancer Cell 2020). In agreement with this, it was found in that PARPi efficacy against Brca1-deficient murine ovarian tumors is significantly reduced in STING-deficient mice. Interestingly, recent studies have reported a role for tumor-intrinsic STING in the recruitment of CD8+ T cells through stimulating cytokine production in lung and breast cancer models. Consequently, loss of tumor STING had no effect on PARPi cytotoxicity in vitro, but rendered resistance to PARPi or other DNA damage response (DDR) inhibition in vivo, which could not be overcome by combining PARPi with PD-1 1/PD-L1 blockade (Wang Z, et al. J Clin Invest 2019; 129 (11): 4850-62). Herein, it was found that loss of tumor cell-intrinsic STING abolished PARPi-elicited tumor cell-intrinsic immunity, but without affecting tumor cell-mediated polarization of macrophages, and rendered complete resistance to PARPi. The loss of tumor cell-intrinsic STING may limit immune activation in the TIME owing to the lack of immune cell infiltration into STING-deficient tumors (Xiao Y, et al. Clin Cancer Res 2019; 25 (16): 5002-14). Strikingly, however, systemic administration of STING agonists overcame the resistance of STING-null tumors to PARPi, demonstrating that tumor STING is not a requirement for systemically administered STING agonists to boost host anti-tumor immunity and synergize with PARPi in priming and recruitment of CD8+ and CD4+ T cells. These findings have important clinical implications, as tumor cell-intrinsic STING signaling is frequently suppressed (Xia T, et al. Cell Rep 2016; 14 (2): 282-97; de Queiroz N, et al. Mol Cancer Res 2019; 17 (4): 974-86), and warrant further clinical investigation of the combination of a systemically administered STING agonist with PARPi in tumors with STING loss.

Given the promising results of many pre-clinical studies, to date the majority of efforts to exploit the immunomodulatory properties of PARPi have focused on the combination of PARPi with immune checkpoint blockade (ICB), which has led to more than 25 clinical trials across different types of HR-deficient cancers, including advanced breast cancer (Takahashi N, et al. Clin Cancer Res 2020; 26 (11): 2452-6). Unexpectedly, however, initial results suggest that the combination may not effectively enhance objective response rate (ORR) compared to historical cohorts that received PARPi as a single-agent treatment. Thus, it is important to better understand the mechanisms that may negate the efficacy of PARP inhibitors. This study indicates that TAMs play an important role in influencing the response of BRCA1-deficient breast tumors to PARP inhibition, supporting the need to assess the TIME in early-phase clinical trials. Indeed, accumulation of immunosuppressive macrophages has been frequently observed in the T cell-excluded or -inactivated TIME of advanced breast cancers (Gruosso T, Gigoux M, Manem V S K, Bertos N, Zuo D, Perlitch I, et al. Spatially distinct tumor immune microenvironments stratify triple-negative breast cancers. J Clin Invest 2019; 129 (4): 1785-800). Importantly, it was found that systemic delivery of STING agonists promotes the activation of anti-tumor T cells when combining with PARPi. Therefore, this combination therapy might prove particularly effective against tumors with low immune infiltration, such as a recently described subtype of advanced breast cancer with HR deficiency and high mutation load, but down-regulated STING signaling and poor immune infiltration. The next generation of STING agonists that can be delivered systemically is currently under clinical development (Ramanjulu J M, et al. Nature 2018; 564 (7736): 439-43; Chin E N, et al. Science 2020; 369 (6506): 993-9; Pan B S, et al. Science 2020; 369 (6506). The finding that systemic delivery of STING agonists synergized with PARP inhibition 1 has the potential to inform the design of future clinical therapies.

### Example 2: Methods for Example 1

### Mice

All the animal experiments described in this study were performed according to animal protocols approved by the DFCI Institutional Animal Care and Use Committee (IACUC). STING knockout mice (C57BL/6J-Tmem 173gt/J, Stock #017537) were purchased from The Jackson Laboratory. Brca1loxP/loxP mouse line was kindly provided by Dr. Jos Jonkers's laboratory at Netherlands Cancer Institute. Trp53loxP/loxP mouse line was obtained from National Cancer Institute Mouse Repository. Brca1loxP/loxP and Trp53loxP/loxP mouse lines were both backcrossed into the FVB/NJ background for more than ten generations, as was reported in (Ding L, et al. Cell Rep 2018; 25 (11): 2972-80 e5). To develop syngeneic genetically engineered mouse models (GEMMs) of Brca1-deficient breast cancer, Brca1loxP/loxP mice were further crossed with Trp53loxP/loxP mice. The resulting Brca1loxP/loxP; Trp53loxP/loxP mice were injected intraductally with adenovirus expressing Cre recombinase under a CMV promoter, which led to the development of mammary tumors driven by concurrent loss of Brca1 and Trp53 (referred as BP).

### Cell Culture

Cells were cultured in a humidified incubator under 5% CO2 at 37° C. MDA-MB-436, HCC1937 and THP-1 human cell lines were obtained from ATCC, tested negative for mycoplasma, and authenticated using short tandem repeat analysis (Promega GenePrint 10 System). MDA-MB-436 and HCC1937 breast cancer cells were cultured in RPMI 1640 medium (Gibco) supplemented with 10% heat-inactivated fetal bovine serum (FBS, Gemini) and 100 μg/mL penicillin-streptomycin (Gibco). THP-1 monocytes were cultured in RPMI 1640 with 10% FBS and 0.055 mM 2-mercaptoethanol (Gibco, #21985023). To differentiate THP-1 monocytes to macrophages, cells were treated with 100 nM PMA (Sigma, #P1585) for 48 h, followed by 24 h recovery with PMA-free medium.

Primary BP tumor cells were derived from mouse BP mammary tumors as described in (Palechor-Ceron N, et al. Am J Pathol 2013; 183 (6): 1862-70). Briefly, single-cell suspensions obtained from dissociated BP tumors were grown in serum-free F-Media [1:3 mixture of Ham's F-12 and DMEM (Gibco) supplemented with 25 ng/mL Hydrocortisone (Sigma), 5 μg/mL Insulin (Thermo Fisher), 8.5 ng/ml Cholera Toxin (Sigma), 0.125 ng/ml EGF (Sigma), 100 μg/mL penicillin-streptomycin (Gibco) and 5 μM of the Rock1 inhibitor Y-27632 (Selleck)]. After tumor cell selection, BP cells were 1 maintained in F-Media supplemented with 10% FBS.

Mouse macrophages and dendritic cells (DCs) were derived from the bone marrow (BM) of FVB/NJ mice by modifying protocols described in (Weischenfeldt J, Porse B. CSH Protoc 2008; Guo X, et al. J Immunol Methods 2016; 432:24-9). For generation of BM-derived macrophages (BMDMs), BM cells were seeded on ultra-low attachment plates (Corning) or petri dishes (Falcon) and cultured in DMEM supplemented with 10 ng/ml M-CSF (BioLegend, #576404), 10% FBS and 100 μg/mL penicillin-streptomycin. Fresh DMEM with 10 ng/ml M-CSF was added after 3 days, and cells were incubated for another 4 days before harvesting adherent cells (BMDMs). For DC differentiation, BM cells were seeded on tissue culture dishes (Corning) and cultured in RPMI 1640 supplemented with 20 ng/mL GM-CSF (Stem Cell Technologies, #78017), 10% FBS and 100 μg/mL penicillin-streptomycin. Fresh RPMI 1640 with 20 ng/mL GM13 CSF was added after 3 days, and non-adherent cells (DCs) were harvested after another 4 days. For preparation of tumor cell-conditioned media (CM), tumor cells were grown to 60% of confluence, washed twice with PBS, and then incubated with fresh DMEM for two days. CM were then harvested and centrifuged to collect the supernatant.

### Tumor Growth and Treatment

Primary mammary tumor cells for orthotopic injection were resuspended in serum-free DMEM containing 40% matrigel (Corning). 5×105 tumor cells in a total volume of 100 μL were injected orthotopically into the thoracic mammary fat pads of 8-week-old female FVB/NJ mice (The Jackson Laboratory). Tumor growth was examined by measuring the greatest longitudinal diameter (length) and the greatest transverse diameter (width) with digital calipers, and tumor volume was calculated by use of the modified ellipsoid formula (0.52×length×width2). Tumors were measured 2-3 times a week. All tumor measurements within single cohorts were performed by the same researcher. Tumor-bearing mice were randomized prior to start of treatment. Drug treatments were started when mean tumor volumes approximated 50-100 mm3. Mice were euthanized by CO2 inhalation when tumor volumes met humane endpoints described in the IACUC (20 mm diameter) protocols or upon severe health deterioration.

Olaparib (MedChem, #HY-10162) was prepared by diluting 100 mg/l mL stocks in DMSO with 10% (2-Hydroxypropyl)-β-cyclodextrin (HPCD, MedChem, #101103) in PBS and administered immediately after drug preparation by intraperitoneal (i.p.) injection at a dose of 50 mg/kg body weight daily. DMXAA (Sigma, #D5817) was reconstituted at 10 mg/mL in 7.5% NaHCO3(Gibco). For intratumoral injection (i.t.), 250 μg DMXAA (approximately 10 mg/kg body weight) was administered once per week for 3 doses. For i.p. injections, DMXAA was administered at a dose of 10 mg/kg body weight once weekly and terminated if tumor size exceeded 600 mm3. Anti-mouse PD-1 antibody (clone 332.8H3, kindly provided by Dr. Gordon Freeman at DFCI) was injected by i.p. at a dose of 10 mg/kg every 3 days. Anti-mouse CSF1R antibody (CS7, Eli Lilly) was dosed at 40 mg/kg via i.p. every 3 days. For IFNAR1 blockade, anti-mouse IFNAR1 antibody (200 μg/mouse; clone MAR1-5A3; BioXcell) was administered via i.p. 72 h and 24 h before start of the combination therapy (olaparib+DMXAA) and every 3 days thereafter. For CD8+ T cell depletion, anti-CD8 antibody (400 μg/mouse; clone YTS 169.4, BioXcell) or isotype control (400 ug/mouse; clone LTF-2, BioXcell) was administered via i.p. 48 hours and 24 hours before the combination therapy (olaparib+DMXAA) and every 4 days thereafter.

### Tissue Digestion

To obtain single-cell suspensions, tumors were excised, minced and dissociated in collagenase/hyaluronidase buffer [DMEM with 5% FBS, 10 mM HEPES (Gibco), 100 μg/mL penicillin-streptomycin, 20 μg/mL DNase I (StemCell) and 1× collagenase/hyaluronidase (StemCell)] for 45 min at 37° C. with agitation, followed by treatment with ammonium-chloride-potassium (ACK) buffer (Lonza) for red blood cell (RBC) lysis, and strained through a 70 μm strainer to remove undigested tumor tissues. Spleens and tumor-draining lymph nodes (TDLNs) were mechanically dissociated by passing the tissues through a 70 μm strainer using the plunger of a 5 ml syringe, and RBCs were lysed as described above.

### Flow Cytometry

For flow cytometry analyses of tumor and TDLN samples, single-cell suspensions were obtained as described above (Tissue Digestion). Cells were stained in cold FACS buffer (PBS containing 0.2% BSA and 5 mM EDTA) with LIVE/DEAD Fixable Aqua Dead Cell Stain (Thermo Fisher) for 30 min on ice, followed by blocking with anti-CD16/32 (BioLegend) for 20 min on ice. Cells were then incubated in FACS buffer for 30 min on ice with antibodies specific to CD45 (clone 30-F11, BioLegend), TCR β chain (clone H57-597, BioLegend), CD38 (clone 145-2C11, BioLegend), CD4 (clone RM4-5, BioLegend), CD8a (clone 53-6.7, BioLegend), PD-1 (clone 29F.1A12, BioLegend), CD44 (clone IM7, BioLegend), CD62L (clone MEL-14, BioLegend), IFN-γ (clone XMG1.2, BioLegend), TNF-α (clone MP6-XT22, BioLegend), Granzyme B (clone NGZB, eBioscience), CD11c (clone N418, BioLegend), I-A/I-E (clone M5/114.15.2, BioLegend), CD80 (clone 16-10A1, BioLegend), CD86 (clone GL-1, BioLegend), CD103 (clone 2E7, BioLegend), CD11b (M1/70, BioLegend), F4/80 (clone BM8, BioLegend), phospho-TBK1 (Ser172) (clone D52C2, Cell Signaling Tech.) or phospho-IRF-3 (Ser396) (clone D601M, Cell Signaling Tech.). For intracellular staining, Foxp3/Transcription Factor Staining Buffer Set (eBioscience, #00-5523-00) was used for fixation and permeabilization. For cytokine analysis, cells were stimulated with Leukocyte Activation Cocktail with protein transport inhibitor Brefeldin A (BD Biosciences, #550583) at 37° C. for 4 hours prior to the staining. Analysis of ϵ-HA2X (Ser139) was performed according to manufacturer's instructions. Briefly, ice-cold 70% ethanol (Decon) was added dropwise to the cell pellet while vortexing. Cells were then incubated at −20° C. for 1 hour and washed three times with cold staining buffer. For the staining, 5 μL p-HA2X antibody (clone 2F3, BioLegend) was added to approximately 1×106 cells in 100 μL staining buffer and incubated at 4° C. for 30 min. For Annexin V and 7-AAD staining, cells were detached with accutase (Sigma, #A6964). The detached cells (from culture medium and accutase treatment) were washed twice with cold PBS, and then incubated with 5 μL FITC Annexin V (BioLegend, #640906) and 10 μL 7-AAD (BioLegend, #420404) in 100 μL Annexin V binding buffer (BioLegend, #422201) at room temperature for 15 min. Flow cytometry was performed on an LSR Fortessa HTS analyzer (BD Biosciences). All data were analyzed using FlowJo software. Gating strategies are shown in the Figures.

### Cell Viability Assay

Tumor cells were seeded in 96-well plates at a density of 5000 cells per well and allowed to adhere overnight. Cells were then treated for 72 hours with indicated drugs at the concentrations shown. Cell viability was measured using CellTiter-Glo® 2.0 Cell Viability Assay (Promega, #G9242) according to the manufacturer's instruction. Growth inhibition was calculated by comparing the absorbance at 490 nm of drug-treated wells to that of untreated controls 1 set at 100%. Dose response curves and IC50 values were generated using a non-linear regression model in GraphPad Prism 8.

### Co-Culture Experiments

For in vitro co-culture of CD8+ T cells and tumor-associated macrophages (TAMs), TAMs (7-AAD-CD45+CD11b+F4/80+) were isolated from BP tumors 14 days after transplantation of tumor cells using a FACSAria™ II cell sorter (BD Biosciences), and CD8+ T cells were isolated from spleens of FVB/NJ mice using a mouse CD8+ T cell isolation kit (StemCell, #19853). In a 96-well plate, 1×105 CD8+ T cells per well were cultured alone or co-cultured with TAMs at a ratio of 1:1 in RPMI 1640 supplemented with 10% FBS, 0.055 mM 2-mercaptoethanol, 2 ng/mL IL-2 (Peprotech), 2.5 ng/ml IL-7 (Peprotech) and 50 ng/ml IL-15 (Peprotech) for 2 days. CD8+ T cells were analyzed by flow cytometry to assess IFNg expression as described above.

For in vitro co-culture of tumor cells with macrophages, 2×105 tumor cells per well were co-cultured with macrophages at a ratio of 1:1 (BP cells: mouse BMDMs) or 1:1.5 (MDA-MB-436 cells: THP-1 macrophages) in 6-well plates with indicated drug treatments or DMSO vehicle control for two days.

For in vitro co-culture of tumor cells with DCs, 2×105 tumor cells per well were seeded on 6-well plates and allowed to adhere overnight, followed by incubation with CM of macrophages and treatment with olaparib or DMSO vehicle control. After two days, tumor cells were washed twice with PBS, and co-cultured with 2×105 mouse BM-derived DCs per well in 1 mL RPMI 1640 supplemented with 20 ng/mL GM-CSF, 10% FBS and lipofectamine 3000 (2 μL/mL, Invitrogen). After 24 hours, co-cultured cells were harvested for flow cytometry, and floating cells (enriched to approximately 90% DCs) were collected for mRNA analysis, as reported in (15).

### Generation of STING-Deficient BP Cells

CRISPR double nickase plasmids with improved editing specificity (67) were used to generate STING-deficient BP tumor cells. Briefly, BP tumor cells cultured in 6-well plates were transfected with 2 μg/well of STING (Tmem 173) double nickase plasmid (Santa Cruz, #SC-428364-NIC) or control double nickase plasmid (Santa Cruz, #SC-1 437281) using lipofectamine 3000 (Invitrogen). Two days after transfection, cells were passaged onto a 10 cm dish and cultured in growth medium containing 3 μg/mL puromycin (Thermo Fisher) for selection. Puromycin-resistant cells were then used for isolation of single clones. After assessment of STING expression by western blot, all of the clones with STING depletion were combined, expanded, and used for tumor generation via allograft transplantation into syngeneic FVB hosts as described above.

### RNA Extraction and Reverse Transcription-Quantitative PCR (RT-qPCR)

Total RNA was extracted using RNeasy® Plus Mini Kit (QIAGEN, #74134). An iScript Reverse Transcription Supermix (Bio-Rad, #1708841) was used for the first-strand cDNA synthesis with 1 μg total RNA. Real-time PCR was performed using SYBR™ Select Master Mix (Thermo Fisher, #4472908) with gene-specific primers (mouse/16, forward 5′-TAGTCCTTCCTACCCCAATTTCC-3′, reverse 5′-TTGGTCCTTAGCCACTCCTTC-3′; mouse//1b, forward 5′-GCAACTGTTCCTGAACTCAACT-3′, reverse 5′-ATCTTTTGGGGTCCGTCAACT-3′; mouse Cxcl1, forward 5′-CCGAAGTCATAGCCACACTCAA-3′, reverse 5′-GCAGTCTGTCTTCTTTCTCCGTTAC-3′; mouse Ifnb, forward 5′-TCCGAGCAGAGATCTTCAGGAA-3′, reverse 5′-TGCAACCACCACTCATTCTGAG-3′; mouse Ccl5, forward 5′-GCTGCTTTGCCTACCTCTCC-3′, reverse 5′-TCGAGTGACAAACACGACTGC-3′; mouse Cxcl10, forward 5′-CCAAGTGCTGCCGTCATTTTC-3′, reverse 5′-GGCTCGCAGGGATGATTTCAA-3′; mouse Actb, forward 5′-CGGTTCCGATGCCCTGAGGCTCTT-3′, reverse 5′-CGTCACACTTCATGATGGAATTGA-3′; human IL6, forward 5′-ACTCACCTCTTCAGAACGAATTG-3′, reverse 5′-CCATCTTTGGAAGGTTCAGGTTG-3′; human IL1B, forward 5′-ATGATGGCTTATTACAGTGGCAA-3′, reverse 5′-GTCGGAGATTCGTAGCTGGA-3′; human CXCL1, forward 5′-AAGTGTGAACGTGAAGTCC-3′, reverse 5′-GGATTTGTCACTGTTCAGCA-3′; human GAPDH, forward 5′-CTCTGCTCCTCCTGTTCGAC-3′, reverse 5′-TTAAAAGCAGCCCTGGTGAC-3′). Relative mRNA levels were calculated using the AACT method. Mouse Actb and human GAPDH were used as endogenous controls for mouse and human samples, respectively.

Cytosolic Double-Stranded 1 DNA (dsDNA) Staining and Imaging

BRCA1-deficient tumor cells were cultured on glass coverslips in 6-well plates. Cells were treated with 5 μM olaparib or DMSO vehicle control for two days in the presence or absence of macrophage-derived CM. Following the treatment, cells were fixed and stained for cytosolic dsDNA as described in (Bakhoum S F, et al. Nature 2018; 553 (7689): 467-72). Briefly, cells were first fixed with 4% paraformaldehyde for 10 min. To stain cytosolic dsDNA, selective plasma membrane permeabilization was performed by incubating the fixed cells with 0.02% saponin (Sigma) in PBS for 5 min. Cells were then blocked with 2.5% normal goat serum in PBS for 30 minutes and stained with an anti-dsDNA antibody (1:200 dilution, Thermo Fisher, #MAB1293M1) in PBS with 1% BSA at 4° C. overnight, followed by staining with goat anti-mouse IgG (H+L) highly cross-adsorbed secondary antibody, Alexa Fluor 594 (Thermo Fisher, #A-11032). Cells were mounted with ProLong Diamond Antifade Mountant with DAPI (Thermo Fisher, #P36966). Staining was imaged using a Leica SP5X laser scanning confocal microscope. Fluorescence intensity of cytosolic dsDNA were analyzed using ImageJ/Fiji software as described in Pantelidou C, et al. Cancer Discov 2019; 9 (6): 722-37.

### Immunoblotting

Cells were pelleted and lysed using ice-cold RIPA buffer supplemented with protease and phosphatase inhibitor cocktail (Thermo Fisher). Protein concentration was determined using DC protein assay (Bio-Rad). Equal amount of protein extracts (40-60 μg) were loaded and separated by SDS-PAGE, and then transferred to polyvinylidene fluoride (PVDF) membranes. Membranes were blocked for 45 min at room temperature with 5% non-fat milk (Bio-Rad) in TBS plus 0.05% Tween 20, followed by incubation with primary antibodies overnight at 4° C., and then incubated with fluorescently-labeled anti-mouse IgG (Rockland Immunochemicals, #RL610-145-002) or anti-rabbit IgG (Molecular Probes, #A-21109) at room temperature for 1 hour. Western blots were visualized on an Odyssey® scanner (LI-COR).

### IFNβ ELISA

Tumor cells were treated with olaparib or DMSO vehicle control for two days. To assess IFNβ production by tumor cells, cell culture supernatants were harvested and subjected to centrifugation at 1,500×g for 10 min at 4° C. to remove floating cells and debris. IFNβ was then detected via mouse IFNβ ELISA Kit (Thermo Fisher, #424001) according to manufacturer's instructions.

The Cancer 1 Genome Atlas (TCGA) Analysis RNA-seq data were obtained from GEO DataSets (GEO: GSE62944), where the TCGA RNA-seq data of 24 cancer types were re-processed by aligning the FASTQ files downloaded from the Cancer Genomics Hub so that the gene expession could be compared across cancer types (Rahman M, et al. Bioinformatics (Oxford, England) 2015;31 (22): 3666-72). The BRCA1 mutation information of patients in TCGA corhorts were retrieved according to a recent study (Riaz N, et al. Nature communications 2017;8 (1): 857). M2 TAM immunosuppressive gene signature were derived as described in (Pan W, et al. Immunity 2017;47 (2): 284-97). Enrichment scores of M2 siganature were generated by single-sample gene set enrichment analysis (ssGSEA), as implemented in the GSVA R package.

### Transcriptome Analysis

Total RNA was isolated by RNeasy® Plus Mini Kit (QIAGEN) and sequenced on an Ion Torrent platform (Thermo Fisher) using an Ion AmpliSeq Custom Panel targeting 4,604 murine genes most relevant to the studies, as described in (15,71). To generate read counts per gene, data were analyzed using Torrent Suite and AmpliSeqRNA analysis plugin (Thermo Fisher). Then differential gene expression was studied using DESeq2 package (Love M1, Huber W, Anders S. Genome Biol 2014; 15 (12): 550) in R software environment. Genes with log 2 (fold change)>1 and P<0.001 were considered differentially expressed genes (DEGs). Volcano plots showing the significance and magnitude of log 2 (fold change) of these DEGs were generated by ggplot2 package in R. Gene ontology (GO) analysis of DEGs was performed using topGO package in R. For GSEA, genes were first ranked according to log 2 (fold change), and then analyzed using GSEAPreranked tool with MSigDB v7.1 HALLMARK gene sets and the ‘classic’ method (Subramanian A, et al. Proc Natl Acad Sci USA 2005; 102 (43): 15545-50).

Heat maps illustrating changes in gene expression were generated using the heatmap.3 package in R.

### Statistical Analyses

Statistical analyses were performed with Prism 8 (GraphPad Software Inc.) as described on each figure legend. Two-way ANOVA with Tukey's multiple comparisons test was used for tumor growth analysis. Log-rank Mantel-Cox test was used for survival analysis. For other analyses, unpaired two-tailed Student's t test (for normally distributed data) and Mann-Whitney nonparametric test (for skewed data that deviate from normality) were used to compare two conditions. One-way ANOVA with Tukey's multiple comparisons test (for normally distributed data) and Kruskal-Wallis nonparametric test (for skewed data) were 1 used to compare three or more means. Differences with P<0.05 were considered statistically significant.

### Data Availability

All data generated during this study are available within the paper. Transcriptomic data that support the findings of this study will be deposited before publication.

### Example 3: Novel Treatments for Ovarian Cancer

PARP inhibitors (PARPi) have demonstrated potent therapeutic efficacy in ovarian cancer treatment. Acquired resistance to PARPi is a major issue in the clinic and therapeutic approaches that can overcome the secondary resistance to PARP inhibition are urgently needed. By using PARPi-resistant mouse models and PDX models of ovarian cancer, a new mechanism underlying the secondary resistance to PAPR inhibition, which is mediated by tumor associated macrophages (TAMs) in the tumor microenvironment (TME) has been identified. Mechanistically, PARP inhibition activates STAT3 signaling pathway in tumor cells and promotes pro-tumor polarization of TAMs in the TME of ovarian cancer. STING agonists reprograms myeloid cells in the TME of ovarian tumor via repolarizing TAMs and increasing myeloid DCs in a STING-dependent manner. It was further shown that STING agonism overcomes the acquired immunosuppressive TME-induced secondary resistance to PARPi in mouse models and human PDXs. The data shown herein elucidates a new mechanism of PARPi resistance and provides a new treatment strategy to overcome acquired therapeutic resistance to PARP inhibition in ovarian cancers.

### INTRODUCTION

Homologous recombination deficiency (HRD), especially the mutations and dysregulation of BRCA1 and BRCA2 are frequently found in a variety of human malignancies, including ovarian, breast, pancreatic and prostate cancers. Based on the concept of synthetic lethality between poly (ADP-ribose) polymerase (PARP) inhibition and BRCA deficiency, PARP inhibitors (PARPi) have been developed for the treatment of BRCA-deficient tumors (Chan et al., 2021; Farmer et al., 2005). Growing number of PARPi have received FDA approval owing to their promising therapeutic efficacy in the clinic, especially in ovarian cancer (Abida et al., 2020 J Clin Oncol 38, 3763-3772; de Bono et al., 2020; New England Journal of Medicine 382, 2091-2102; Gong et al., 2021 Oncology (Williston Park) 35, 119-125; González-Martin et al., 2019 New England Journal of Medicine 381, 2391-2402; Ledermann et al., 2012 New England Journal of Medicine 366, 1382-1392; Mullard, 2017 PARP inhibitors plough on. Nat Rev Drug Discov 16, 229; Robson et al., 2017 New England Journal of Medicine 377, 523-533). Notably, recent studies showed that maintenance with a PARP inhibitor improved progression-free survival (PFS) in all subsets of patients with platinum-sensitive, recurrent, high-grade ovarian cancer, with a greatest benefit in the patients harboring BRCA1/2 mutation (Lee et al., 2021 A meta-analysis. Cancer 127, 2432-2441; Mirza et al., 2016; New England Journal of Medicine 375, 2154-2164). It has been reported that, in addition to synthetic lethality, PARPi elicit potent anti-tumor immune responses, which can be further enhanced by immune checkpoint blockade (Ding et al., 2018 Cell Rep 25, 2972-2980.e2975; Pantelidou et al., 2019 Cancer Discov 9, 722-737; Sen et al., 2019 Cancer Discov 9, 646-661). These preclinical findings are further supported by recent results from clinical trials of PARPi and ICB (Domchek et al., 2020 Lancet Oncol 21, 1155-1164; Konstantinopoulos et al., 2019 JAMA Oncol 5, 1141-1149; Lee et al., 2018 Annals of Oncology 29, viii334). While PARPi has changed the landscape of ovarian cancer treatment, resistance to PARPi, including both primary and secondary, has become a major problem in the clinic and appropriate management of patients with PARPi-resistant tumors are pressing concerns. A number of mechanisms underlying resistance to PARP inhibition have been identified from both clinical and preclinical studies, including restoration of homologous recombination, decreased PARP trapping, dysregulation of cell cycle and enhanced drug efflux (D'Andrea, 2018 Mechanisms of PARP inhibitor sensitivity and resistance. DNA Repair (Amst) 71, 172-176; Pantelidou et al., 2019 Cancer Discov 9, 722-737; Pettitt et al., 2020 Cancer Discovery 10, 1475). Reversion mutations of BRCA1/2 have been discovered as a major molecular mechanism of HR restoration and PARPi resistance in ovarian cancer. Of note, recent studies report that about 20-50% of recurrent ovarian cancers acquire reversion mutation of BRCA1/2 (Lin et al., 2019; Norquist et al., 2011). While most of studies focused on tumor cell-intrinsic resistance to PARPi, some recent findings suggest the role of tumor microenvironment or host immune system in the PARPi resistance. For example, PARPi are found to induce immunosuppression via up-regulating PD-LI expression or enhancing both anti- and pro-tumor features of macrophages in breast cancer (Jiao et al., 2017 Clinical Cancer Research 23, 3711; Mehta et al., 2021 Nature Cancer 2, 66-82). It remains unclear whether the host immune system also plays a role in PARPi resistance in ovarian cancer.

Using preclinical mouse models that acquired secondary resistance to PARP inhibition, a new mechanism of PAPRi resistance was elucidated that mediated by tumor cell-intrinsic STAT3 signaling and TAMs in BRCA1-deficient ovarian tumors. It was further demonstrated that STING agonism efficiently reprogramed myeloid cells in TME and overcome the TME-dependent secondary resistance to PARP inhibition in both mouse and human ovarian cancer models. Thus, new treatment options are provided for some ovarian cancer patients with acquired resistance to PARP inhibition.

### BRCA1-Deficient Ovarian Tumors Developed Secondary Resistance to PARP Inhibition

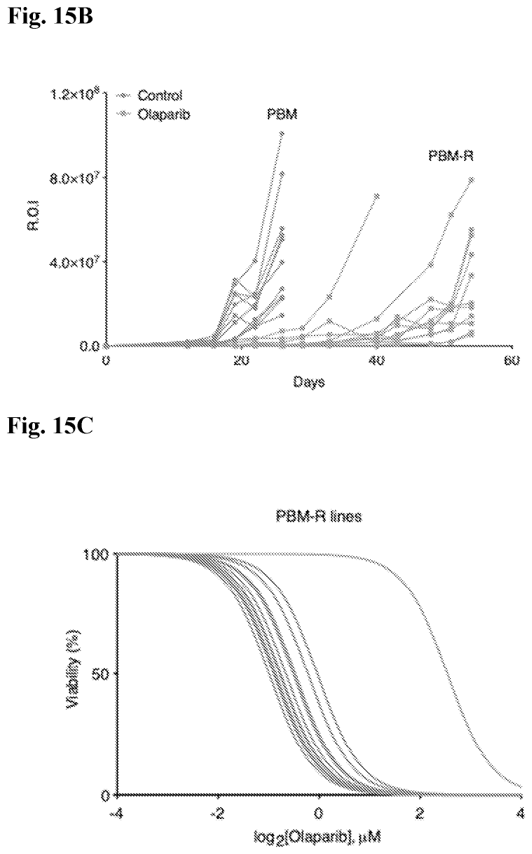

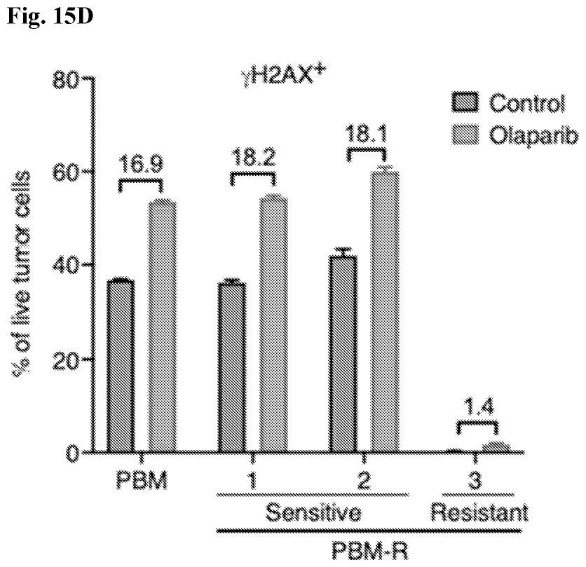

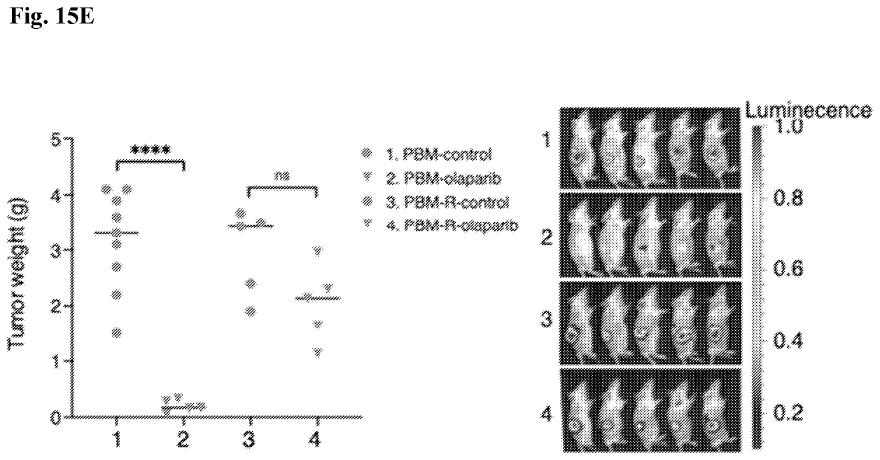

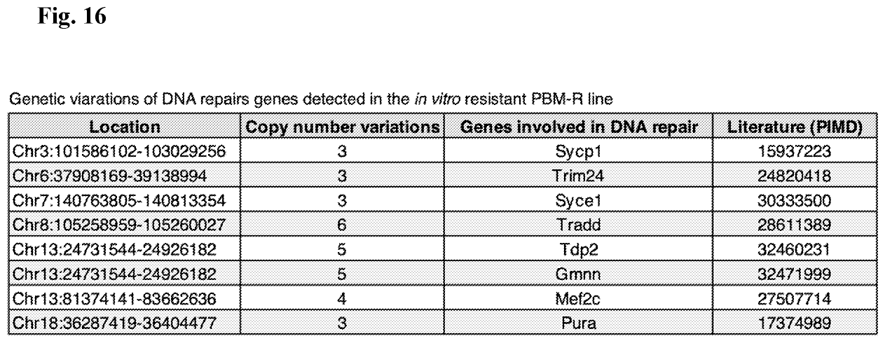

A syngeneic, genetically engineered mouse model of ovarian cancer driven by concurrent ablation of Brca1 and Trp53 and overexpression of c-Myc, termed PBM, which recapitulates highly aggressive serous carcinomas of human ovarian cancer was generated (Ding et al., 2018 Cell Rep 25, 2972-2980.e2975). While these PBM tumors had initial robust response to PARP inhibition, most of the tumors eventually progressed on olaparib treatment (termed PBM-R, FIGS. 15A and 15B) (Ding et al., 2018 Cell Rep 25, 2972-2980.e2975). To investigate the mechanism underlying this secondary resistance to PARPi in BRCA1-deficient ovarian tumor model, 12 PBM-R tumors were harvested and established their primary cell lines in culture (FIG. 15B). Strikingly, 11 out of 12 PBM-R tumor lines are sensitive to PARPi in vitro with IC50 values comparable to PBM tumor cells (FIG. 15C). Only PBM-R3 tumor cells were truly resistant to olaparib with a IC50 4.19 μM, 7.48 folds of 0.56 μM. Consistently, PBM-R tumor cells have significantly increased DNA damage upon Olaparib, while PBM-R3 cells have a little DNA damage signaling as measured by histone H2AX phosphorylation at Seine 139 (γ-H2AX), an early cellular response marker to DNA DSBs (FIG. 1D). Whole exome sequencing (WES) analysis revealed that PBM-R3 tumor cells had copy number alterations, specifically, copy number gains in multiple genes involved in DNA repair pathways, which were not detected in the PBM-R1 and -R2 (FIG. 16). It has been shown that compromised DNA damaging and/or increased DNA repairment, i.e. BRCA reversion mutations and copy number gains of DNA repair genes, are associated with resistance to PAPRi (FIG. 16). Since majority of secondary resistant PBM-R tumors were responsive to olaparib in vitro, indicating an involvement of a tumor extrinsic resistance mechanism. It was further confirmed that these PBM-R tumors were indeed resistance to PARPi in vivo when orthotopically re-transplanted to syngeneic host mice (FIG. 15E).

### Pro-Tumor Macrophages Enriched in the TME of PARPi-Resistant Ovarian Tumors

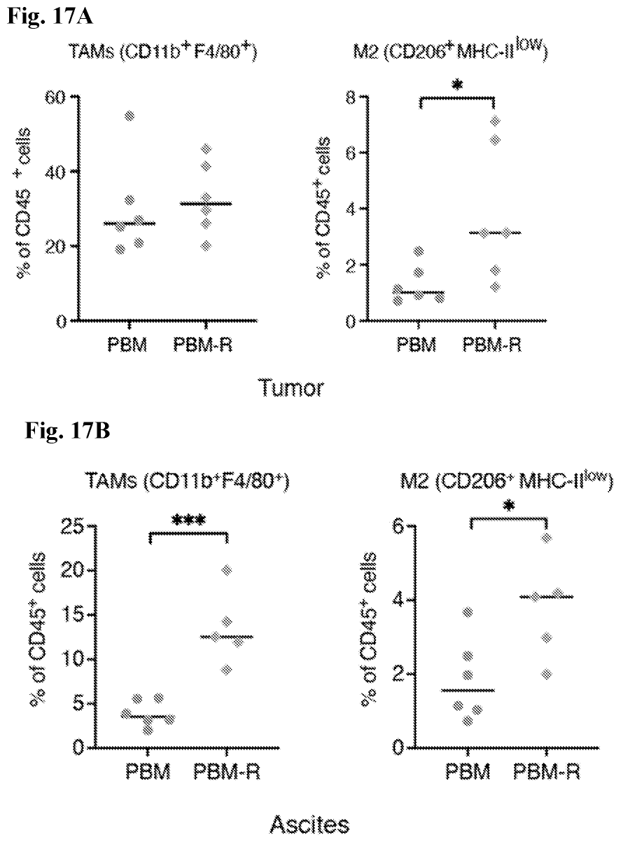

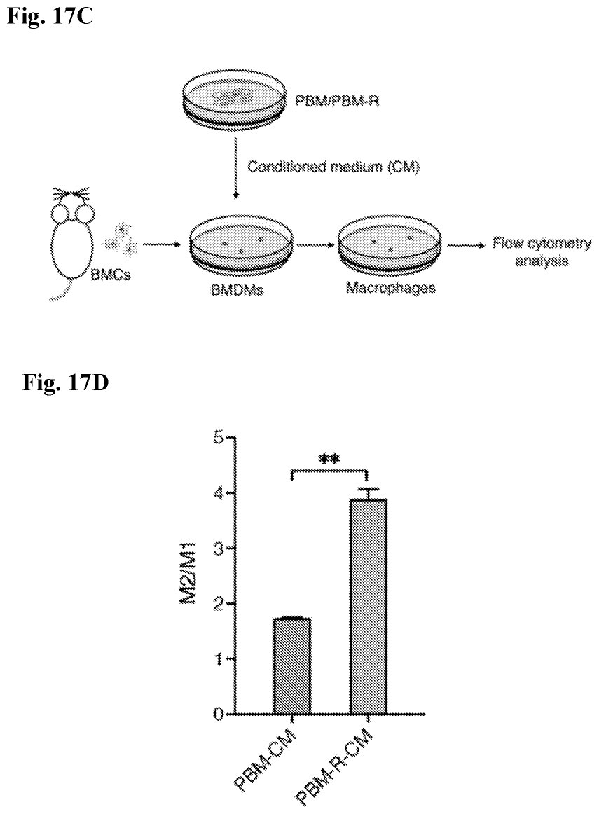

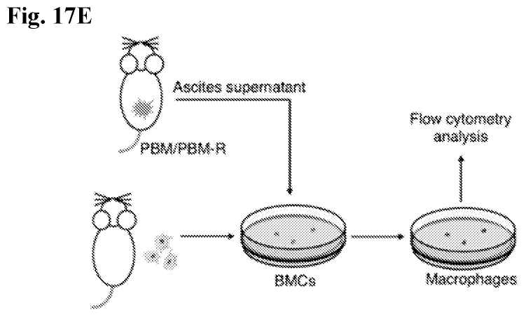

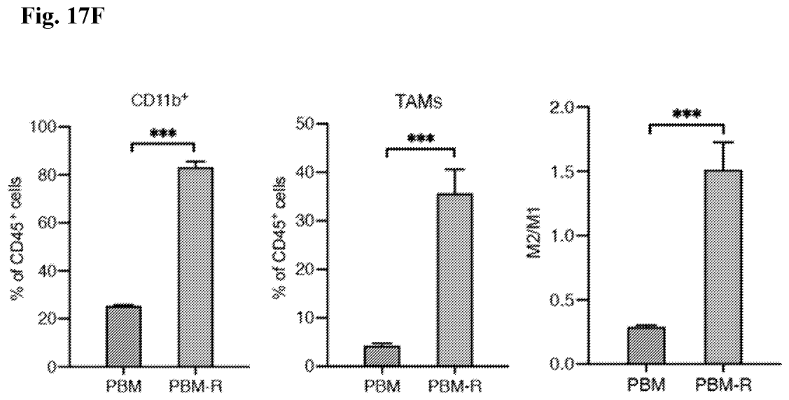

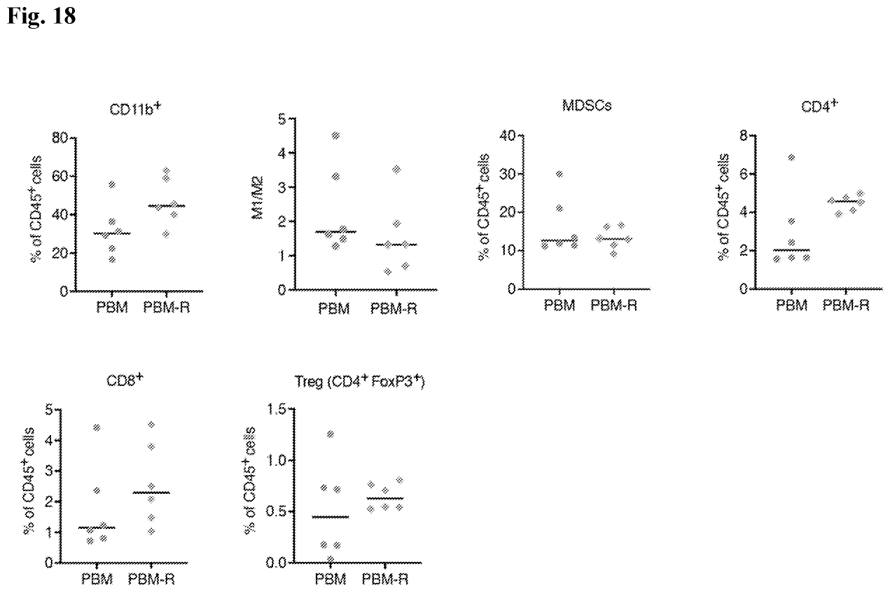

To explore the tumor cell-extrinsic mechanism underlying PAPRi resistance in Brca1-deficient ovarian tumors, immune cells were assessed in the TME of PBM tumors and PBM-R tumors by flow cytometry analysis. This data revealed that, whereas the populations of total CD11b⁺, TAMs and MSDCs were similar in PBM and PBM-R tumors, the proportion of pro-tumor (M2-like, MHC-II^{Low}CD206⁺) macrophages significantly increased in PBM-R tumors as compared to PBM tumors (FIG. 17A and FIG. 18A-18C). No significant changes of tumor-infiltrating CD4⁺, CD8⁺ and Treg cells were observed between PBM and PBM-R tumors (Figure S2D-S2F). Since ascites is an important specific microenvironment that contributes to ovarian tumor growth, immune cells were further analyzed in ascites and found that both TAMs and M2-like macrophages were significantly increased in PBM-R tumor-bearing mice compared with PBM-tumor bearing mice (FIG. 17B). These results indicate that PBM-R tumors might have acquired the ability to promote the polarization of pro-tumor macrophages.

To assess the effects of PBM and PBM-R tumor cells on macrophage polarization, bone marrow-derived macrophages (BMDMs) were incubated in the conditioned medium (CM) from PBM (PBM-CM) or PBM-R (PBM-R-CM). PBM-R-CM could promote M2-like polarization of TAMs ex vivo and showed a much stronger effect on M2-like TAM polarization (FIGS. 17C and 17D). Bone marrow cells (BMCs) in ascites were further cultured in supernatant collected from PBM or PBM-R tumor-bearing mice, the result showed that PBM-R ascites supernatant not only induced differentiation of myeloid cells (CD11b⁺) and TAM, but also promoted M2-like polarization ex vivo (FIGS. 17E and 17F). These data suggest that PBM-R tumor cells can strongly promote M2-like macrophage polarization both in the ex vivo co-culture system and in vivo in the TME of tumor-bearing mice.

PARP Inhibition Upregulates STAT3 Signaling Pathway in Tumor Cells, which in Turn Promotes Pro-Tumor Macrophage Polarization

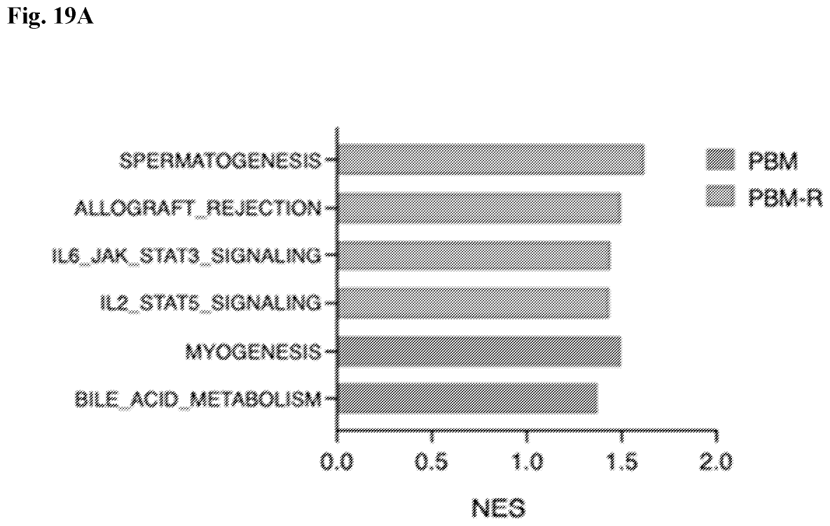

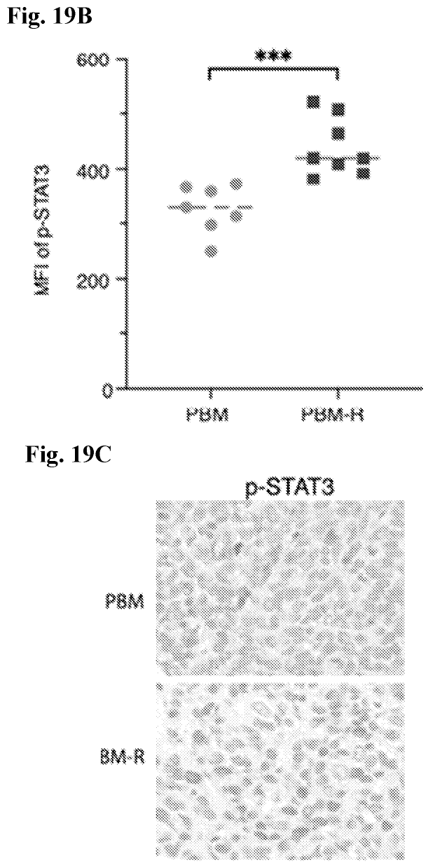

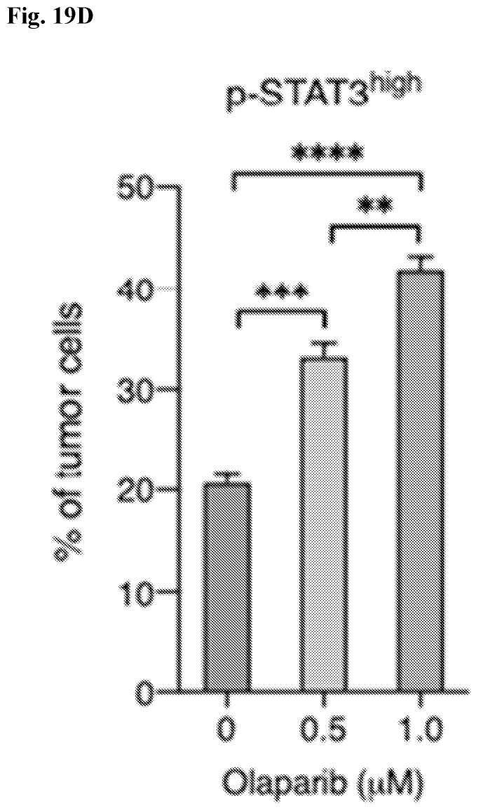

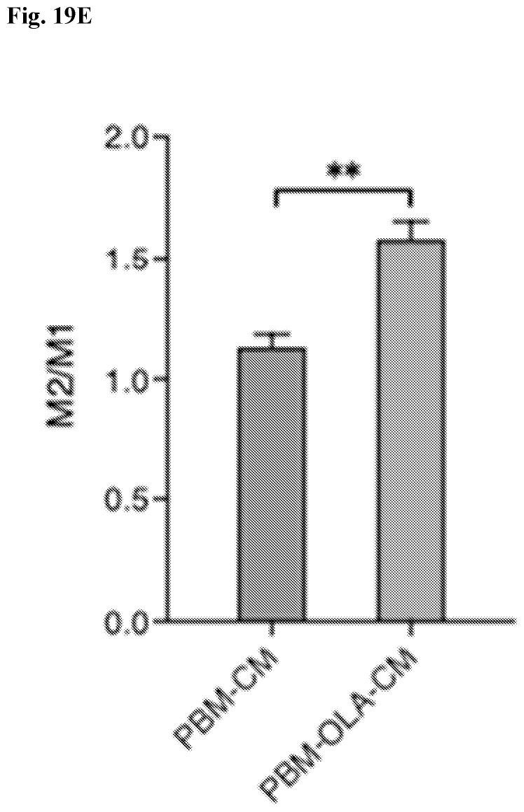

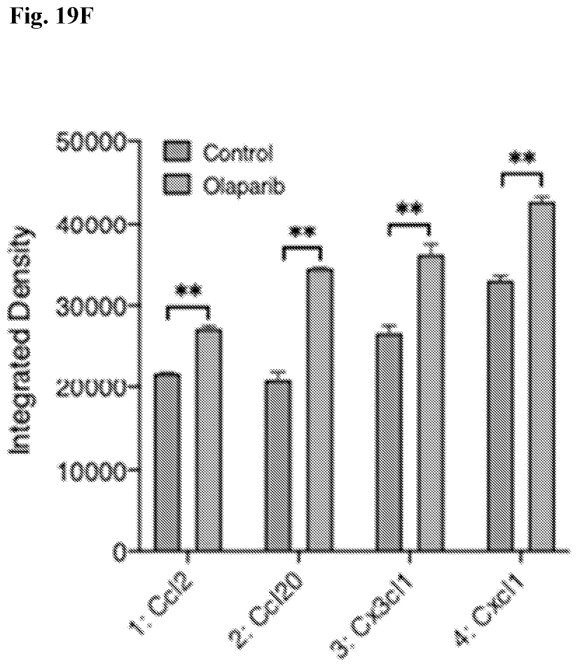

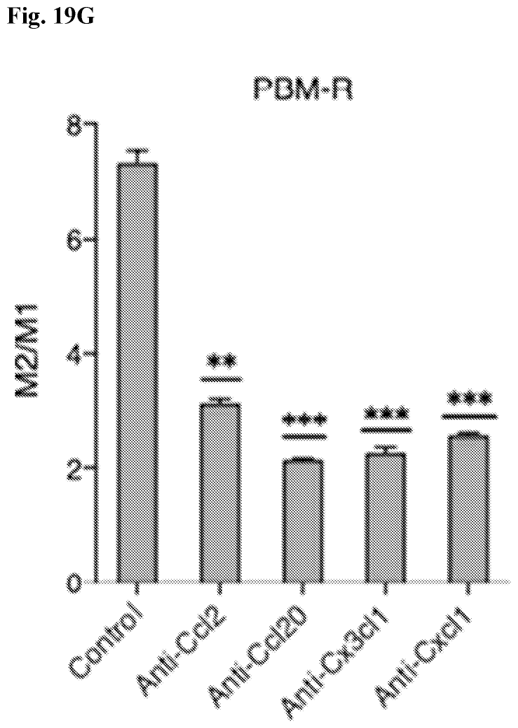

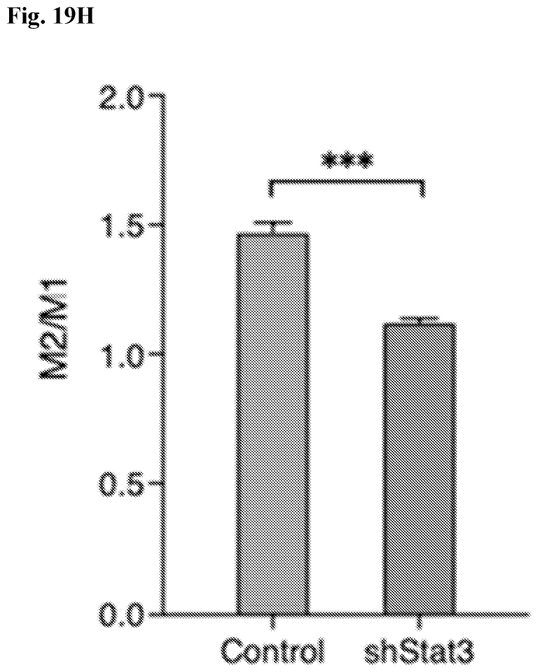

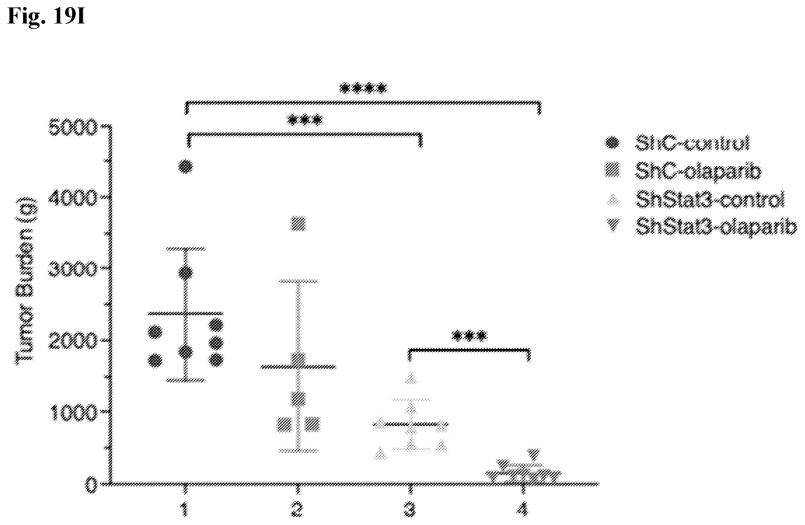

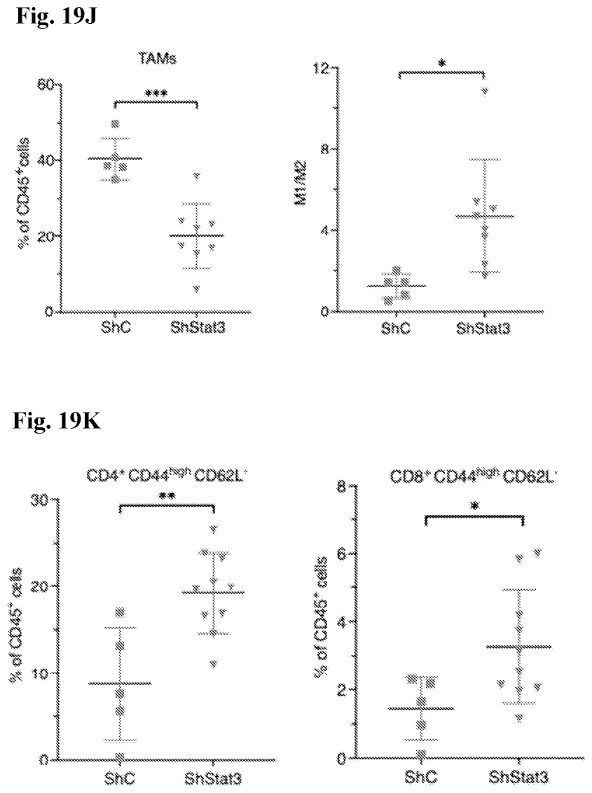

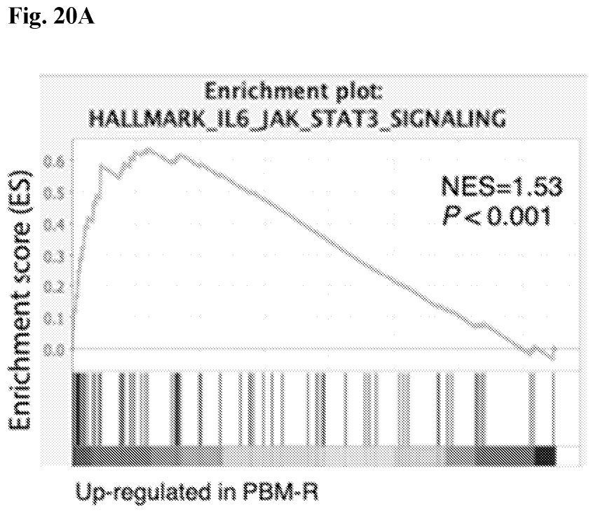

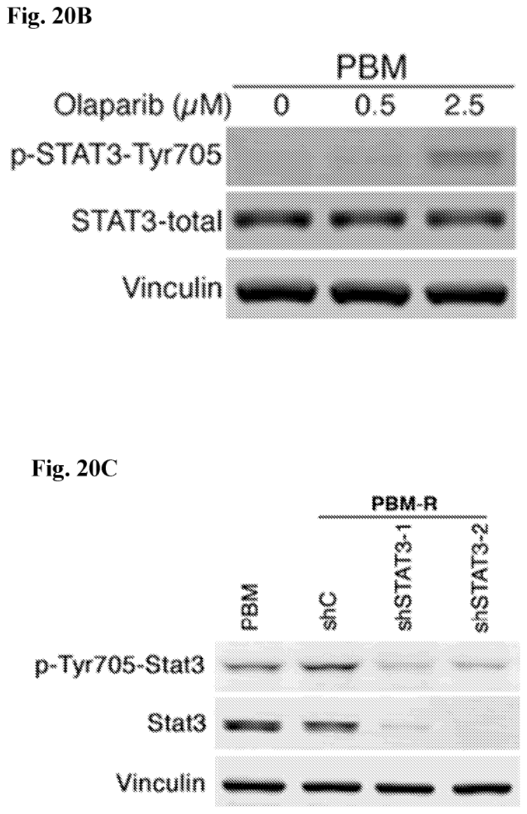

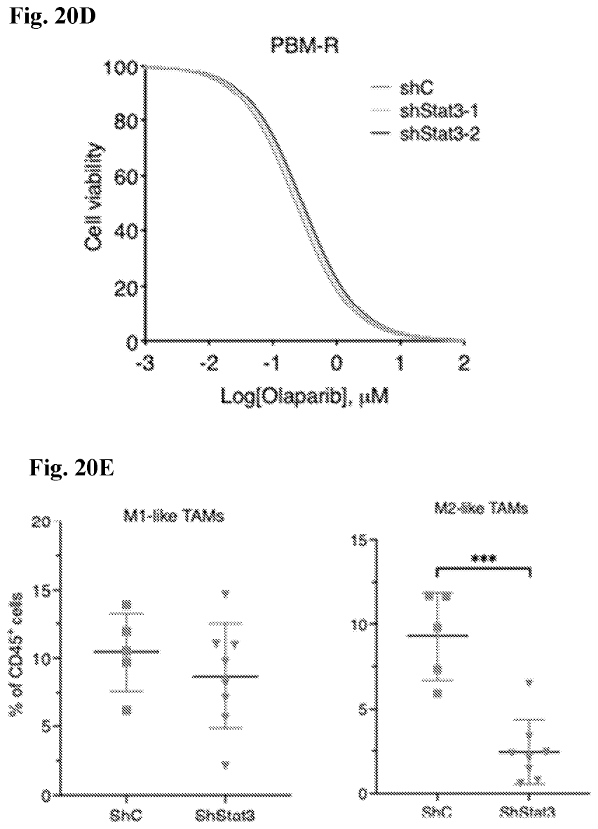

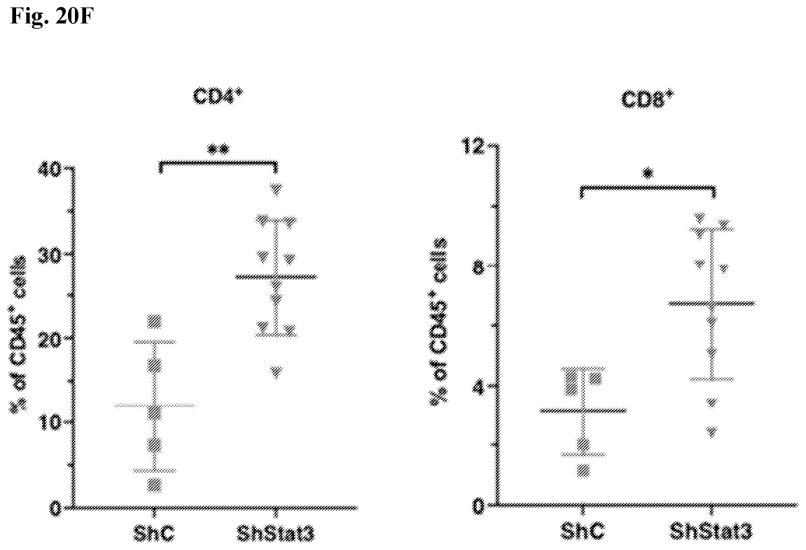

To investigate the underlying mechanism of PBM-R cells' ability that causes M2-like macrophage polarization, RNA-seq analysis of PBM and PBM-R tumors contained >95% cancer cells was performed. GSEA analysis revealed that STAT3 signaling pathway was significantly activated in PBM-R tumors when compared to the PBM tumors (FIG. 19A and FIG. 20A). Flow cytometry analysis also showed that PBM-R tumor cells had a higher level of phosphorylated STAT3 (Y705) than PBM tumor cells (FIG. 19B). This observation was further confirmed by immunohistochemistry analysis of p-STAT3 in PBM and PBM-R tumors (FIG. 19C).

Next, it was asked whether PAPRi can directly induce phosphorylation of STAT3 in tumor cells. Treatment of PAPRi-naïve PBM tumor cells with olaparib followed by flow cytometry and Western blot analyses revealed that increased phosphorylation levels of STAT3 in cultured PBM cells in a dose-dependent manner (FIGS. 19D and 20B). Notably, conditioned medium (CM) collected from olaparib-treated PBM cells (PBM-OLA-CM) showed stronger effect on M2-like TAM polarization of BMDMs than PBM-CM (FIG. 19E). Cytokine array analysis indicated that several STAT3-regulated chemokines (CCL2, CX3CL1, CCL20 and CXCL1) were significantly increased in the CM of olaparib-treated PBM cells (FIG. 19F) (Ikeda et al., 2016 Oncotarget 7, 13563-13574; Liu et al., 2013 PLOS ONE 8, e75804; Shen et al., 2018 Neurochem Res 43, 556-565; Yang et al., 2016 Cancer Research 76, 4124-4135). Blockage of these chemokines partially abrogated the effect of olaparib-treated PBM cells on promotion of pro-tumor macrophage polarization (FIG. 19G). These data suggest that PARPi-induced tumor cell STAT3 activation may contribute to M2-like polarization of macrophages.

To further evaluate whether the activation of STAT3 in tumor cells is required for M2-like macrophage polarization in PARPi-resistant BRCA1-deficient ovarian cancer, STAT3 was silenced by introduction of two individual lentivirus-mediated shRNAs targeting different regions of STAT3 gene in PBM-R tumor cells (FIG. 20C). Knockdown of STAT3 in PBM-R cells decreased the ratio of M2/M1 that induced by CM from PBM-R tumor cells (PBM-R CM) (FIG. 19H). Notably, while STAT3 knockdown in PBM-R cells did not affect tumor cells response to olaparib treatment in vitro (FIG. 20D), PBM-R-shStat3 tumors became sensitive to olaparib treatment in vivo. Flow cytometry analysis indicated that olaparib treatment induced antitumor activity and immune modulatory properties were restored in PBM-R-shStat3 tumors as reflected by increased ratio of M1/M2 and decreased M2-like TAMs, as well as increased total and effector CD4⁺ and CD8⁺ T cells (FIGS. 19J and 19K, FIGS. 20E and 20F). These data suggest that STAT3 activation in PBM-R tumors is responsible for inducing M2-like TAM polarization in the TME.

### STING Agonists Reprogram TAMs and Activate Myeloid DCs in the TME of PBM-R Tumors

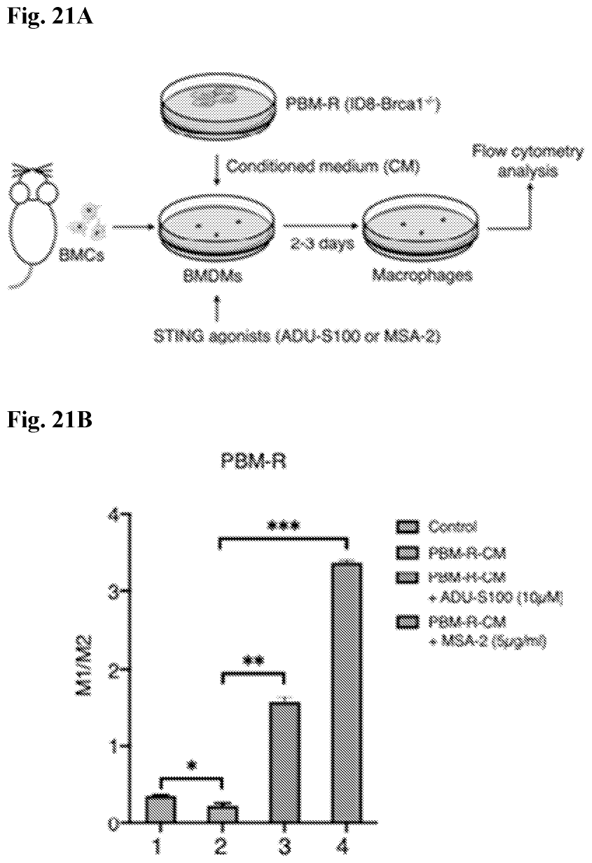

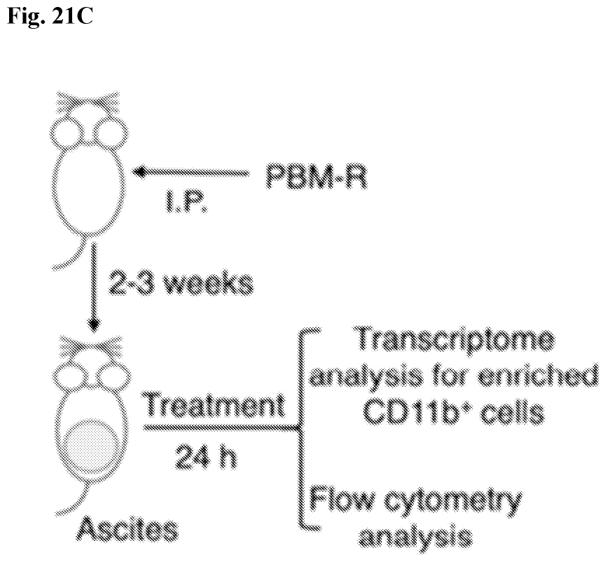

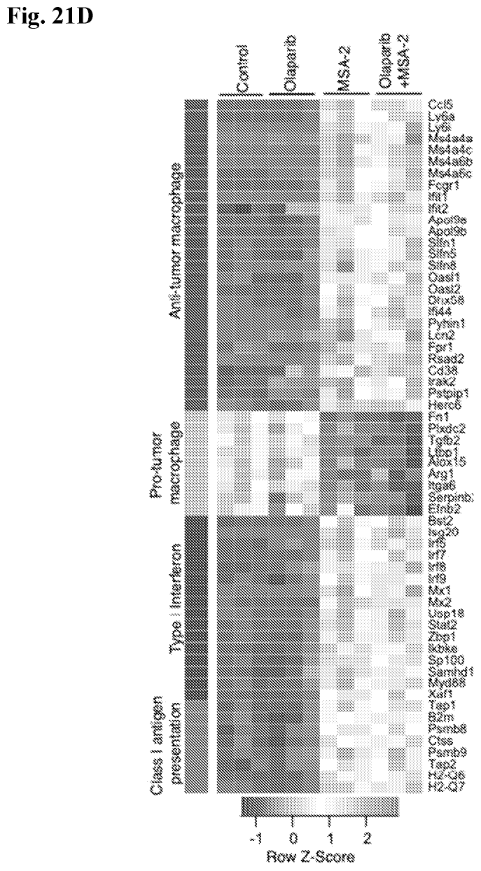

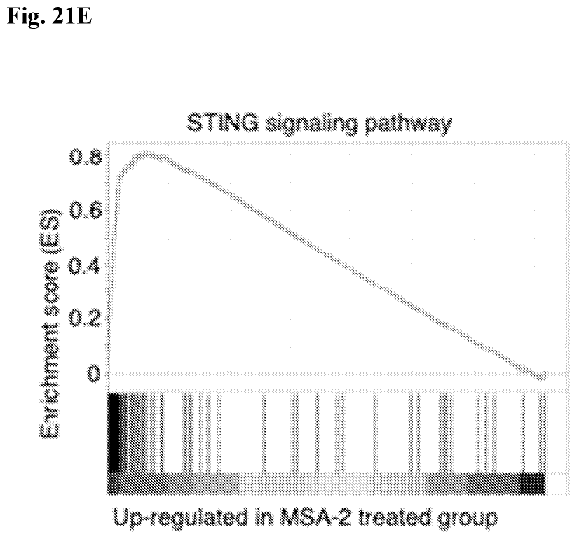

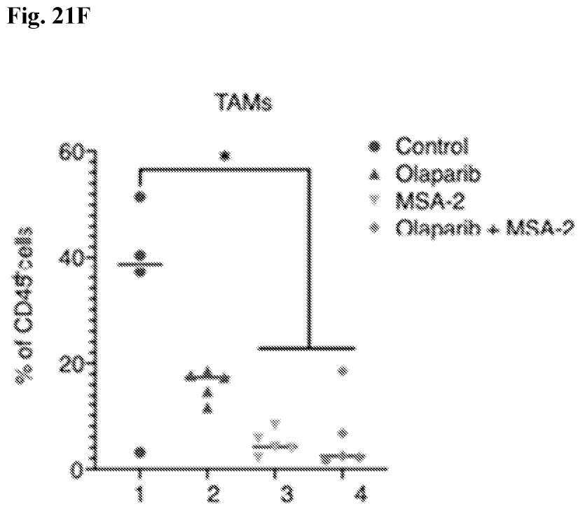

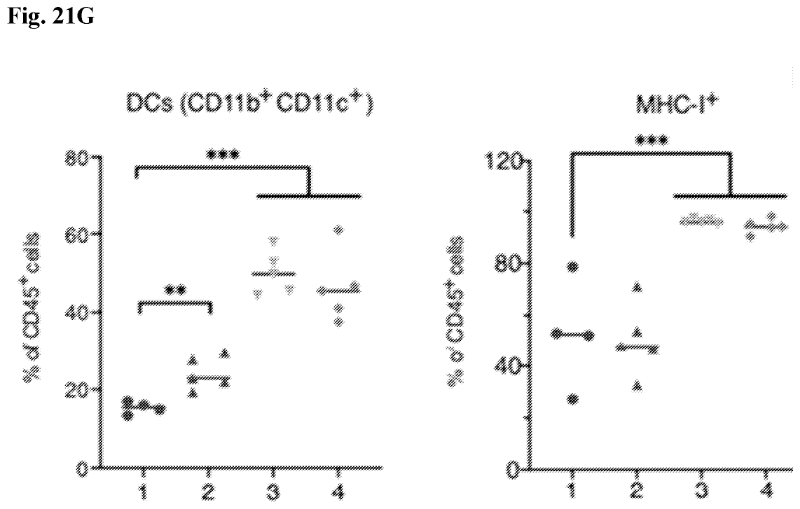

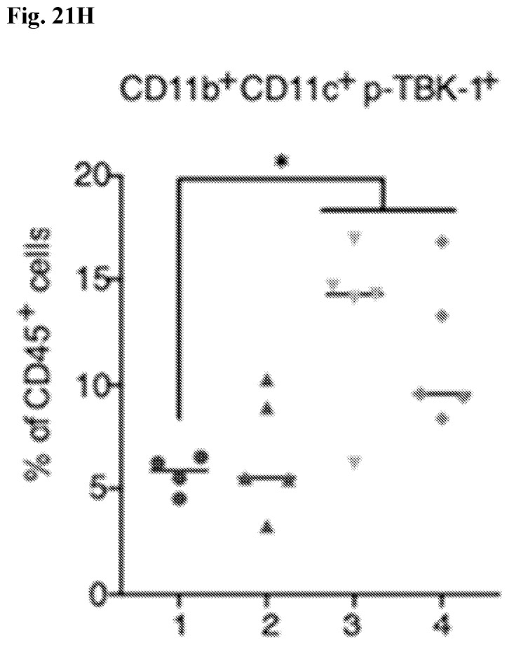

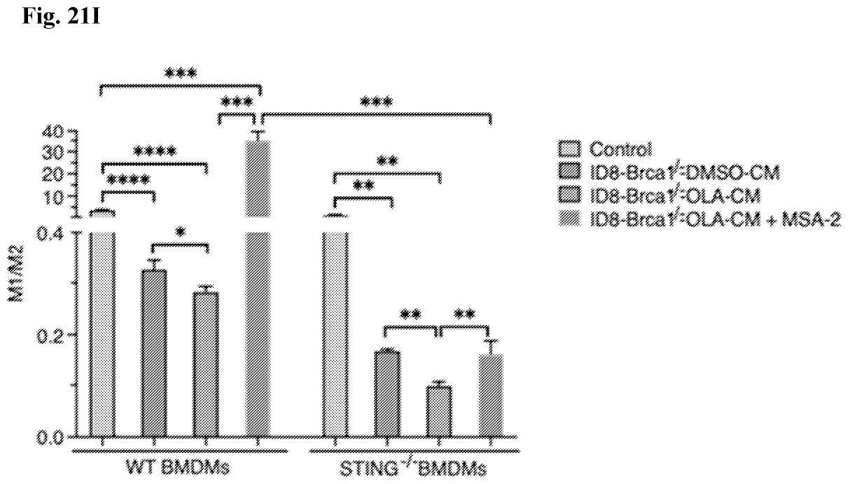

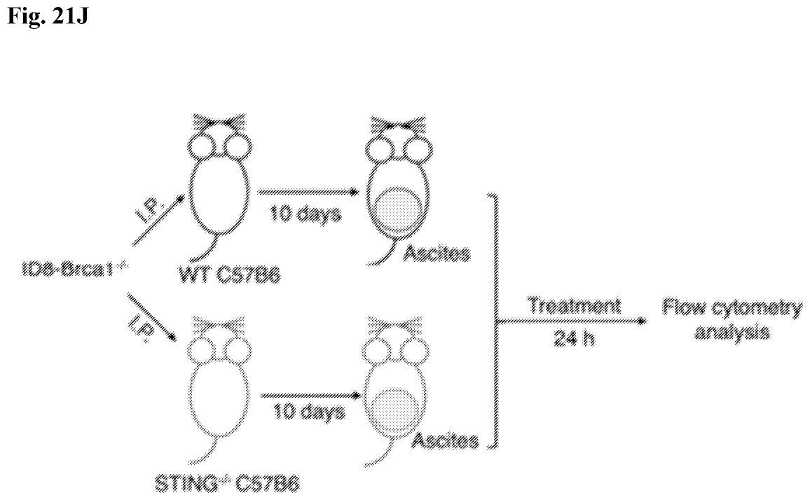

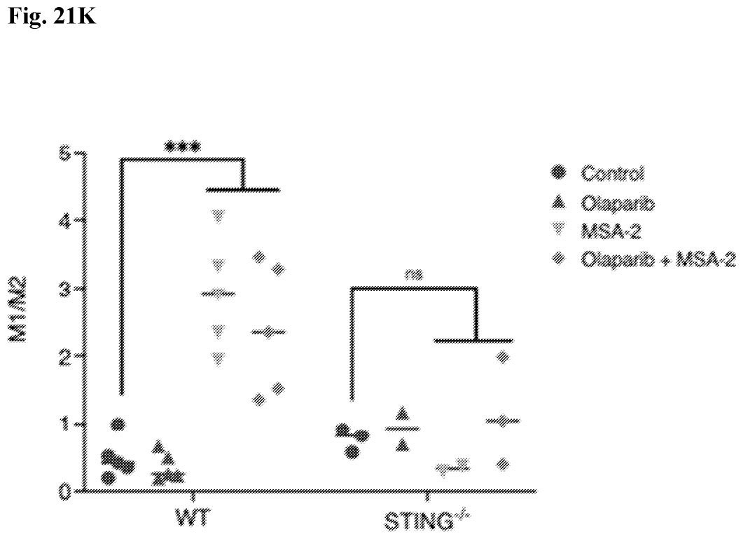

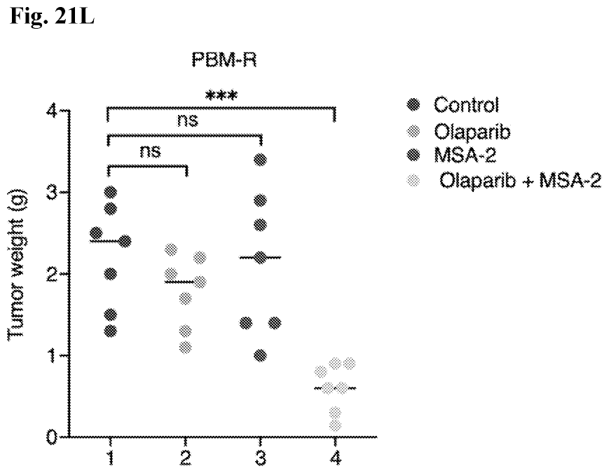

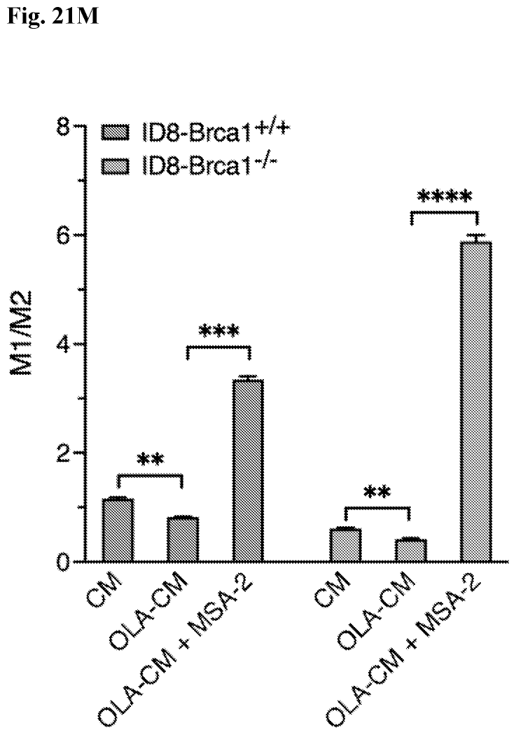

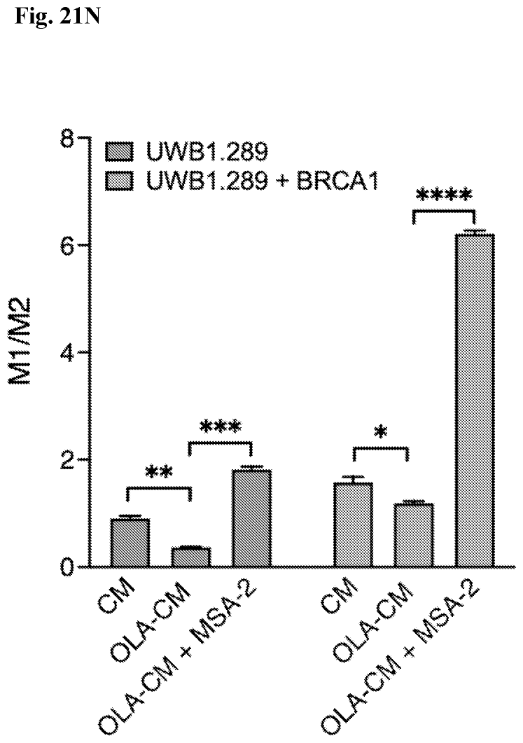

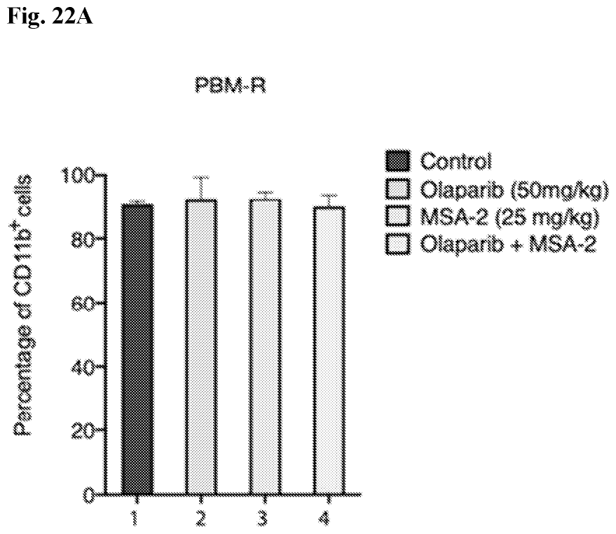

Increasing evidence indicates that activation of STING signaling can remodel the TME by antagonizing MDSC expansion and reprogramming immunosuppressive macrophages into immune-activating subtypes (Downey et al., 2014 PLOS ONE 9, e99988; Jassar et al., 2005 Cancer Res 65, 11752-11761; Jing et al., 2019; Zhang et al., 2019 Therapy of Cancer 7, 115). It was tested whether STING agonists can alter PARPi-induced immunosuppressive TME to overcome the resistance of PBM-R tumors to PARPi. The effect of STING agonists on macrophage polarization in vitro was first tested. BMDMs generated from FVB/NJ mice were incubated with PBM-R-CM and treated with vehicle control or STING agonists, MSA-2 or ADU-S100 (FIG. 21A). As expected PBM-R-CM decreased M1/M2 ratio (FIG. 21B). Addition of either MSA-2 or ADU-S100 significantly increased M1/M2 ratio (FIG. 21B), suggesting STING agonists shifted macrophages towards M1-like.

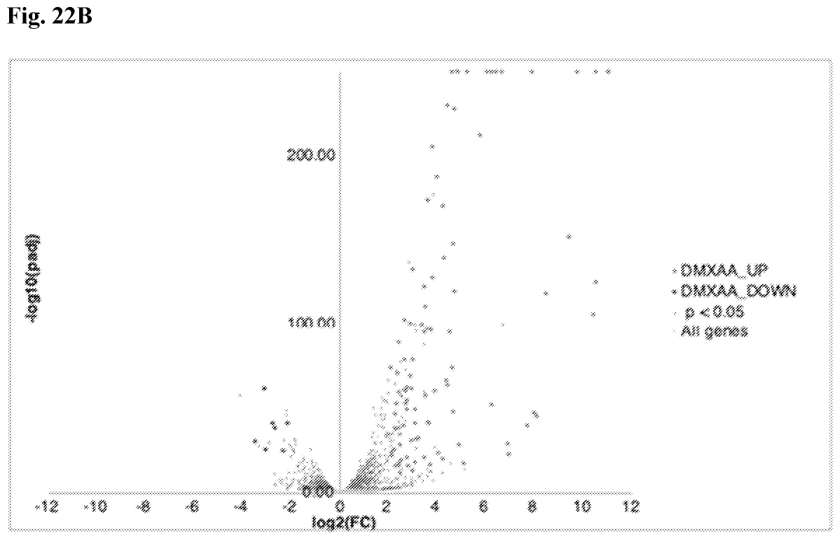

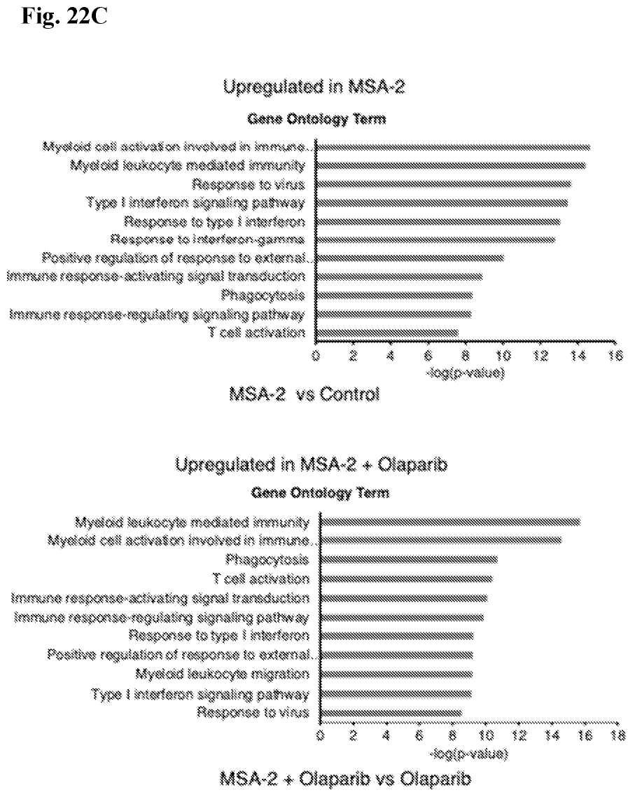

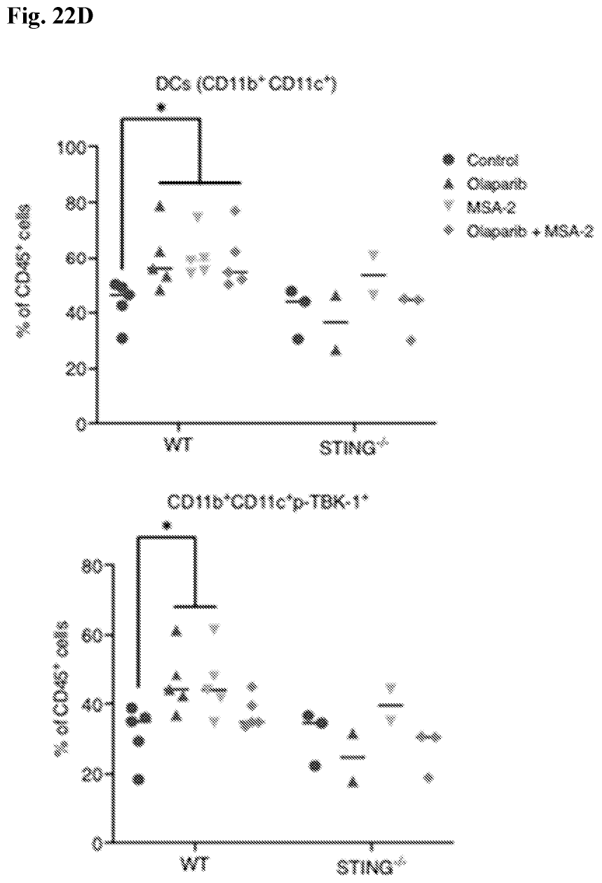

To investigate this further in vivo, PBM-R tumor cells were intraperitoneally injected into FVB/NJ mice to induce immunosuppressive TME in the ascites. PBM-R tumor bearing mice with olaparib and MSA-2 as a single-agent or in combination for 24 hours was then tested (FIG. 21C). Myeloid cells (CD45⁺CD11b⁺) were isolated from the ascites of PBM-R tumor-bearing mice after treatment by using a CD11b⁺ positive selection kit and the purity of isolated myeloid cells was confirmed by flow cytometry analysis (FIG. 22A). Transcriptomic analysis of the isolated myeloid cells was performed, and it was found that, compared to control groups, MSA2 treatment not only inhibited the expression of genes (Fn1, Plxdc2, Tgfb2, Ltbp1, Alox15, etc.) associated with pro-tumorigenic M2 polarization, but also strongly up-regulated expression of M1-like/anti-tumorigenic genes (Ccl5, Ly6a, Ly6i, Il18 bp, Ifi44, etc.) (FIG. 21D).

To derive a gene expression signature characteristic of cGAS-STING pathway activation, in vitro cultured murine DCs were treated with DMSO control, DMXAA or DMXAA in combination with a STING pathway inhibitor-BX795. RNA-sequencing analysis was preformed and a gene expression signature that comprise 94 upregulated genes and 7 downregulated genes were identified for activation of cGAS-STING pathway in DCs upon STING agonist treatment (FIG. 22B). By using the cGAS-STING pathway signature generated in DCs, this data further revealed that myeloid cells from MSA-2 treated PBM-R tumors have significantly higher enrichment scores of cGAS-STING pathway gene signature than control mice, indicating that MSA-2 activated cGAS-STING signaling pathway in myeloid cells (FIG. 21D). Interestingly, MSA-2 also strongly stimulated expression of genes associated with antigen processing and presentation (e.g., Flg2, Tap1, Psmb8, Ctss, Psmb9, Tap2 and B2m) (FIG. 21D). Gene ontology (GO) analysis revealed that MSA-2 significantly increased myeloid leukocyte mediated immunity, neutrophil activation, response to virus, type I interferon signaling pathway and T cell activation signals (FIG. 22C). Flow cytometry analysis further showed that MSA-2 treatment not only decreased population of TAMs (FIG. 21F), but also increased the population of CD11b⁺CD11c⁺ myeloid DCs and enhances class I antigen presentation in this subset of DCs (FIG. 21G). Further analysis confirmed that STING pathway was indeed activated in activated myeloid DCs (FIG. 21H). These data suggest that STING agonism reprograms myeloid cells by promoting re-polarization of M2-like TAMs to an M1-like status and enhancing class I antigen presentation of myeloid DCs in the TME of BRCA1-deficient ovarian tumor-bearing mice. To further investigate whether STING pathway is required for STING agonist induced TAM repolarization, BMDMs generated from STING knock out mice (STING^{-/-} BMDMs) and wild type mice (WT BMDMs) were cultured in CM from a syngeneic BRCA1-deficient cell line (ID8-Brca1^{-/-}) with or without olaparib treatment (FIG. 21I). The results showed that MSA-2 successfully reversed the M2-like TAM polarization of WT BMDMs induced by control or olaparib-treated ID8-Brca1^{-/-} cells, but not STING^{-/-} BMDMs (FIG. 21I). The in vivo data further revealed that MSA-2 successfully activated myeloid DCs and repolarized M2-like TAM to M1-like status in WT mice, but not STING^{-/-} mice (FIGS. 21J and 4K, FIG. 22D). These data demonstrated that STING agonism reprograms myeloid cells in a STING-dependent manner.

### STING Agonism Overcomes Immune Suppressive TME Induced Secondary Resistance to PARPi in Mouse Models of Brca1-Deficient Ovarian Cancer

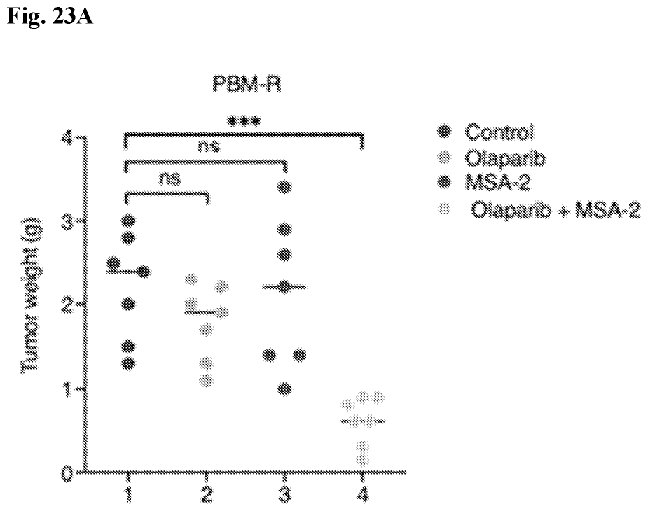

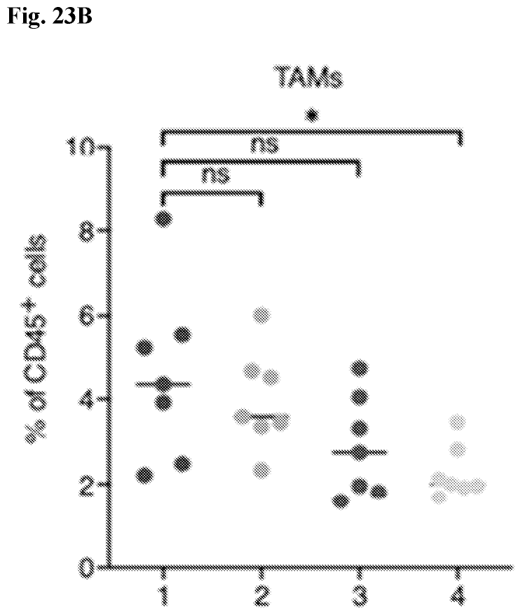

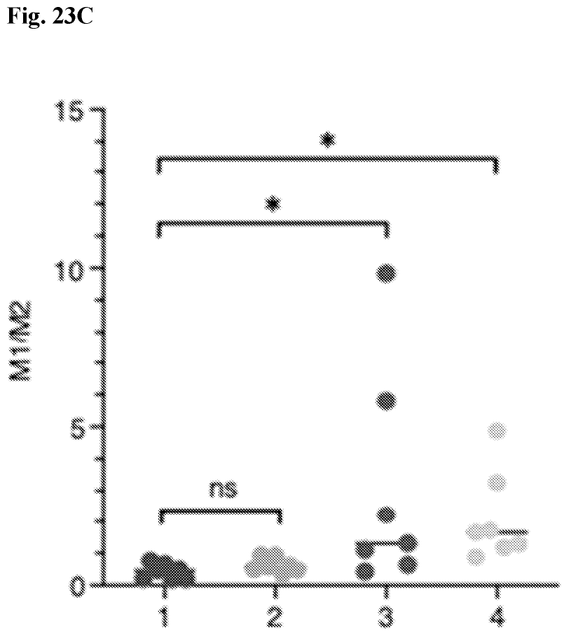

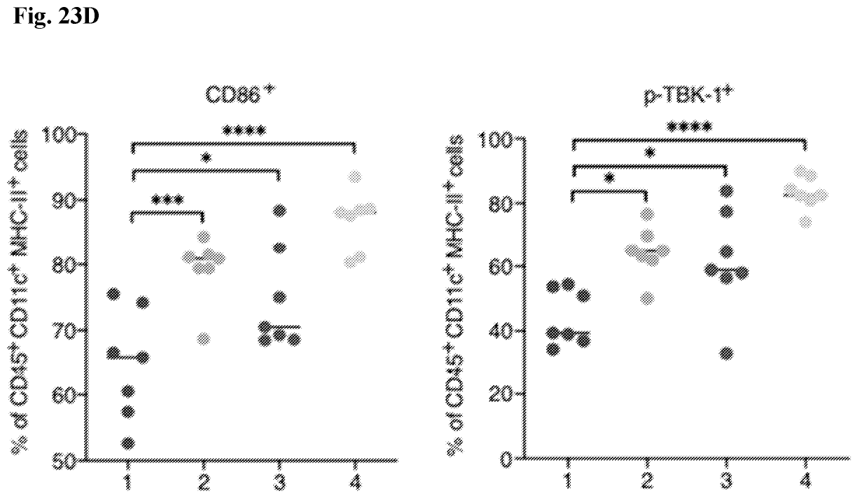

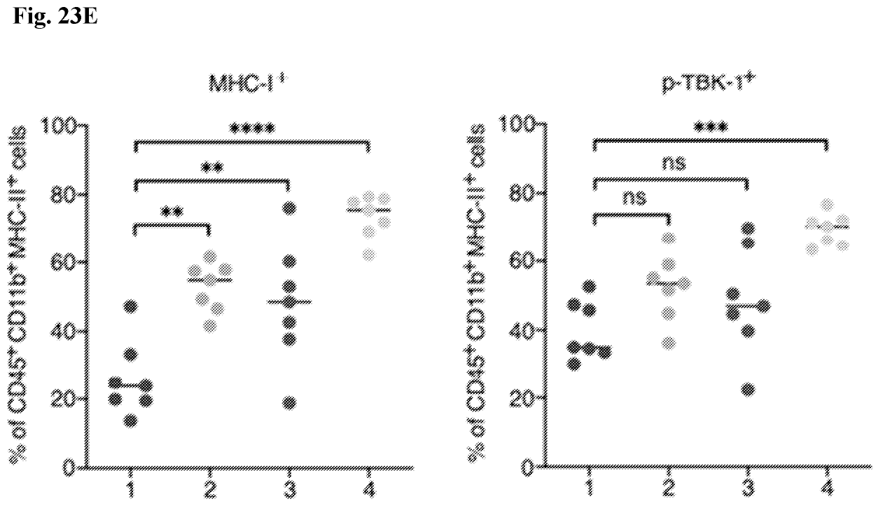

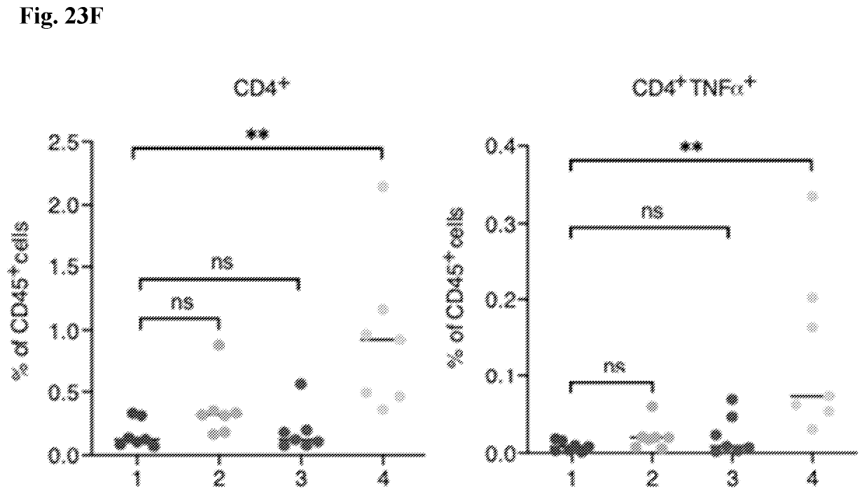

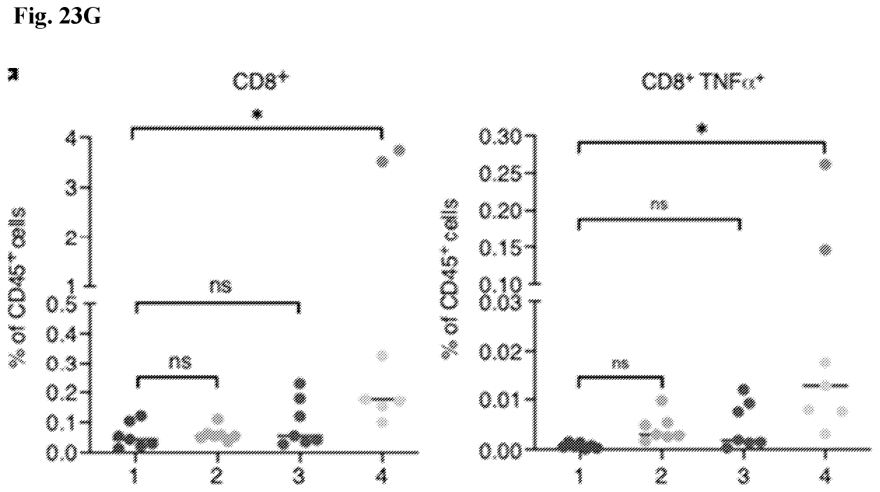

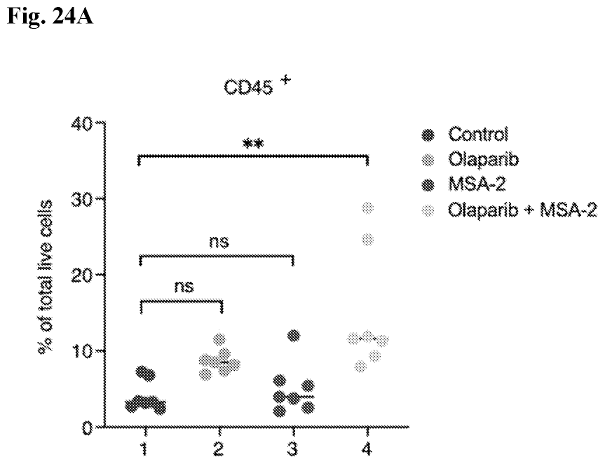

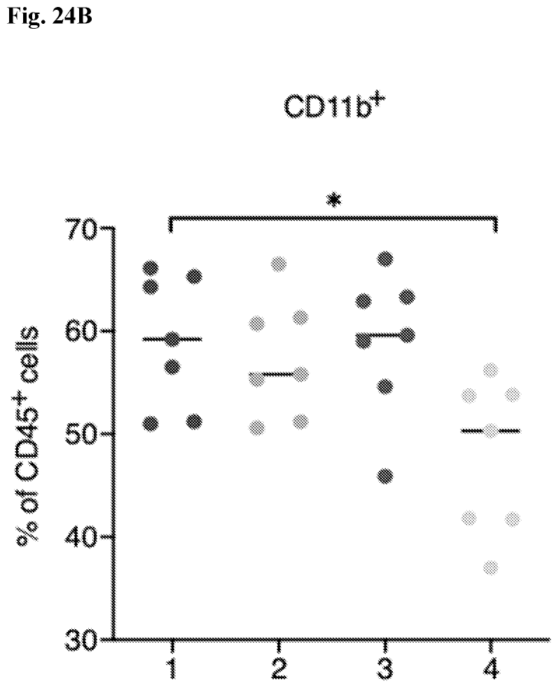

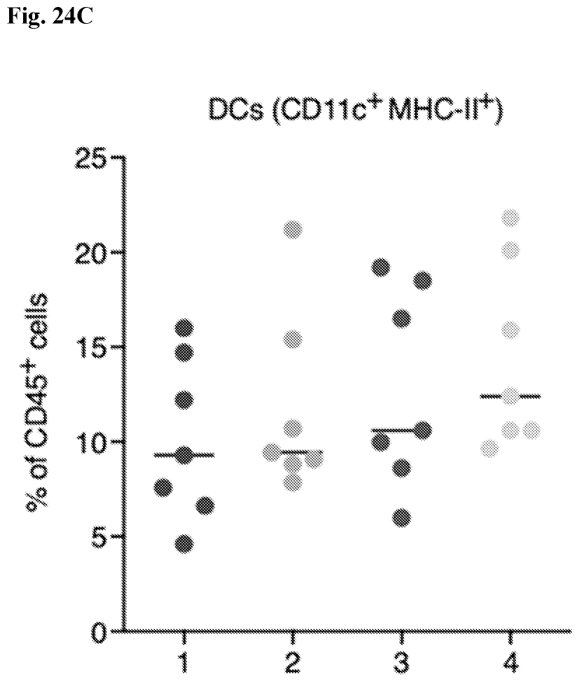

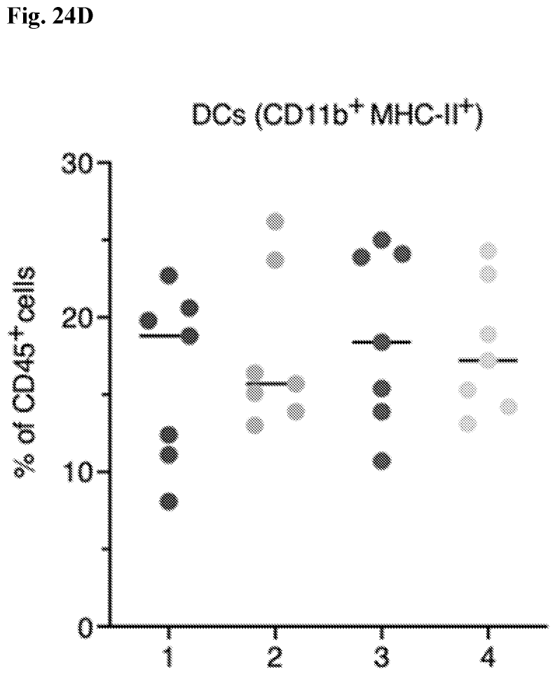

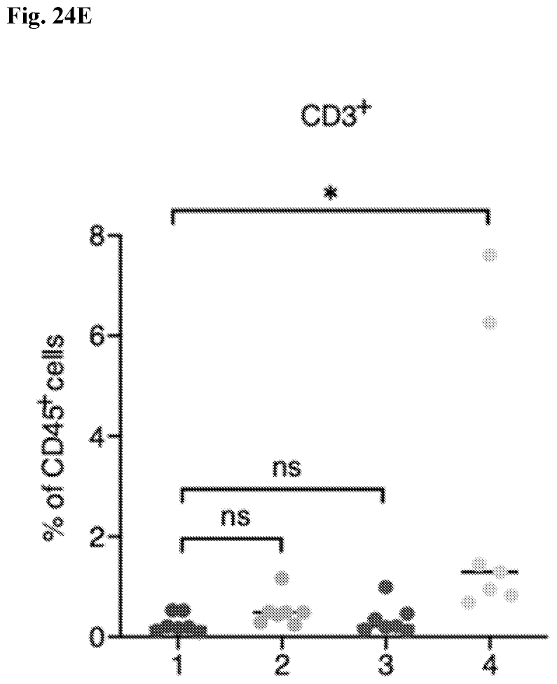

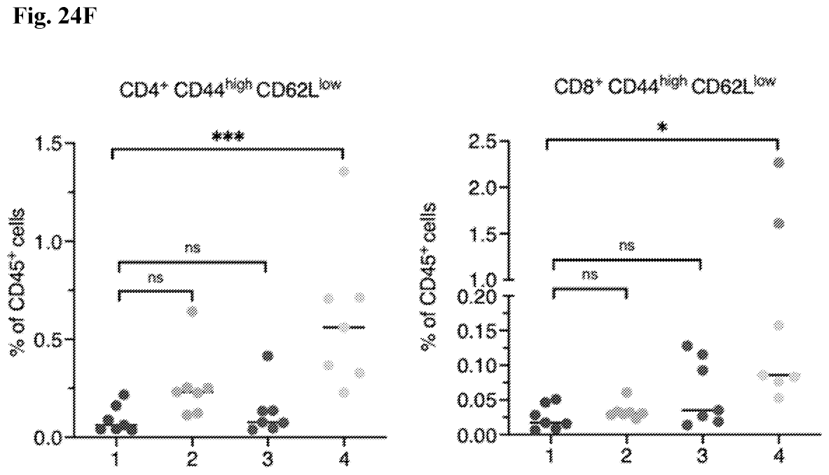

Since STING agonism can reprogram immunosuppressive myeloid cells to an anti-tumor state, it was next investigated whether STING agonism is able to overcome the acquired TME-dependent PARPi resistance in BRCA1-deficient mouse models of ovarian cancer. PBM-R tumor cells were orthotopically injected into FVB/NJ mice. Tumor-bearing mice were randomized into four groups and subjected to control, olaparib, MSA-2 or combination treatment. While MSA-2 or olaparib alone had little or no effect on tumor growth inhibition, combined MSA-2 with olaparib treatment significantly suppressed PBM-R tumor growth, with 60-70% inhibitory effect when comparing to the control group (FIG. 23A). Analysis of tumor-infiltrating immune cells in each group of mice revealed that proportion of TAMs dramatically decreased in the mice treated with MSA-2, along with an increase ratio of M1/M2 (FIGS. 23B and 23C). On the other hand, treatment with MSA-2 or olaparib alone activated the STING pathway and increased tumor-infiltrating activated (CD86⁺) conventional DCs (cDCs), and combined MSA-2 and olaparib further enhance this effect (FIG. 23D and FIG. 24C). Similarly, MSA2 in combination with olaparib activated STING pathway in intratumoral myeloid DCs and enhanced their antigen presentation in PBM-R tumor-bearing mice (FIG. 23E and FIG. 24D). Moreover, the population and cytokine production of tumor-infiltrating CD4⁺ and CD8⁺ significantly increased in the combined MSA-2 and olaparib treatment group (FIGS. 23F and 23G, FIG. 24E). Meanwhile, both CD4⁺ and CD8⁺ effector T cells (CD44^{High} CD62L^{Low}) significantly accumulated in the combination treatment group (FIG. 24F). Taken together, these data show that STING agonist can overcome the immune suppressive TME-induced resistance to PARPi in BRCA1-deficient ovarian tumors by reprograming immunosuppressive myeloid cells.

### STING Agonism Re-Sensitized PARPi-Resistant Ovarian Cancer PDXs to PARP Inhibition

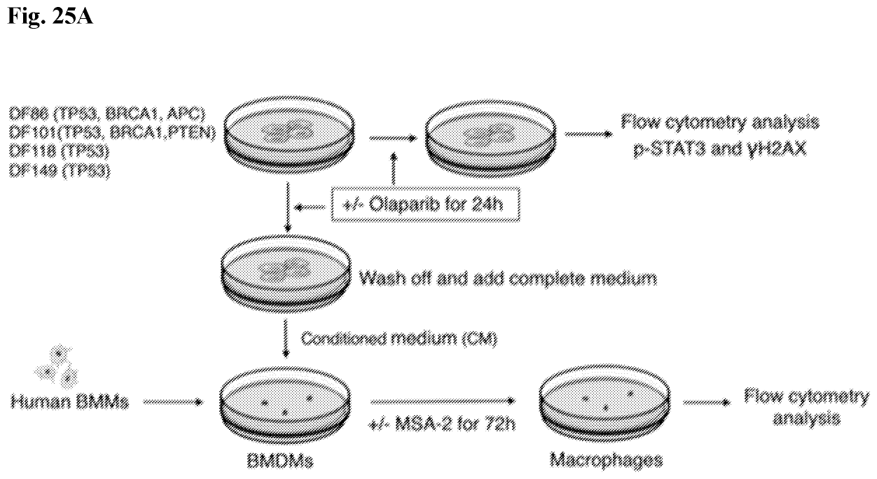

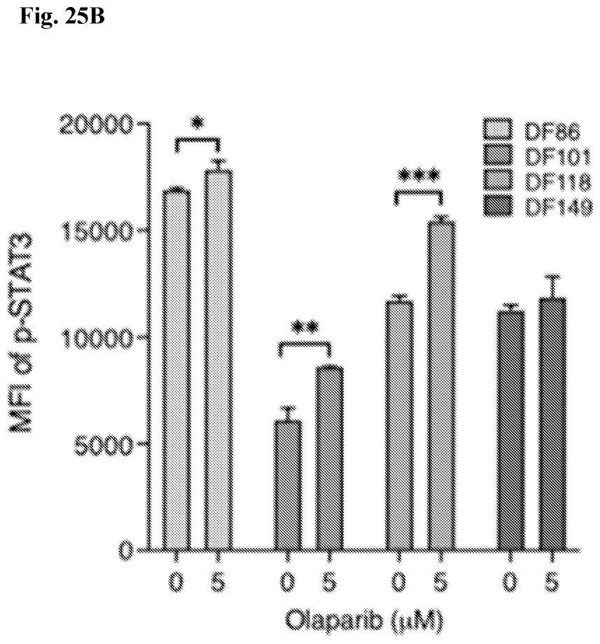

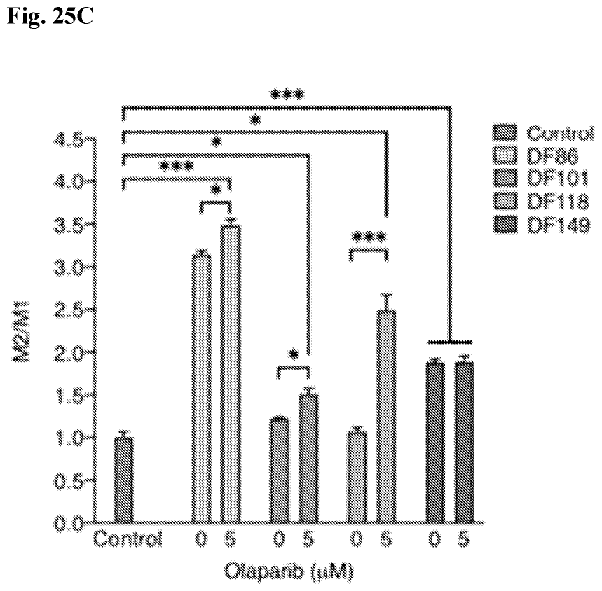

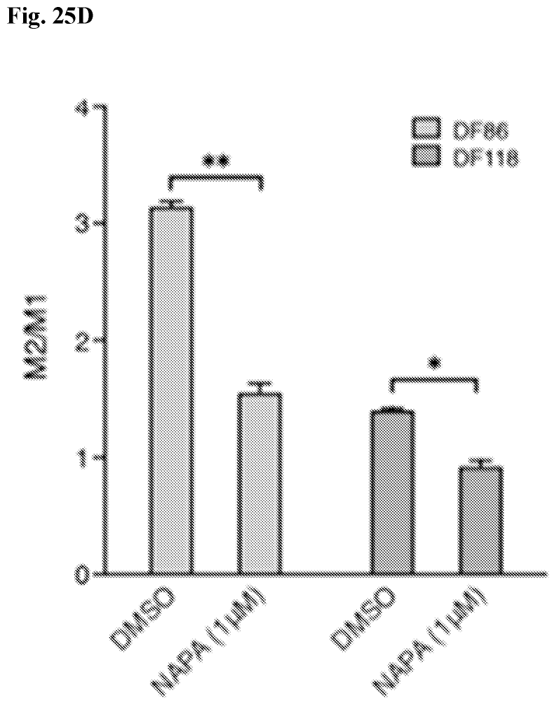

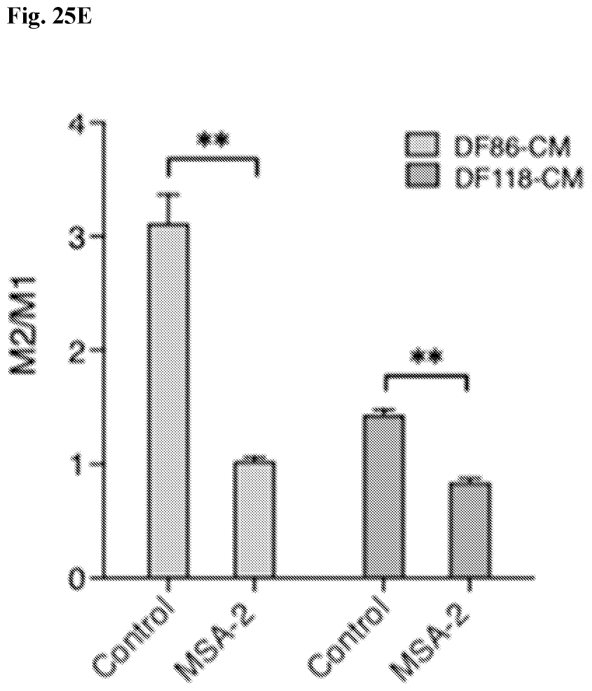

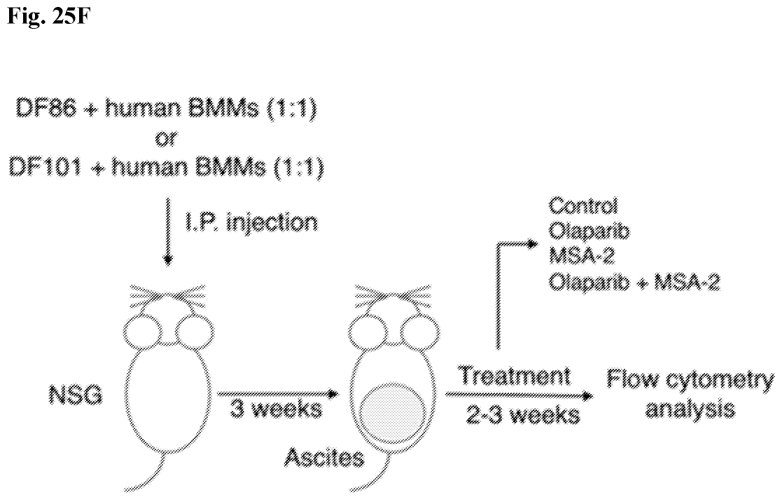

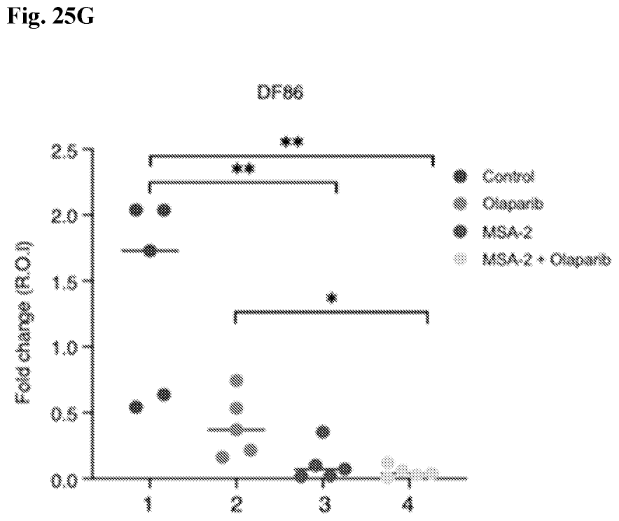

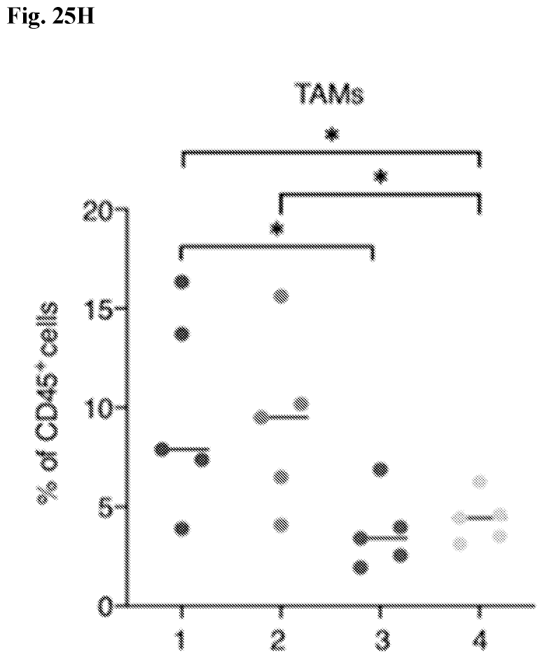

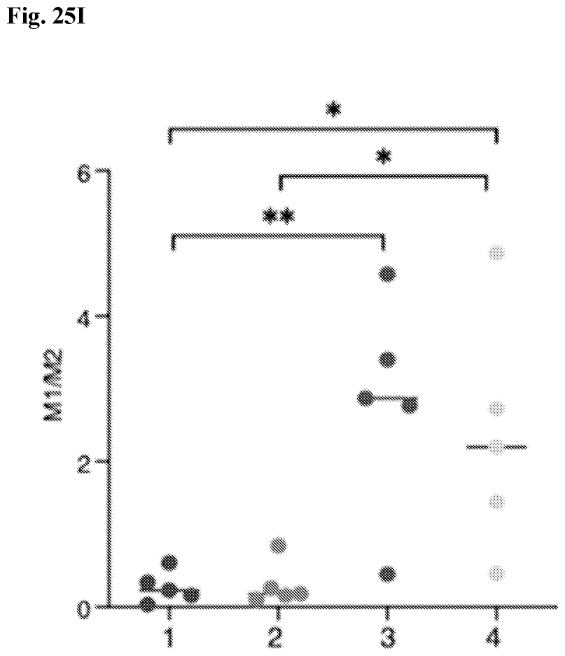

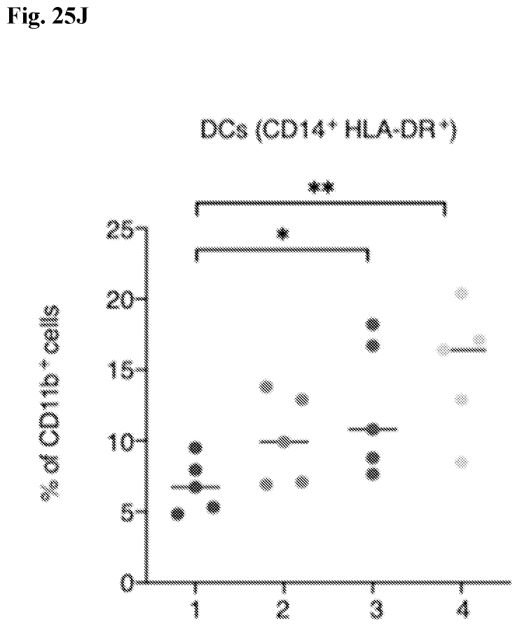

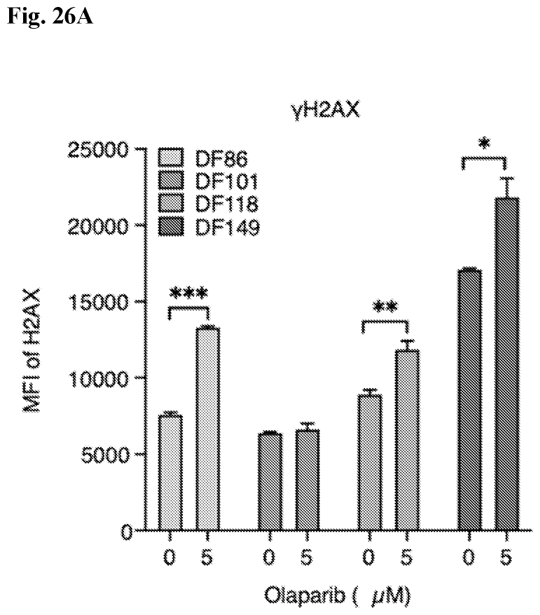

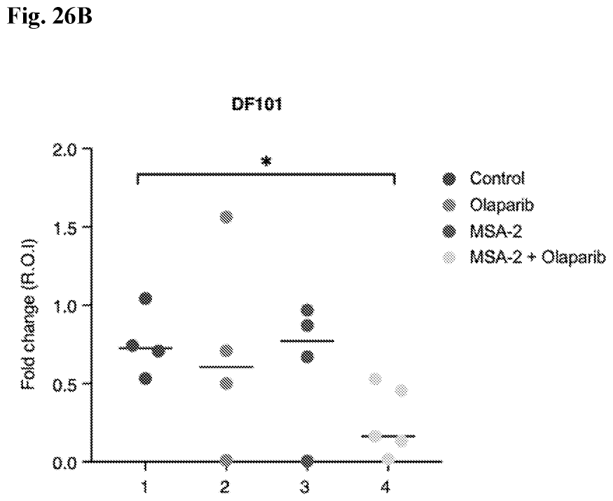

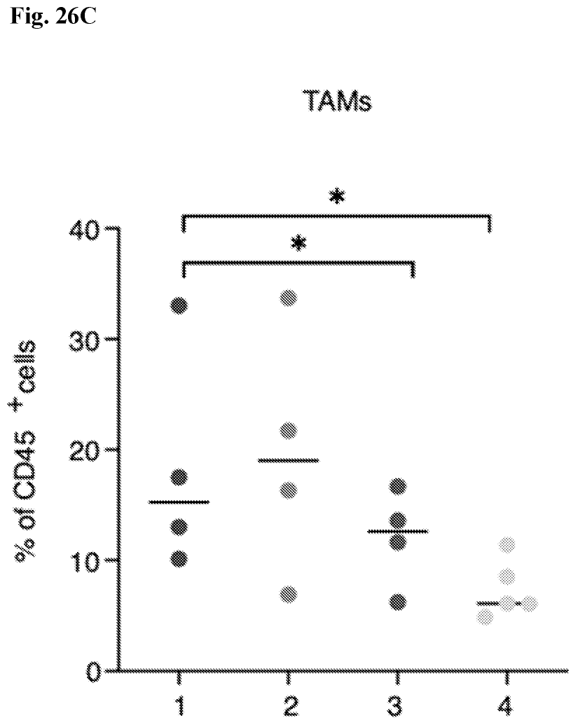

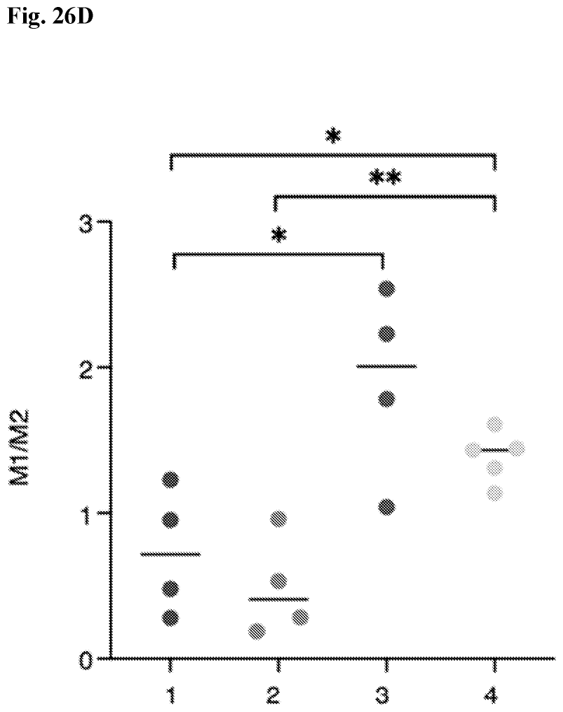

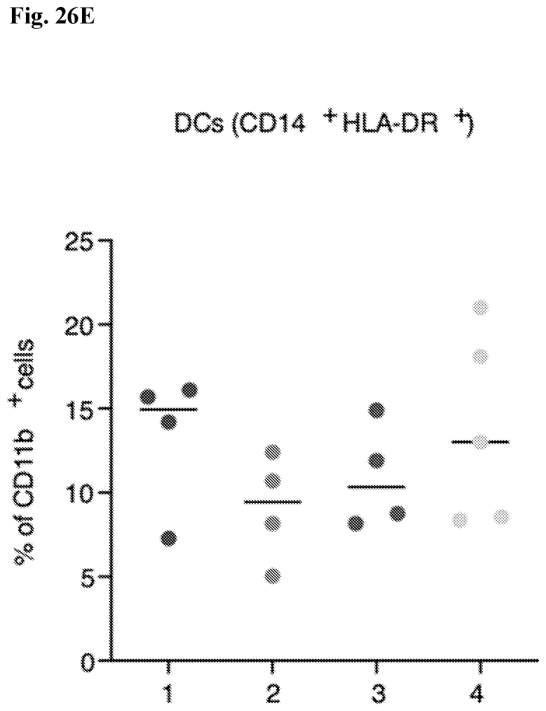

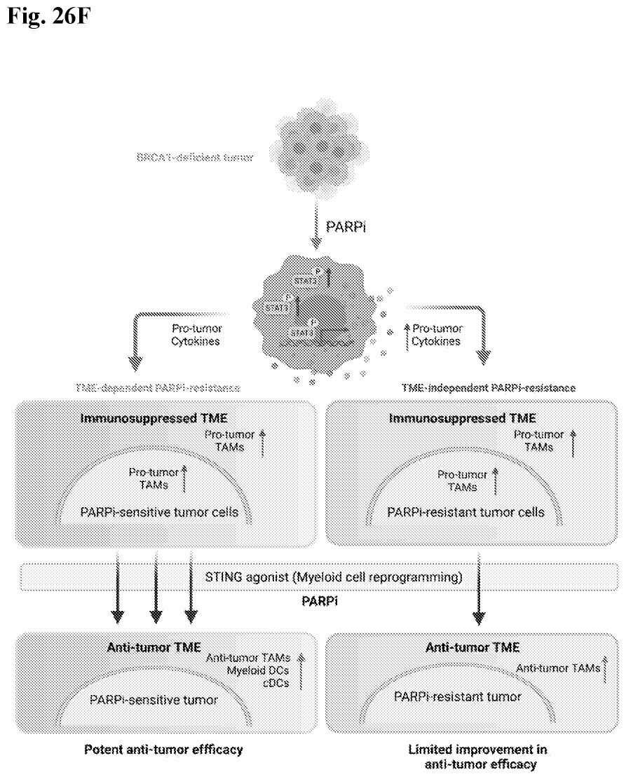

Ovarian patient-derived xenograft (PDX) models were previously generated by transplantation of cancer cells from patients' ascites into immunodeficient mice (Liu et al., 2017 Clinical cancer research: an official journal of the American Association for Cancer Research 23, 1263-1273). PARPi-resistant ovarian PDXs, DF86 and DF101 (BRCA1-deficient), DF118 and DF149 (BRCA1-proficient), were collected from ascites of NSG mice and cultured in vitro for 24 hours and then treated with 5 μM olaparib for 24 hours, increased phosphorylation level STAT3 was detected in 3 out of 4 PDX cells after olaparib treatment (FIGS. 25A and 25B). In parallel, human BMDMs were cultured in the CM collected from ovarian cancer PDXs with or without olaparib treatment. Consistent with previous findings in mouse and human ovarian cancer cell lines, CM from most ovarian PDXs significantly promote M2-like macrophage polarization, activation of STAT3 signaling pathway induced by olaparib treatment further enhance the M2-like polarization induced by ovarian PDXs (FIG. 25C). Inhibition of STAT3 signaling pathway with a STAT3 inhibitor napabucasion partially diminished the M2-like macrophage polarization induced by CM from DF86 and DF118 (FIG. 25D). Addition of MSA-2 in the co-culture system reversed the M2-like macrophage polarization that was induced by ovarian PDXs (FIG. 25E). To assess whether STING agonism can reprogram myeloid cells and overcome immunosuppression in ovarian PDX models in vivo, DF86 cells that were relatively sensitive to PARPi treatment in vitro (FIG. 26A), were mixed with primary human bone marrow monocytes (BMMs) and injected to NSG mice. About three weeks after injection, when the high immunosuppressive TME formed, DF86 tumor cell-bearing mice were grouped and treated with vehicle control, olaparib, MSA-2 or combined olaparib and MSA-2 (FIG. 25F). MSA-2 treatment significantly decreased tumor burden of DF86 tumor cell-bearing mice NSG mice when compared to control or olaparib treated mice, reflected by the fold change of intensity of luciferase signal (FIG. 25G). Flow cytometry analysis of human immune cells in ascites revealed that MSA-2 treated groups of mice had a decreased proportion of TAMs and an increased M1/M2 ratio (FIGS. 25H and 25I, FIG. 26B). Notably, CD14⁺ myeloid DCs (CD14⁺HLA-DR⁺) significantly increased in the STING agonist-treated groups (FIG. 25J). The effect of MSA-2 in DF101 PDX model was also evaluated, which is more resistant to olaparib in vitro compared to DF86 (FIG. 26A). The result showed that MSA-2 greatly decreased the proportion of TAMs and reprogramed M2-like TAMs to M1-like in the ascites of DF101 tumor cell-bearing mice, although the DF101 tumor cell-bearing mice are less responsive to MSA-2 treatment alone or in combination with olaparib as compared to DF86 PDX tumors (FIG. 26C-E).

### Example 4: Methods for Example 3

### Generation of PARPi-Resistant Ovarian Cancer Mouse Models

The Brca1-deficent ovarian cancer mouse model-PBM (Trp53−/−; Brca1−/−; c-Myc) was previously developed in FVB/NJ mice (Ding et al., 2018 Cell Rep 25, 2972-2980.e2975). PBM tumor cells were orthotopically transplanted into syngeneic FVB/NJ mice and treated with vehicle control or olaparib (AZD2281) 6 days a week by i.p. injection at dose of 50 mg/kg body weight. The PBM tumors initially response well but relapsed on olaparib treatment after long-term treatment. The tumor cells derived from refractory tumor-bearing mice (one cell line from each treated mouse), terms PBM-R, were cultured in MOT media (DMEM/F12, 0.6% FBS, 10 ng/ml EGF, hydrocortisone lug/ml, cholera toxin 1 ng/ml, 100 μg/ml penicillin-streptomycin, 5 μM Y27632) for further evaluation.

### Cell Lines and PDX Models

UWB1.289 and UWB1.289+BRCA1 were purchased from ATCC and cultured in epithelial complete growth medium (50% ATCC-formulated RPMI-1640 medium, 50% MEGM medium and 3% fetal bovine serum) as described previously (Ding et al., 2018, Cell Rep 25, 2972-2980.e2975). ID8-Brca^{+/+} and ID8-Brca^{-/-} cells were previously generated by CRISPR-Cas9 technology. The ovarian cancer patient-derived tumor xenografts (PDX) were established at Dana-Farber Cancer Institute by intraperitoneally implanting tumor cells that isolated from patients' ascites into irradiated nude mice (Liu et al., 2017; Clinical cancer research: an official journal of the American Association for Cancer Research 23, 1263-1273). The established PDX models were maintained by intraperitoneally transplantation in NOD/SCID IL2Rgnull mice (NSG, The Jackson Laboratory). The ovarian PDX cells could be cultured in the epithelial complete growth medium for about 3 to 4 days for in vitro experiments.

### Measurement of IC50 Value in Tumor Cells

Tumor cells were seeded in 96-well plates at a density of 2000-3000/well and allowed to adhere overnight. Cells were then exposed to appropriate concentrations of therapeutic agents (or vehicle control) with continuous exposure for 72 h. Growth inhibition was measured by CellTiter-Glo® 2.0 Cell Viability Assay from Promega according to manufactory's introduction. IC50 values were calculated using non-linear regression model (logarithmic inhibitor vs. normalized response-variable slope) in Graphpad Prism 9.

### Tumor Growth and Treatment

The PBM and PBM-R tumor cells were transplanted orthotopically into syngeneic FVB/NJ mice to generated tumors for drug evaluation. Tumor-bearing mice were equivalently divided into control and treatment groups according to the luminescent intensity as previously described (Ding et al., 2018; Cell Rep 25, 2972-2980.e2975). Olaparib (AZD2281) was administered daily by i.p. injection at dose of 50 mg/kg body weight. Anti-PD-1 antibody (clone, 332.8H3) was diluted in PBS (250 μg/100 μl/mouse) and injected by i.p. every 3 days. MSA-2 was prepared by diluting 50 mg/ml stock in DMSO with PBS (pH 8.0) and administered every other day (three times a week, two weeks on followed by one week off) by i.p. injection at dose of 25 mg/kg body weight. The endpoints were determined by tumor burden and ascites.

For the PDX in vivo experiments, about 3×10⁶ PDX tumor cells were mixed with 3×10⁶ human bone marrow mononuclear cells in serum-free DMEM/F12 medium containing 50% Matrigel (CAT #70001, STEMCELL Technology) and intraperitoneally transplanted into NOD/SCID IL2Rgnull mice (NSG, The Jackson Laboratory). About three weeks after injection, PDX-bearing mice were equivalently divided into 4 groups according to the luminescent intensity and treated with vehicle control, olaparib, MSA-2 and olaparib in combination with MSA-2 using the same dosing and schedule that have been described above. Ascites was harvested for analysis after three weeks' treatment.

### Flow Cytometry Analysis

Tumors were chopped and digested in collagenase buffer as described previously (Ding et al., 2018 Cell Rep 25, 2972-2980.e2975). Single cell suspensions of tumor and ascites were obtained by filtering through 70 um strainers and treated with 1× eBioscience RBC lysis buffer (Thermo Fisher) before staining. Single cells suspensions were incubated with LIVE/DEAD Fixable Aqua Dead Cell Stain (Life Technologies, Cat #L34965) for 30 min and then blocked with anti-CD16/32 (Biolegend, clone 93) for 20 min on ice. Samples were then incubated with appropriate antibodies for 30 min on ice. Foxp3 staining buffer set (eBioscience, Cat #00-5523-00) was applied for intracellular markers staining. For the intracellular cytokine analysis, cells were stimulated with Leukocyte Activation Cocktail (BD Biosciences, Cat #550583) at 37° C. for 4-6 hours prior to FACS staining. The following antibodies were used in this study: antibodies were purchased from BioLegend unless otherwise indicated: CD45 (clone 30-F11), CD3ε (clone 145-2C11), CD4 (clone RM4-5), CD8 (clone 53-6.7), CD44 (clone IM7), CD62L (MEL-14), CD25 (PC61), IFNγ (clone XMG1.2), TNFα (clone MP6-XT22), CD11b (clone M1/70), CD11c (clone BM8), F4/80 (clone BM8), Ly-6C (clone HK1.4), Ly-6G (clone 1A8), MHC-II (clone M5/114.15.2), CD80 (clone 16-10A1), CD86 (clone GL-1), MHC-I (clone KH114), FoxP3 (clone FJK-16 s; eBioscience), Phospho-IRF-3 (Ser396) (clone D601M, Cell signaling technology) and Phospho-TBK1/NAK (Ser172) (clone D52C2, Cell signaling technology). The following human antibodies were used in this study: cell surface markers includes CD45 (clone H130), CD11b (clone M1/70), CD11c (clone Bu15), CD80 (clone 2D10), CD86 (clone IT2.2), CD14 (clone 63D3), CD15 (clone 30-F11), HLA-DR (clone L243) and HLA-A,B,C (clone W6/32); intracellular markers include CD163 (clone GHI/61), CD68 (clone Y1/82A) and CD206 (clone 15-2). Flow cytometry was performed on an LSRII (BD Biosciences) or Fortessa HTS (BD Biosciences) at DFCI Flow Cytometry Core, and all the data were analyzed using FlowJo software.

Analysis of p-STAT3 and γ-H2AX (p-HA2X-Ser139) was performed according to a two-step protocol for intracellular phosphorylated signaling proteins (Thermo Fisher). Briefly, cells were incubated with LIVE/DEAD Fixable Aqua Dead Cell Stain for 30 min. After washing, cells were suspended in 100 μl PBS and then fixed by adding an equal volume of IC Fixation Buffer (CAT #00-822-49, Thermo Fisher) directly to cells and incubated at room temperature for 20 min followed by fixing with ice-cold 90% methanol in PBS for 30 min. The fixed cells were blocked and stained with p-STAT3 or γ-H2AX antibodies for flow cytometry analysis as described above.

### Cytokine Array Analysis

The PBM tumor cells were cultured in 6-well plate for 24 hrs and treated with olaparib or vehicle control. Drug was removed after 24 hrs' treatment, and cells were cultured in fresh medium for another 48 hrs. Cell culture supernatants were obtained by centrifugation at 1,500 g for 5 min at 4° C. to remove all the debris and cells then subjected to Cytokine array analysis (ARY028, R&D system) according to manufactories introduction. Briefly, the cell culture supernatants were mixed with a cocktail of biotinylated detection antibodies, and then incubated with the Mouse Cytokine Array. The array was then incubated with streptavidin-horseradish peroxidase followed by chemiluminescent detection. Array images were analyzed using the Image J software.

### Lentivirus-Mediated Knockdown of Stat3 in PBM-R Tumor Cells

Control shRNA and Stat3 shRNAs plasmids (sh-Stat3-1: TRCN0000071456 and sh-Stat3-2: TRCN0000071453) were acquired from Sigma-Aldrich. The Stat3 shRNAs and control shRNA plasmids were co-transfected with pCMV-delta8.9 and pVSVG at the ratio of 2:2:1 into HEK293T cells by PEI (1 μg/μl) (4:1 to DNA). The medium was changed 24 hr after transfection and the viral supernatants were collected 48 hr later by filtering through a 0.45-μm filter. PBM-R cells were cultured in a 6-well plate and infected with Stat3-shRNA lentiviral particles, puromycin (3 μg/mL) was added to the culture for selection. Puromycin resistant cells were selected and expanded. Western blot analysis was performed to evaluate the silencing effect of lentiviral Stat3 in PBM-R tumor cells.

### Western Blot Analysis

Tumor cells were harvested and lysed with ice-cold RIPA buffer supplemented with protease phosphatase inhibitor cocktail (Thermo Fisher). Protein concentration was determined using a Pierce BCA Protein Assay Kit (Thermo Fisher). About 50 μg protein extracts were loaded and separated by SDS-PAGE, and then transferred to polyvinylidene fluoride (PVDF) membranes. After blocking with 5% non-fat milk (Bio-Rad) in PBST (PBS plus 0.2% Tween 20) at room temperature for 1 hr, membranes were incubated with primary antibodies overnight at 4° C. Fluorescently-labeled anti-mouse IgG (Rockland Immunochemicals, #RL610-145-002) or anti-rabbit IgG (Molecular Probes, #A-21109) were used as second antibodies and the western blots were visualized on an Odyssey scanner (LI-COR).

### Co-Culture Experiments

For ex vivo culture of bone marrow cells in ascites supernatants, bone marrow cells were isolated from FVB/NJ mice and cultured in the conditioned medium containing 50% of ascites supernatant and 50% of complete DMEM medium (90% DMEM and 10% FBS), supplied with 100 μg/ml penicillin-streptomycin. The cells were incubated in conditioned medium for 6 days with media replacement on day 3. The components of the cells were analyzed by on flow cytometry analysis.

For ex vivo culture of mouse macrophages, bone marrow cells were isolated from FVB/NJ mice and cultured in DMEM containing 10% FBS, 55 μM 2-Mercaptoethanol and 20 ng/ml M-CSF. BMDMs (bone marrow derived macrophages) were harvested on day 7 and further cultured in 2.0 ml control medium (90% DMEM, 10% FBS, 55 μM 2-Mercaptoethanol and 5 ng/ml M-CSF) described above or the medium containing 50% tumor cell conditioned medium for 72 hrs before analysis. For the tumor cell conditioned medium, about 3×10⁵ tumor cells were cultured in 6-well plate for 24 hrs and then treated with DMSO or olaparib. After 24 hrs incubation with DMSO or olaparib, the tumor cells were washed twice with PBS and cultured in DMEM containing 10% FBS for 48 hrs. The tumor cell conditioned medium was collected by centrifugation at 1,500 g for 5 min at 4° C. to remove all the debris and cells. For the cytokine blocking experiment, monoclonal antibodies that specific to each cytokine were added to the tumor cell conditioned medium before it was applied to culture macrophages.

For ex vivo culture of human macrophages, human BMDMs were generated from bone marrow mononuclear cells (BMMs) (CAT #70001) obtained from STEMCELL Technologies. Briefly, BMMs were cultured in DMEM containing 10% FBS, 55 μM 2-Mercaptoethanol (CAT #21985023, Thermo Fisher) and 50 ng/ml M-CSF for 5-7 days with media replacement every 3 days to obtain matured macrophages (BMDMs). BMDMs were further cultured in the medium with or without addition of 50% conditioned medium obtained from human ovarian cancer cell lines for 72 hrs. Flow cytometry analysis was preformed to analyze the phenotypes of macrophages for both mouse and human BMDMs.

### Transcriptome Analysis

Transcriptome analysis of tumor samples: total RNA was isolated from bulk tumors by RNeasy Plus Mini Kit (QIAGEN) and sequenced on an Ion Torrent platform (Thermo Fisher) using an Ion AmpliSeq Custom Panel targeting 4,604 murine genes. To generate read counts per gene, data were analyzed using Torrent Suite and AmpliSeqRNA analysis plugin (Thermo Fisher). Differential gene expression analyses were carried out using DESeq2 with default parameters to obtain log 2 fold change (MAP) and adjusted p-values (Benjamini-Hochberg procedure) Genes were ranked by log 2 fold change (MAP), and GSEA were carried out using the GSEA Preranked tool.

Transcriptome analysis of myeloid cells: about 1 x106 PBM-R tumor cells were intraperitoneally injected to FVB/NJ mice. About 2-3 weeks after injection, mice were grouped and treated with control, olaparib, MSA-2 and MSA-2 in combination with MSA-2 for 24 hrs. After treatment, myeloid cells (CD45⁺CD11b⁺) were isolated from the ascites of each mouse (n=3 per group). Total RNAs was isolated and sequenced on the Ion Torrent platform (Thermo Fisher) that described above using the Ion AmpliSeq Transcriptome Mouse Gene Expression Panel. Gene ontology (GO) analysis of DEGs was performed using topGO package in R. GSEA analysis was performed as described above. Heat maps illustrating changes in gene expression were generated using the heatmap.3 package in R.

### Quantification and Statistical Analysis

Statistical analysis was performed using Prism 9 (Graphpad Software Inc.). Unpaired two-tailed Student's t-test for normally distributed data and Mann-Whitney nonparametric test for skewed data that deviate from normality were used to compare two conditions. One-way ANOVA with Bonferroni's post-hoc test for normally distributed data and Kruskal-Wallis nonparametric test for skewed data were used to compare three or more means. Differences with P<0.05 were considered statistically significant.

### Example 5: Targeting Tumor Immune Microenvironment to Overcome the Resistance in Lung Cancer

Osimertinib (AZD9291) is a third-generation EGFR tyrosine kinase inhibitor (TKI) for patients with non-small cell lung cancer (NSCLC) with EGFR-activating mutations or the acquired T790M mutation resistant to earlier generation EGFR-TKIs. Emergence of resistance to osimertinib is inevitable, and overcoming such resistance remains a key challenge in the clinic. As provided herein, the inventors used a syngeneic genetically-engineered mouse (GEM) model of lung cancer driven by a mutant EGFR to show that while EGFR-mutant tumors are highly sensitive to osimertinib at early stage of tumor growth in a T cell-dependent manner, they become resistant as they progress. It is further shown that presence of immunosuppressive tumor-associated macrophages (TAMs) render tumors resistance to osimertinib. Depletion of TAMs in these tumors partially rescues the efficacy of osimertinib. Reprograming TAMs with a newly developed STING agonist MSA-2 reinvigorates antitumor immunity, and lead to durable regression of resistant tumors in mice when combined with osimertinib. The results shown herein suggest that suppressive tumor immune microenvironment can drive resistance of EGFR-mutant tumors to osimertinib, providing a new rationale strategy to overcome resistance and improve the therapeutic outcomes.

Lung cancer, with non-small cell lung cancer (NSCLC) as the predominant subtype, remains one of the most prevalent malignant disease with high mortality worldwide (de Groot et al., 2018, Transl Lung Cancer Res 7, 220-233). Genes encoding epidermal growth factor receptor (EGFR) is one of the most common oncogenes whose mutations frequently occur in NSCLC especially lung adenocarcinoma, with the incidence being up to 15% among Caucasian patients and 50% among Asian patients (Rosell et al., 2009, N Engl J Med 361, 958-967; Shi et al., 2014, J Thorac Oncol 9, 154-162). The identification of EGFR-activating mutations and the subsequent development of tyrosine kinase inhibitors (TKIs) targeting the mutated EGFR had drastically revolutionized the treatment landscape of EGFR-mutated NSCLC. However, despite the remarkable therapeutic response to EGFR-TKIs, resistances inevitably develop in the majority patients, with the average progression-free survival ranging from 9 to 15 months (Recondo et al., 2018 Nat Rev Clin Oncol 15, 694-708).

The development of mutation at the ‘gatekeeper’ site in EGFR exon 20 (T790M) is one of the most common mechanisms mediating resistance to the first and second generation of EGFR-TKIs, i.e. gefitinib or erlotinib (Lim et al., 2018, Cancer Treat Rev 65, 1-10). To overcome resistance driven by T907M mutation, osimertinib was developed as the third generation EGFR-TKI that can irreversibly bind to the mutated EGFR regardless of the presence of T790M mutation (Cross et al., 2014, Cancer Discov 4, 1046-1061; Janne et al., 2015, N Engl J Med 372, 1689-1699). Due to its superior therapeutic efficacy, osimertinib has been approved as first line therapy or next line therapy after progression on first -or second-generation EGFR-TKIs in the treatment of advanced NSCLC harboring EGFR mutations (Goss et al., 2016, Lancet Oncol 17, 1643-1652; Ramalingam et al., 2018, Nat Rev Clin Oncol 15, 694-708; Soria et al., 2018, N Engl J Med 378, 113-125). However, even with the robust clinical benefit derived from osimertinib treatment, patients still will develop therapeutic resistance eventually. Tremendous efforts had been devoted to unravel the underlying mechanisms mediating resistance to EGFR-TKIs especially osimertinib. One of the widely recognized mechanisms is the emergence of tertiary EGFR mutations like C797S substitution, which account for 6-10% and 10-26% of the resistance when osimertinib is applied as a first- or second-line treatment respectively (Leonetti et al., 2019, Br J Cancer 121, 725-737; Thress et al., 2015, Nat Med 21, 560-562). Other mechanisms conferring resistance to EGFR-TKIs include EGFR gene amplification, activation of alternative bypass pathways, novel fusion events, and phenotypic transformation etc. However, a significant fraction of lung cancer patients harboring EGFR mutants are not sensitive to Osimertinib with unknown underlying mechanisms (Leonetti et al., 2019, Br J Cancer 121, 725-737).

While the tumor cell intrinsic resistant mechanisms have been explored extensively, the effects of tumor immune microenvironment on therapeutic responses to EGFR-TKIs are less well understood. Immune microenvironment is a complex entity comprising of various infiltrating immune populations like T cells, B cells, myeloid cells etc., which can exert either pro- or anti-tumorigenic capacities and are associated with the therapeutic outcome of many anti-tumor treatments. For example, macrophages have been reported as a major immune suppressive component that block T cell-mediated response and compromised the therapeutic outcome of chemotherapy (Ruffell et al., 2014, Cancer Cell 26, 623-637). Another publication also reports the capability of EGFR-TKIs in eliciting interferon responses, which are associated with improved therapeutic outcome in patients with EGFR-mutated NSCLC (Gurule et al., 2021, NPJ Precis Oncol 5, 41). While these studies suggest the role of immune activation in EGFR-TKI treatments, whether the immune suppressive microenvironment in aggressive tumors contributes to the resistance to EGFR-TKIs, and how to modulate immune cells to maximize the therapeutic benefit is yet to be determined.

In the data disclosed herein, it was found that osimertinib induced activation of T cells in a syngeneic GEM model of lung cancer harboring EGFR exon19del/T790M mutation, which is required for osimertinib-induced tumor regression. Predominance of immunosuppressive TAMs in more advanced (larger in volume here) tumors resulted in T cell exclusion and resistance to osimertinib, which could be partially rescued by macrophage depletion. Furthermore, reprogramming TAMs from a pro-tumorigenic M2-like macrophage phenotype to an anti-tumor M1-like state with a STING agonist reversed the immune suppressive microenvironment and circumvented the therapeutic resistance to osimertinib. These findings reveal a mechanistic understanding of resistance to an EGFR-TKI caused by tumor immune microenvironment and provide a new approach to overcome the resistance by targeting the immunosuppressive microenvironment.

### Results

### Osimertinib-Elicited Activation of T Cells is Necessary for its Therapeutic Efficacy In Vivo

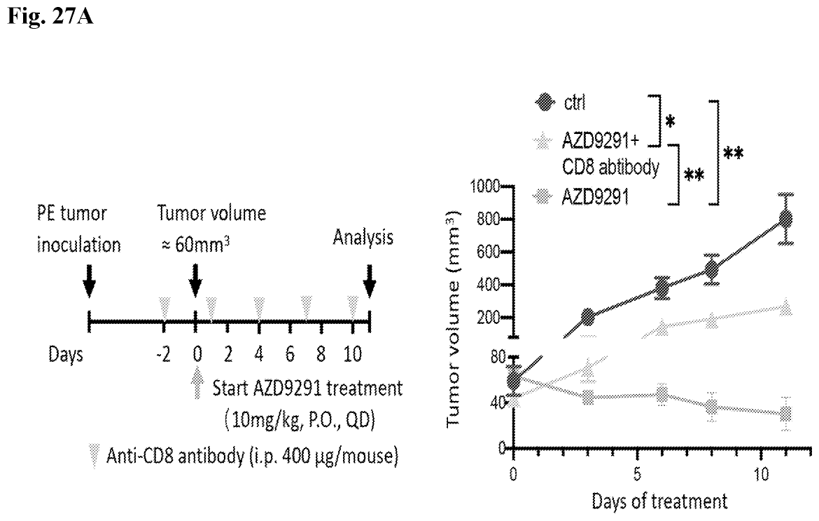

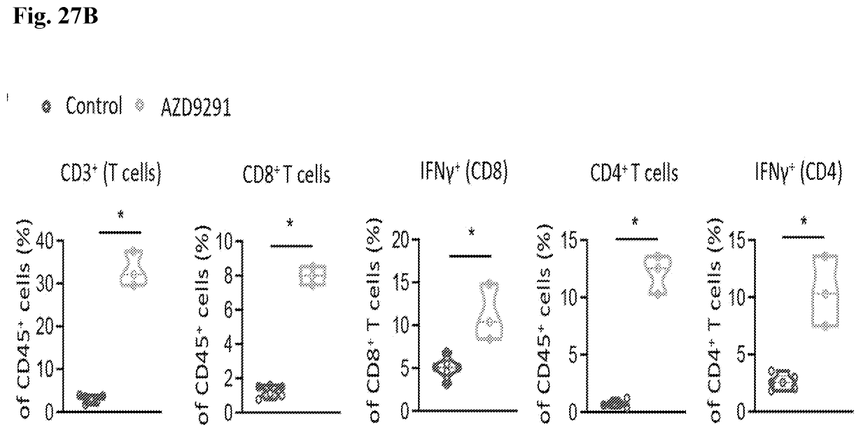

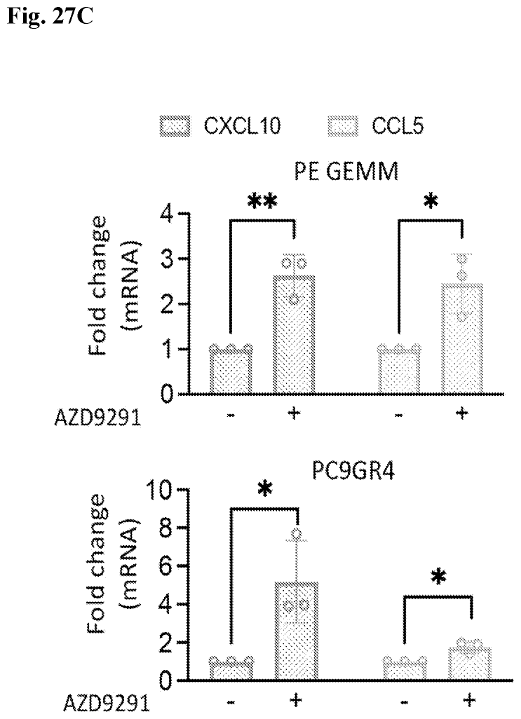

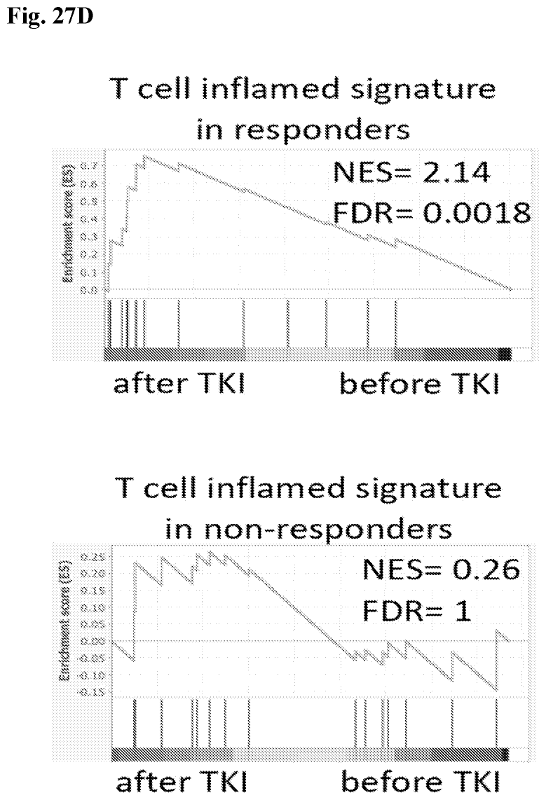

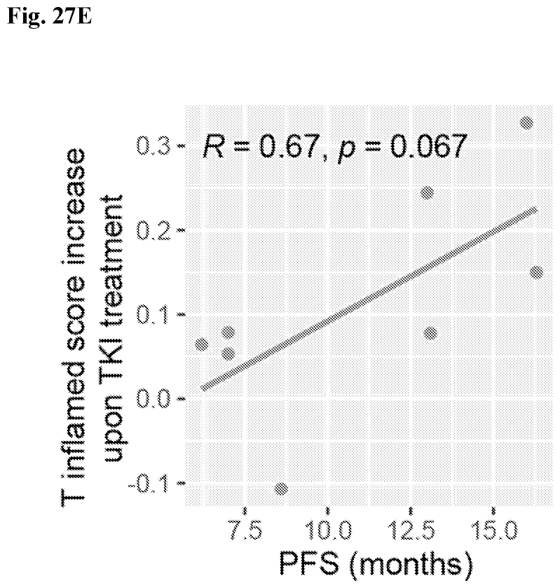

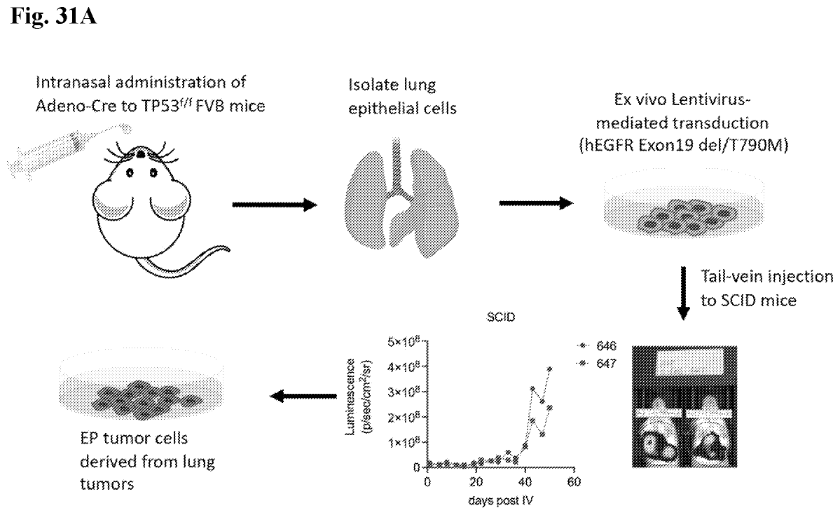

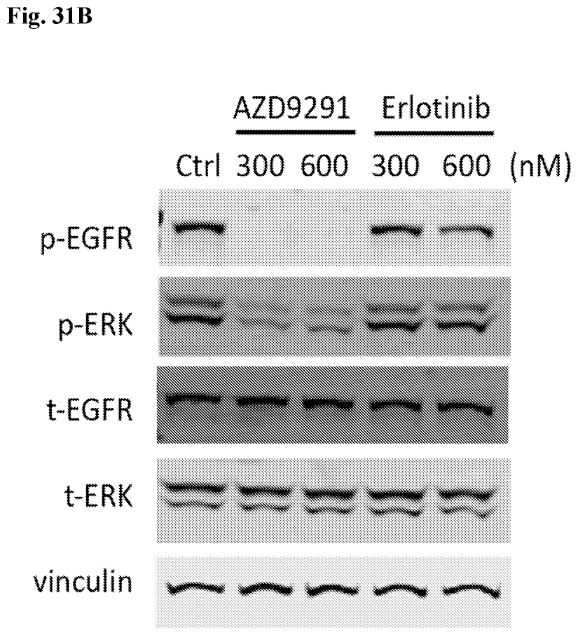

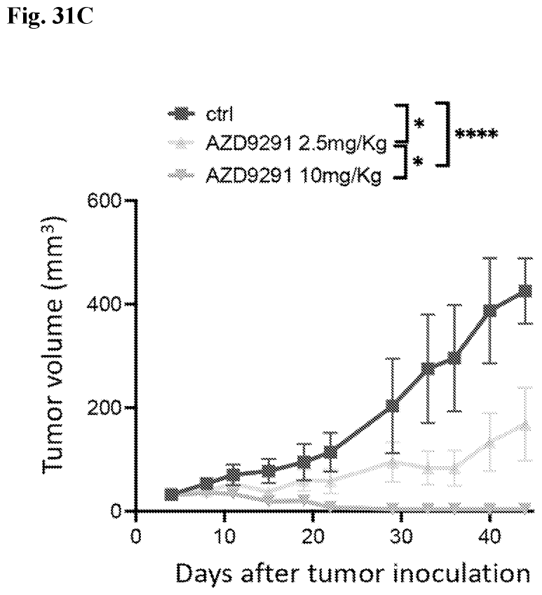

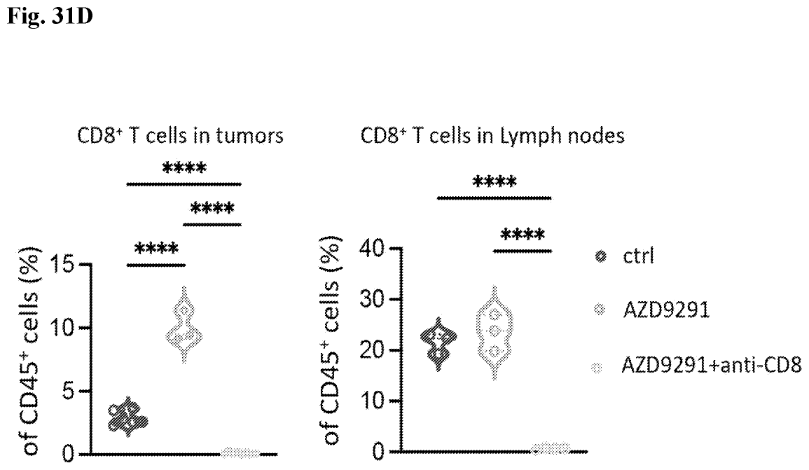

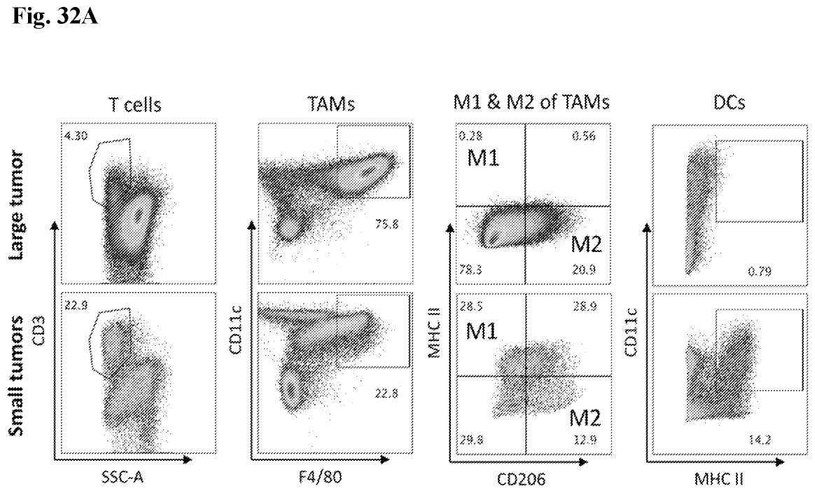

To investigate the role of immune responses to EGFR-TKI, a syngeneic genetically engineered mouse (GEM) model of lung cancer driven by exon19del/T790M EGFR and loss of Trp53 (referred as PE) in immunocompetent FVB mice was developed (FIG. 31A). Primary tumors cells derived from PE tumor demonstrated constitutive activation of EFGR/ERK signaling pathway, which can be inhibited by osimertinib but not erlotinib (FIG. 31B). The response of PE tumors to osimertinib in vivo was then evaluated. Treatment was started when tumor volume reached approximately 60 mm³. Notably, while 2.5 mg/kg (p.o., q.d.) osimertinib significantly slowed PE tumor growth, 10 mg/kg (p.o., q.d.) osimertinib resulted in tumor regression (FIG. 31C and FIG. 27A). A recent study reports that blockade of EGFR with erlotinib in EGFR-mutated lung adenocarcinomas induces CD8⁺ T cell infiltration (Sugiyama et al., 2020, Sci Immunol 5). To determine whether immune responses play a role in the antitumor activity of osimertinib, CD8⁺ T cells were depleted using an anti-CD8 antibody in PE tumor-bearing FVB mice (FIG. 27A and FIG. 31D). Indeed, therapeutic effect of osimertinib was significantly mitigated by CD8⁺ T cell depletion (FIG. 27A), suggesting T cell-mediated cytotoxicity is important for osimertinib-induced PE tumor regression. Consistently, analysis of the tumor immune microenvironment showed that osimertinib induced the recruitment of CD8⁺ and CD4⁺ T cells, which was accompanied by an increase of IFNg-positive CD8⁺ and CD4⁺ T cells (FIG. 27B). Moreover, the expression of CXCL10 and CCL5 was analyzed, which are important pro-inflammatory cytokines to attract CD8⁺ T cells in multiple cancer types including lung cancer (Sugiyama et al., 2020, Sci Immunol 5). The results showed that osimertinib significantly elevated expression of CXCL10 and CCL5 in both PE tumor cells and a human exon19del/T790M EGFR lung cancer cell line PC9GR4 (FIG. 27C).

To further demonstrate the relevance of T cell activation in therapeutic efficacy of EGFR-TKIs, a clinical cohort of 8 patients with EGFR-mutated advanced NSCLC was analyzed (Gurule et al., 2021, NPJ Precis Oncol 5, 41). These patients received erlotinib or osimertinib as first line treatment and had tumor biopsies before and after a short-term of treatment. Tumor RNA-seq data showed that T cell inflamed signature was significantly enriched after treatment in TKI responders (PFS >8 months) but not the non-responders (PFS <8 months) (FIG. 27D). Moreover, the increase of T cell inflamed score upon TKI treatment was positively correlated with PFS (FIG. 27E). These findings collectively highlight the role of immune activation in antitumor activity of osimertinib.

### An Immune-Suppressive TME Inhibits Therapeutic Efficacy of Osimertinib

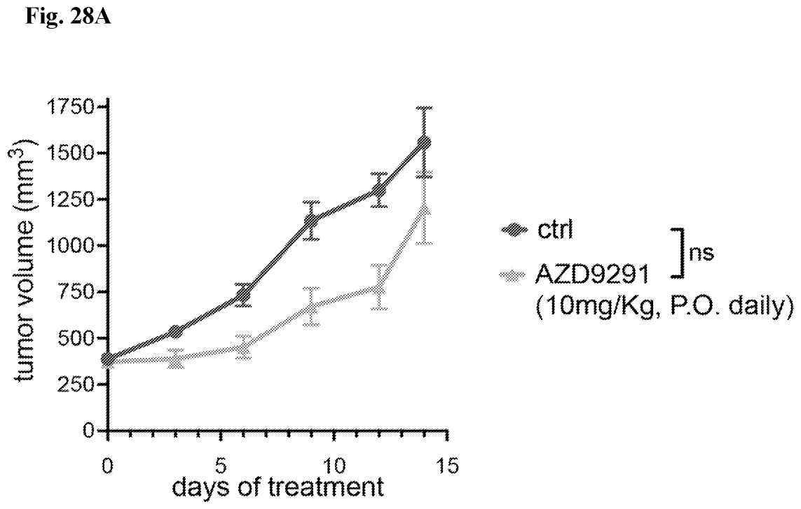

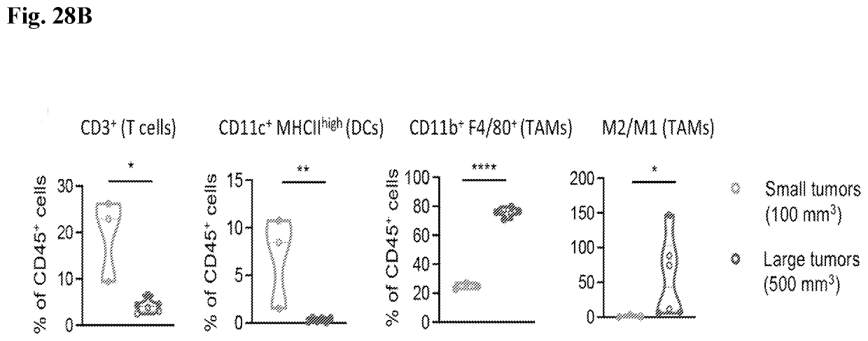

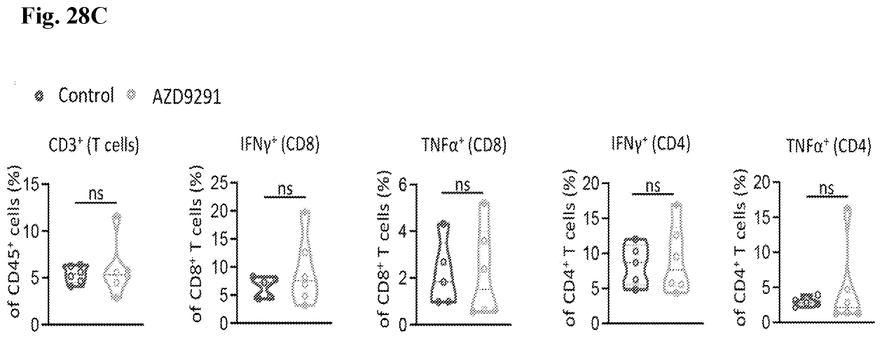

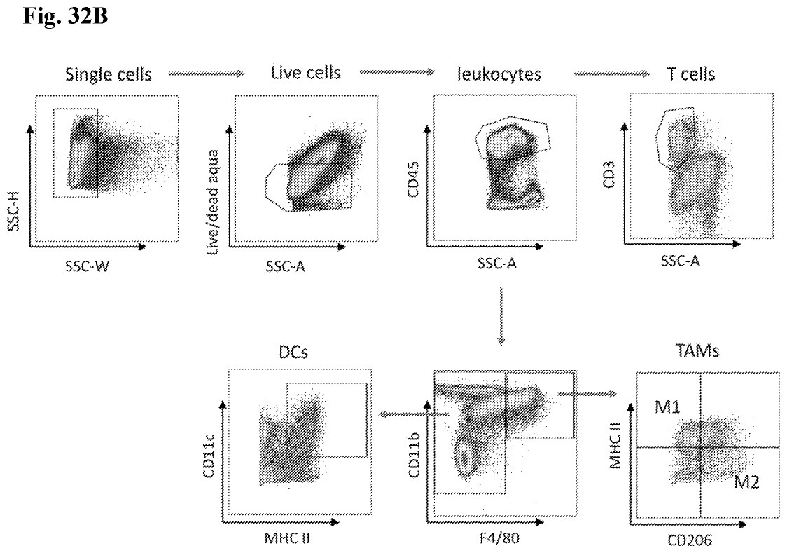

Emerging evidence suggests that more advanced tumors have a more immunosuppressive TME that impedes cancer treatment (Kim et al., 2020, Front Immunol 11, 629722). It was hypothesized that more advanced PE tumors with a larger tumor volume and delayed treatment have a more immunosuppressive TME and are less responsive to osimertinib. To test this hypothesis, osimertinib treatment was delayed when PE tumor size reached approximately 300-500 mm³. Notably, these tumors progressed through osimertinib treatment and presented growth rates comparable to control (FIG. 28A). Analyses of tumor infiltrating immune cells harvested from small (100 mm³) and large (500 mm³) PE tumors in treatment-naive FVB mice revealed that the numbers of T cells and DCs were significantly reduced in large tumors compared to small tumors (FIG. 28B). Notably, in large tumors, the portion of tumor-associated macrophages (TAMs) was strongly expanded and accounted for more than 70% of CD45⁺ cells (FIG. 28B and FIG. 32A). Further analysis showed that the TAMs in large tumors were mostly M2-like macrophages (MHC II^{low}, CD206^{high}) (FIG. 28B and FIG. 32A). These data suggest that large PE tumors developed an immunosuppressive TME dominated by M2-like TAMs. Importantly, treating large PE tumors with osimertinib did not lead to increase of T cells or IFNg⁺/TNFa⁺ T cells in tumors (FIG. 28A and FIG. 32B), suggesting that osimertinib monotherapy was not able to overcome the immunosuppressive TME of large PE tumors and exert therapeutic efficacy.

### TAMs Suppress CD8⁺ T Cell Activation and Compromise Therapeutic Efficacy of Osimertinib in Advanced Tumors

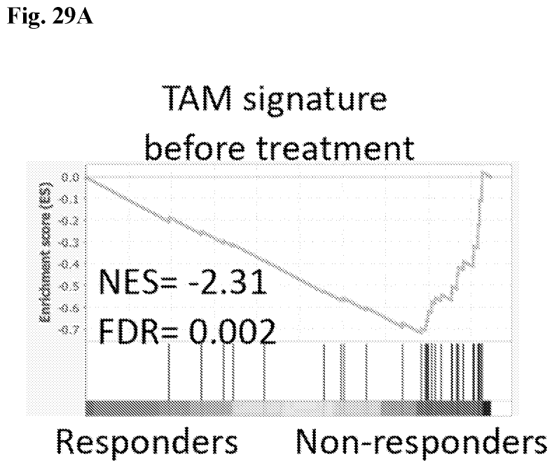

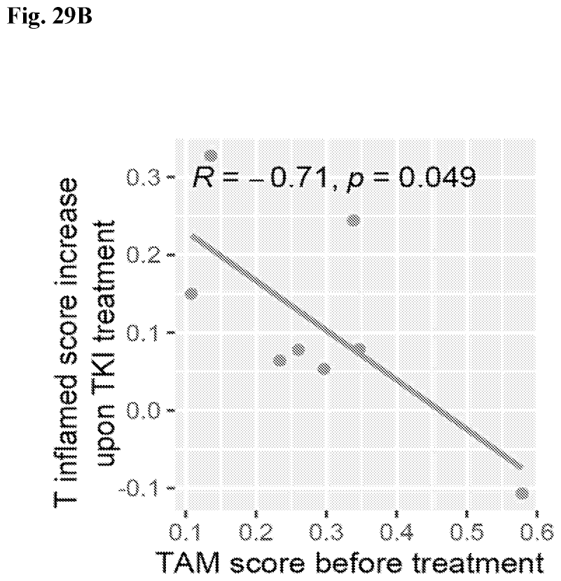

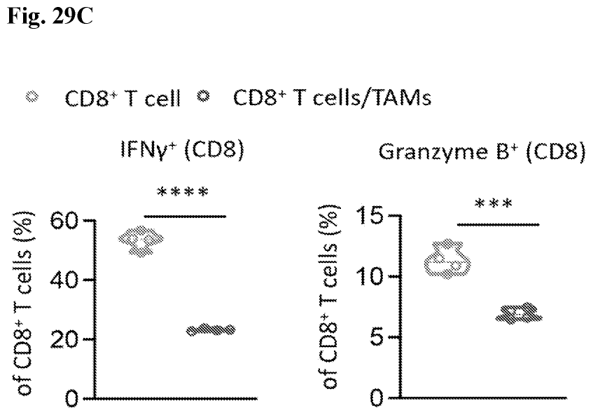

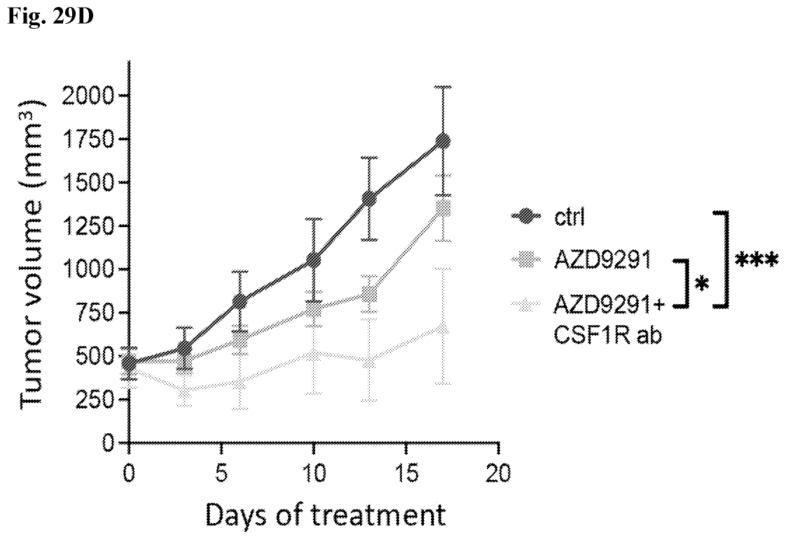

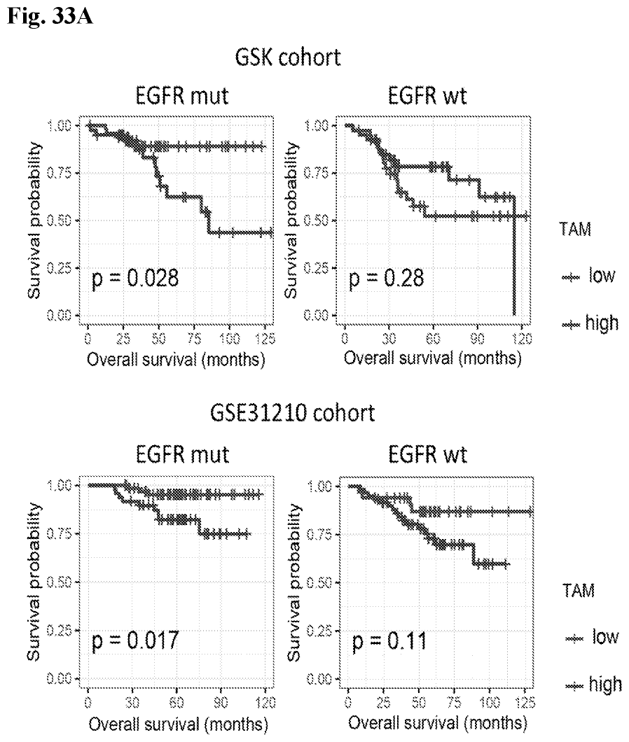

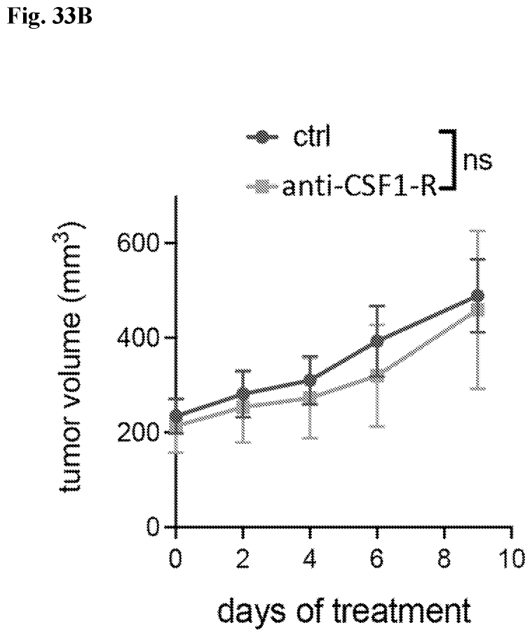

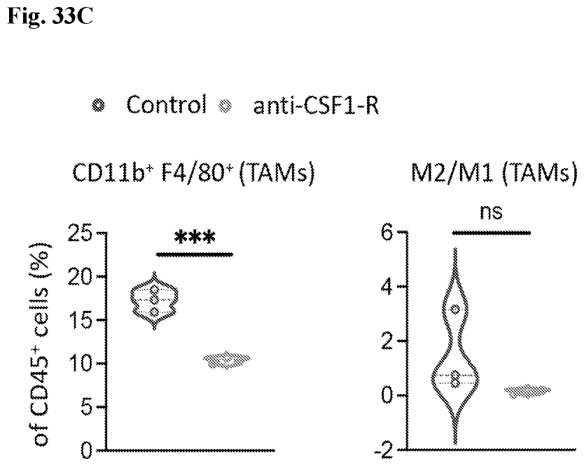

To evaluate the significance of TAMs, the prognostic relevance of TAMs in patients with EGFR-mutated or EGFR wild-type (EGFR-wt) NSCLC in two clinical datasets was analyzed, including GSK cohort and GSE31210 cohort. It was found that high TAM abundance is associated with worse overall survival in patients with EGFR-mutated but not EGFR-wt NSCLC (FIG. 33A). To assess the role of TAMs in EGFR-TKIs treatment outcome, clinical data from a recent study was then analyzed (Gurule et al., 2021, NPJ Precis Oncol 5, 41). This study provides whole exome sequencing (WES) data of eight matched patient tumor tissues biopsied before and after osimertinib or erlotinib treatments, thus allowing us to correlate the TAM signatures of these tumors with their treatment outcome. Analysis of WES data of tumors before EGFR-TKI treatment revealed that TAM signature was significantly enriched in non-responders (PFS <8 months) relative to responders (PFS >8 months) (FIG. 29A). Moreover, the enrichment scores of TAM signature were negatively correlated with the T cell infiltration scores in patients treated with osimertinib or erlotinib (FIG. 29B), suggesting that TAMs may inhibit T cells and compromise tumors' response to EGFR-TKI.

In agreement with patient data, it was found that TAMs isolated from PE tumors significantly inhibited IFNg and Granzyme B production of CD8⁺T cells in an in vitro coculture system (FIG. 29C). To further demonstrate the role of TAMs in antitumor efficacy of osimertinib in vivo, TAMs were depleted by treating PE tumor-bearing mice with an anti-CSF1-R antibody. Results showed that depletion of TAMs significantly improved the antitumor efficacy of osimertinib in large established PE tumors (FIG. 29D). Of note, TAM depletion alone did not significantly affect the tumor growth (FIG. 33B-C). Collectively, this data highlight the importance of modulation of TAMs to improve the therapeutic efficacy of osimertinib in large established tumors.

Combination of a STING Agonist with Osimertinib Induces Tumor Regression of PE Tumors

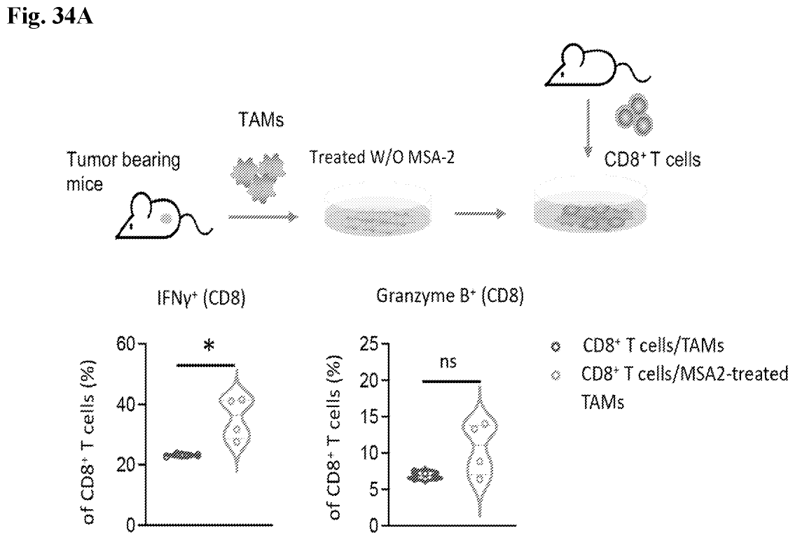

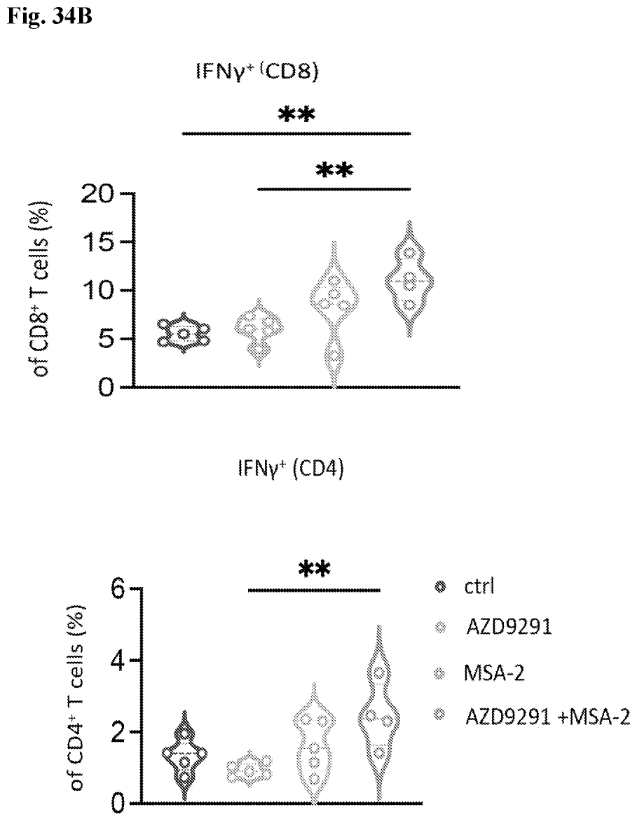

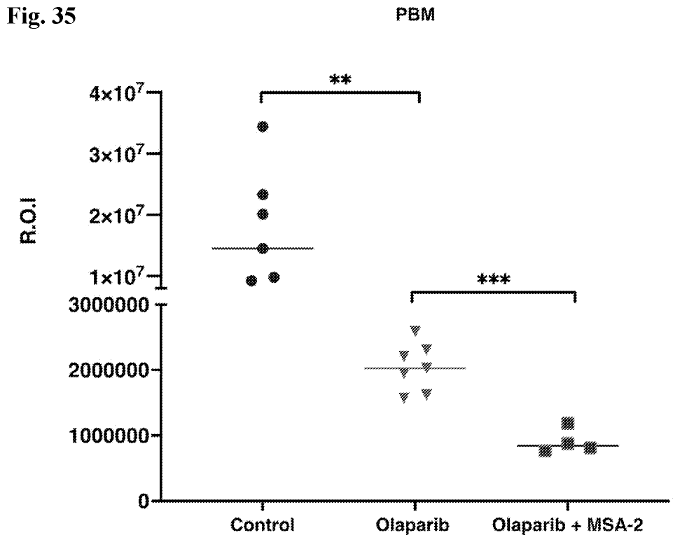

Combined osimertinib with depletion of TAMs inhibited tumor growth of large PE tumors but did not lead to tumor regression. Given the recent study demonstrating that TAMs can be reprogrammed from M2-like protumor state to M1-like antitumor state by a STING (stimulator of interferon genes) agonist, it was hypothesized that reprogramming TAMs into an anti-tumor state may be superior to TAM depletion by CSF1R antibody to improve treatment outcome when combining with osimertinib. Here, a newly developed STING agonist MSA-2 was employed, which was suitable for systemic administration (Pan et al., 2020). MSA-2 reversed TAM-mediated suppression of CD8⁺ T cells in an in vitro coculture system (FIG. 34A).

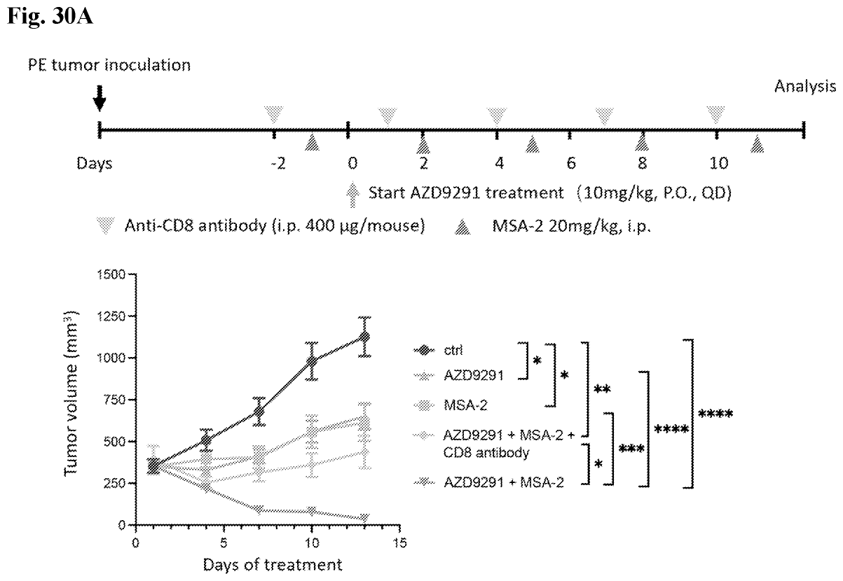

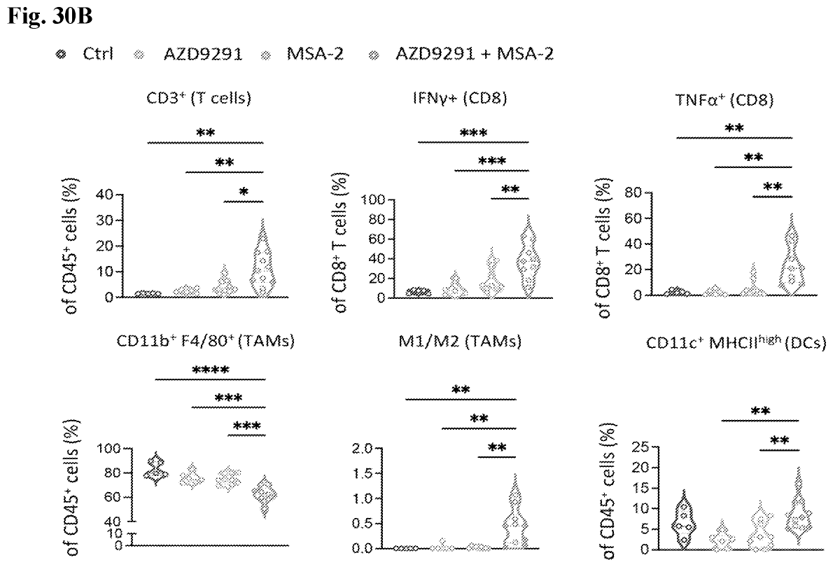

To test the efficacy of MSA-2 in vivo, PE tumor-bearing FVB mice were subjected to osimertinib, MSA-2 or to a combination treatment with osimertinib and MSA-2 when tumor size reached about 400 mm³. It was found that the combination treatment resulted in complete tumor regression, while osimertinib or MSA-2 monotherapy showed only modest therapeutic effects (FIG. 30A). Moreover, treating tumor-bearing mice with a CD8 blocking antibody significantly compromised, although not completely abrogated, the efficacy of the combination therapy (FIG. 30A). These data suggest that osimertinib in combination with MSA-2 has superior therapeutic efficacy than osimertinib combined with anti-CSF1-R in PE tumor model. Immune profiling of TME showed that single-agent treatment with osimertinib or MSA-2 did not significantly affect tumor immune microenvironment of large PE tumors (FIG. 30B). By contrast, combination of osimertinib with MSA-2 strongly induced T cell and DC recruitment and increased IFNg-positive CD8⁺ and TNFa-positive CD8⁺ T cells in TME (FIG. 30B). On the other hand, TAM abundance was also reduced by the combination treatment (FIG. 30B). Notably, phenotypes of TAMs were shifted from M2-like (MHC II^{low}, CD206^{high}) to M1-like (MHC II^{high}, CD206^{low}) in combination therapy-treated tumors (FIG. 30B). The activation of CD8⁺ or CD4⁺ T cells in the tumor-drainage lymph nodes (TDLNs) was then evaluated. As shown in FIG. 34B, compared to control or osimertinib or MSA-2 single agent treatment, combination treatment significantly up-regulated production of IFNg in both CD8⁺ T cells and CD4⁺ T cells. These results suggest that combined osimertinib with MSA-2 reprogrammed TME of large tumors, induced local and systemic anti-tumor immune responses, and lead to tumor regression of large and more aggressive tumors.

### Example 6: Methods for Example 5

Generation of NSCLC GEMM Driven by EGFR exon19del/T790M and Loss of Trp53

Adenovirus expressing Cre recombinase was intranasally injected into pulmonary airway of FVB/N mice carrying homozygously floxed alleles of Trp53 (Trp53L/L). These mice were sacrificed one week following the adenovirus administration and their lung tissues were harvested for alveolar epithelial (AE) cells isolation. AE cells were cultured for 48 hours, followed by the introduction of lentiviruses carrying EGFR exon19del/T790M mutation, and then subjected to 3 days antibiotic selection with blasticidin. AE cell were then collected and intravenously injected to 6- to 7-week-old female severe combined immunodeficient mice (SCID mice), which formed tumors in the lungs in approximately one month. The primary tumors were digested and then transplanted to FVB/NJ mice, leading to the development of tumors driven by EGFR exon19del/T790M and loss of Trp53 (referred as PE, FIG. 31A).

### Cell Culture

Cells were cultured in a humidified incubator under 5% CO₂ at 37° C. Tumor cells isolated from PE tumors were cultured in PDX medium [Ham's F-12 and DMEM (Gibco) supplemented with 0.6% FBS (Gibco), 1 mg/mL Hydrocortisone (Sigma), 4 μg/mL Insulin (Thermo Fisher), 5 ng/ml Cholera Toxin (Sigma), 10 mg/mL EGF (Sigma), 100 μg/mL penicillin-streptomycin (Gibco)]. A human cell line, PC9GR4 (Exon19del/T790M), was kindly provided by Dr. Pasi A. Jänne at Dana-Farber Cancer Institute (DFCI), was grown in RPMI-1640 containing 10% FBS (Gibco).

### Western Blotting

Whole cell lysates were prepared using ice-cold RIPA buffer supplemented with protease and phosphatase inhibitor cocktail (Thermo Fisher). Equal amount of proteins were separated by 10% SDS-PAGE gel, and were transferred to polyvinylidene fluoride (PVDF) membranes. After blocking for 45 minutes with 5% non-fat milk (Bio-Rad) in TBS plus 0.05% Tween 20 at room temperature, the membrane was subjected to incubation in primary antibody at 4° C. overnight, washing and followed by incubation with fluorescently-labeled anti-mouse IgG (Rockland Immunochemicals, #RL610-145-002) or anti-rabbit IgG (Molecular Probes, #A-21109) at room temperature for 1 hour. Western blots were visualized on an Odyssey scanner (LI-COR).

### Quantitative Real Time RT-PCR

Cultured cells were lysed in 1 mL TRIZOL® reagent supplied with 200 μL chloroform, after which, samples were vortexed vigorously for 15s and incubated at room temperature for 2 to 3 minutes. The samples were then centrifuged at 12000 g for 15 minutes at 4° C., and collected aqueous phase only. RNA were precipitated by adding 0.5 mL of isopropanol to the aqueous phase, and then washed with 75% ethanol after centrifugation. The yielded RNA samples were reversed-transcribed to complementary DNA (cDNA) using Supermix (Bio-Rad, #1708841) according to the manufacturer's instructions. Real-time PCR was performed using SYBR™ Select Master Mix (Thermo Fisher, #4472908) with gene-specific primers (mouse Ccl5, forward 5′-GCTGCTTTGCCTACCTCTCC-3′, reverse 5′-TCGAGTGACAAACACGACTGC-3′; mouse Cxcl10, forward 5′-CCAAGTGCTGCCGTCATTTTC-3′, reverse 5′-GGCTCGCAGGGATGATTTCAA-3′; mouse Actb, forward 5′-CGGTTCCGATGCCCTGAGGCTCTT-3′, reverse 5′-CGTCACACTTCATGATGGAATTGA-3′; human GAPDH, forward 5′-CTCTGCTCCTCCTGTTCGAC-3′, reverse 5′-TTAAAAGCAGCCCTGGTGAC-3′; human CCL5, forward 5′-CCAGCAGTCGTCTTTGTCAC-3′, reverse 5′-CTCTGGGTTGGCACACACTT-3′; human CXCL10, forward 5′-GTGGCATTCAAGGAGTACCTC-3′, reverse 5′-TGATGGCCTTCGATTCTGGATT-3′). Relative mRNA levels were calculated using the AACT method. Mouse Actb and human GAPDH were used as endogenous controls for mouse and human samples, respectively.

### Tumor Growth and Treatment

ETP cells were resuspended in serum-free DMEM containing 40% matrigel (Corning) and subcutaneously injected into the flank fat pads of 6 to 8-week-old mice. 1×106 ETP tumor cells in a total volume of 100 μL were injected into fat pads of female FVB/N mice. Tumor growth was monitored by measuring the tumor size with digital calipers every three days, starting from the 5th day after the injection. Greatest longitudinal diameter (length) and the greatest transverse diameter (width) were measured, based on which tumor, volume was calculated by using the modified ellipsoid formula (0.50×length×width2). All tumor measurements within single cohorts were performed by the same researcher. Mice were euthanized by CO₂ inhalation when tumor volumes met humane endpoints described in the IACUC protocols (20 mm diameter) or upon severe health deterioration.

For pharmacodynamic studies, mice were grouped based on initial tumor volumes to ensure equal distribution across groups. Osimertinib was reconstituted in HPMC solution (0.05N HCL+0.5% HPMC [sigma 9262]) at the concentration of 2.5 mg/ml, and was administrated within one week of preparation by gavage at a dosage of 10 mg/kg body weight daily. MSA-2 was prepared by diluting 50 mg/ml stock in DMSO with PBS and administrated immediately after drug preparation by intraperitoneal (i.p.) injection at a dosage of 20 mg/kg body weight every three days. Anti-CD8 antibody (i.p. 400 μg/mouse; clone YTS 169.4, BioXcell) was administered every 3 days to deplete CD8⁺ T cell, starting 48 hours ahead of other treatments. As for macrophage depletion, anti-mouse CSF1R antibody (clone AFS98, BioXcell) was dosed at 40 mg/kg via i.p. every 2 days, starting 48 hours ahead of osimertinib treatment.

### Co-Culture Experiments

Tumor associated macrophages (TAMs) were isolated from ETP tumors using a mouse CD11b⁺ myeloid cell isolation kit (StemCell, #18970) and seeded in 48-well plate at the density of 1×10⁵ cells/well, cultured in DMEM growth medium (DMEM+10% FBS+100 μg/mL penicillin-streptomycin) supplemented with 10 ng/ml mouse M-CSF (BioLegend, #576404) and allowed to adhere overnight. TAMs were then treated with or without MSA-2 at the concentration of 33 μM in the median mentioned above for 2 days. MSA-2 was washed off before coculturing TAM with CD8⁺ T cells. Mouse CD8⁺ T cells were isolated from spleens of FVB/NJ mice using a mouse CD8+ T cell isolation kit (StemCell, #19853) and seeded in the same 48-well plate at the density of 1×10⁵ cells/well, cultured alone or cocultured with TAMs in RPMI 1640 supplemented with 10% FBS, 10 ng/ml mouse M-CSF (BioLegend, #576404), 0.055 mM 2-mercaptoethanol, 2 ng/ml IL-2 (Peprotech), 2.5 ng/ml IL-7 (Peprotech) and 50 ng/ml IL-15 (Peprotech) for 2 days. After that, T cells were collected for flow cytometry analysis.

### Tissue Cells Dissociation and Flow Cytometry Analysis

To obtain single-cell suspension from tumor mass, tumors were excised, minced and dissociated in collagenase buffer (DMEM supplemented with 5% FBS, 10 mM HEPES [Gibco), 100 μg/mL penicillin-streptomycin, 20 μg/mL DNase I [StemCell] and 1× collagenase/hyaluronidase [StemCell]) at 37° C. for 45 min. Tumor-draining lymph nodes were isolated from tumor-bearing mice and were mashed through 70 μm strainer using plunger of a syringe to obtain single-cell suspension. Red blood cells (RBC) were removed from the dissociated tissue cells with RBC lysis buffer (Life Technologies #00-4333-57), after which, cells were washed and resuspended in FACS buffer (PBS containing 0.2% BSA and 5 mM EDTA).

To profile immune population in tissues, the dissociated cells were incubated with conjugated anti-mouse antibodies targeting all kind of biomarkers, which included leukocyte biomarker (CD45 [30-F11, BioLegend]), T cells biomarkers (CD3 [145-2C11, BioLegend], CD8 [53-6.7, BioLegend], CD4 [RM4-5, BioLegend], PD-1 [29F.1A12, BioLegend], FoxP3 [MF-14, BioLegend], TNF-α [MP6-XT22, BioLegend], IFN-γ [XMG1.2, BioLegend], Granzyme B [NGZB, Invitrogen], and myeloid cells biomarkers (CD11b [M1/70, BioLegend], CD11c [N⁴¹⁸, BioLegend], F4/80 [BM8, BioLegend], Gr-1 [RB6-8C5, BioLegend], CD86 [GL-1, BioLegend], MHC II [M5/114.15.2, BioLegend], CD206 [C068C2, BioLegend], CD40 [3/23, BioLegend]). For the cytokine detection (TNF-α, IFN-γ), tissue cells were stimulated with Leukocyte Activation Cocktail (BD Biosciences, #550583) in RPMI medium (10% FBS) at the manufacture's recommended concentration for 4 hours at 37° C./5% CO2 prior to FACS staining. For FACS staining, tissue cells were first stained with LIVE/DEAD Fixable Aqua Dead Cell Stain (Thermo Fisher) for 30 min on ice, followed by blocking with anti-CD16/32 (BioLegend) for 20 minutes on ice. Afterwards, cells were incubated with antibodies targeting surface biomarkers in FACS buffer for 30 minutes on ice. For intracellular staining, cells were then fixed and permeabilized with Foxp3/Transcription Factor Staining Buffer Set (eBioscience, #00-5523-00) before incubation with antibodies targeting intracellular biomarkers like CD206, TNF-α and IFN-γ in permeabilization buffer for 30 minutes on ice.

### Patient Data and Bioinformatic Analysis

Whole exome RNA-seq data of tumor tissues biopsied from 8 patients with EGFR mutant lung cancers before and after TKI treatment (Osimertinib or Erlotinib) were download from Gene Expression Omnibus (GEO) repository via the following accession: https://identifiers.org/geo: GSE165019. Corresponding clinical information was obtained from the published study (Gurule et al., 2021, NPJ Precis Oncol 5, 41). All the patients were diagnosed at advanced stage (Stage IIIB/or IV) and received TKIs as the first line treatment. Tumors were biopsied before any treatment, and re-biopsied within three months of TKI treatment. Progression-free survival (PFS) of the 8 patients ranged from 6.2 months to 16.3 months, with 8 months as the cut-off value to discriminate responders (PFS >8 months, n=4) and non-responders (PFS <8 months, n=4).

Another two clinical datasets (GSK cohort and GSE31210 cohort) involving surgery-treated patients with NSCLC with or without EGFR mutations were also analyzed. GSK cohort (Chen et al., 2020, Nat Genet 52, 177-186) data were obtained from cbioportal (http://www.cbioportal.org). GSE31210 (Okayama et al., 2012) data were obtained from GEO database (found at the World Wide Web at ncbi.nlm.nih.gov/geo/).

Gene signatures of tumor-associated macrophage (TAM) (Cassetta et al., 2019, Cancer Cell 35, 588-602.e510) and a T cell inflamed signature (Ayers et al., 2017, J Clin Invest 127, 2930-2940) were obtained from previous studies. Gene set enrichment analysis (GSEA) (Subramanian et al., 2005, Proc Natl Acad Sci USA 102, 15545-15550) was performed to compare the enrichment of certain gene signatures between two groups. As for GSEA, genes were first ranked according to log 2 (fold change), which was generated by DESeq2 package in R software environment (Love et al., 2014, Genome Biol 15, 550) and then analyzed using GSEAPreranked tool with the ‘classic’ method. Enrichment score of TAM signature and T cell inflamed signature for each sample were inferred based on RNA-seq data using single-sample gene set enrichment analysis (ssGSEA), which was implemented by the GSVA R package.

### Statistical Analysis

Statistical analysis was performed using GraphPad Prism® v8. Unpaired t test was applied for the comparison of two sets of measurement which complied normalized distribution. When comparing two sets of samples whose variances were not equal, Wilcoxon rank-sum test was used instead. For the comparison of three or more sets of unpaired measurements, One-way ANOVA with Tukey's multiple comparisons test (for normally distributed data) and Kruskal-Wallis nonparametric test (for skewed data) were applied. P value less than 0.05 was considered statistically significant.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims

What is claimed is:

1. A method of improving effectiveness of PARP inhibition in a subject with cancer, comprising administering to the subject an effective amount of a STING agonist conjointly with an effective amount of a PARP inhibitor, an effective amount of a TK inhibitor, and/or an effective amount of a DNA synthesis inhibitor.

2. The method of claim 1, wherein said administering comprises a systemic delivery of the STING agonist.

3. The method of claim 1 or 2, wherein said administering is oral, intravenous, or intraperitoneal.

4. The method of any one of claims 1 to 3, wherein the STING agonist is a modified nucleotide STING agonist.

5. The method of any one of claims 1 to 3, wherein the STING agonist is selected from DMXAA, MSA-2, SR-717, FAA, CMA, α-Mangostin, BNBC, DSDP, diABZI, bicyclic benzamides, and benzothiophenes.

6. The method of any one of claims 1 to 5, wherein
i) the PARP inhibitor is selected from the group consisting of olaparib, rucaparib, niraparib, talazoparib, veliparib, pamiparib, CEP 9722, E7016, AG014699, MK4827, BMN-673, iniparib, and 3-aminobenzamide,
ii) the TKI inhibitor is a EGFR-TKI inhibitor is selected from the group consisting of afatinib, dacomitinib, osimertinib, rociletinib (CO-1686), olmutinib (HM61713), nazartinib (EGF816), naquotinib (ASP8273), mavelertinib (PF-0647775), almonertinib, TY-9591, gefitinib, erlotinib and AC0010, and/or
iii) the DNA synthesis inhibitor is gemcitabine, sapacitabine, a cytidine analog, cytarabine, tezacitabine, troxacitabine, DMDC, CNDAC, ECyD, clofarabine, or decitabine.

7. The method of any one of claims 1 to 6, wherein said administering conjointly comprises administering the STING agonist before the PARP inhibitor, the TK inhibitor, and/or DNA synthesis inhibitor.

8. The method of any one of claims 1 to 6, wherein said administering conjointly comprises administering the STING agonist concurrently with the PARP inhibitor, the TK inhibitor, and/or DNA synthesis inhibitor.

9. The method of any one of claims 1 to 8, wherein said cancer comprises a tumor with an M2 enrichment score higher than 0.27, or TAM M2/M1 ratio higher than 1.

10. The method of any one of claims 1 to 8, wherein said cancer comprises head and neck squamous cell carcinoma (HNSC); a lung cancer, such as non-small cell lung cancer (NSCLC) or lung squamous cell carcinoma (LUSC); liver cancer, such as hepatocellular carcinoma (HCC); colon cancer; prostate cancer; pancreatic cancer; skin cutaneous melanoma (SKCM); glioblastoma multiforme (GBM); breast invasive carcinoma (BRCA); lung adenocarcinoma (LUAD); kidney renal clear cell carcinoma (KIRC); cervical squamous cell carcinoma and endocervical adenocarcinoma (CESC); diffuse large B-cell lymphoma (DLBC); stomach adenocarcinoma (STAD) or ovarian cancer, such as high-grade serous ovarian carcinoma (HGSOC) ovarian cancer, such as high-grade serous ovarian carcinoma (HGSOC), homologous recombination proficient (HRP) ovarian cancer; or a homologous recombination deficient (HRD) ovarian cancer.

11. The method of any one of claims 1 to 8, wherein said cancer comprises breast cancer carrying a BRCA mutation, such as an advanced breast cancer carrying germline BRCA1/2 mutations.

12. The method of any one of claims 1 to 8, wherein said cancer comprises a lung cancer comprising an EGFR mutation, such as non-small cell lung cancer comprising an EGFR activating mutation.

13. The method of any one of claims 1 to 8, wherein said cancer comprises a sub-population of tumors with an M2 enrichment score higher than 0.27.

14. The method of any one of claims 1 to 8, wherein said cancer comprises a tumor that has acquired an M2 enrichment score higher than 0.27 over a treatment course.

15. The method of any one of claims 1 to 14, wherein the subject is a rodent, primate, human, or animal model of cancer, optionally wherein the subject is human.

16. The method of any one of claims 1 to 14, wherein the subject has a deficiency in activating STING signaling in tumor cells.

17. The method of any one of claims 1 to 16, wherein the PARP inhibitor is administered at a dosage of 50 mg/kg body weight per day, the EGFR-TKI inhibitor is administered at a dosage of 5-50 mg/kg of body weight per day or the DNA synthesis inhibitor is administered at 2,000 mg/m2 per week.

18. The method of any one of claims 1 to 16, wherein the STING agonist is administered at a dosage of 10 mg/kg body weight per week.

19. The method of any one of claims 1 to 18, wherein the STING agonist is administered 2-3 times.

20. The method of any one of claims 1 to 19, further comprising an additional therapy.

21. The method of claim 20, wherein the additional therapy comprises radiation therapy.

22. The method of claim 20, wherein the additional therapy comprises chemotherapy.

23. The method of claim 22, wherein the chemotherapy comprises paclitaxel, a platinum-based drug, cisplatin, oxaliplatin, an inhibitor of topoisomerase, etoposide, a DNA intercalator, doxorubicin, a DNA alkylating agent, or temozolomide.

24. The method of claim 20, wherein the additional therapy comprises a DNA damage response (DDR)-targeting agent.

25. The method of claim 24, wherein the DDR-targeting agent comprises ATMi, ATRi, CHK1/2i, or Weeli.

26. A method of polarizing pro-tumor macrophages in a subject with cancer into anti-tumor macrophages, comprising administering to the subject an effective amount of a STING agonist.

27. A method of preventing or reversing drug resistance in a subject with cancer, wherein the drug resistance is a result of polarization of anti-tumor macrophages into pro-tumor macrophages, comprising administering to the subject an effective amount of a STING agonist.

28. The method of claim 26 or claim 27, wherein the STING agonist activates STING signaling in macrophages.

29. The method of any one of claims 26 to 28, wherein the intra-tumor STING agonists (e.g. tumor cell's cytosolic dsDNAs/cGAMP or intra-tumoral delivered STING agonists) do not activate STING signaling in intra-tumoral dendritic cells, or the subject has a deficient STING signaling pathway in tumor cells.

30. The method of any one of claims 26 to 29, wherein the pro-tumor macrophages are M2-like.

31. The method of any one of claims 26 to 30, wherein the anti-tumor macrophages are M1-like.

32. The method of any one of claims 26 to 31, wherein said administering comprises a systemic delivery of the STING agonist.

33. The method of any one of claims 26 to 31, wherein said administering is oral, intravenous, or intraperitoneal.

34. The method of any one of claims 26 to 33, wherein the STING agonist is a modified nucleotide STING agonist.

35. The method of any one of claims 26 to 33, wherein the STING agonist is selected from DMXAA, MSA-2, SR-717, FAA, CMA, α-Mangostin, BNBC, DSDP, diABZI, bicyclic benzamides, and benzothiophenes.

36. The method of any one of claims 26 to 33, wherein said cancer comprises a tumor with an M2 enrichment score higher than 0.27.

37. The method of any one of claims 26 to 33, wherein said cancer comprises head and neck squamous cell carcinoma (HNSC); lung squamous cell carcinoma (LUSC); non-small cell lung cancer (NSCLC); liver cancer, such as hepatocellular carcinoma (HCC); colon cancer; prostate cancer; pancreatic cancer; skin cutaneous melanoma (SKCM); glioblastoma multiforme (GBM); breast invasive carcinoma (BRCA); lung adenocarcinoma (LUAD); kidney renal clear cell carcinoma (KIRC); cervical squamous cell carcinoma and endocervical adenocarcinoma (CESC); diffuse large B-cell lymphoma (DLBC); stomach adenocarcinoma (STAD), or ovarian cancer, such as high-grade serous ovarian carcinoma (HGSOC) ovarian cancer, such as high-grade serous ovarian carcinoma (HGSOC), homologous recombination proficient (HRP) ovarian cancer; or a homologous recombination deficient (HRD) ovarian cancer.

38. The method of any one of claims 26 to 35, wherein said cancer comprises breast cancer carrying a BRCA mutation.

39. The method of any one of claims 26 to 35 wherein said cancer comprises advanced breast cancer carrying germline BRCA1/2 mutations.

40. The method of any one of claims 26 to 35, wherein said cancer comprises a lung cancer carrying an EGFR mutation, such as an EGFR activating mutation or a T790M mutation.

41. The method of any one of claims 26 to 35, wherein said cancer comprises a non-small cell lung cancer carrying an EGFR mutation, such as an EGFR activating mutation or a T790M mutation.

42. The method of claim 27, wherein the drug resistance is resistance to PARP inhibition or resistance to EGFR-TK inhibition.

43. The method of any one of claims 26 to 35, wherein said cancer comprises a sub-population of tumors with an M2 enrichment score higher than 0.27.

44. The method of any one of claims 26 to 35, wherein said cancer comprises a tumor that has acquired an M2 enrichment score higher than 0.27 over a treatment course.

45. The method of any one of claims 26 to 44, wherein the subject is a rodent, primate, human, or animal model of cancer, optionally wherein the subject is human.

46. The method of any one of claims 26 to 45, wherein the subject has a deficient STING signaling pathway in tumor cells, therefore intra-tumoral STING agonists (e.g. dsDNAs/cGAMP released from tumor cells or intra-tumoral delivered STING agonists) cannot activate intra-tumoral dendritic cells and macrophages.

47. The method of any one of claims 26 to 46, wherein the STING agonist is administered at a dosage of 10 mg/kg body weight per week.

48. The method of any one of claims 26 to 47, wherein the STING agonist is administered 2-3 times.

49. The method of any one of claims 26 to 48, further comprising an additional therapy.

50. The method of claim 49, wherein the additional therapy comprises a PARP inhibitor.

51. The method of claim 50, wherein the PARP inhibitor is selected from the group consisting of olaparib, rucaparib, niraparib, talazoparib, veliparib, pamiparib, CEP 9722, E7016, AG014699, MK4827, BMN-673, iniparib, and 3-aminobenzamide.

52. The method of claim 49, wherein the additional therapy comprises a TK inhibitor.

53. The method of claim 52, wherein the TK inhibitor is a EGFR-TK inhibitor selected from the group consisting of afatinib, dacomitinib, osimertinib, rociletinib (CO-1686), olmutinib (HM61713), nazartinib (EGF816), naquotinib (ASP8273), mavelertinib (PF-0647775), almonertinib, TY-9591, gefitinib, erlotinib and AC0010.

54. The method of claim 49, wherein the additional therapy comprises radiation therapy.

55. The method of claim 49, wherein the additional therapy comprises chemotherapy.

56. The method of claim 55, wherein the chemotherapy comprises paclitaxel, a platinum-based drug, cisplatin, oxaliplatin, an inhibitor of topoisomerase, etoposide, a DNA intercalator, doxorubicin, a DNA alkylating agent, or temozolomide.

57. The method of claim 49, wherein the additional therapy comprises a DNA damage response (DDR)-targeting agent.

58. The method of claim 57, wherein the DDR-targeting agent comprises ATMi, ATRi, CHK1/2i, or Weeli.

59. A method of selecting a subject with cancer for treatment with a STING agonist, comprising detecting an M2 enrichment score for a tumor from the subject, and selecting the subject if the score is higher than 0.27.

60. The method of claim 59, wherein the cancer comprises head and neck squamous cell carcinoma (HNSC); lung squamous cell carcinoma (LUSC); non-small cell lung cancer (NSCLC), liver cancer, such as hepatocellular carcinoma (HCC); colon cancer; prostate cancer; pancreatic cancer; skin cutaneous melanoma (SKCM); glioblastoma multiforme (GBM); breast invasive carcinoma (BRCA); lung adenocarcinoma (LUAD); kidney renal clear cell carcinoma (KIRC); cervical squamous cell carcinoma and endocervical adenocarcinoma (CESC); diffuse large B-cell lymphoma (DLBC); stomach adenocarcinoma (STAD), or ovarian cancer, such as high-grade serous ovarian carcinoma (HGSOC) ovarian cancer, such as high-grade serous ovarian carcinoma (HGSOC), homologous recombination proficient (HRP) ovarian cancer; or homologous recombination deficient ovarian cancer.

61. The method of claim 60, wherein the cancer comprises breast cancer carrying a BRCA mutation.

62. The method of claim 60, wherein the cancer comprises advanced breast cancer carrying germline BRCA1/2 mutations.

63. The method of claim 60, wherein said cancer comprises a lung cancer carrying an EGFR mutation, such as an EGFR activating mutation or a T790M mutation.

64. The method of claim 60, wherein said cancer comprises a non-small cell lung cancer carrying an EGFR mutation, such as an EGFR activating mutation or a T790M mutation.

65. The method of any one of claims 59 to 64, wherein the subject is a rodent, primate, human, or animal model of cancer, optionally wherein the subject is human.

66. The method of any one of claims 59 to 64, wherein the subject has a deficiency in activating STING signaling in tumor cells.

67. A method of treating a subject with advanced breast cancer carrying germline BRCA1/2 mutations, wherein the cancer comprises a tumor with an M2 enrichment score higher than 0.27, comprising systemically administering to the subject, optionally wherein the administration is about 10 mg/kg body weight of a STING agonist conjointly with about 50 mg/kg body weight of a PARP inhibitor.

68. A method of treating a subject with non-small cell lung cancer carrying germline EGFR mutations, wherein the cancer comprises a tumor with an M2 enrichment score higher than 0.27, comprising administering to the subject a STING agonist conjointly with an EGFR-TK inhibitor.
